SEQUENCE LISTING (6 sequences)

SEQ ID NO: 1 - DNA, 20 bp, Synthetic construct
  source 1..20: mol_type = other DNA
ccaagccaaagaagtccaag

SEQ ID NO: 2 - DNA, 20 bp, Synthetic construct
  source 1..20: mol_type = other DNA
caagcagattccctgacaca

SEQ ID NO: 3 - DNA, 19 bp, Synthetic construct
  source 1..19: mol_type = other DNA
gccaaagaagtccaagtcg

SEQ ID NO: 4 - DNA, 20 bp, Synthetic construct
  source 1..20: mol_type = other DNA
ggactccaggctgatgatct

SEQ ID NO: 5 - DNA, 20 bp, Synthetic construct
  source 1..20: mol_type = other DNA
atcaccattggcaatgagcg

SEQ ID NO: 6 - DNA, 20 bp, Synthetic construct
  source 1..20: mol_type = other DNA
ttgaaggtagtttcgtggat

(19) United States
(12) Patent Application Publication
(10) Pub. No.: US 2025/0188168 A1
(43) Pub. Date: Jun. 12, 2025

(54) TREATMENT OF ALVEOLAR SOFT PART SARCOMA (ASPS) WITH ATEZOLIZUMAB

(71) Applicant: The United States of America, as represented by the Secretary, Department of Health & Human Services, Bethesda, MD (US)

(72) Inventors: Alice P. Chen, Potomac, MD (US); Elad Sharon, Brookline, MA (US); Geraldine O'Sullivan-Coyne, Dublin (IE); James H. Doroshow, Bethesda, MD (US)

(73) Assignee: The United States of America, as represented by the Secretary, Department of Health & Human Services, Bethesda, MD (US)

(21) Appl. No.: 18/974,439

(22) Filed: Dec. 9, 2024

Related U.S. Application Data
(60) Provisional application No. 63/608,127, filed on Dec. 8, 2023.

(51) Int. Cl. C07K 16/28; A61K 39/00; A61K 45/06; A61P 35/00; C07K 16/22

(52) U.S. Cl. CPC C07K 16/2827; A61K 45/06; A61P 35/00; C07K 16/22; A61K 2039/507; A61K 2039/545

(57) ABSTRACT

Provided herein are methods of using the anti-PD-L1 monoclonal antibody atezolizumab to treat alveolar soft part sarcoma (ASPS).

### CROSS REFERENCE TO RELATED APPLICATION

This application claims priority to U.S. Provisional Patent Application No. 63/608,127, filed on Dec. 8, 2023, which is incorporated herein by reference in its entirety.

### ACKNOWLEDGMENT OF GOVERNMENT SUPPORT

This invention was made with government support under 75N91019D00024 and HHSN261201500003I awarded by the National Cancer Institute. The government has certain rights in the invention.

### FIELD

This disclosure concerns the use of the anti-PD-L1 monoclonal antibody atezolizumab in methods of treating alveolar soft part sarcoma (ASPS).

### INCORPORATION OF SEQUENCE LISTING

The Sequence Listing is submitted as an XML file in the form of the file named “Sequence.xml” (6,202 bytes), which was created on Dec. 2, 2024, which is incorporated by reference herein.

### BACKGROUND

ASPS is among the rarest cancers, with about 80 cases diagnosed each year in the United States, most commonly among young adults (such as patients between 15-35 years old). ASPS tumors can grow slowly and asymptomatically, leading to most cases being diagnosed in advanced stages. Historically, treatment for ASPS has been limited to surgery for the primary location where the sarcoma arises, and systemic therapy is frequently the only option when the tumor spreads. Patients can live with ASPS for years and even decades, undergoing repeated surgeries to keep the disease at bay; however, recurrences are common. Thus, new treatments for ASPS are needed.

### SUMMARY

Disclosed herein are methods of treating ASPS using a therapeutically effective amount of the monoclonal antibody atezolizumab. Atezolizumab (also sold under the brand name Tecentriq®) is a monoclonal antibody designed to specifically bind programmed cell death ligand-1 (PD-L1). Atezolizumab binds to PD-L1 expressed on tumor cells and tumor-infiltrating immune cells, blocking its interactions with both PD-1 and B7.1 receptors. This releases the PD L1/PD 1 mediated inhibition of the immune response, including activation of the anti-tumor immune response without inducing antibody-dependent cellular cytotoxicity. PD L1 may be expressed on tumor cells and/or tumor infiltrating immune cells and can contribute to the inhibition of the anti-tumor immune response in the tumor microenvironment. Binding of PD L1 to the PD 1 and B7.1 receptors found on T cells and antigen presenting cells suppresses cytotoxic T-cell activity, T-cell proliferation and cytokine production.

In one aspect, the subject treated is a mammal, such as a human or veterinary subject. In some aspects, the subject treated is a human adult patient, such as one at least 18 years old. In some examples, the subject treated is a human geriatric patient, such as one at least 65 years old. In some examples, the subject treated is a human adolescent patient, such as one 12-17 years old. In some examples, the subject treated is a human pediatric patient, such as one at least 2 years old. In some examples, the human pediatric patient is 2 years to <17 years. In some examples, the subject treated is a human patient at least 6 years old.

In some aspects, atezolizumab is administered every 2 weeks, every 3 weeks, or every 4 weeks. Each administration is a “cycle”. In some aspects, atezolizumab is administered as a single dose for at least 2 cycles, at least 3 cycles, at least 4 cycles, at least 5 cycles, at least 10 cycles, at least 20 cycles, at least 100 cycles, or more, such as 2, 3, 4, 5, 6, 7, 8, 9, 10, 15, 20, 25, 30, 40, 50 or 100 cycles. In some examples, the subject is administered multiple cycles/doses of atezolizumab, for example over a period of at least 1 year, at least 1.5 years, at least 2 years, at least 3 years, at least 4 years, at least 5 years or more, such as 1-5 years, 1-2 years, 2-8 years, 2-10 years, or 3-5 years. In some aspects, each dose/cycle of atezolizumab is administered as a single intravenous injection. In some aspects, the intravenous injection is administered over 30 minutes (±10 minutes) or 60 minutes (±15 minutes). In some aspects, the first cycle or dose is administered over 60 minutes (±15 minutes). In some aspects, the second and subsequent cycles or doses are administered over 30 minutes (±10 minutes). In some aspects, the intravenous injection is not administered as a push or bolus.

In one aspect, the subject treated is a human adult at least 18 years of age, and administering atezolizumab includes intravenously administering atezolizumab at a dose of 1200 mg every 3 weeks; intravenously administering atezolizumab at a dose of 840 mg every 2 weeks; or intravenously administering atezolizumab at a dose of 1680 mg every 4 weeks.

In one aspect, the subject treated is a human pediatric subject less than 18 years of age, and administering atezolizumab comprises intravenously administering atezolizumab at a dose of 15 mg per kilogram of body weight every 21 days. In some aspects, a human pediatric subject is not administered more than 1200 mg of atezolizumab per dose. In some aspects, a human pediatric subject is at least 2 years of age.

In one aspect, prior to administering atezolizumab to the subject, the atezolizumab is diluted to 3.2 mg/ml to 16.8 mg/ml into an infusion bag containing 0.9% sodium chloride injection, USP.

In one aspect, the method does not include co-administering other drugs through an intravenous line used for intravenous administration of the atezolizumab.

In one aspect, the mammalian subject does not have a known primary central nervous system (CNS) malignancy or symptomatic CNS metastases, known clinically significant liver disease, or history of idiopathic pulmonary fibrosis, pneumonitis, organizing pneumonia, or evidence of active pneumonitis on screening chest computed tomography (CT) scan.

In one aspect, the mammalian subject has unresectable ASPS.

In one aspect, the ASPS has Type 1 ASPL-TFE3 fusion transcripts. In one aspect, the ASPS has Type 2 ASPL-TFE3 fusion transcripts.

In one aspect, the mammalian subject treated is a human adult at least 18 years old with an ASPS having Type 1 ASPL-TFE3 fusion transcripts, who was not previously treated with a tyrosine kinase inhibitor (TKI).

In one aspect, the mammalian subject treated is a human child at least 2 years old but younger than 18 years old with an ASPS having Type 1 ASPL-TFE3 fusion transcripts, who was not previously treated with a tyrosine kinase inhibitor (TKI).

In one aspect, the mammalian subject was previously treated with surgery for ASPS.

In one aspect, the mammalian subject was previously treated with radiotherapy, chemotherapy, or both. In one aspect, the mammalian subject was previously treated with pazopanib. In one aspect, the mammalian subject was previously treated with tyrosine kinase inhibitor therapy. In one aspect, the mammalian subject was not previously treated with a TKI therapy.

In one aspect, the disclosed methods further include obtaining a tumor biopsy from the subject prior to administering atezolizumab. In one aspect, the disclosed methods further include obtaining a tumor biopsy from the subject after administering atezolizumab. For example, the method can include obtaining the tumor biopsy after one or more of treatment cycle 1, 2, 3, 4, 5, 6, 7, 8, 9, or 10.

In one aspect, the disclosed methods further include obtaining circulating tumor cells (CTCs) from the mammalian subject prior to administering atezolizumab. In one aspect, the disclosed methods further include obtaining circulating tumor cells (CTCs) from the mammalian subject after administering atezolizumab. For example, the method can include obtaining CTCs after one or more of treatment cycle 1, 2, 3, 4, 5, 6, 7, 8, 9, or 10. In some examples, the CTCs are analyzed for PD-L1 expression, for example using a PD-L1 antibody (such as a labeled antibody). some examples, the CTCs are analyzed for PD-L1 expression, using PD-L1 specific nucleic acid primers, PD-L1 specific nucleic acid probes, or both. In some examples, the CTCs are analyzed for PD-L1 expression, for example using PCR, RT-PCR, ELISA, flow cytometry or microscopy.

In one aspect, the disclosed methods further include obtaining tumor measurements before administering atezolizumab. In one aspect, the disclosed methods further include obtaining tumor measurements after administering atezolizumab. For example, the method can include obtaining tumor measurements after one or more of treatment cycle 1, 2, 3, 4, 5, 6, 7, 8, 9, or 10.

In one aspect, the disclosed methods result in the treated human subject having an objective response within 2 months, 3 months, 4 months, 5 months, 6 months, 7 months, 8 months, 9, months, 10 months, 11 months, 12 months, 13 months, 14 months, or 15 months of a first cycle of administering atezolizumab.

In one aspect, at least 30%, at least 31%, at least 32%, at least 33%, at least 34%, at least 35%, at least 36%, or at least 37% of the human subjects administered the atezolizumab have an objective response within 2 months, 3 months, 4 months, 5 months, 6 months, 7 months, 8 months, 9, months, 10 months, 11 months, 12 months, 13 months, 14 months, or 15 months of a first cycle of administering atezolizumab.

In one aspect, the disclosed methods result in stable disease (SD), a complete response (CR), or a partial response (PR). For example, the SD can be a lack of progression of ASPS tumor lesions and pathological lymph nodes. In some aspects, SD is determined according to Response Evaluation Criteria in Solid Tumors, version 1.1. In some aspects, the treated human subject has SD within at least 2 months, at least 3 months, at least 4 months, at least 5 months, least 6 months, at least 12 months, at least 18 months, at least 24 months, at least 30 months, at least 36 months, at least 48 months, or at least 60 months of a first cycle of administering atezolizumab. For example, the CR can be a disappearance of all ASPS tumor lesions and any pathological lymph nodes have reduction in short axis to <10 mm (<1 cm). For example, the PR can be at least a 30% decrease in the sum of the diameters of ASPS lesions, taking as reference the baseline sum diameters. In one aspect, the treated human subject has a CR within 11 months, 12 months, 13 months, 14 months, or 15 months of a first cycle of administering atezolizumab. In one aspect, the treated human subject has a PR within 11 months, 12 months, 13 months, 14 months, or 15 months of a first cycle of administering atezolizumab. In one aspect, the treated human subject has a duration of response (DOR) of at least 6 months, at least 12 months, at least 18 months, at least 24 months, at least 30 months, at least 36 months, at least 48 months, or at least 60 months of a first cycle of administering atezolizumab. In one aspect, the treated human subject has a progression-free survival of at least 6 months, at least 12 months, at least 18 months, at least 20 months, at least 24 months, at least 30 months, at least 36 months, at least 48 months, or at least 60 months of a first cycle of administering atezolizumab.

In one aspect, the disclosed methods reduce ASPS tumor size or volume by at least 20%, at least 30%, at least 40%, at least 50%, at least 60%, at least 70%, at least 80%, at least 90%, or even 100%, for example as compared to a tumor size or volume prior to administering the atezolizumab.

In one aspect, the disclosed methods further include administering a therapeutically effective amount of a chemotherapy, biologic, radiation therapy, surgery, or combinations thereof. In one aspect, the biologic is a monoclonal antibody that is not atezolizumab.

In one aspect, the disclosed methods further include administering a therapeutically effective amount of the monoclonal antibody bevacizumab. For example, the method can include administering bevacizumab intravenously after administering atezolizumab, such as at dose of 10 mg/kg or 15 mg/kg. In some aspects, the bevacizumab is administered 30 minutes (±10 min) after administering atezolizumab. In some aspects, the bevacizumab is administered over 90 minutes (±15 minutes), over 60 minutes (±10 minutes) or over 30 minutes (±10 minutes). In one aspect, bevacizumab is administered if the subject treated with atezolizumab does not achieve SD, PR, or CR, but instead the ASPS progresses.

The foregoing and other features of this disclosure will become more apparent from the following detailed description of several aspects which proceeds with reference to the accompanying figures.

### BRIEF DESCRIPTION OF THE DRAWINGS

FIG. 1: Patient Responses to Atezolizumab. Time on study (months) as of Jul. 31, 2022, is shown for each patient. Letters A, B, C, and D indicate the best response for each patient, and the time of first complete or partial response is indicated by a diamond or circle, respectively. The dashed line indicates the median time to first response (3.55 months). * indicates an unconfirmed partial response. ** indicates a partial response by Immune Response Evaluation Criteria in Solid Tumors.

FIG. 2: Best Target Lesion Response. The best percent lesion size change from baseline is shown for each patient. Letters A, B, C, and D indicate the best response for each patient, and the dashed line represents a target lesion size decrease of ≥30%. Patient 14 (*) had an unconfirmed partial response. Patient 29 (**) demonstrated a partial response according to iRECIST criteria but a best response of stable disease per RECIST 1.1 criteria due to a >20% increase in target lesion size prior to subsequent shrinkage (i.e., pseudoprogression). Patient #37 (***) achieved radiographic complete response of the target lesion but bone abnormalities persisted.

FIG. 3: Kaplan-Meier Analysis of Progression-Free Survival. Kaplan-Meier estimates of progression-free survival are shown as of Jul. 31, 2022. The dashed line indicates the median progression-free survival, and tick marks indicate unique censoring times. The gray-shaded areas indicate 95% pointwise confidence intervals, with standard errors obtained using Greenwood's formula [computed based on log(survival)].

FIG. 4: Pharmacodynamic Biomarkers in Tumor Biopsies Reveal an ASPS Immunological Landscape Primed for Immune Checkpoint Inhibitor Response. Multiplex immunofluorescence microscopy of core needle tumor biopsies revealed atezolizumab's molecular target (PD-L1+ cells) and immune effector cells (CD8+ CTLs), some of which also exhibited phosphorylation of the (chain of the T cell receptor that is associated with antigen recognition (example cells indicated by arrows), dispersed among ASPS tumor cells expressing nuclear TFE3. The nuclei of all TFE3-negative cells are DAPI stained. In contrast to other malignancies in which lymphocytes accumulate at tumor margins, note that CD8+ CTLs are widely dispersed into all areas of the ASPS tumor microenvironment in the four representative tumors shown here, which is consistent with unimpeded immune cell migration: (from top to bottom) Patient #37, in the PR group, exhibiting 9- to 10-fold treatment-induced increases in PD-L1 and CTL from pre-treatment baseline (left) to Cycle 3 Day 1 of treatment (right); Patient #24, in the PR group, exhibiting persistent presence of PD-L1 and CTL at baseline (left) and Cycle 3 Day 1 (right); Patient #49 in the SD group, exhibiting persistent presence of PD-L1 and CTL at baseline (left) and Cycle 3 Day 1 (right); and Patient #31 in the SD group, exhibiting the highest tumor density of PD-L1 and CTL among all patients with evaluable biopsies. The presence of molecular and cellular components required to respond to atezolizumab's mechanism of action in nearly all patients with evaluable tumor biopsies indicates this malignancy is primed to respond to ICI therapy and is consistent with the high degree of clinical benefit conferred by atezolizumab therapy. Quantitation of each biomarker is presented in FIGS. 12A-12C.

FIG. 5: Demographic and Clinical Characteristics of Evaluable Patients at Baseline. † The ASPL-TFE3 fusion type could not be determined due to lack of a decipherable gel band after reverse transcriptase polymerase chain reaction (9 patients) or lack of sufficient biopsy or blood specimens to test (9 patients).

FIG. 6: Target lesion response over time for patients evaluable for objective response to atezolizumab. The percent lesion size change from baseline is shown for each patient as a function of time on study. Solid lines indicate patients who had received one or more lines of systemic therapy before starting atezolizumab treatment in this study; dashed lines indicate patients who had not received any other systemic therapy before beginning this study. The response trajectories of several patients are highlighted: Patient 17 (a complete response, indicated with “A”); Patient 29 (treated beyond RECIST disease progression and subsequently designated as iPR by iRECIST, indicated with “B”); Patient 34 (stable disease for more than one year before experiencing a PR, indicated with “C”); and Patient 37 (complete resolution of the target lesion but persistent bone abnormalities, indicated with “D”).

FIGS. 7A-7D: Longitudinal PD-L1 expression in circulating tumor cells. Panels A and B show the percent of PD-L1-positive CTCs in blood specimens collected over time (expressed in cycles on study) for patients with a RECIST or iRECIST partial response and high (persistently ≥70%; A) or low (<70%; B) levels of PD-L1 CTC positivity at baseline (or, for patients without a baseline blood specimen, at the earliest timepoint that a specimen was collected). Panel C shows the percent of PD-L1-positive CTCs in blood specimens collected over time (expressed in cycles on study) for patients with a best RECIST response of stable disease or progressive disease. Panel D shows early longitudinal % PD-L1-positive CTC values (left) and the number of PD-L1-expressing cells per mm2 in pre-treatment and Cycle 3 Day 1 tumor biopsy specimens (right) for all patients who had both assessable biopsy specimens and assessable CTC specimens; arrows along the x-axis indicate the times of biopsy. For all panels, large circles indicate the time of first partial response for each patient (if applicable). In panels A and D, the asterisk indicates patient 29, who experienced iRECIST unconfirmed progressive disease (iUPD; indicated by “X”) prior to an iRECIST partial response (large circle); according to standard RECIST criteria, this patient had a best response of stable disease. PD, best response of progressive disease; PR, partial response; Pt., patient; SD, best response of stable disease.

FIG. 8: PD-L1 expression in tumor biopsies scored with the VENTANA PD-L1 (SP142) diagnostic assay performed by CellCarta/HistoGeneX (Naperville, IL). Analysis of FFPE pre-treatment tissue sections via the VENTANA PD-L1 (SP142) diagnostic immunohistochemistry assay did not return any PD-L1-positive scores. Application of the diagnostic assay was ceased after the first 12 samples analyzed were all scored as PD-L1-negative. After formal delivery of the diagnostic assay's results, CellCarta that VENTANA Medical Systems, Inc. (Roche) had determined that the VENTANA PD-L1 (SP142) antibody lot used to analyze study samples was sub-optimal. Specifically, the antibody lot had been found to produce light staining that could affect borderline positive versus negative test results. Before receiving this communication, there were other reports of the SP142 assay's low sensitivity (Tsao et al., J Thorac Oncol 2018; 13:1302-1311; Torlakovic et al., Mod Pathol 2020; 33:4-17; Grabenstetter et al., Am J Surg Pathol 2021; 45:1274-1281), which underscores the importance of evaluating PD-L1 expression with a more sensitive assay coupled with a different detection reagent like the quantitative mIFA employed in this study, which used monoclonal antibody clone 73-10 to detect PD-L1. The 73-10 clone is under consideration as the detection reagent in a companion diagnostic assay for avelumab (Dirix et al., Breast Cancer Res Treat 2018; 167:671-686; Park et al., J Thorac Oncol 2021; 16:1369-1378); it was found to be a more sensitive detection reagent than antibody clones 22C3, 28-8, and SP263 although staining patterns generally correlate (Tsao et al., J Thorac Oncol 2018; 13:1302-1311; Grote et al., J Thorac Oncol 2020; 15:1306-1316; Heo et al., Pathol Res Pract 2021; 218:153338); and its recognition of PD-L1 is less affected by extent of N-linked glycosylation (Mei et al., Mol Cancer 2021; 20:11). CID1 Pre: Cycle 1 Day 1 pre-treatment, iPR: partial response according to iRECIST criteria, PD: progressive disease, PR: partial response, SD: stable disease.

FIG. 9: Representativeness of Study Participants. The statements in this table are based on “(P 042) A SEER-based analysis of alveolar soft part sarcoma from 1975-2018: Incidence, patterns of presentation, and trends in survival” which was presented by Sankaran, H, et al. at the CTOS Virtual Meeting 2021. The characteristics are consistent with other published reports (Wang et al., J Surg Oncol 2016; 113:581-6; Martinez-Trufero et al., Cancer Treat Rev 2021; 99:102260; Fujiwara et al., BMC Cancer 2022; 22:891).

FIGS. 10A-10B: ASPL-TFE3 fusion types identified by RT-PCR. CR: complete response, iPR: partial response according to iRECIST criteria, NR: not reportable (due to lack of a decipherable band after RT-PCR), PD: progressive disease, PR: partial response, SD: stable disease.

FIG. 11: Adverse events related to atezolizumab.

FIGS. 12A-12C: Pharmacodynamic biomarker-positive cell densities measured by mIFA analysis of tumor biopsy specimens. Best Resp.: Best tumor response per RECIST, ID: identification number, iPR: partial response according to iRECIST criteria, NA: tissue specimen not available for analysis, NT: not tested, OnTx: on-treatment biopsy, PD: progressive disease, PFS: progression-free survival (in months, *indicates patient was still on study as of the data cut off), PR: partial response, PreTx: pretreatment biopsy, Pts: patients, SD: stable disease, TQ: tissue quality insufficient for analysis, T_{REG}: regulatory T cell, +: positive. Marker+ Pts: reports the fraction (and percentage) of patients, within a given RECIST response group, whose analyzed biopsy specimen was positive for the indicated biomarker. An individual biopsy specimen was considered positive for a particular biomarker if it contained ≥1 biomarker-expressing cells per mm² of analyzed tissue area (or a ratio <1 in the case of the T_{RE}G/cytotoxic T cell ratio). All but one ASPS tumor with an evaluable biopsy exhibited a T_{REG}/CTL ratio <1, which has been reported in other malignancies as an indication of a permissive immune environment (Kareva et al., Stem Cells 2019; 37:1273-1280).

FIGS. 13A-13D: Tumor areas analyzed and total nuclei counts in mIFA analysis of tumor biopsy specimens. NA: tissue specimen not available for analysis, NT: not tested, TQ: tissue quality insufficient for analysis.

FIGS. 14A-14C: Detection of Circulating Tumor Cells and ASPL-TFE3 Fusion Transcripts in Venous Blood Samples from Individual Patients. CR: complete response, CTC: circulating tumor cell, iPR: partial response according to iRECIST criteria, N: no, NR: not reportable due to lack of a decipherable band by reverse transcriptase polymerase chain reaction, NT: not tested, N/A: not applicable (no sample collected), PBMCs: peripheral blood mononuclear cells, PR: partial response, Pt: patient, uPR: unconfirmed PR, Y: yes.

Of the 25 patients with blood specimens evaluated by imaging flow cytometry for CTCs, 20 (80%) had a CTC-positive specimen (i.e., ≥6 ASPS CTCs, defined as TFE3-positive, CD45-negative cells in the isolated PBMC fraction) at one or more time points. Not all collected specimens/time points were analyzed. Twenty-five other patients contributed blood specimens but did not have any of those specimens analyzed by imaging flow cytometry for CTCs due to sample sufficiency or resource issues. However, 7 of those patients had the ASPL-TFE3 fusion transcript which is not expressed in normal cells-detected in their blood specimens using RT-PCR, indicating that those analyzed blood specimens contained some CTCs.

FIG. 15: Schema of Atezolizumab Monotherapy Arm

FIG. 16: Schema of Atezolizumab+Bevacizumab Combination Arm

FIG. 17: Performance Status Criteria.

FIG. 18: Serum creatinine based on age and gender.

FIG. 19: Hypertension Management Guidelines. Elevations in blood pressure (BP) are based on systolic or diastolic pressures. 1) Elevated BP measurements should be repeated on the same day to confirm the elevation. Patients with elevated BP at any time should have BP measurements performed at least twice weekly until BP within the Upper Limit of Normal (ULN). 2) For adult patients (ages ≥18 years) the ULN is 140/90 mmHg. 3) If BP >25 mm Hg above ULN (verified) or Grade 4 HTN at any time, hold drug. Study drug should also be held for BP≤25 mm Hg above the ULN age for >14 days or 35 mm Hg above baseline for >14 days. Antihypertensive agents can be used to control hypertension as clinically indicated after study drug is held. 4) Anti-hypertensive therapy should be prescribed as clinically indicated, including the use of multiple anti-hypertensive agents.

### DETAILED DESCRIPTION

### I. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “a subject” includes singular or plural subject and can be considered equivalent to the phrase “at least one subject.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

Administration: To provide or give a subject an agent, such as atezolizumab, by any effective route. Exemplary routes of administration include, but are not limited to, oral, injection (such as subcutaneous, intramuscular, intradermal, intraperitoneal, intravenous, and intratumoral), sublingual, rectal, transdermal, intranasal, vaginal and inhalation routes. In one example, administration is intravenous.

Alveolar soft part sarcoma (ASPS): A rare soft-tissue sarcoma, that generally comprises less than 1% of sarcomas. ASPS arises mainly in children and young adults and can metastasize, typically in the lungs and the brain. Typically, ASPS arises in muscles and deep soft tissue of the leg (e.g., thigh), but can also appear in the upper extremities (e.g., hands, neck, and head). While ASPS is a soft tissue sarcoma, it can also spread and grow inside the bones. In some examples, ASPS is characterized by an unbalanced translocation that results from gene fusion of the transcription factor 3 (TFE3) gene at Xp11 to the ASPSCR1 gene at 17q25 (der(17)t(X;17)(p11;q25)), creating an ASPL-TFE3 chimeric fusion protein that is believed to function as an aberrant transcription factor implicated in ASPS pathogenesis. The ASPL-TFE3 fusion protein has been shown to activate MiT (microphthalmia transcription factor) resulting in over-expression of MET. MET is the receptor for hepatocyte growth factor (HGF), affecting cell survival, adhesion, invasion and migration, and angiogenesis. ASPS is a highly vascular tumor as evidenced by radiologic studies and gene expression studies showing upregulation of genes involved in angiogenesis.

Depending on whether the ASPL gene is joined in-frame upstream of either the third or fourth exon of TFE3 there are two distinct ASPL-TFE3 fusion transcripts, Type 1 and Type 2, with the latter harboring an additional exon encoding the TFE3 activation domain. This difference is believed to have a contributory role in the type-2 fusion protein displaying stronger transactivation properties.

Antibody: A polypeptide ligand comprising at least one variable region that recognizes and binds (such as specifically recognizes and specifically binds) an epitope of an antigen. Mammalian immunoglobulin molecules are composed of a heavy (H) chain and a light (L) chain, each of which has a variable region, termed the variable heavy (V_{H}) region and the variable light (V_{L}) region, respectively. Together, the V_{H} region and the V_{L} region are responsible for binding the antigen recognized by the antibody. There are five main heavy chain classes (or isotypes) of mammalian immunoglobulin, which determine the functional activity of an antibody molecule: IgM, IgD, IgG, IgA and IgE. Antibody isotypes not found in mammals include IgX, IgY, IgW and IgNAR. IgY is the primary antibody produced by birds and reptiles and is functionally similar to mammalian IgG and IgE. IgW and IgNAR antibodies are produced by cartilaginous fish, while IgX antibodies are found in amphibians.

Antibody variable regions contain “framework” regions and hypervariable regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of well-known numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., JMB 273,927-948, 1997; the “Chothia” numbering scheme), Kunik et al. (see Kunik et al., PLoS Comput Biol 8:e1002388, 2012; and Kunik et al., Nucleic Acids Res 40(Web Server issue):W521-524, 2012; “Paratome CDRs”) and the ImMunoGeneTics (IMGT) database (see, Lefranc, Nucleic Acids Res 29:207-9, 2001; the “IMGT” numbering scheme). The Kabat, Paratome and IMGT databases are maintained online. In addition, the AbRSA tool can be used to determine the CDR boundaries according to Kabat, IMGT or Chothia (online at aligncdr.labshare.cn/aligncdr/abrsa.php).

A “single-domain antibody” refers to an antibody having a single domain (a variable domain) that is capable of specifically binding an antigen, or an epitope of an antigen, in the absence of an additional antibody domain. Single-domain antibodies include, for example, V_{H} domain antibodies, V_{NAR} antibodies, camelid V_{H}H antibodies, and V_{L} domain antibodies. V_{NAR} antibodies are produced by cartilaginous fish, such as nurse sharks, wobbegong sharks, spiny dogfish and bamboo sharks. Camelid V_{H}H antibodies are produced by several species including camel, llama, alpaca, dromedary, and guanaco, which produce heavy chain antibodies that are naturally devoid of light chains.

A “monoclonal antibody” is an antibody produced by a single clone of lymphocytes or by a cell into which the coding sequence of a single antibody has been transfected. Monoclonal antibodies are produced by methods known to those of skill in the art. Monoclonal antibodies include humanized monoclonal antibodies.

A “chimeric antibody” has framework residues from one species, such as human, and CDRs (which generally confer antigen binding) from another species.

A “humanized” antibody is an immunoglobulin including a human framework region and one or more CDRs from a non-human (for example a mouse, rabbit, rat, shark or synthetic) immunoglobulin. The non-human immunoglobulin providing the CDRs is termed a “donor,” and the human immunoglobulin providing the framework is termed an “acceptor.” In one aspect, all CDRs are from the donor immunoglobulin in a humanized immunoglobulin. Constant regions need not be present, but if they are, they must be substantially identical to human immunoglobulin constant regions, such as at least about 85-90%, such as about 95% or more identical. Hence, all parts of a humanized immunoglobulin, except possibly the CDRs, are substantially identical to corresponding parts of natural human immunoglobulin sequences. A humanized antibody binds to the same antigen as the donor antibody that provides the CDRs. Humanized or other monoclonal antibodies can have additional conservative amino acid substitutions which have substantially no effect on antigen binding or other immunoglobulin functions.

Anti-microtubule agent: A type of drug that blocks cell growth by stopping mitosis. Anti-microtubule agents, also referred to as “anti-mitotic agents,” are used to treat cancer, and in some aspects are used in combination with atezolizumab to treat ASPS.

Atezolizumab: A human monoclonal antibody designed to specifically bind programmed cell death ligand-1 (PD-L1). Atezolizumab was engineered to eliminate Fc-effector function via a single amino acid substitution at position 298 on the heavy chain, which results in a non-glycosylated antibody that has minimal binding to Fc receptors and, consequently, eliminates detectable Fc-effector function. By eliminating Fc-effector function and antibody dependent cell-mediated cytotoxicity (ADCC), antibody-mediated clearance of activated effector T cells is also eliminated. Atezolizumab binds to PD-L1 expressed on tumor cells and tumor-infiltrating immune cells, blocking its interactions with both PD-1 and B7.1 receptors. Thus, atezolizumab is a PD-L1 blocking antibody. Tecentriq® (atezolizumab) injection for intravenous use is a sterile, preservative-free, colorless to slightly yellow solution in single-dose vials. Each 20 mL vial of Tecentriq® contains 1200 mg of atezolizumab and is formulated in glacial acetic acid (16.5 mg), L-histidine (62 mg), polysorbate 20 (8 mg), and sucrose (821.6 mg), with a pH of 5.8. Each 14 mL vial of Tecentriq® contains 840 mg of atezolizumab and is formulated in glacial acetic acid (11.5 mg), L-histidine (43.4 mg), polysorbate 20 (5.6 mg), and sucrose (575.1 mg) with a pH of 5.8.

Bevacizumab: A recombinant humanized anti-vascular endothelial growth factor (VEGF) monoclonal antibody consisting of 93% human and 7% murine amino acid sequences. Bevacizumab blocks the binding of VEGF to its receptors resulting in inhibition of angiogenesis. Bevacizumab is composed of human IgG framework and murine antigen binding complementarity-determining regions. One specific product is Avastin® injection, which is supplied as a clear to slightly opalescent, sterile liquid for parenteral administration. Each 400 mg (25 mg/ml-16 mL fill) glass vial contains bevacizumab with phosphate, trehalose, polysorbate 20, and Sterile Water for Injection, USP. In some aspects, the initial dose of bevacizumab is administered iv over a minimum of 90 minutes (±15 minutes). If no adverse reactions occur, administer the second dose over a minimum of 60 minutes (±10 minutes). If no adverse reactions occur after the second dose, administer subsequent doses over a minimum of 30 minutes (±10 minutes).

Chemotherapeutic agent: Any chemical agent with therapeutic usefulness in the treatment of diseases characterized by abnormal cell growth. Such diseases include tumors, neoplasms, and cancer as well as diseases characterized by hyperplastic growth. In one aspect, a chemotherapeutic agent is an agent of use in treating a ASPS, for example alone or in combination with other agents, such as atezolizumab. In one aspect, a chemotherapeutic agent is a radioactive compound. A skilled person can readily identify a chemotherapeutic agent of use (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer, L., Berkery, R. (eds.): Oncology Pocket Guide to Chemotherapy, 2nd ed. St. Louis, Mosby-Year Book, 1995; Fischer, D. S., Knobf, M. F., Durivage, H. J. (eds): The Cancer Chemotherapy Handbook, 4th ed. St. Louis, Mosby-Year Book, 1993). Combination chemotherapy is the administration of more than one agent to treat cancer. One example is the administration of atezolizumab used in combination with a radioactive or chemical compound, or other biologic. In one example, a chemotherapeutic agent is a biologic, such as a therapeutic antibody (e.g., therapeutic monoclonal antibody), such as atezolizumab, as well as other anti-cancer antibodies, such as anti-PD1 or other anti-PDL1 (e.g., pembrolizumab and nivolumab), anti-CTLA4 (e.g., ipilimumab), anti-EGFR (e.g., cetuximab), anti-VEGF (e.g., bevacizumab), or combinations thereof (e.g., anti-PDL-1 and anti-CTLA-4 or anti-PDL-1 and anti-VEGF).

Contacting: Placement in direct physical association; includes both in solid and liquid form.

Cytotoxic agent: Any drug or compound that kills cells.

Cytotoxicity: The toxicity of a molecule, such as an immunotoxin, to the cells intended to be targeted, as opposed to the cells of the rest of an organism. In contrast, the term “toxicity” refers to toxicity of an immunotoxin to cells other than those that are the cells intended to be targeted by the targeting moiety of the immunotoxin, and the term “animal toxicity” refers to toxicity of the immunotoxin to an animal by toxicity of the immunotoxin to cells other than those intended to be targeted by the immunotoxin.

Drug: Any compound used to treat, ameliorate or prevent a disease or condition in a subject. In some aspects herein, the drug is an anti-cancer agent, for example a cytotoxic agent, such as an anti-mitotic or anti-microtubule agent.

Effector molecule: The portion of a chimeric molecule that is intended to have a desired effect on a cell to which the chimeric molecule is targeted. Effector molecule is also known as an effector moiety (EM), therapeutic agent, diagnostic agent, or similar terms. Therapeutic agents (or drugs) include such compounds as nucleic acids, proteins, peptides, amino acids or derivatives, glycoproteins, radioisotopes, photon absorbers, lipids, carbohydrates, or recombinant viruses. Nucleic acid therapeutic and diagnostic moieties include antisense nucleic acids, derivatized oligonucleotides for covalent cross-linking with single or duplex DNA, and triplex forming oligonucleotides. Alternatively, the molecule linked to a targeting moiety, such as atezolizumab, may be an encapsulation system, such as a liposome or micelle that contains a therapeutic composition such as a drug, a nucleic acid (such as an antisense nucleic acid), or another therapeutic moiety that can be shielded from direct exposure to the circulatory system. Means of preparing liposomes attached to antibodies are known (see, for example, U.S. Pat. No. 4,957,735; and Connor et al., Pharm Ther 28:341-365, 1985). Diagnostic agents or moieties include radioisotopes and other detectable labels. Detectable labels useful for such purposes are also well known and include radioactive isotopes such as ³⁵, ¹¹C, ¹³N, ¹⁵O, ¹⁸F, ¹⁹F, ^{99m}Tc, ¹³¹I, ³H, ¹⁴C, ¹⁵N, ⁹⁰Y, ⁹⁹Tc, ¹¹¹In and ¹²⁵I, fluorophores, chemiluminescent agents, and enzymes.

Epitope: An antigenic determinant. These are particular chemical groups or peptide sequences on a molecule that are antigenic (that elicit a specific immune response). An antibody specifically binds a particular antigenic epitope on a polypeptide, such as PD-L1.

Framework region: Amino acid sequences interposed between CDRs. Framework regions of an immunoglobulin molecule include variable light and variable heavy framework regions.

Heterologous: Originating from a separate genetic source or species.

Immune response: A response of a cell of the immune system, such as a B cell, T cell, or monocyte, to a stimulus. In one aspect, the response is specific for a particular antigen (an “antigen-specific response”). In one aspect, an immune response is a T cell response, such as a CD4+ response or a CD8+ response. In another aspect, the response is a B cell response, and results in the production of specific antibodies.

Interstrand crosslinking agent: A type of cytotoxic drug capable of binding covalently between two strands of DNA, thereby preventing DNA replication and/or transcription.

Isolated: An “isolated” biological component, such as a nucleic acid, protein (including antibodies) or organelle, has been substantially separated or purified away from other biological components in the environment (such as a cell) in which the component occurs, for example other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Label: A detectable compound or composition that is conjugated directly or indirectly to another molecule, such as an antibody or a protein, to facilitate detection of that molecule. Thus, in some aspects, atezolizumab includes one or more labels. Specific, non-limiting examples of labels include fluorescent tags, enzymatic linkages, and radioactive isotopes. In one example, a “labeled antibody” refers to incorporation of another molecule in the antibody. For example, the label is a detectable marker, such as the incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ⁵S, ¹¹C, ¹³N, ¹⁵O, ¹⁸F, ¹⁹F, ^{99m}Tc, ¹³¹I, ³H, ¹⁴C, ¹⁵N, ⁹⁰Y, ⁹⁹Tc, ¹¹¹In and ¹²⁵I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some aspects, labels are attached by spacer arms of various lengths to reduce potential steric hindrance.

Pharmaceutically acceptable carriers: The pharmaceutically acceptable carriers of use are conventional. Remington: The Science and Practice of Pharmacy, The University of the Sciences in Philadelphia, Editor, Lippincott, Williams, & Wilkins, Philadelphia, PA, 21^{st} Edition (2005), describes compositions and formulations suitable for pharmaceutical delivery of the antibodies and other compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop, such as a reduction in tumor burden or a decrease in the number of size of metastases. “Ameliorating” refers to the reduction in the number or severity of signs or symptoms of a disease, such as ASPS.

Purified: The term purified does not require absolute purity; rather, it is intended as a relative term. Thus, for example, a purified peptide preparation is one in which the peptide or protein is more enriched than the peptide or protein is in its natural environment within a cell. In one aspect, a preparation is purified such that the protein or peptide represents at least 50% of the total peptide or protein content of the preparation. Substantial purification denotes purification from other proteins or cellular components. A substantially purified protein is at least 60%, 70%, 80%, 90%, 95% or 98% pure. Thus, in one specific, non-limiting example, a substantially purified protein is 90% free of other proteins or cellular components.

Pyrrolobenzodiazepine (PBD): A class of sequence-selective DNA minor-groove binding crosslinking agents originally discovered in Streptomyces species. PBDs are significantly more potent than systemic chemotherapeutic drugs. The mechanism of action of PBDs is associated with their ability to form an adduct in the minor groove of DNA, thereby interfering with DNA processing. In the context of the present disclosure, PBDs include naturally produced and isolated PBDs, chemically synthesized naturally occurring PBDs, and chemically synthesized non-naturally occurring PBDs. PBDs also include monomeric, dimeric and hybrid PBDs (for a review see Gerratana, Med Res Rev 32(2):254-293, 2012).

Recombinant: A recombinant nucleic acid or protein is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, for example, by genetic engineering techniques.

Sample (or biological sample): A biological specimen containing genomic DNA, RNA (including mRNA), protein, or combinations thereof, obtained from a subject. Examples include, but are not limited to, peripheral blood, tissue, cells, urine, semen, saliva, tissue biopsy, fine needle aspirate, surgical specimen, and autopsy material. In one example, a sample includes a tumor biopsy, such as an ASPS biopsy. In one aspect, a sample includes a lymph node biopsy.

Subject: Living multi-cellular vertebrate organisms, a category that includes both human and veterinary subjects, including human and non-human mammals. In one aspect, a subject to be treated with the methods provided herein is a human adult at least 18 years of age having ASPS. In another aspect, a subject to be treated with the methods provided herein is a human child under the age of 18, but at least 2 years old having ASPS.

Therapeutically effective amount: A quantity of a specific substance sufficient to achieve a desired effect in a subject being treated, such as an amount of atezolizumab. For instance, this can be the amount of atezolizumab needed to inhibit or suppress growth of a tumor, such as ASPS. In one aspect, a therapeutically effective amount is the amount necessary to eliminate, reduce the size or volume, or prevent metastasis of, ASPS, such as reduce a ASPS tumor size and/or volume by at least 10%, at least 20%, at least 50%, at least 75%, at least 80%, at least 90%, at least 95%, or even 100%, and/or reduce the number and/or size/volume of ASPS metastases by at least 10%, at least 20%, at least 50%, at least 75%, at least 80%, at least 90%, at least 95%, or even 100%, for example as compared to a size/volume/number prior to atezolizumab treatment. When administered to a subject, a dosage will generally be used that will achieve target tissue concentrations (for example, in ASPS tumors) that has been shown to achieve a desired in vitro effect.

### II. Methods of Treatment

Alveolar soft part sarcoma (ASPS) is a rare cancer that accounts for <1% of all soft tissue sarcomas. With a global incidence of <1 per 1,000,000, ASPS has been classified by the Connective Tissue Oncology Society as an ultra-rare sarcoma (Stacchiotti et al., Cancer 2021; 127:2934-2942). The disease typically occurs in adolescents and young adults. ASPS generally displays an indolent course but exhibits a high tendency for early metastatic spread which is associated with decreased 5-year overall survival rates ranging from 20% to 46% (Coyne et al., Curr Prob Cancer 2021; 45:100775). The defining molecular event in ASPS is a chromosomal translocation, der(17)t(X;17)(p11;q25), involving ASPL (also known as ASPSCR1) and TFE3 (Ladanyi et al., Oncogene 2001; 20:48-57). Two distinct ASPL-TFE3 fusion proteins have been identified that reportedly differ in transcriptional activity (Kobos et al., J Pathol 2013; 229:743-754). However, to date, no clinical significance of Type 1 versus Type 2 fusions with respect to prognosis has been described (Williams et al., Virchows Arch 2011; 458:291-300).

ASPS is largely resistant to traditional chemotherapies. Initial management via surgical resection and/or systemic treatment is rarely curative (Coyne et al., Curr Prob Cancer 2021; 45:100775). The tyrosine kinase inhibitor pazopanib is approved by the United States Food and Drug Administration (FDA) for soft tissue sarcoma in general and is a treatment option for ASPS patients. In December 2022, based on independently reviewed results from the study reported herein, the FDA approved atezolizumab, an immune checkpoint inhibitor (ICI) targeting PD-L1, for the treatment of adult and pediatric patients ≥2 years of age with unresectable or metastatic ASPS (Label Information for TECENTRIQ® (atezolizumab) injection).

The clinical and pharmacodynamic results of the largest single-arm phase 2 clinical trial of an immunotherapy agent, atezolizumab, for the treatment of patients with advanced ASPS is described herein. Encouraging clinical responses of ASPS to other ICIs disrupting PD-L1 binding to its PD-1 receptor, with or without a tyrosine kinase inhibitor, have been reported (Groisberg et al., J Immunother Cancer 2017; 5:100-100; Groisberg et al., Mol Cancer Ther 2020; 19:1165-1172; Conley et al., J Glob Oncol 2017; 4:1-6; Wilky et al., Lancet Oncol 2019; 20:837-848; Shi et al., Clin Cancer Res 2020; 26:6445-6452; Martin-Broto et al., J Immunother Cancer 2020; 8:e001561-e001561; Blay et al. J Clin Oncol 2021; 39:11520-11520; Conry et al., J Adolesc Young Adult Oncol 2020; 9:449-452; Yang et al., Eur J Cancer 2020; 130:182-192; Mittal et al., J Adolesc Young Adult Oncol 2021; 10:501-502; Mariuk-Jarema et al., Tumori J 2020; 106:Np9-Np13; Su et al., Invest New Drugs 2021; 39:1411-1418; Dorman et al., Anti-cancer Drugs 2023, 34:311-316; Xu et al., Ann Palliat Med 2021; 10:785-792). However, most sample sizes were small and the molecular mechanisms behind the responses were unclear. Tumor mutational burden and microsatellite instability, which are associated with the presence of tumor neoantigens and a high rate of response to immune checkpoint inhibition in many cancer histologies (Palmeri et al., Esmo Open 2022; 7:100336), are low in ASPS (Groisberg et al., Mol Cancer Ther 2020; 19:1165-1172; Shi et al., Clin Cancer Res 2020; 26:6445-6452; Chang et al., Clin Cancer Res 2016; 22:3810-3820; Lewin et al., Cancer Immunol Res 2018; 6:1001-1007). Additionally, some ICI-responsive ASPS tumors reportedly lack the expression of PD-L1 or PD-1 proteins that are associated with response to immune checkpoint blockade (Conley et al., J Glob Oncol 2017; 4:1-6; Wilky et al., Lancet Oncol 2019; 20:837-848; Shi et al., Clin Cancer Res 2020; 26:6445-6452; Lewin et al., Cancer Immunol Res 2018; 6:1001-1007; Vander et al., Radiol Case Rep 2018; 13:882-885). Therefore, the present methods were developed to evaluate the efficacy of atezolizumab treatment for ASPS and to investigate the hypothesis that the mechanism of ICI activity in this disease involves induced expression of missing immune checkpoint components.

Provided herein are methods of using atezolizumab to treat ASPS, such as ASPS not curable by surgery. Atezolizumab is a human monoclonal antibody directed against programmed death-ligand 1 (PD-L1) with potential immune checkpoint inhibitory and antineoplastic activities. Atezolizumab targets immune cells or tumor cells and prevents interaction with either programmed death-1 (PD-1) receptor or B7.1 (CD80), both of which function as inhibitory receptors expressed on T cells. Interference of the PD-L1:PD-1 and PDL1: B7.1 interactions may enhance the magnitude and quality of the tumor-specific T-cell response through increased T-cell priming, expansion, and/or effector function.

Atezolizumab is described in WHO Drug Information (International Nonproprietary Names for Pharmaceutical Substances (INN)) Proposed INN: List 112, Vol. 28, No. 4, 2014, pp. 488-489 and WHO Drug Information (International Nonproprietary Names for Pharmaceutical Substances (INN)), Recommended INN: List 74, Vol. 29, No. 3, 2015, p. 387. Atezolizumab is an Fc-engineered, humanized, non-glycosylated IgG1 kappa immunoglobulin that binds PD-L1 and comprises the heavy chain sequence of SEQ ID NO: 1 and the light chain sequence of SEQ ID NO: 2. Atezolizumab comprises a single amino acid substitution (asparagine to alanine) at position 297 on the heavy chain (N297A) using EU numbering of Fc region amino acid residues, which results in a non-glycosylated antibody that has minimal binding to Fc receptors.

In some instances, the anti-PD-L1 antibody comprises:
(a) an HVR-H1, HVR-H2, and HVR-H3 sequence of GFTFSDSWIH (SEQ ID NO: 3), AWISPYGGSTYYADSVKG (SEQ ID NO: 4) and RHWPGGFDY (SEQ ID NO: 5), respectively, and
(b) an HVR-L1, HVR-L2, and HVR-L3 sequence of RASQDVSTAVA (SEQ ID NO: 6), SASFLYS (SEQ ID NO: 7) and QQYLYHPAT (SEQ ID NO: 8), respectively.

In one embodiment, the anti-PD-L1 antibody comprises:
(a) a heavy chain variable region (VH) comprising the amino acid sequence:

(SEQ ID NO: 9)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSS,

and
(b) the light chain variable region (VL) comprising the amino acid sequence:

(SEQ ID NO: 10)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKR.

In one embodiment, the anti-PD-L1 antibody comprises atezolizumab, which comprises:
(a) the heavy chain amino acid sequence:

(SEQ ID NO: 1)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYASTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPG,

and
(b) the light chain amino acid sequence:

(SEQ ID NO: 2)
DIQMTQSPSSLSASVGDRVTITCRASQDVSTAVAWYQQKPGKAPKLLIY
SASFLYSGVPSRFSGSGSGTDFTLTISSLOPEDFATYYCQQYLYHPATF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

An exemplary atezolizumab formulation comprises glacial acetic acid, L-histidine, polysorbate 20, and sucrose, with a pH of 5.8. For example, atezolizumab may be provided in a 20 mL vial containing 1200 mg of atezolizumab that is formulated in glacial acetic acid (16.5 mg), L-histidine (62 mg), polysorbate 20 (8 mg), and sucrose (821.6 mg), with a pH of 5.8. In another example, atezolizumab may be provided in a 14 mL vial containing 840 mg of atezolizumab that is formulated in glacial acetic acid (11.5 mg), L-histidine (43.4 mg), polysorbate 20 (5.6 mg), and sucrose (575.1 mg) with a pH of 5.8.

In some aspects, the mammalian subject to be treated has measurable ASPS disease, such as at least one lesion that can be accurately measured in at least one dimension (longest diameter to be recorded for nonmodal lesions and short axis for nodal lesions} as ≥20 mm (≥2 cm) by chest x-ray or as ≥10 mm (≥1 cm) with CT scan, MRI, or calipers by clinical exam. In some aspects, the mammalian subject to be treated has a newly diagnosed, unresectable, metastatic and measurable ASPS and shows clinical evidence of disease progression (including history and increasing physical symptoms). In some aspects, the mammalian subject to be treated does not have a known primary CNS malignancy or symptomatic CNS metastases.

In one aspect, the mammalian subject to be treated has unresectable ASPS.

In one aspect, the ASPS to be treated has Type 1 ASPL-TFE3 fusion transcripts. In one aspect, the ASPS to be treated has Type 2 ASPL-TFE3 fusion transcripts.

In some aspects, the mammalian subject to be treated has an ECOG performance status of less than or equal to 2 (e.g., Karnofsky or Lansky ≥70%). In some aspects, the mammalian subject to be treated has normal organ and marrow function.

In some aspects, the mammalian subject to be treated is not pregnant.

In some aspects, the mammalian subject to be treated does not have a known primary central nervous system (CNS) malignancy or symptomatic CNS metastases, known clinically significant liver disease, or history of idiopathic pulmonary fibrosis, pneumonitis, organizing pneumonia, or evidence of active pneumonitis on screening chest computed tomography (CT) scan.

In some aspects, the mammalian subject to be treated has previously been treated with other agents, such as radiation therapy, chemotherapy, or both, such as least 2 weeks, at least 4 weeks, oat least 6 weeks, or at least 12 weeks prior to administration of the atezolizumab. In some aspects, the mammalian subject to be treated has previously been treated with a nitrosoursea, doxorubicin, mitomycin C, interferon-alpha, interleukin-2, a monoclonal antibody therapy that is not atezolizumab (e.g., an anti-CTLA-4 mAb, anti-PD-1 mAb, or antibi-PC-L1-1 mAb), an immunosuppressive medication (including, but not limited to, prednisone, cyclophosphamide, azathioprine, methotrexate, thalidomide, and anti-tumor necrosis factor [anti-TNF]agents), or combinations thereof. In some aspects, the mammalian subject to be treated has previously been treated with pazopanib prior to administration of the atezolizumab. In some aspects, the mammalian subject to be treated has previously been treated with a tyrosine kinase inhibitor therapy prior to administration of the atezolizumab. In some aspects, the mammalian subject to be treated was previously treated with surgery for ASPS.

In one aspect, the subject treated is a mammal, such as a human or veterinary subject. In some aspects, the subject treated is a human adult patient, such as one at least 18 years old. In some examples, the subject treated is a human geriatric patient, such as one at least 65 years old. In some examples, the subject treated is a human adolescent patient, such as one 12-17 years old. In some examples, the subject treated is a human pediatric patient, such as one at least 2 years old. In some examples, the human pediatric patient is 2 years to <17 years. In some examples, the subject treated is a human patient at least 6 years old.

In some aspects, atezolizumab is administered every 2 weeks, every 3 weeks, or every 4 weeks. Each administration is a “cycle”. In some aspects, atezolizumab is administered as a single dose for at least 2 cycles, at least 3 cycles, at least 4 cycles, at least 5 cycles, at least 10 cycles, at least 20 cycles, at least 100 cycles, or more, such as 2, 3, 4, 5, 6, 7, 8, 9, 10, 15, 20, 25, 30, 40, 50 or 100 cycles. In some examples, atezolizumab is administered as a single dose for at least 2 cycles, at least 3 cycles, at least 4 cycles, at least 5 cycles, at least 10 cycles, at least 20 cycles, followed by a period where no atezolizumab is administered (e.g., a “rest” period of at least 1 month, at least 2 months, at least 3 months, at least 4 months, at least 5 months, at least 6 months, at least 9 months, at least 12 months, at least 24 months, at least 36 months, at least 48 months, at least 60 months, at least 72 months, or at least 96 months, such as 1-60 months, 12-24 months, 36-60 months, or 36-96 months, for example if a complete response, partial response, or stable disease is achieved), followed by a period where atezolizumab is administered using the methods provided herein. In some aspects, each dose/cycle of atezolizumab is administered as a single intravenous injection. In some aspects, the intravenous injection is administered over 30 minutes (±10 minutes) or 60 minutes (±15 minutes). In some aspects, the first cycle or dose is administered over 60 minutes (±15 minutes). In some aspects, the second and subsequent cycles or doses are administered over 30 minutes (±10 minutes). In some aspects, the intravenous injection is not administered as a push or bolus.

In one aspect, the subject treated is a human adult at least 18 years of age, and administering atezolizumab includes intravenously administering atezolizumab at a dose of 1200 mg every 3 weeks; intravenously administering atezolizumab at a dose of 840 mg every 2 weeks; or intravenously administering atezolizumab at a dose of 1680 mg every 4 weeks.

In one aspect, the subject treated is a human pediatric subject less than 18 years of age, and administering atezolizumab comprises intravenously administering atezolizumab at a dose of 15 mg per kilogram of body weight every 21 days. In some aspects, a human pediatric subject is not administered more than 1200 mg of atezolizumab per dose. In some aspects, a human pediatric subject is at least 2 years of age.

In one aspect, prior to administering atezolizumab to the subject, the atezolizumab is diluted to 3.2 mg/ml to 16.8 mg/ml into an infusion bag containing 0.9% sodium chloride injection, USP.

In one aspect, the method does not include co-administering other drugs through an intravenous line used for intravenous administration of the atezolizumab.

In one aspect, the disclosed methods further include obtaining an ASPS tumor biopsy from the subject prior to administering atezolizumab. In one aspect, the disclosed methods further include obtaining an ASPS tumor biopsy from the subject after administering atezolizumab. For example, the method can include obtaining the tumor biopsy after one or more of treatment cycle 1, 2, 3, 4, 5, 6, 7, 8, 9, or 10. In one aspect, ASPS tumor biopsies are performed at baseline and just before cycle 3. In some aspects, ASPS tumor biopsies are analyzed by microscopy, such as immunofluorescence microscopy. In some aspects ASPS tumor biopsies are analyzed for the presence of PD-L1/CD8, PD-1/CD3, CD4/FOXP3, and/or CD8/CD3ζ pY142/TFE3). In some examples, the PD-L1 monoclonal antibody clone 73-10 is used for detection of PD-L1 in the tumor sample.

In one aspect, the disclosed methods further include obtaining circulating tumor cells (CTCs) from the mammalian subject prior to administering atezolizumab. In one aspect, the disclosed methods further include obtaining circulating tumor cells (CTCs) from the mammalian subject after administering atezolizumab. For example, the method can include obtaining CTCs after one or more of treatment cycle 1, 2, 3, 4, 5, 6, 7, 8, 9, or 10. In one aspect, CTCs are obtained from patients at baseline, on cycle 1 before the atezolizumab dose is administered and at the start of each subsequent cycle, and at a time of disease progression. In some aspects, PD-L1 expression in CTCs is analyzed, for example by flow cytometry.

In some aspects, the method includes analyzing ASPS tumor samples and/or CTCs for expression of type 1 and type 2 ASPL-TFE3 fusions, for example from RNA extracted from such samples.

In one aspect, the disclosed methods further include obtaining ASPS tumor measurements before administering atezolizumab. In one aspect, the disclosed methods further include obtaining ASPS tumor measurements after administering atezolizumab. For example, the method can include obtaining ASPS tumor measurements after one or more of treatment cycle 1, 2, 3, 4, 5, 6, 7, 8, 9, or 10. In one aspect, ASPS tumor measurements are made at the end of cycle 3, every two cycles thereafter for the first year, every three cycles during the second year, and every four cycles after that. In some aspects, tumors are measured using computed tomography, magnetic resonance imaging or positron-emission tomography-computed tomography. Confirmatory scans can be performed at least 4 weeks after initial documentation of objective response,

In one aspect, the disclosed methods result in the treated human subject having an objective response within 2 months, 3 months, 4 months, 5 months, 6 months, 7 months, 8 months, 9, months, 10 months, 11 months, 12 months, 13 months, 14 months, or 15 months of a first cycle of administering atezolizumab.

In one aspect, at least 30%, at least 31%, at least 32%, at least 33%, at least 34%, at least 35%, at least 36%, or at least 37% of the human subjects administered the atezolizumab have an objective response within 2 months, 3 months, 4 months, 5 months, 6 months, 7 months, 8 months, 9, months, 10 months, 11 months, 12 months, 13 months, 14 months, or 15 months of a first cycle of administering atezolizumab.

In one aspect, the disclosed methods result in a complete response (CR), or a partial response (PR). For example, the CR can be a disappearance of all ASPS tumor lesions and any pathological lymph nodes have reduction in short axis to <10 mm (<1 cm). For example, the PR can be at least a 30% decrease in the sum of the diameters of ASPS lesions, taking as reference the baseline sum diameters. In one aspect, the treated human subject has a CR within 11 months, 12 months, 13 months, 14 months, or 15 months of a first cycle of administering atezolizumab. In one aspect, the treated human subject has a PR within 11 months, 12 months, 13 months, 14 months, or 15 months of a first cycle of administering atezolizumab. In one aspect, the treated human subject has a duration of response (DOR) of at least 6 months, at least 12 months, at least 18 months, at least 24 months, at least 30 months, at least 36 months, at least 48 months, or at least 60 months of a first cycle of administering atezolizumab. In one aspect, the treated human subject has a progression-free survival of at least 6 months, at least 12 months, at least 18 months, at least 20 months, at least 24 months, at least 30 months, at least 36 months, at least 48 months, or at least 60 months of a first cycle of administering atezolizumab.

In one aspect, the disclosed methods reduce ASPS tumor size or volume by at least 20%, at least 30%, at least 40%, at least 50%, at least 60%, at least 70%, at least 80%, at least 90%, or even 100%, for example as compared to a tumor size or volume prior to administering the atezolizumab.

In one aspect, the disclosed methods further include administering a therapeutically effective amount of a chemotherapy, biologic, radiation therapy, surgery, or combinations thereof. In one aspect, the biologic is a monoclonal antibody that is not atezolizumab.

In one aspect, the disclosed methods further include administering a therapeutically effective amount of the monoclonal antibody bevacizumab. For example, the method can include administering bevacizumab intravenously after administering atezolizumab, such as at dose of 10 mg/kg or 15 mg/kg. In some aspects, the bevacizumab is administered 30 minutes (±10 min) after administering atezolizumab. In some aspects, the bevacizumab is administered over 90 minutes (±15 minutes), over 60 minutes (±10 minutes) or over 30 minutes (±10 minutes).

In some aspects, the disclosed methods of treating ASPS include administering to the subject a therapeutically effective amount of of atezolizumab.

In some aspects, an antigen-binding fragment of atezolizumab is used, such as an Fab fragment, an Fab′ fragment, an F(ab)′₂ fragment, a single chain variable fragment (scFv) or a disulfide stabilized variable fragment (dsFv).

In some aspects, atezolizumab further includes a constant region, such as an IgG constant region, for example, an IgG1 constant region, such as a human IgG1 constant region. In some examples, the constant region includes at least one amino acid modification to increase the half-life, stability and/or function of atezolizumab.

### III. Compositions and Administration

Compositions are provided that include atezolizumab in a carrier, such as 0.9% saline. The compositions can be prepared in unit dosage form for administration to a subject. The amount and timing of administration are at the discretion of the treating clinician to achieve the desired outcome. The antibody can be formulated for systemic or local administration, such as intravenous administration.

The compositions for administration can include a solution of atezolizumab in a pharmaceutically acceptable carrier, such as an aqueous carrier. A variety of aqueous carriers can be used, for example, buffered saline and the like. These solutions are sterile and generally free of undesirable matter. These compositions may be sterilized by conventional sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

In one aspect, an atezolizumab pharmaceutical composition for intravenous administration includes about 500 to 1500 mg of an antibody, such as 840 or 1200 mg, per subject per cycle (such as a 2, 3, or 4 week cycle). In one aspect, one dose is 840 mg atezolizumab/14 mL carrier or 1,200 mg atezolizumab/20 mL carrier. In some aspects, dosages of 840 mg every two weeks, 1200 mg every 3 weeks, or 1680 mg every 4 weeks are used for adults. In some aspects, dosages of a 15 mg atezolizumab per kilogram of body weight every 3 weeks (not to exceed 1200 mg atezolizumab) are used for children under 18 years old and older than 2 years old. Additional information on methods for preparing administrable compositions can be found in publications such as Remington: The Science and Practice of Pharmacy, The University of the Sciences in Philadelphia, Editor, Lippincott, Williams, & Wilkins, Philadelphia, PA, 21^{st} Edition (2005).

Atezolizumab (or atezolizumab conjugates, or nucleic acid molecules encoding such molecules) may be provided in lyophilized form and rehydrated with sterile water or saline before administration. The antibody solution can be added to an infusion bag containing 0.9% sodium chloride, USP. Antibodies can be administered by slow infusion, rather than in an intravenous push or bolus. In one aspect, an initial dose of atezolizumab is administered slower than subsequent doses. In one aspect, a first dose or cycle of atezolizumab is administered over 60 minutes (±15 minutes), while subsequent doses/cycles are administered over 30 minutes (±10 minutes) if tolerated.

Controlled release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, PA, (1995). Particulate systems include, for example, microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, J., Colloidal Drug Delivery Systems, J. Kreuter, ed., Marcel Dekker, Inc., New York, NY, pp. 219-342 (1994); and Tice & Tabibi, Treatise on Controlled Drug Delivery, A. Kydonieus, ed., Marcel Dekker, Inc. New York, NY, pp. 315-339, (1992).

Polymers can be used for ion-controlled release of atezolizumab-based compositions disclosed herein. Various degradable and nondegradable polymeric matrices for use in controlled drug delivery are known (Langer, Accounts Chem. Res. 26:537-542, 1993). For example, the block copolymer, poloxamer 407, exists as a viscous yet mobile liquid at low temperatures but forms a semisolid gel at body temperature. It is an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, PA (1993)). Numerous additional systems for controlled delivery of therapeutic proteins are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

### IV. Therapeutic Methods

The atezolizumab and compositions disclosed herein that include atezolizumab can be administered to slow or inhibit the growth of ASPS tumor cells or inhibit the metastasis of ASPS tumor cells. In these applications, a therapeutically effective amount of a composition is administered to a subject in an amount sufficient to reduce growth, replication or metastasis of ASPS cancer cells, or to reduce a sign or a symptom of ASPS. Suitable subjects may include those diagnosed with ASPS, such as a mammal, such as a human or veterinary subject.

Provided herein are methods of treating ASPS in a subject by administering to the subject a therapeutically effective amount of atezolizumab composition that includes atezolizumab disclosed herein. Also provided herein is a method of reducing ASPS tumor growth or metastasis of a ASPS in a subject by administering to the subject a therapeutically effective amount of atezolizumab or composition that includes atezolizumab disclosed herein.

The ASPS tumor does not need to be completely eliminated or inhibited for the method to be effective. For example, the method can decrease tumor size (e.g., volume) or metastasis by for example by at least 10%, at least 20%, at least 30%, at least 40%, at least 50%, at least 60%, at least 70%, at least 80%, at least 90%, at least 95%, at least 98%, at least 99%, or even 100% as compared to the absence of the treatment with ASPS (e.g., before a first dose/cycle of atezolizumab is administered).

A therapeutically effective amount of atezolizumab or composition that includes atezolizumab disclosed herein may depend upon the severity of ASPS and the general state of the patient's health. A therapeutically effective amount of the atezolizumab-based composition is that which provides either subjective relief of a symptom(s) or an objectively identifiable improvement as noted by the clinician or other qualified observer. Atezolizumab and conjugates thereof can be administered, for example, by intravenous infusion. In some aspects, dosages of 840 mg atezolizumab every two weeks, 1200 mg atezolizumab every 3 weeks, or 1680 mg atezolizumab every 4 weeks are used for adults. In some aspects, dosages of a 15 mg atezolizumab per kilogram of body weight every 3 weeks (not to exceed 1200 mg atezolizumab) are used for children under 18 years old and older than 2 years old. The atezolizumab or conjugate can be administered according to a dosing schedule determined by a medical practitioner. In some examples, the atezolizumab or conjugate is administered weekly, every two weeks, every three weeks, or every four weeks.

In some aspects, a subject is administered DNA or RNA encoding atezolizumab to provide in vivo antibody production, for example using the cellular machinery of the subject. Any suitable method of nucleic acid administration may be used; non-limiting examples are provided in U.S. Pat. Nos. 5,643,578, 5,593,972 and 5,817,637. U.S. Pat. No. 5,880,103 describes several methods of delivery of nucleic acids encoding proteins to an organism. One approach to administration of nucleic acids is direct administration with plasmid DNA, such as with a mammalian expression plasmid. The nucleotide sequence encoding atezolizumab, or antigen binding fragments thereof, can be placed under the control of a promoter to increase expression. The methods include liposomal delivery of the nucleic acids. Such methods can be applied to the production of atezolizumab or antigen binding fragments thereof.

In several aspects, a subject (such as a human subject with ASPS) is administered an effective amount of a viral vector that includes one or more nucleic acid molecules encoding atezolizumab. The viral vector is designed for expression of the nucleic acid molecules encoding atezolizumab, and administration of the effective amount of the viral vector to the subject leads to expression of an effective amount of atezolizumab in the subject. Non-limiting examples of viral vectors that can be used to express a disclosed antibody or antigen binding fragment in a subject include those provided in Johnson et al., Nat. Med., 15(8):901-906, 2009 and Gardner et al., Nature, 519(7541):87-91, 2015.

In one aspect, a nucleic acid encoding atezolizumab, or conjugate thereof, is introduced directly into tissue. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter.

Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

Single or multiple administrations of a composition including atezolizumab or atezolizumab conjugate, or nucleic acid molecule encoding such molecules, can be administered depending on the dosage and frequency as required and tolerated by the patient. The dosage can be administered once but may be applied periodically until either a desired result is achieved or until side effects warrant discontinuation of therapy. Generally, the dose is sufficient to reduce growth or metastasis of ASPS without producing unacceptable toxicity to the patient.

The atezolizumab, atezolizumab conjugate, nucleic acid molecule encoding such molecules, or a composition including such molecules, can be administered to subjects in various ways, including local and systemic administration, such as, e.g., by injection subcutaneously, intravenously, intra-arterially, intraperitoneally, intramuscularly, intradermally, intratumorally, or intrathecally. In some aspects, the composition is administered by inhalation, such as by using an inhaler. In one aspect, atezolizumab, antigen binding fragment, or nucleic acid molecule encoding such molecules, or a composition including such molecules, is administered by a single subcutaneous, intravenous, intra-arterial, intraperitoneal, intramuscular, intradermal or intrathecal injection every 2 weeks, every 3 weeks, or every 4 weeks. The atezolizumab, antigen binding fragment, bispecific antibody, conjugate, or nucleic acid molecule encoding such molecules, or a composition including such molecules, can also be administered by direct injection at or near the site of a ASPS tumor. A further method of administration is by osmotic pump (e.g., an ALZET pump) or mini-pump (e.g., an ALZET mini-osmotic pump), which allows for controlled, continuous and/or slow-release delivery of atezolizumab, atezolizumab conjugate, or nucleic acid molecule encoding such molecules, or a composition including such molecules, over a pre-determined period. The osmotic pump or mini-pump can be implanted subcutaneously, or near a target site.

In one example, atezolizumab is conjugated to IR700, and photoimmunotherapy is used to treat ASPS. For example, such a method can include administering to the subject with ASPS a therapeutically effective amount of one or more atezolizumab-IR700 conjugates. Following administration of the conjugate, irradiation is performed at a wavelength of 660 to 740 nm (such as 660 to 710 nm, for example, 680 nm) and at a dose of at least 1 J cm⁻² (such as at least 1 to about 50 J cm⁻²) thereby treating the ASPS cancer in the subject. In some examples, the ASPS tumor is irradiated at a wavelength of 660 to 740 nm (such as 660 to 710 nm, for example, 680 nm) at a dose of at least 1 J cm⁻² (such as at least 1 J cm⁻², at least 4 J cm-2, at least 10 J cm-2, at least 50 J cm⁻², or at least 100 J cm⁻²) thereby treating the ASPS in the subject. In some examples, multiple rounds of treatment are performed, such as 2, 3, 4, 5, 6, 7, 8, 9 or 10 treatment cycles. In particular examples, a therapeutically effective dose of a atezolizumab-IR700 conjugates is at least 0.5 milligram per 60 kilogram (mg/kg), at least 5 mg/60 kg, at least 10 mg/60 kg, at least 20 mg/60 kg, at least 30 mg/60 kg, at least 50 mg/60 kg, for example 0.5 to 50 mg/60 kg, such as a dose of 1 mg/60 kg, 2 mg/60 kg, 5 mg/60 kg, 20 mg/60 kg, or 50 mg/60 kg, for example when administered intravenously. In another example, a therapeutically effective dose of an atezolizumab-IR700 conjugates is at least 10 μg/kg, such as at least 100 μg/kg, at least 500 μg/kg, or at least 500 μg/kg, for example 10 μg/kg to 1000 μg/kg, such as a dose of 100 μg/kg, 250 μg/kg, about 500 μg/kg, 750 μg/kg, or 1000 μg/kg, for example when administered i.p. In one example, a therapeutically effective dose of an atezolizumab-IR700 conjugates is at least 1 μg/ml, such as at least 500 μg/ml, such as between 20 μg/ml to 100 μg/ml, such as 10 μg/ml, 20 μg/ml, 30 μg/ml, 40 μg/ml, 50 μg/ml, 60 μg/ml, 70 μg/ml, 80 μg/ml, 90 μg/ml or 100 μg/ml administered in a topical solution.

In some aspects, the treatment methods further include administration of other anti-cancer agents or therapeutic treatments. Any suitable anti-cancer agent can be administered in combination with atezolizumab. Exemplary anti-cancer agents include, but are not limited to, chemotherapeutic agents, such as, for example, mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones (e.g., anti-androgens) and anti-angiogenesis agents. Other anti-cancer treatments include radiation therapy and other antibodies that specifically target cancer cells.

Non-limiting examples of alkylating agents that can be used in combination with atezolizumab include nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine).

Non-limiting examples of antimetabolites that can be used in combination with atezolizumab include folic acid analogs (such as methotrexate), pyrimidine analogs (such as 5-FU or cytarabine), and purine analogs, such as mercaptopurine or thioguanine.

Non-limiting examples of natural products that can be used in combination with atezolizumab include vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitomycin C), and enzymes (such as L-asparaginase).

Non-limiting examples of other agents that can be used in combination with atezolizumab include platinum coordination complexes (such as cis-diamine-dichloroplatinum II also known as cisplatin), substituted ureas (such as hydroxyurea), methyl hydrazine derivatives (such as procarbazine), and adrenocrotical suppressants (such as mitotane and aminoglutethimide).

Non-limiting examples of hormones and antagonists that can be used in combination with atezolizumab include adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testosterone propionate and fluoxymesterone). Exemplary chemotherapy drugs that can be used in combination with atezolizumab include Adriamycin, Alkeran, Ara-C, BiCNU, Busulfan, CCNU, Carboplatinum, Cisplatinum, Cytoxan, Daunorubicin, DTIC, 5-FU, Fludarabine, Hydrea, Idarubicin, Ifosfamide, Methotrexate, Mithramycin, Mitomycin, Mitoxantrone, Nitrogen Mustard, Taxol (or other taxanes, such as docetaxel), Velban, Vincristine, VP-16, Gemcitabine (Gemzar), Herceptin, Irinotecan (Camptosar, CPT-11), Leustatin, Navelbine, Rituxan STI-571, Taxotere, Topotecan (Hycamtin), Xeloda (Capecitabine), Zevelin and calcitriol.

Non-limiting examples of immunomodulators that can be used in combination with atezolizumab include AS-101, bropirimine, gamma interferon, GM-CSF (granulocyte macrophage colony stimulating factor), IL-2, human immune globulin, IMREG, SK&F 106528, and TNF (tumor necrosis factor).

Exemplary biologics that can be used in combination with atezolizumab include one or more additional monoclonal antibodies (mAbs) used to treat cancer, such as mAbs specific for EGFR (e.g., cetuximab), VEGF (e.g., bevacizumab), PD-1 (e.g., nivolumab, JTX-4014 by Jounce Therapeutics, nivolumab, pembrolizumab, pidilizumab, cemiplimab, spartalizumab (PDR001), camrelizumab (SHR1210), sintilimab (IBI308), tislelizumab (BGB-A317), toripalimab (JS 001, dostarlimab (TSR-042, WBP-285), INCMGA00012 (MGA012), AMP-224, or AMP-514), CD25 (e.g., daclizumab or basiliximab), CD20 (e.g., Tositumomab (Bexxar®); Rituximab (Rituxan, Mabthera); Ibritumomab tiuxetan (Zevalin, for example in combination with yttrium-90 or indium-111 therapy); Ofatumumab (Arzerra®), veltuzumab, obinutuzumab, ublituximab, ocaratuzumab), CD22 (e.g., narnatumab, inotuzumab ozogamicin, moxetumomab pasudotox) or CTLA4 (e.g., ipilimumab, tremelimumab). In some examples, the additional therapeutic agent administered is an anti-cancer monoclonal antibody, for example one or more of: 3F8, Abagovomab, Adecatumumab, Afutuzumab, Alacizumab, Alemtuzumab, Altumomab pentetate, Anatumomab mafenatox, Apolizumab, Arcitumomab, Bavituximab, Bectumomab, Belimumab, Besilesomab, Bevacizumab, Bivatuzumab mertansine, Blinatumomab, Brentuximab vedotin, Cantuzumab mertansine, Capromab pendetide, Catumaxomab, CC49, Cetuximab, Citatuzumab bogatox, Cixutumumab, Clivatuzumab tetraxetan, Conatumumab, Dacetuzumab, Detumomab, Ecromeximab, Eculizumab, Edrecolomab, Epratuzumab, Ertumaxomab, Etaracizumab, Farletuzumab, Figitumumab, Galiximab, Gemtuzumab ozogamicin, Girentuximab, Glembatumumab vedotin, Ibritumomab tiuxetan, Igovomab, Imciromab, Intetumumab, Inotuzumab ozogamicin, Ipilimumab, Iratumumab, Labetuzumab, Lexatumumab, Lintuzumab, Lorvotuzumab mertansine, Lucatumumab, Lumiliximab, Mapatumumab, Matuzumab, Mepolizumab, Metelimumab, Milatuzumab, Mitumomab, Morolimumab, Nacolomab tafenatox, Naptumomab estafenatox, Necitumumab, Nimotuzumab, Nofetumomab merpentan, Ofatumumab, Olaratumab, Oportuzumab monatox, Oregovomab, Panitumumab, Pemtumomab, Pertuzumab, Pintumomab, Pritumumab, Ramucirumab, Rilotumumab, Rituximab, Robatumumab, Satumomab pendetide, Sibrotuzumab, Sonepcizumab, Tacatuzumab tetraxetan, Taplitumomab paptox, Tenatumomab, TGN1412, Ticilimumab (tremelimumab), Tigatuzumab, TNX-650, Trastuzumab, Tremelimumab, Tucotuzumab celmoleukin, Veltuzumab, Volociximab, Votumumab, Zalutumumab, or combinations thereof. In a specific example, the disclosed methods are used in combination with another anti-PD-L1 antibody (e.g., avelumab, durvalumab, cosibelimab, KN035 (envafolimab), BMS-936559, BMS935559, MEDI-4736, MPDL-3280A, or MEDI-4737).

In some examples, the methods further include surgical treatment, for example surgical resection of the ASPS or a portion of it. In some examples, the methods further include administration of radiotherapy, for example administration of radioactive material or energy (such as external beam therapy) to the tumor site to help eradicate the tumor or shrink it prior to surgical resection.

### EXAMPLES

The following examples are provided to illustrate particular features of certain aspects of the disclosure, but the scope of the claims should not be limited to those features exemplified.

### Example 1: Treatment of ASPS with Atezolizumab: Materials and Methods

Trial Design and Oversight: Eligible patients were ≥2 years old with histologically or cytologically confirmed ASPS (including newly diagnosed, unresectable or metastatic, and measurable disease with clinical evidence of disease progression) who had no prior anti-PD-1/anti-PD-L1 therapy. Atezolizumab was administered intravenously at a dose of 1200 mg (adult patients, ≥18 years old) or 15 mg/kg with a 1200 mg cap (pediatric patients, <18 years old) on the first day of each 21-day cycle. Atezolizumab doses were allowed to be held for up to 12 weeks for toxicity resolution; dose modifications were not permitted, given the lack of a clear atezolizumab dose-response relationship. Details regarding eligibility, dose interruptions, and treatment discontinuation can be found in the protocols in Examples 3 and 4. Tumor biopsies, which were made mandatory later in the trial, were collected at baseline and just prior to Cycle 3 Day 1 from patients ≥18 years old. Circulating tumor cells were collected from patients ≥14 years of age at baseline, on Cycle 1 Day 8, prior to the atezolizumab dose at the start of each subsequent cycle, and at disease progression. Patients on treatment for more than two years had the option of taking a break from treatment while retaining the chance to resume therapy at the discretion of the study investigators or upon evidence of progressive disease. Patient monitoring and response assessments continued throughout the break. Patients who completed two years of a treatment break without resuming atezolizumab were removed from the treatment portion of the trial, with continued assessments every 6 months.

This trial was conducted at multiple sites (ClinicalTrials.gov Identifier: NCT03141684). The trial was conducted in full accordance with Good Clinical Practice guidelines, the Declaration of Helsinki, and all applicable regulations, guidance, and local policies. Written informed consent was obtained from each adult patient and from the parent or guardian of each pediatric patient. Pediatric patients were included in discussions about the trial and their verbal or written assent was obtained. End Points and Assessments: To determine the objective response rate (ORR), duration of response, and progression-free survival (PFS), tumor response to drug was assessed by investigator review at each trial site using Response Evaluation Criteria in Solid Tumors (RECIST) version 1.1 (Eisenhauer et al., Eur J Cancer 2009; 45:228-47). Immune RECIST (iRECIST) criteria, which reflect the propensity of immune modulating agents to elicit robust tumor responses after initial evidence of disease progression (Seymour et al., Lancet Oncol 2017; 18:e143-e152), were also employed as an exploratory endpoint. Tumor measurements for response assessment were conducted at baseline, at the end of Cycle 3, every two cycles thereafter for the first year, every three cycles during the second year, and every four cycles after that. Tumors were measured using computed tomography or, in select cases, magnetic resonance imaging or positron emission tomography-computed tomography. Confirmatory scans were performed ≥4 weeks following initial documentation of objective response. Patients showing evidence of clinical benefit could continue atezolizumab treatment following RECIST 1.1 progression if they met protocol-specified criteria for drug continuation. For those patients treated beyond RECIST-assessed progression, all subsequent responses were assessed using iRECIST.

Adverse events were reported according to NCI Common Toxicity Criteria version 4.0 until Mar. 31, 2018, when version 5.0 was subsequently implemented and utilized; all prior adverse events were mapped to version 5.0 before the final analysis.

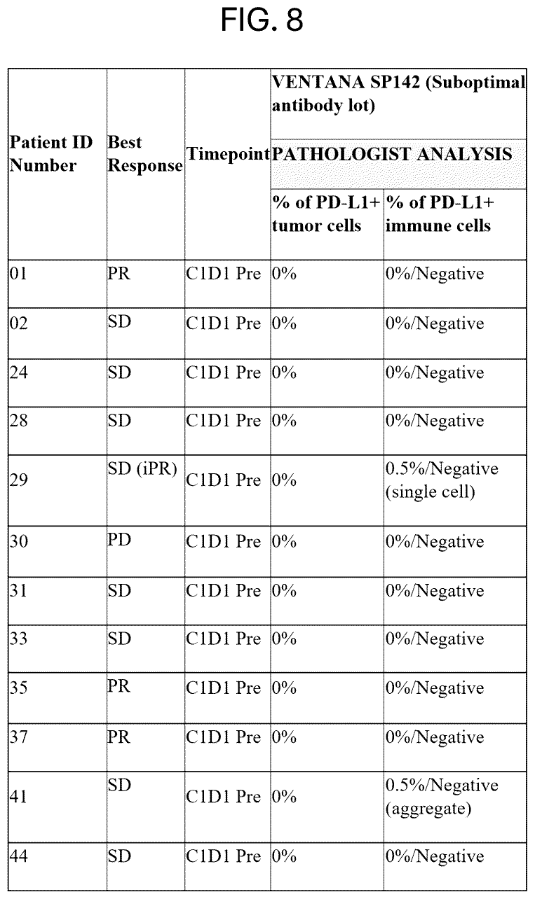

Paired biopsies (pre-treatment and Cycle 3 Day 1) were flash-frozen upon collection, thawed under formalin fixative, and processed for analysis of sections of paraffin blocks in a manner that preserves labile phosphoproteins (Herrick et al., Mol Cancer Ther 2021; 20:749-760; Srivastava et al., Ann Transl Med 2017; 5:3). Immunofluorescence microscopy of biopsy sections stained with multiplexed antibody panels (PD-L1/CD8; PD-1/CD3; CD4/FOXP3; CD8/CD3ζpY142/TFE3) utilized the PD-L1 monoclonal antibody clone 73-10, which provides greater sensitivity than the VENTANA SP142 clone employed in the diagnostic assay (FIG. 8; Tsao et al., J Thorac Oncol 2018; 13:1302-1311). Image analysis algorithms identified and quantified biomarker-positive cells per mm² within the entire tumor area and at stroma interfaces. PD-L1 expression in circulating tumor cells was analyzed by imaging flow cytometry.

Type 1 and Type 2 ASPL-TFE3 fusions were identified retrospectively using ribonucleic acid extracted from formalin-fixed, paraffin-embedded tissue and paraformaldehyde-fixed peripheral blood samples.

Statistical Analysis: This phase 2 study employed a Simon two-stage optimal design; the trial was to be stopped for futility if no responses were measured among the first nine patients. Otherwise, 15 additional patients were to be enrolled. If at least three responses were observed among the 24 total patients, the regimen was to be considered worthy of further testing in this disease. This design has 90% power to reject a null response rate of 0.05 when the true response rate is 0.25 (with one-sided type-I error of 0.093). All eligible patients who received at least one dose of study medication were included in the interim and primary analyses of the response rate. Proceeding to the second stage of the two-stage design required approval from the study sponsor, principal investigator, and statistician. After completion of the initial two-stage design, the accrual ceiling was increased to 53 to ensure collection of at least ten evaluable research biopsy pairs to confirm drug mechanism of action. This new accrual ceiling accounted for the possibilities of unpaired biopsies, biopsies of insufficient quality, and enrollment of patients who might not contribute biopsies (e.g., pediatric patients).

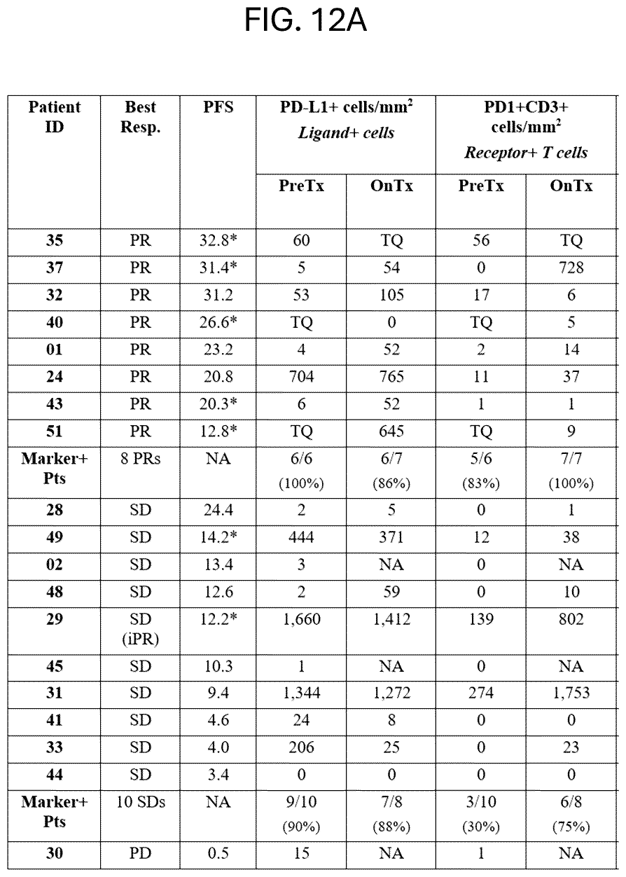

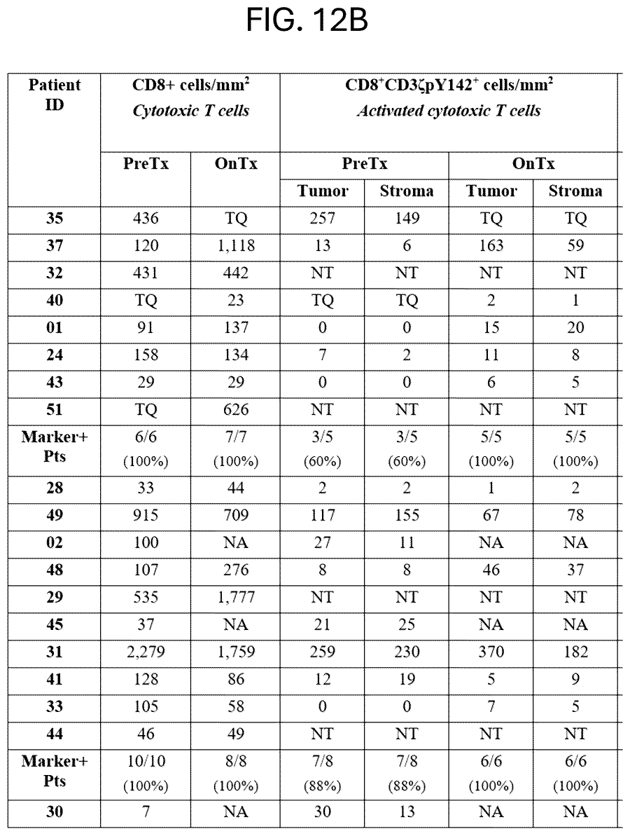

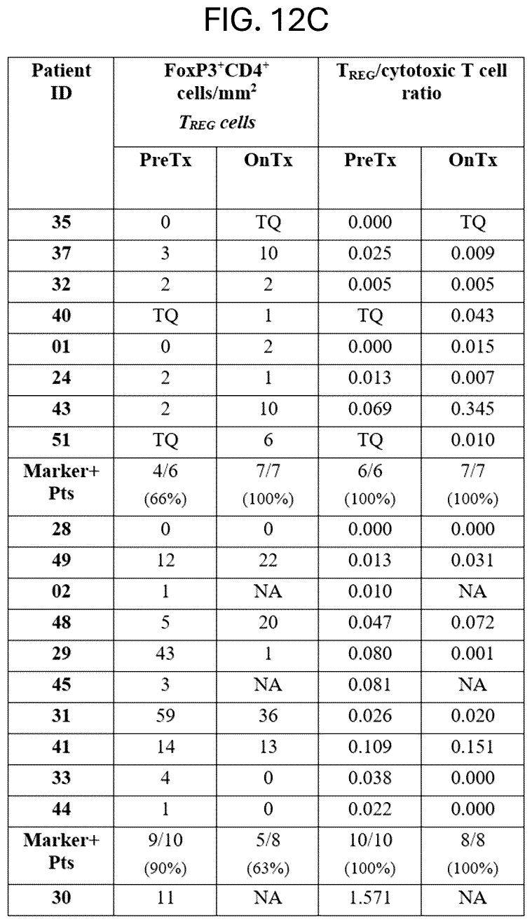

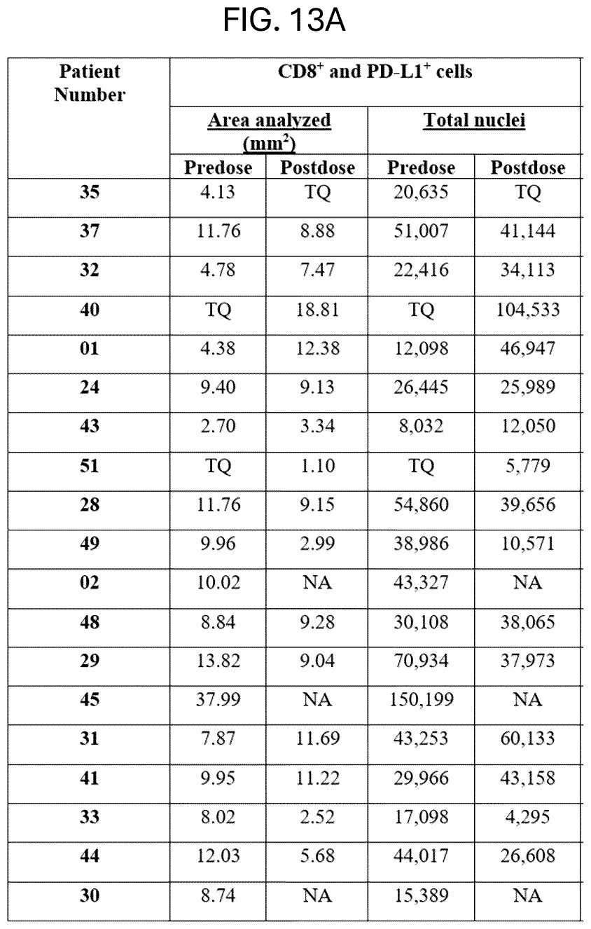

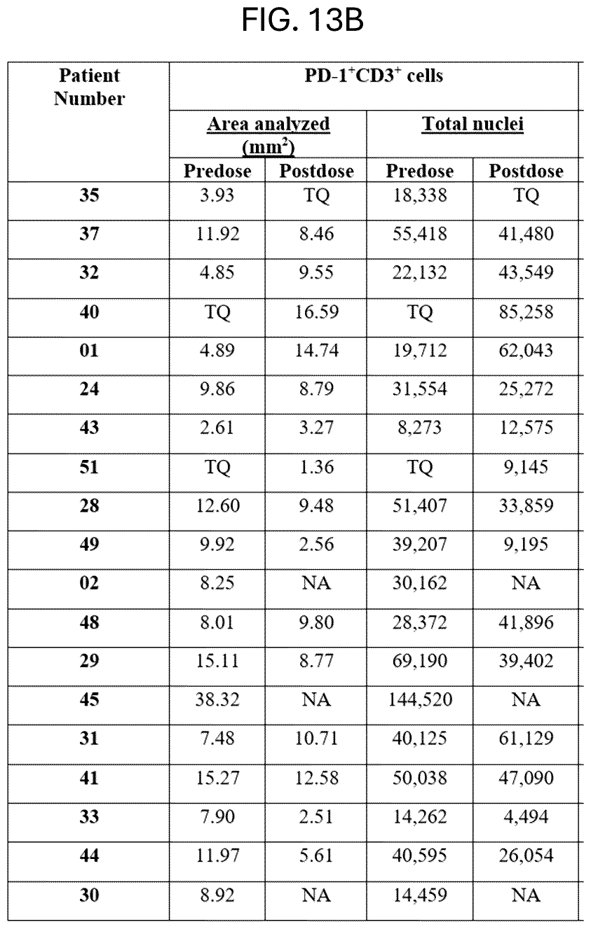

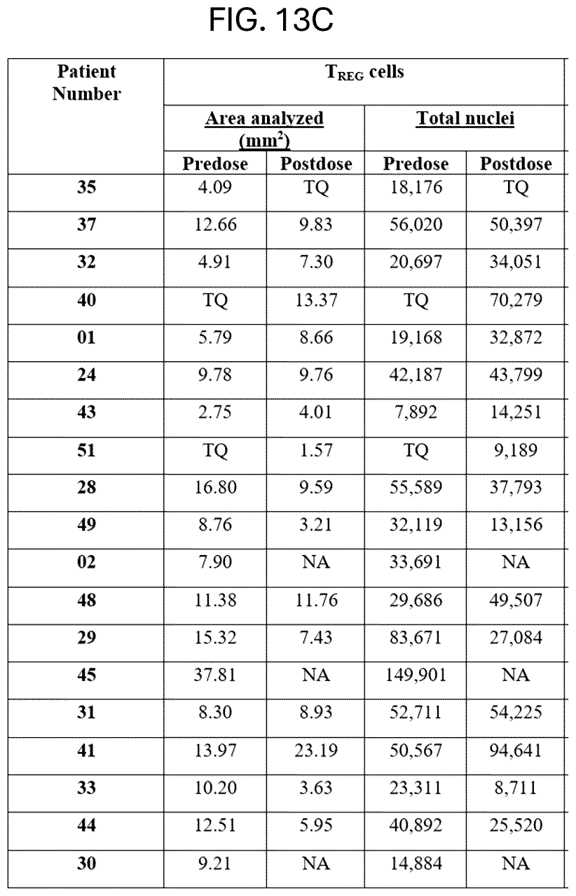

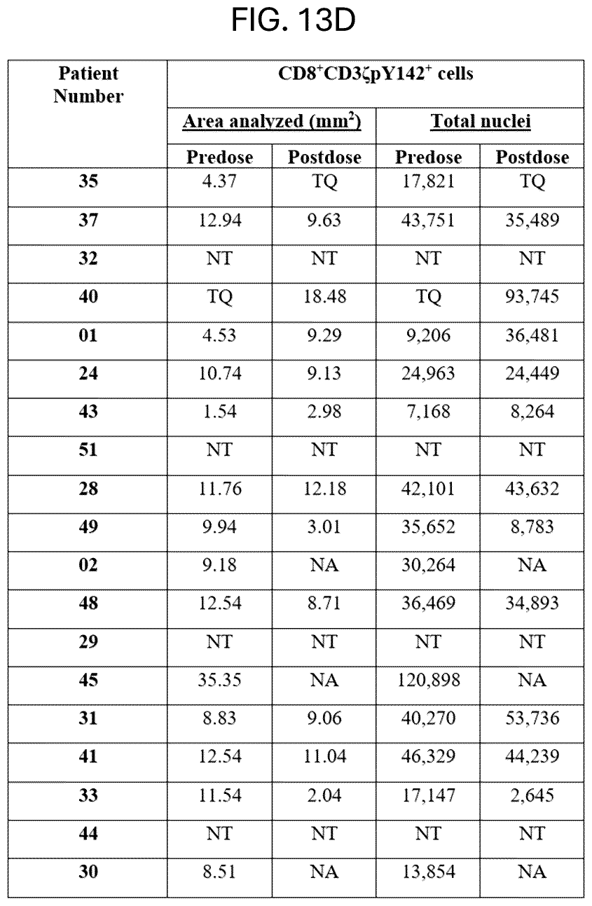

Multiplex immunofluorescence microscopy assay (mIFA) analysis of immune checkpoint inhibitor-related pharmacodynamic biomarkers in tumor biopsy specimens: Tumor biopsies were collected at baseline (after study enrollment but before the first atezolizumab dose) and prior to treatment on Cycle 3 Day 1 (18-gauge needle cores, 1-5 cores per patient per timepoint). All specimens were placed in pre-chilled vials and flash-frozen within 2 minutes of collection then stored at or below −80° C. until processing. Biopsy cores representing each timepoint were processed in parallel together with control tissues by thawing in 10% neutral buffered formalin, followed by formalin-fixed paraffin-embedded (FFPE) processing, per DCTD SOP 340550 that preserves labile phosphoproteins (Herrick et al., Mol Cancer Ther 2021; 20:749-760; Srivasta et al., Ann Transl Med 2017; 5:3), except that human tonsil control tissue and ParaPro XLT paraffin (StatLab) were used. Tissue sections (4 m each) were stained with PD-L1, PD-1, or T-Reg FixVUE™ panels (Ultivue), which utilize a proprietary technology employing DNA barcode-conjugated primary monoclonal antibodies—PD-L1 (73-10), CD8 (C8/144B), CD3 (BC33), PD-1 (CAL20), CD4 (SP35), and FoxP3 (236A/E7)—as well as 4′6-diamidino-2-phenylindole (DAPI) and the BOND RX automated slide stainer (Leica Biosystems), as has been described previously (Wu et al., J Clin Pathol 2022; 75:452-458; Wu et al., Modern Pathology 2022; 35:218-227). Two custom markers were also evaluated using the Ultivue platform: (i) tyrosine 142-phosphorylated CD3ζ(CD3ζ-pY142), assessed using monoclonal antibody K25-407.69 (BD Biosciences); and (ii) nuclear TFE3 (MRQ-37, Cell Marque Corporation), which was used for image segmentation of ASPS tumor tissue from the surrounding non-tumor tissue (Argani et al., Am J Surg Pathol 2003; 27:750-61; Folpe et al., J Clin Pathol 2006; 59:1127-32). Each staining run included prescreened human tonsil tissue as a control for PD-L1+, PD1+CD3+, CD8+, FoxP3+CD4+, or CD3ζ-pY142+CD8+ cells and ASPS-1 cell pellets as a control for TFE3+ and PD-L1+ cells. The entire area of each tissue section was scanned at 20× magnification using a ZEISS Axio Scan.Z1 Slide Scanner and ZEISS Colibri 7 LED light source (Carl Zeiss Spectroscopy GmbH). Regions of interest covering the entire tumor area and its invasive margins in each tissue section were extracted and imported into Definiens Architect (Definiens AG) for image analysis employing nuclear segmentation (using the DAPI layer) and customized cellular analysis with marker thresholding to determine cellular densities (cells/mm²) for PD-L1+, PD-1+CD3+, CD8+, CD4+ and FoxP3+CD4+ cell populations. Each cell density value was calculated as the total number of biomarker-positive cells within a tissue section, divided by the entire area (mm²) of viable tumor and invasive margin in the section (FIGS. 13A-13D), and each reported cell density value represents the measurement from 1-2 tissue cores (1 tissue section per core). Analyzed areas per section ranged from 1.1 to 23 mm², and analyzed cell numbers per section (based on total nuclei counted) ranged from 2,645 to 104,533. An individual biopsy specimen was considered positive for each biomarker if it contained ≥16 PD-L1+ cells, ≥3 PD1+CD3+ cells, ≥61 CD8+ cells, and >5 Treg cells, which converts mathematically into the uniform cutoff of ≥1 biomarker-expressing cell per mm² of analyzed tissue area given the smallest specimen that was evaluable with each biomarker (FIGS. 12A-12C, 13A-13D). Images stained with a custom multiplex panel employing nuclear TFE3 as a marker of ASPS tumor cells and HALO and HALO AI software platforms (Indica Labs) were used to develop a tissue classifier algorithm to segment tumor and surrounding stromal areas in collaboration with Indica Labs. The custom multiplex panel included CD3ζ-pY142, enabling quantitation of the cellular density of CD8+ cells with activated T cell receptor signaling (CD3ζ-pY142+CD8+ cells) within the tumor microenvironment.

Circulating tumor cell PD-L1 analysis: Staining and gating controls for circulating tumor cell (CTC) analysis were established using a human alveolar soft part sarcoma cell line (ASPS-1) as a positive control for PD-L1-expressing cells, as well as peripheral blood mononuclear cells (PBMCs) isolated from healthy donor whole blood specimens, via the AccuCyte Sample Preparation System (RareCyte) as described below. The ASPS-1 cell line was obtained from the NCI Division of Cancer Treatment and Diagnosis (DCTD) Tumor Repository. ASPS-1 cells were cultured in DMEM/F-12 supplemented with 10% FBS. Cell cultures were maintained at 37° C. with 5% CO₂ and 100% humidity. Cells were fixed with 2% formaldehyde and stored in 70% ethanol at −20° C. prior to analysis. Whole blood from consenting healthy donors was obtained from the Research Donor Program, Occupational Health Services, Frederick National Laboratory for Cancer Research (FNCLR).

For CTC analysis of clinical trial samples, 7.5-mL venous blood specimens were collected from patients prior to atezolizumab administration on cycle 1 day 1 (C1D1), prior to atezolizumab administration at the start of C2 and every subsequent cycle, and at time of disease progression. Blood specimens were collected into Cell-Free DNA BCT RUO blood collection tubes (Streck) and shipped and processed within 48 hours of collection, according to DCTD SOP LHTP003.08.19 (dctd.cancer.gov/researchresources/researchresources-biomarkers.htm). For each blood specimen, the PBMC fraction was isolated from 7.5 mL whole blood using the density-based AccuCyte Sample Preparation System (RareCyte) per the manufacturer's instructions. Cells were fixed with 2% formaldehyde and stored in 70% ethanol at −20° C. prior to analysis.

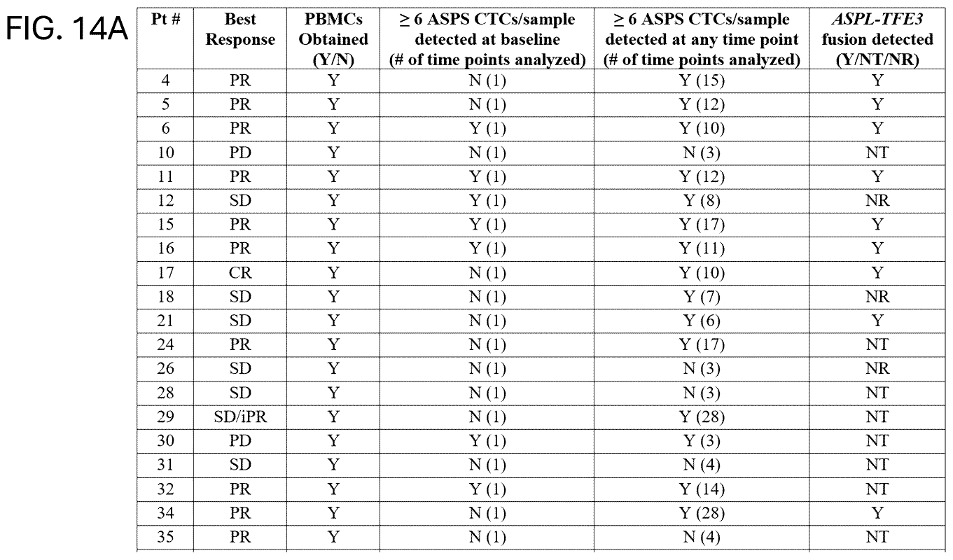

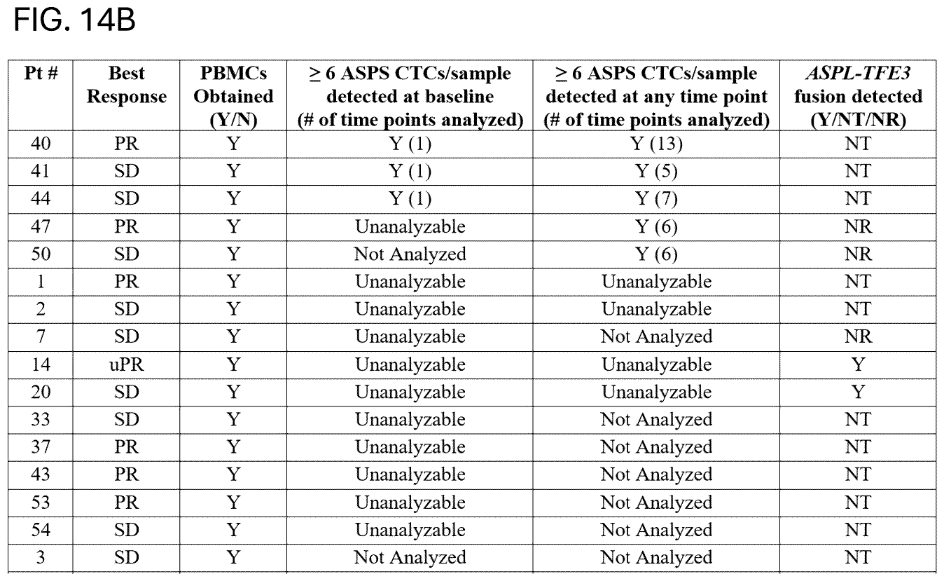

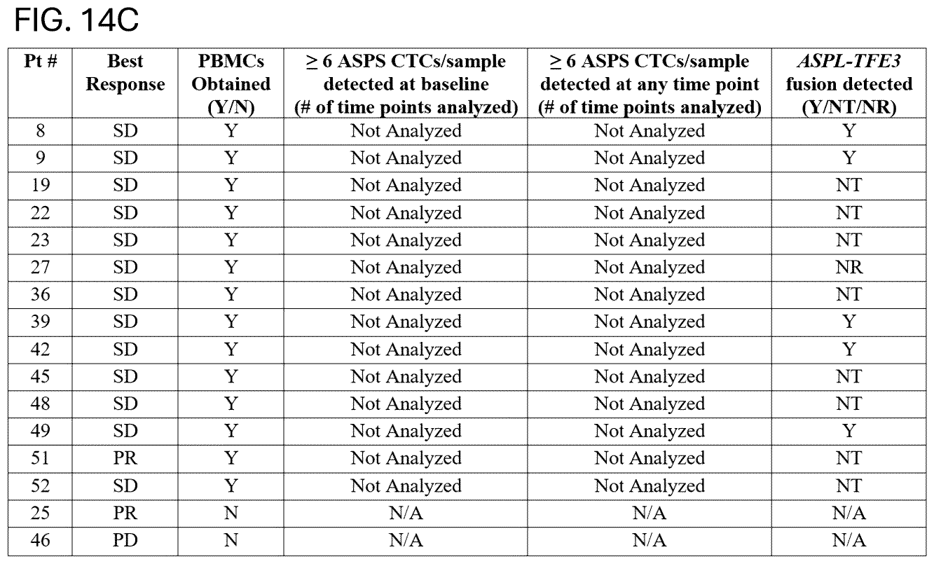

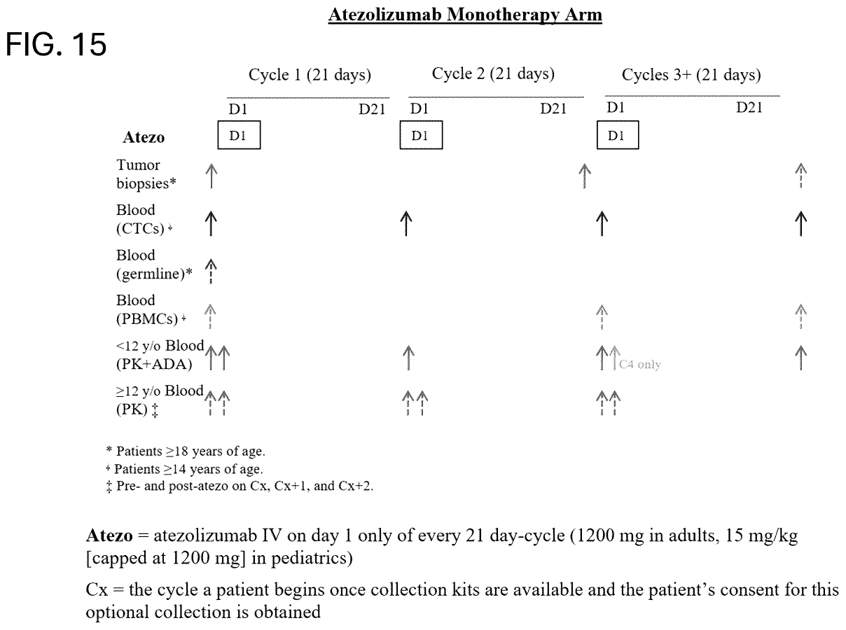

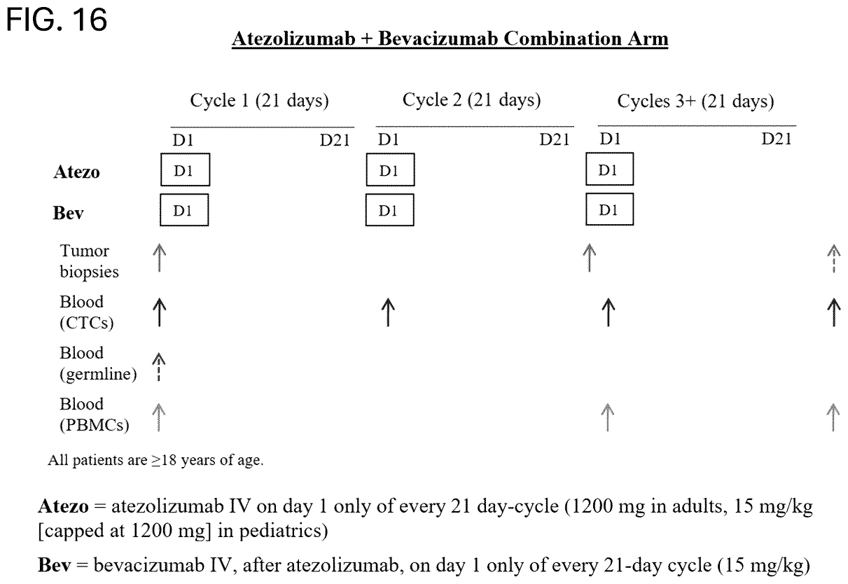

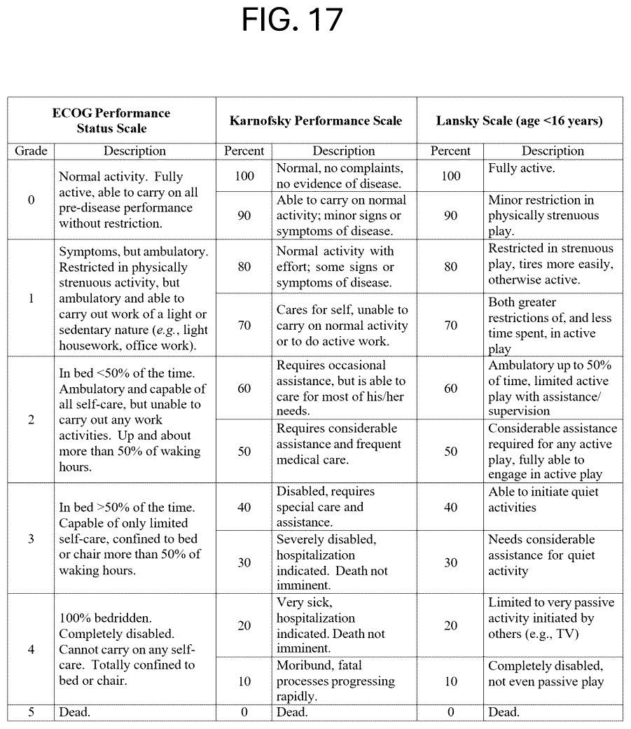

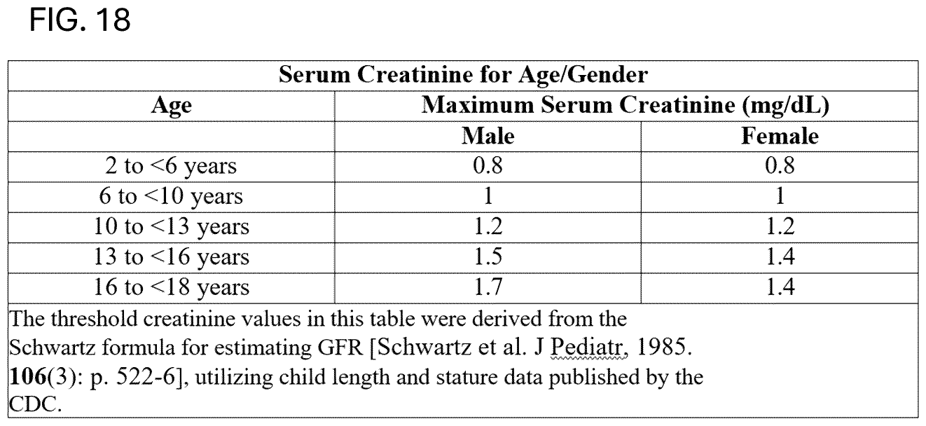

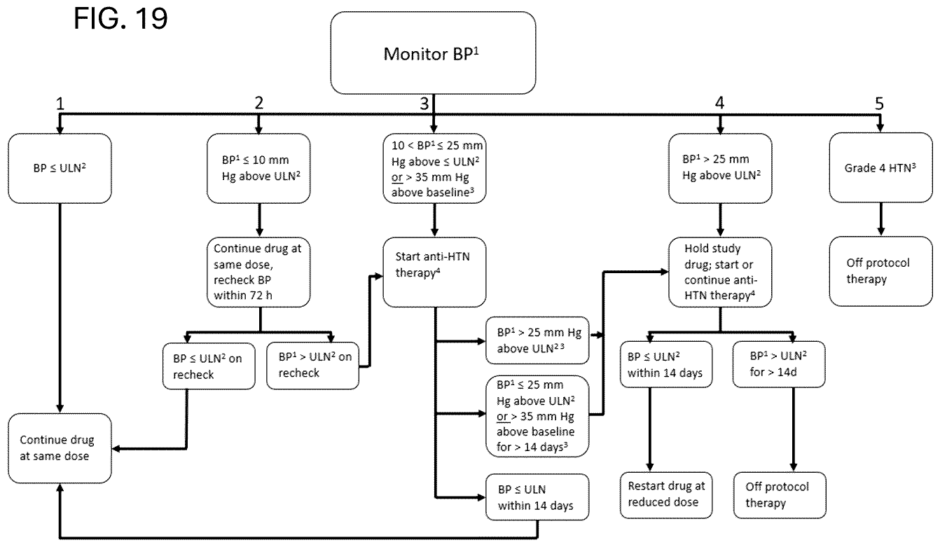

For immunofluorescence microscopy-based imaging flow cytometry analysis of CTCs and measurement of PD-L1 expression, antibodies to the following proteins were purchased from commercial vendors: TFE3 (clone MRQ-37, OriGene), CD45 (Brilliant Violet 510-conjugated clone HI30, BioLegend), and PD-L1 (Alexa Fluor 647-conjugated clone 28-8, Abcam). The TFE3 antibody was custom-conjugated to Alexa Fluor 488 (Invitrogen). Cell samples stored in 70% ethanol were rehydrated in DPBS (Gibco) and then stained in suspension. Samples were blocked with Human Fc Block (BD Pharmingen) and 2% BSA (bovine serum albumin, Thermo Scientific catalog #37525), stained with a cocktail of all three fluorescence-conjugated antibodies, and resuspended in a DAPI solution (Invitrogen) to stain nuclei. For patient specimens, 30-50% of each sample (corresponding to 2.25-3.75 mL blood) was stained and imaged. Images were acquired at 40× magnification using an Amnis ImageStreamX Mk II Imaging Flow Cytometer (Luminex) equipped with 405, 488, 561, and 642 nm lasers plus brightfield and side scatter. Analysis was performed using Amnis IDEAS software (Luminex). Cell markers were gated based on total intensity. CTCs were defined as cells that contained a nucleus (DAPI-positive) and were tumor marker (TFE3)-positive (Argani et al., Am J Surg Pathol 2003; 27:750-61; Folpe et al., J Clin Pathol 2006; 59:1127-32) and pan-leukocyte marker (CD45)-negative. CTCs were further divided into subpopulations based on PD-L1 expression. All CTC counts are expressed as number of cells per 7.5 mL blood. As previously established in prior clinical studies of pharmacodynamic responses of p16 and γH2AX biomarkers in CTCs of other tumor histologies (Coyne et al., Cancer Chemother Pharmacol 2020; 85:979-993; Wang et al., Clin Cancer Res 2010; 16:1073-84; Kummar et al., Clin Cancer Res 2012; 18:1726-34), each blood specimen was prospectively considered evaluable for the PD-L1 biomarker if it contained ≥6 ASPS CTCs (defined as TFE3-positive, CD45-negative cells in the isolated PBMC fraction) (FIGS. 14A-14C). PBMC fractions that were positive for ASPS CTCs were submitted for follow-on analysis of tumor cell expression of PD-L1; ASPS-CTC-negative specimens were not analyzed further. A patient was designated positive for PD-L1-expressing ASPS if there was persistent, high expression of PD-L1 in ASPS CTCs-defined as ≥70% PD-L1-positive ASPS CTC cells (i.e., >4 PD-L1-positive CTCs in the minimum of 6 ASPS CTCs) for at least 2 consecutive cycles of therapy.

Detection of Type 1 and Type 2 ASPL-TFE3 fusions via RT-PCR: ASPL-TFE3 fusion types were identified by RT-PCR using total RNA extracted from tumor tissue biopsy orif tissue was not available—from the buffy coat fraction of circulating tumor cell-positive venous blood specimens. ASPL-TFE3 fusion typing from venous blood samples was facilitated by the fact that the fusion gene is not present in normal cells and by the relatively high levels of circulating tumor cells in these ASPS samples, compared to what has been observed in various carcinomas (Coyne et al., Cancer Chemother Pharmacol 2020; 85:979-993; Wang et al., Clin Cancer Res 2010; 16:1073-84).

Total RNA was extracted from RBC-depleted fixed peripheral blood mononuclear cell fractions (PBMCs) using the Absolutely RNA FFPE Kit (without deparaffinization reagents) (Agilent technologies, Cat #400811) following the manufacturer protocol. Total RNA extraction from FFPE scrapes or curls (depending on available material) was conducted using Qiagen's AllPrep DNA/RNA FFPE Kit and the manufacturer's protocol. cDNA was synthesized using TaqMan™ Reverse Transcription reagent following the manufacturer's protocol (ThermoFisher Scientific).

For RT-PCR, the sequences for primer set 1, primer set 2, and beta-actin were as described (Williams et al., Virchows Arch 2011; 458:291-300). 0.2-20 ng cDNA derived from biopsy specimens was used per PCR reaction. 75 ng-1 μg cDNA derived from fixed PBMCs was used per PCR reaction. RT-PCR for both fusion transcripts was conducted using 2×PCR Master Mix (cat #K0171; ThermoFisher Scientific). The details of each PCR reaction are listed in Table 1; the annealing temperature was 57° C. and each reaction was 40 cycles.

TABLE 1
PCR Reactions

| Type 1/Type 2 | | | Expected PCR |
|---|---|---|---|
| fusion primer set | Forward primer | Reverse primer | product size |
| ASPL/TFE3 Type | CCAAGCCAAAGAAGTCCAAG | CAAGCAGATTCCCTGACACA | Type 1: 120 bp |
| 1/2 (Primer set 1) | (SEQ ID NO: 1) | (SEQ ID NO: 2) | Type 2: 225 bp |
| ASPL/TFE3 Type | GCCAAAGAAGTCCAAGTCG | GGACTCCAGGCTGATGATCT | Type 2: 130 bp |
| 2 (Primer set 2) | (SEQ ID NO: 3) | (SEQ ID NO: 4) | |
| β-actin control | ATCACCATTGGCAATGAGCG | TTGAAGGTAGTTTCGTGGAT | 98 |
| | (SEQ ID NO: 5) | (SEQ ID NO: 6) | |

PCR product was diluted 1:10 in 1×E-Gel™ Sample Loading Buffer (cat #10482055) before gel electrophoresis using a 2% E-Gel™ EX Agarose Gel (cat #G401002) on the E-Gel™ Power Snap Electrophoresis System (cat ##G8300, ThermoFisher Scientific) with E-Gel Low Range (cat #12373031, ThermoFisher) and E-Gel™ 50 bp (cat #10488099, ThermoFisher) DNA ladders.

After fusion-typing results were reviewed for the first 18 patients, data was collected for 25 additional patients to enable a post hoc exploratory analysis.

### Example 2: Treatment of ASPS with Atezolizumab—Study Results

The details of the results described herein can also be found in Chen et al., Atezolizumab for advance alveolar soft part sarcoma, NEJM, 389:911-21, 2023, including the supplementary materials, all herein incorporated by reference in their entireties.

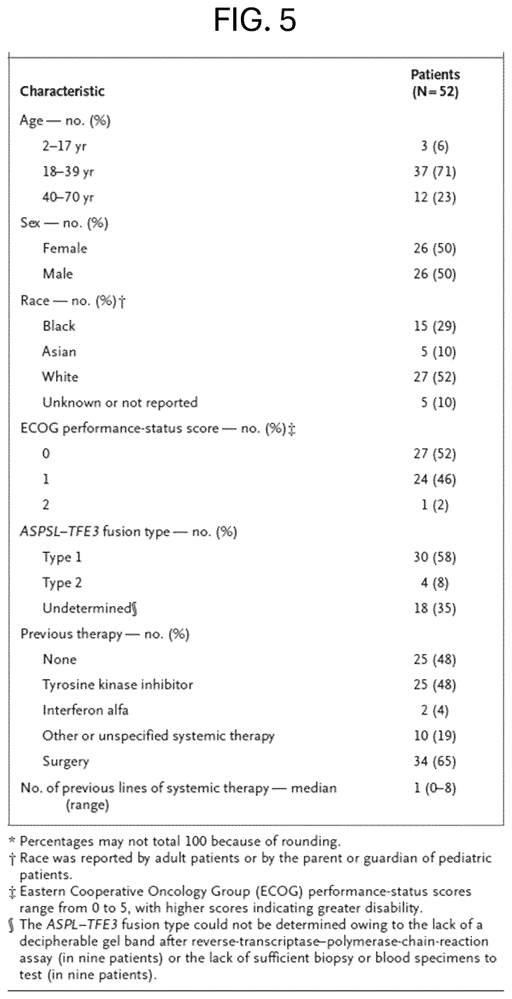

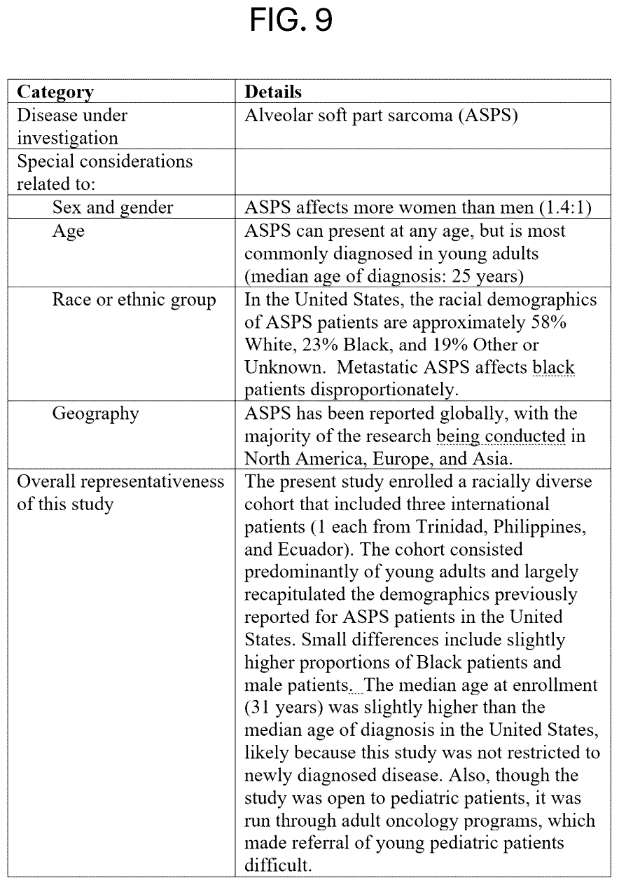

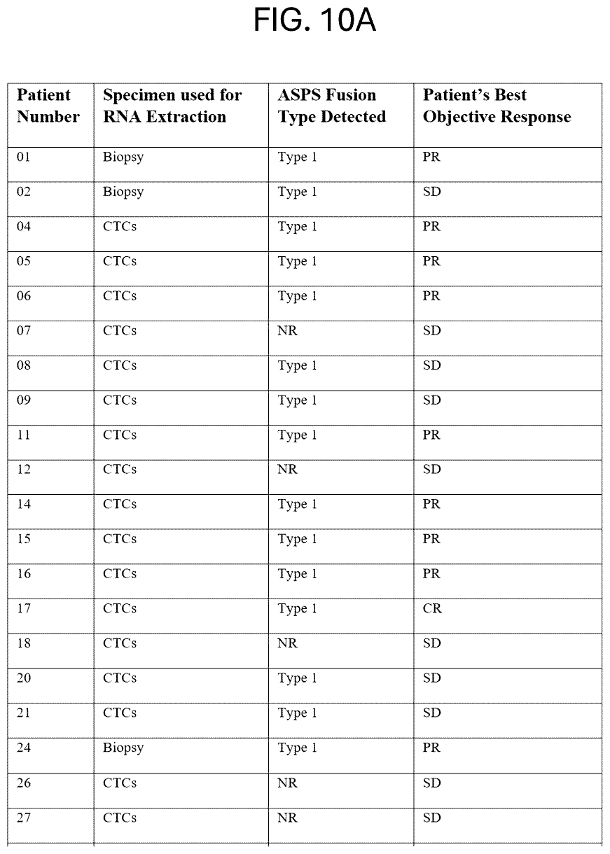

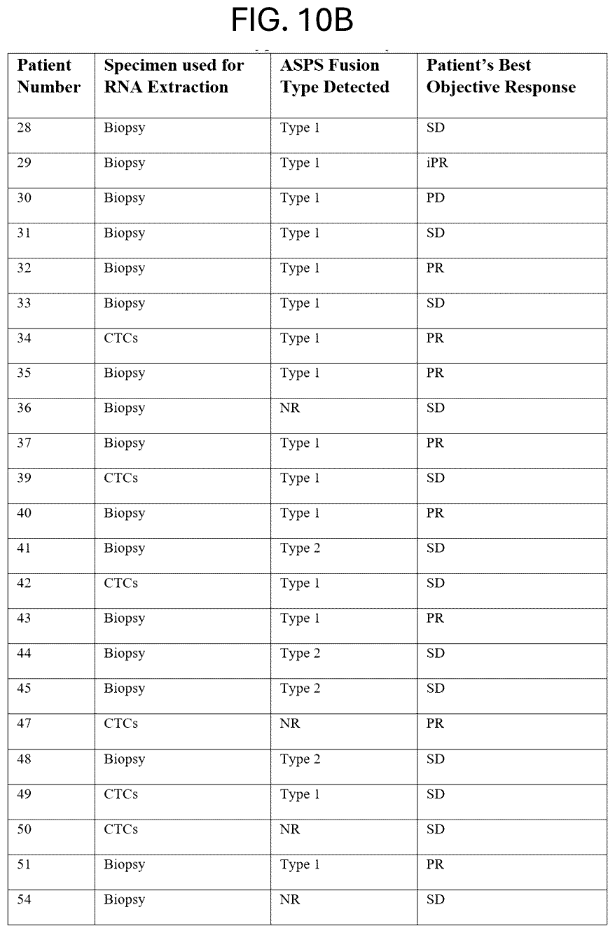

Patients: From April 2017 through July 2022, 53 patients were enrolled across 17 centers; 52 initiated treatment (FIG. 5). The racial diversity within the cohort reflects the racial distribution of patients with advanced ASPS, with black patients disproportionately affected (FIG. 9). The median age of the adult patients at enrollment was 33 years (18-70). Three pediatric patients enrolled; their ages ranged from 12-17 years. Thirty-four patients (65%) had undergone prior resection of their primary or metastatic disease. Twenty-seven (52%) patients had received at least one line of systemic therapy prior to enrollment; 93% of those had prior tyrosine kinase inhibitor therapy. As an exploratory objective, the ASPL-TFE3 fusion type of 34 patients was determined retrospectively; 30 patients (88%) expressed the Type 1 fusion and 4 patients (12%) expressed the Type 2 fusion (FIGS. 10A-10B).

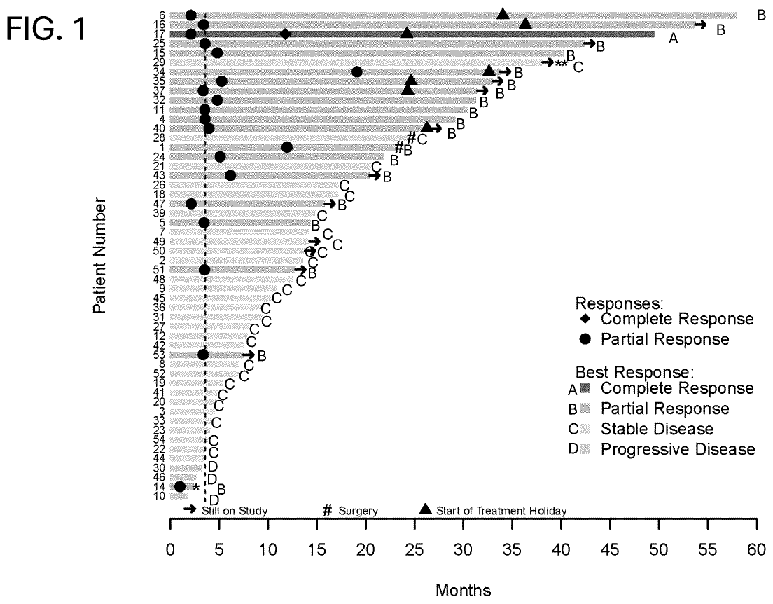

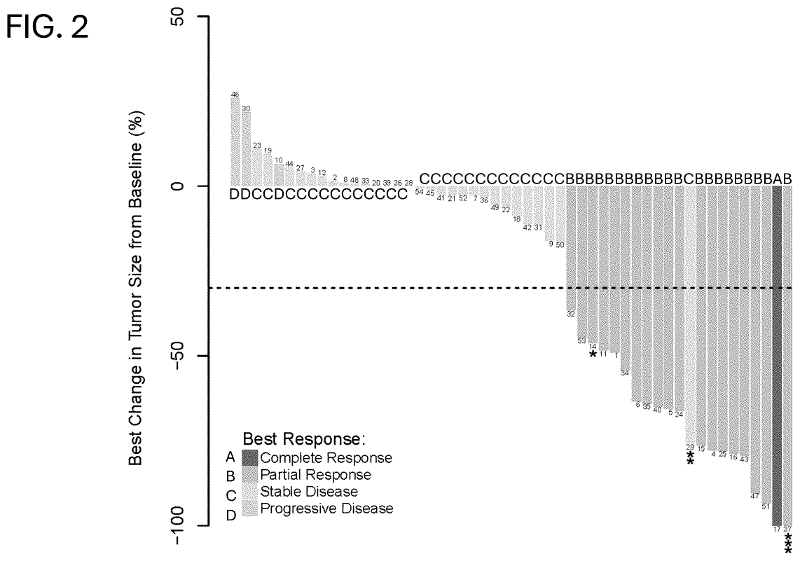

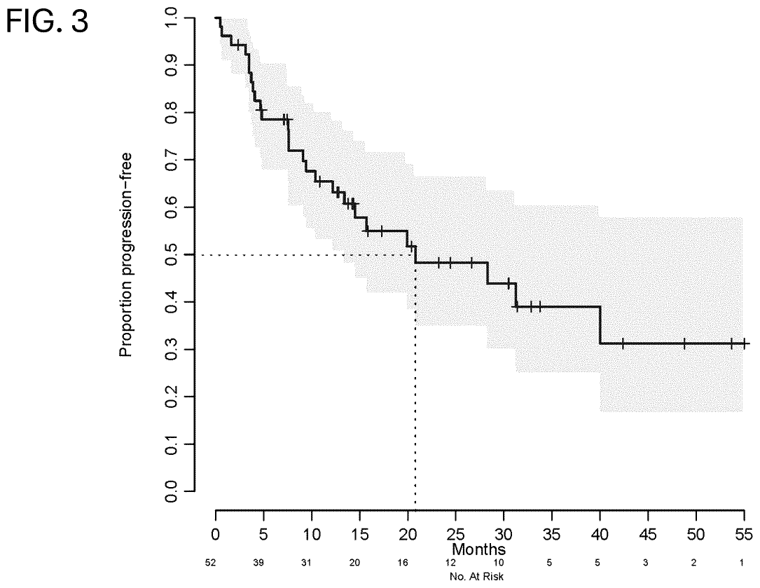

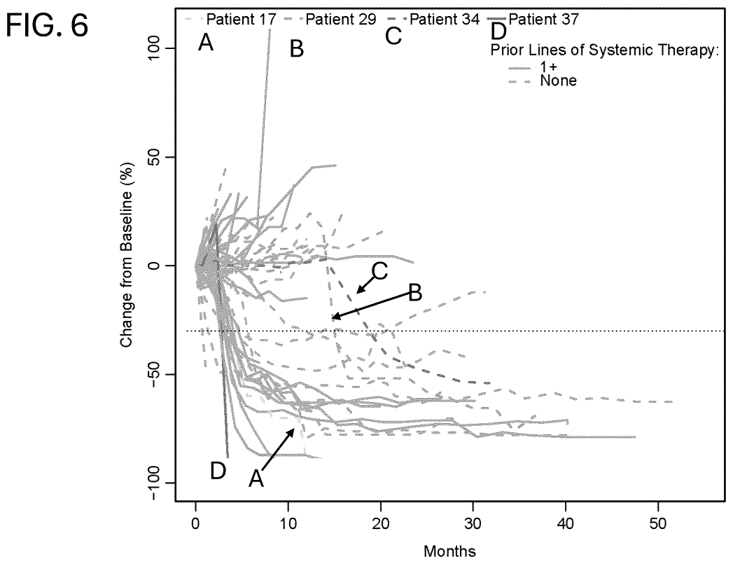

Efficacy: The trial was determined to be positive after eight confirmed responses were observed among the first 19 patients. Accrual continued to allow collection of biopsies to assess the mechanism of action of atezolizumab in ASPS. As of the Jul. 31, 2022 data cutoff, the median time on study was 13.2 months (range, 1.8-58.0) (FIG. 1). Seven patients took a treatment break after two years of therapy (range of break durations, 0.4-25.3 months [two completed, five ongoing]). Among the 52 evaluable patients, the ORR was 36.5% (95% binomial CI: 23.6%, 51.0%), with 18 patients experiencing a confirmed PR as their best response and 1 patient experiencing a confirmed CR (FIG. 1). The time to CR was 11.8 months; after the data cutoff date for this publication, a second patient achieved a CR 13.7 months into treatment. Among the 19 patients experiencing a confirmed response by RECIST 1.1, the median time to response was 3.6 months (range, 2.1-19.1), the median duration of response was 24.7 months (range, 4.1-55.8), and the median best change from baseline tumor size was −66.2% (range, −36.6% to −100%) (FIG. 2). Best clinical outcomes for the other 33 patients included a PR that was not confirmed before the patient came off study (1), a PR by iRECIST (iPR) (1), SD (28), and disease progression (PD; 3). Median PFS was 20.8 months (FIG. 3). A post-hoc assessment of ORR by prior exposure to tyrosine kinase inhibitors was also performed (FIG. 6). The ORR of patients with prior tyrosine kinase inhibitor treatment (36.0%, 95% binomial CI: 18.0%, 57.5%) was similar to the ORR of those without (37.0%, 95% binomial CI: 19.4%, 57.6%).

Many patients' tumors continued to shrink over long periods of time (FIG. 6). Two patients received treatment for approximately 12 months before their disease trajectory improved suddenly. One of these patients (#29) continued treatment beyond RECIST progression and was assessed subsequently by iRECIST, which resulted in a designation of iPR four cycles later. The other patient (#34) had stable disease for more than one year before experiencing a PR after a bout of viral-induced vestibulitis.

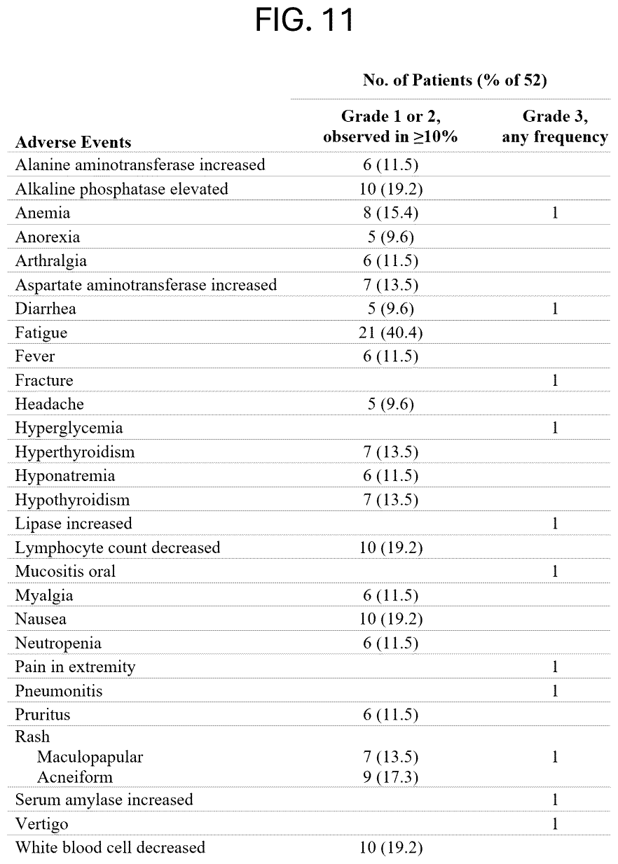

Safety: Atezolizumab was well tolerated, with an adverse event profile consistent with those previously reported for atezolizumab monotherapy (FIG. 11); 96.1 percent ( 50/52) of patients experienced grade 1 or 2 toxicity; grade 3 adverse events potentially related to atezolizumab were reported in 8 different patients (15.4%). No treatment-related grade 4 or 5 events were reported. None of the patients discontinued treatment because of adverse events.

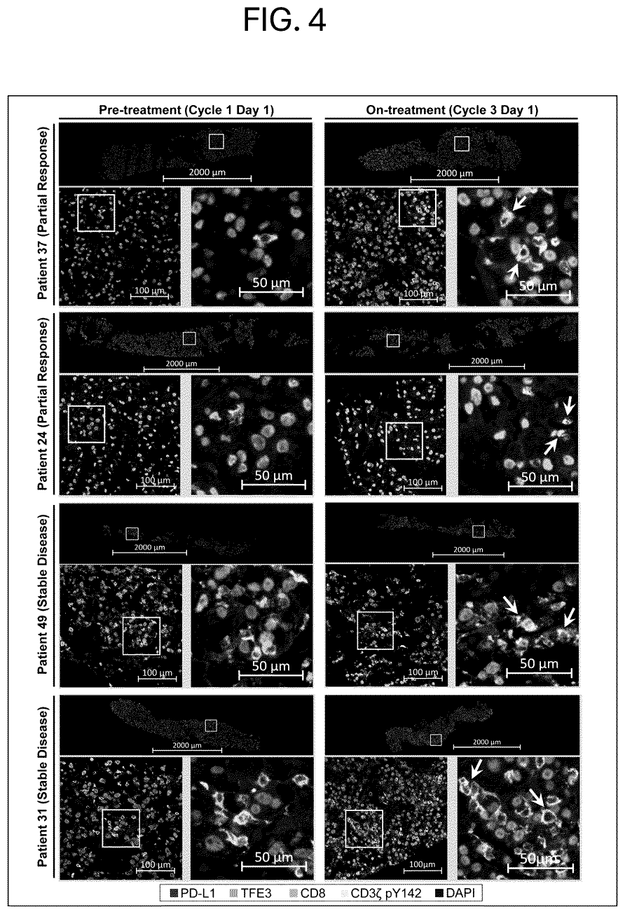

Pharmacodynamics: Analyzable ASPS tumor biopsies from patients with a best response of PR (8 patients), SD (10 patients), or PD (1 patient) were evaluated for required components of atezolizumab's mechanism of action using a multiplexed immunofluorescence assay (FIGS. 4, 12A-12C, 13C-13D). The study was not powered to compare pharmacodynamic results across the three response groups; the results within each response group are described below.

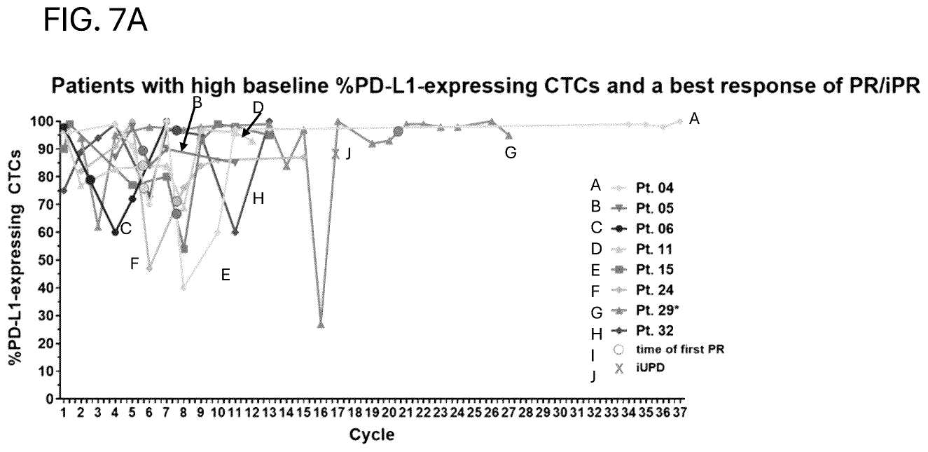

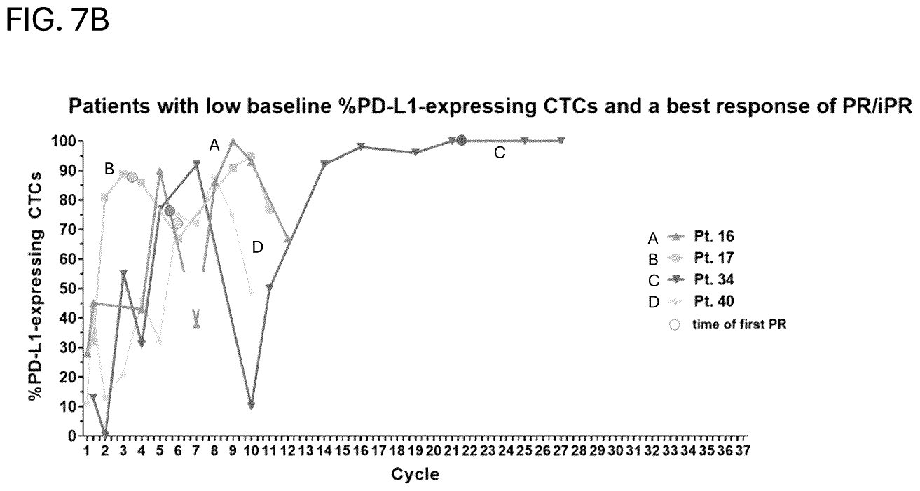

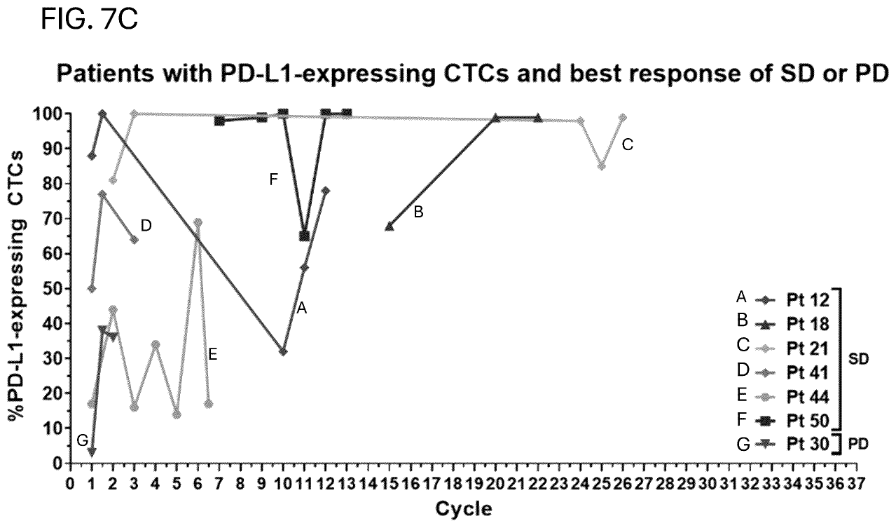

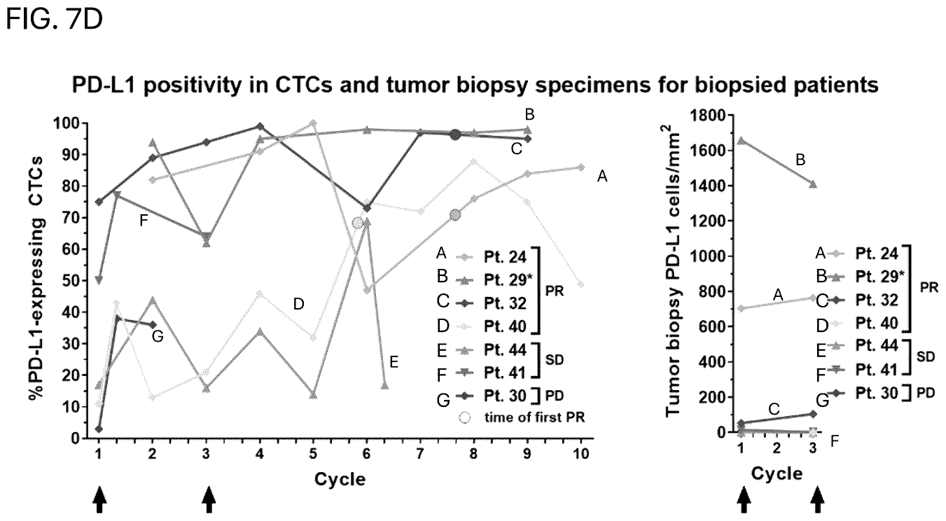

In the PR group, analysis of pre-treatment tumor biopsies found 6/6 tumors positive for PD-L1, ⅚ positive for CD3+ lymphocytes expressing the receptor PD-1, 6/6 positive for CD8+ cytotoxic T lymphocytes (CTL), and 6/6 with regulatory T cell (T_{REG})/CTL ratio <1−a ratio which indicates a permissive immune environment associated with tumor regression and overall survival advantages in other malignancies (Kareva et al., Stem Cells 2019; 37:1273-1280). At Cycle 3, Day 1, tumor biopsies from 6/7 patients in the PR group were positive for all four mechanistic components, including one Patient (#37) whose tumor converted from negative to positive for PD-1+ lymphocytes during atezolizumab therapy (FIGS. 4, 12A-12C). The sole PD-L1-negative tumor in the PR group at Cycle 3 (Patient #40) converted to PD-L1-positive at Cycle 6, based on longitudinal assessments of circulating tumor cells (FIGS. 7A-7D). At baseline, CD3ζ phosphorylation of the T cell receptor, a biomarker of antigen recognition, was positive in 4/6 tumors, and the two other tumors (Patients #01 and #43) converted from negative to positive by Cycle 3 Day 1 (FIGS. 12A-12C). In contrast to many carcinomas (Pai et al., Cancer Treat Res 2020; 180:173-195; Burandt et al., Cancers 2022; 14:4246; Craig et al., Nat Rev Gastroenterol Hepatol 2020; 17:139-152), activated CTLs were distributed throughout tumor and stroma without exclusion at invasive margins, indicating unimpeded movement within the tumor microenvironment.

In the SD group, analysis of pre-treatment biopsies revealed 9/10 tumors positive for PD-L1, 10/10 positive for intratumoral CTLs, and 10/10 with a favorable T_{REG}/CTL ratio <1 (FIGS. 12A-12C). One patient (#44) was PD-L1-negative in the biopsy specimen and throughout longer-term circulating tumor cell monitoring into Cycle 6 (FIGS. 7A-7D). ASPS tumors in the SD group were more likely negative for PD-1-positive lymphocytes at baseline ( 7/10 patients) than the PR group (⅙ patients), and the observed response rate was higher among patients with PD-1-positive lymphocytes in the tumor microenvironment before treatment ( 5/9 patients) than those without (⅛) (FIGS. 12A-12C). Three patients in the SD group had tumors that converted from PD-1-negative to PD-1 positive by Cycle 3 Day 1. Like the PR group, all evaluable pre-treatment and on-treatment biopsies were positive for CD3ζ-pY142 except in Patient #33 whose tumor converted to positive by Cycle 3 Day 1.

Of note, ASPS tumors in 3 of the 4 biopsied patients with the shortest PFS either lacked both PD-L1 and PD-1 expression (Patient #44), lacked PD-1 expression (Patient #41, SD), or had an unfavorably high T_{REG}/CD8 ratio (Patient #30, PD).

### DISCUSSION

These results demonstrated robust, durable anticancer activity of atezolizumab in a diverse cohort of patients with advanced ASPS that reflects the disease population. Objective RECIST 1.1 responses, including one complete response, occurred in 36.5% of the patients, many of whom had progressed on prior systemic therapy. Responses were durable, and several were maintained for years after the final atezolizumab dose. Tumor assessments were performed less frequently in patients who remained on this study for more than 1 year, potentially impacting PFS results by 3-6 weeks. Still, the durations of response and PFS achieved with atezolizumab therapy are uncommon with other therapies employed in ASPS, even in settings like this one where patients may have been enrolled with indolent disease (Shi et al., Clin Cancer Res 2020; 26:6445-6452; Blay et al., J Clin Oncol 2021; 39:11520-11520; Stacchiotti et al., Oncologist 2018; 23:62-70; Liu et al., Ann Transl Med 2020; 8:1493). Using these methods, tumor responses to atezolizumab typically occurred within the first 3-5 months, but three patients were on treatment for one year or longer before experiencing a partial response. Late-onset responses of ASPS to other ICIs have been reported (Wilky et al., Lancet Oncol 2019; 20:837-848; Shi et al., Clin Cancer Res 2020; 26:6445-6452).

The results from prior multi-histology trials evaluating ICIs indicated that ASPS may be especially sensitive to immune checkpoint inhibition (Groisberg et al., J Immunother Cancer 2017; 5:100-100; Wilky et al., Lancet Oncol 2019; 20:837-848; Blay et al., J Clin Oncol 2021; 39:11520-11520; Yang et al., Eur J Cancer 2020; 130:182-192; Somaiah et al., Lancet Oncol 2022; 23:1156-1166). However, interpretation of those findings is limited by the small size of each ASPS cohort and, in some cases, the use of combined drug regimens. The present phase 2 study enrolled only patients with ASPS to evaluate the efficacy of atezolizumab monotherapy in this ultra-rare histology. The response rate in this study closely resembles that observed among 10 ASPS patients treated with combined PD-L1 and CTLA4 inhibitors (Somaiah et al., Lancet Oncol 2022; 23:1156-1166). However, the combination was associated with more frequent and more serious toxicity than was observed with PD-L1 inhibition alone.

The pharmacodynamic data shown herein reveal that many of the tumors harbored the molecular and cellular elements required for atezolizumab response (PD-1 and PD-L1, unhindered movement of CTL with activated T cell receptor, and a permissive tumor microenvironment) at baseline while other tumors lacked one or more such components before treatment but converted to a responsive phenotype during atezolizumab administration. Given the possibility of conversion to an immunotherapy-responsive phenotype during treatment, patient selection based on pre-treatment PD-1/PD-L1 expression status may exclude ASPS patients with the potential to respond to ICI treatment.

The observation that ASPS is poised biologically to respond to ICIs targeting PD-L1/PD-1 may explain the high degree of clinical benefit in this trial. Transcriptomic profiling results indicate that ASPS tumors are among the most highly lymphocyte-infiltrated pediatric solid tumors (Brohl et al., Cell Rep 2021; 37:110047). Using multiplex immunofluorescent analyses, dense and unimpeded CTL tumor infiltrates in the adult ASPS population were detected. Furthermore, intratumoral CTLs with activated T cell receptor (CD3ζ phosphorylated at tyrosine-142) were present by Cycle 3 in all ASPS tumors with evaluable biopsy specimens, indicative of antigen recognition.

The results of this phase 2 clinical trial, which formed the basis of the recent FDA approval, support the use of atezolizumab as a well-tolerated and highly effective treatment for advanced ASPS.

### Example 3: Protocol for NCT03141684. Version 10/26/2022

### I. Overview

### A. Objectives

Determine the objective response rate (ORR) of atezolizumab in patients with advanced ASPS in adult subjects ≥18 years and in pediatric/adolescent subjects ≥2 years. Determine the ORR using RECIST v 1.1 of atezolizumab and bevacizumab in subjects ≥18 years old with advanced ASPS that has progressed atezolizumab monotherapy. Determine duration of response (DOR) to atezolizumab monotherapy or atezolizumab plus bevacizumab using RECIST v 1.1 and/or change in clinical symptoms. Measure PFS time for patients receiving atezolizumab monotherapy or atezolizumab plus bevacizumab as determined by investigator using RECIST v 1.1. Assess the number of activated CD8+ T cells infiltrating the tumor before and after treatment with atezolizumab monotherapy or atezolizumab plus bevacizumab, and correlate treatment-induced changes with clinical response. Measure and describe the atezolizumab pharmacokinetics (PK) and anti-drug antibodies (ADA) in patients ages 2-11 years old. Compare RECIST v 1.1 vs immune RECIST (iRECIST) in patients with ASPS on atezolizumab and atezolizumab+bevacizumab. Assess the post-progression response rate among patients who progress while on treatment holiday and then resume treatment. Analyze the genomic and immune profiles of tumors expressing Type 1 or Type 2 ASPL-TFE3 fusions and correlate findings with response to atezolizumab or atezolizumab in combination with bevacizumab. Measure and describe the atezolizumab pharmacokinetics (PK) in patients ≥12 years old.

### B. Eligibility

Patients with histologically or cytologically confirmed ASPS that is not curable by surgery. To be eligible for atezolizumab monotherapy, subjects must be ≥2 years of age at the NCI clinical center (≥14 years at other participating sites). To be eligible for atezolizumab in combination with bevacizumab, subjects must be ≥18 years of age and must have progressed on P10005 atezolizumab monotherapy.

### C. Study Design

Patients will receive atezolizumab at a fixed dose of 1200 mg IV for adults and 15 mg/kg for pediatric patients once every 21 days, in 21-day cycles. Patients in the atezolizumab plus bevacizumab arm will receive bevacizumab at 15 mg/kg IV after each dose of atezolizumab.

Tumor biopsies (mandatory at all sites) will be collected from adult patients (≥18 years of age) at baseline and prior to cycle 3 day 1 (±3 days), or at any point where there is clinical evidence that the patient is responding to drug, to evaluate any changes in either tumor or blood targets of immune checkpoint blockade.

An optional tumor biopsy may be collected from adult patients (≥18 years of age), on either treatment arm, at the time of disease progression (or pre-progression, following any restaging at which a 10-19% increase in tumor volume is observed). At the PI's discretion, a progression biopsy collected while a patient is on the monotherapy arm may be utilized as the baseline biopsy for the combination arm, provided the patient does not receive any anti-cancer agents or immunotherapy in the interim.

The trial will be halted unless there is at least one response in the initial 9 patients accrued to the monotherapy arm. Adult patients who progress on atezolizumab monotherapy may cross over to receive atezolizumab in combination with bevacizumab. The monotherapy arm will remain open to pediatric patients until 5 pediatric patients have contributed Cycle 3 Day 1 blood samples for the optional PK+ADA study, 13 pediatric patients have enrolled (with or without blood sample collections), or the atezolizumab plus bevacizumab arm is closed to accrual, whichever happens sooner.

### II. Summary of Collections and Measurements

A schema of the Atezolizumab Monotherapy Arm and Atezolizumab and Bevacizumab Combination Arm are provided in FIGS. 15 and 16. Tumor biopsies (mandatory for patients in which biopsies are deemed safe and feasible) will be collected from adult patients (≥18 years of age) at baseline and prior to cycle 3 day 1 (±3 days), or at any point where there is clinical evidence that the patient is responding to drug. If a patient is crossing over directly from monotherapy to the combination after <4 monotherapy cycles, that patient's monotherapy cycle 3 day 1 biopsy will be used as their baseline biopsy for the combination treatment arm.

An optional tumor biopsy may be collected from adult patients (≥18 years of age), on either treatment arm, at the time of disease at progression (or pre-progression, following any restaging at which a 10-19% increase in tumor volume is observed). At the PI's discretion, a progression biopsy can be collected while a patient is on the monotherapy arm may be utilized as the baseline biopsy for the combination arm, provided the patient does not receive any anti-cancer agents or immunotherapy in the interim. Mandatory collection of CTCs from patients ≥14 years of age at baseline, on cycle 2 day 1, at the start of every subsequent cycle (before drug administration), at disease progression, and at the time of each response assessment during treatment holidays and the subsequent follow-up period. While CTC collection is required at all of these timepoints, the requirement is waived if a collection time point falls on a Friday or too late on a Thursday to ensure same-day FedEx pickup, due to shipment and processing requirements. CTC collections will be repeated if a patient crosses over or re-enrolls to the combination arm. Plasma obtained during the isolation of CTCs will be retained to enable genetic analysis of cell-free DNA.

One additional blood sample will be collected for germline analysis at baseline (or any other time on study) from patients ≥18 years of age who consent to the optional sequencing component.

In some aspects, PBMCs (optional) will be collected from patients ≥14 years of age at baseline, on cycle 3 day 1 (or the next restaging visit if cycle 3 day 1 has already passed), 12 weeks (±1 week) and 24 weeks (±1 week) later, and at disease progression or response.

Blood (mandatory) can be collected from pediatric patients <12 years of age* at the following time points for measurement of pharmacokinetics (PK) and anti-drug antibodies (ADA), as detailed in Table 2.

TABLE 2
Time points for measurement of PK and ADA*

| Time point | PK | ADA |
|---|---|---|
| C 1 D 1 | Baseline (predose) | predose |
| C 1 D 1 | 30 min after the | — |
| | end of infusion | |
| C 2 D 1 | predose | predose |
| C 3 D 1 | predose | predose |
| C 4 D 1 | predose | predose |
| C 4 D 1 | 30 min after the | — |
| | end of infusion | |
| C 8 D 1 | predose | predose |
| C 12 D 1 | predose | predose |
| C 16 D 1 | predose | predose |
| Progression/Study drug | Any time | Any time |
| discontinuation | during visit | during visit |

*If a patient turns 12 years old while still receiving study treatment, blood will continue to be collected at the time points above.

Blood can be collected from patients on the monotherapy arm who are 12 years of age at the time of enrollment for measurement of PK. These optional blood collections will begin on Day 1 of the next cycle that a patient begins once collection kits are available and the patient's consent for this optional collection is obtained. The collections may continue for up to three cycles, total, as detailed in Table 3:

TABLE 3
Three Collection Cycles

| Time point | PK |
|---|---|
| Cx D 1 | predose |
| Cx D 1 | 30 min after the end of atezolizumab infusion |
| Cx + 1 D 1 | predose |
| Cx + 1 D 1 | 30 min after the end of atezolizumab infusion |
| Cx + 2 D 1 | predose |
| Cx + 2 D 1 | 30 min after the end of atezolizumab infusion |

Cx = the cycle a patient begins once collection kits are available and the patient's consent for this optional collection is obtained

Patients will undergo a CT scan at the end of cycle 3 and every two cycles thereafter (every 3 cycles for patients on study for >1 year; every 4 cycles for patients on study for >2 years). Patients on the atezolizumab monotherapy arm will have a CT scan at the end of cycle 1 which will be used to assess for early response only; no treatment decisions will be made unless clinically warranted.

Adult patients who progress on atezolizumab monotherapy may cross over to receive atezolizumab in combination with bevacizumab.

### III. Patient-Selection

### A. Eligibility Criteria

Patients must have histologically or cytologically confirmed Alveolar Soft Part Sarcoma that is not curable by surgery. Diagnosis of malignancy must be confirmed by the department of pathology at the institution where the patient is enrolled prior to patient enrollment. To be eligible for atezolizumab in combination with bevacizumab, the patient must have atezolizumab-refractory/resistant disease that has progressed (definitive clinical progression or iCPD) on prior P10005 atezolizumab monotherapy.

Patients must have measurable disease, defined as at least one lesion that can be accurately measured in at least one dimension (longest diameter to be recorded for non-nodal lesions and short axis for nodal lesions) as ≥20 mm (≥2 cm) by chest x-ray or as ≥10 mm (≥1 cm) with CT scan, MRI, or calipers by clinical exam. Note: Once the primary endpoint of the trial has been met, pediatric patients may enroll with evaluable non-measurable disease and will not be required to have measurable disease. Evaluable non-measurable disease is that which is not measurable by RECIST1.1 but can be evaluated by imaging (e.g., CT, bone scan, or ultrasound) or other methods.

Patients with newly diagnosed, unresectable, metastatic and measurable ASPS will also be eligible for atezolizumab monotherapy if they show clinical evidence of disease progression (including history and increasing physical symptoms). On-study documentation will include a physician's rationale that supports evidence of clinical disease progression (i.e., increasing tumor pain).

To be eligible for atezolizumab monotherapy, subjects must be ≥2 years of age. To be eligible for atezolizumab in combination with bevacizumab, subjects must be ≥18 years of age.

ECOG performance status ≤2 (Karnofsky or Lansky ≥70%, see FIG. 17).

Life expectancy of greater than 3 months.

Patients must have normal organ and marrow function as defined below: absolute neutrophil count ≥1,500/mcL, platelets ≥100,000/mcL, hemoglobin ≥8 g/dL, total bilirubin ≤1.5×institutional upper limit of normal (ULN) (however, patients with known Gilbert disease who have serum bilirubin level 3×ULN may be enrolled), AST(SGOT)/ALT(SGPT)≤3×ULN (AST and/or ALT ≤5×ULN for patients with liver involvement), alkaline phosphatase ≤2.5×ULN (5×ULN for patients with documented liver involvement or bone metastases), 8) creatinine clearance (For adult patients (≥18 years of age): ≥30 mL/min/1.73 m² by Cockroft-Gault). Creatine clearance is determined by the following formula ((140−age)×(weight in kg))/(72×(serum creatine in mg/dL) (×0.85 if female). For pediatric patients (<18 years of age), serum creatinine based on age and gender are shown in FIG. 18).

Administration of atezolizumab or bevacizumab may have an adverse effect on pregnancy and poses a risk to the human fetus, including embryo-lethality. Female patients of child-bearing potential and male patients must agree to use adequate contraception (hormonal or barrier method of birth control; abstinence) prior to study entry, for the duration of study participation, and for 5 months (150 days) after the last dose of atezolizumab or 6 months (180 days) after the last dose of bevacizumab. Should a woman become pregnant or suspect she is pregnant while she or her partner is participating in this study, she should inform her treating physician immediately. Ability to understand and the willingness to sign a written informed consent document or a parent/guardian able to do the same.

Willingness to provide biopsy samples for research purposes (patients 18 years of age only). At the PI's discretion, archival tissue that was collected and preserved in a manner consistent with the tissue processing details in the Biopsy Collection and Shipment section may be used in lieu of a baseline biopsy, provided no anti-cancer agents or immunotherapies were received since collection of the archival tissue.

Patients who have received live attenuated vaccines within 30 days of the first dose of trial treatment are eligible at the discretion of the investigator. All seasonal influenza vaccines and vaccines intended to prevent SARS-CoV-2 and coronavirus disease 2019 (COVID-19) are allowed. 13) For patients not receiving therapeutic anticoagulation: INR or aPTT ≤1.5×ULN for bevacizumab patients.

For patients receiving therapeutic anticoagulation: stable anticoagulant regimen for bevacizumab patients.

### B. Exclusion Criteria

1) Any prior therapy must have been completed ≥4 weeks or, if known, ≥5 half-lives of the prior agent (whichever is shorter) prior to enrollment on protocol (minimum of 1 week between prior therapy and study enrollment), and the participant must have recovered to eligibility levels from prior toxicity. Note: For patients being treated on the atezolizumab plus bevacizumab arm, there is no washout requirement for prior anti-PD-1 or anti-PD-L1 agents. Patients should be at least 6 weeks out from nitrosoureas and mitomycin C. Prior definitive radiation should have been completed ≥4 weeks or palliative radiation should have been completed ≥2 weeks prior to study enrollment or crossover and all associated toxicities resolved to eligibility levels (patients on study may be eligible for palliative radiotherapy to non-targeted lesions after 2 cycles of therapy at the PI's discretion). Patients who have had prior monoclonal antibody therapy must have completed that therapy ≥6 weeks (or 3 half-lives of the antibody, whichever is shorter) prior to enrollment on protocol (minimum of 1 week between prior therapy and study enrollment). A patient who has received a cumulative dose of >350 mg/m² of anthracycline (regardless of cardioprotectant) may only be enrolled if their ejection fraction measured by an echocardiogram is within normal institutional limits.
2) Prior treatment with anti-PD-1, or anti-PD-L1 therapeutic antibody or pathway-targeting agents. Patients who have received prior treatment with anti-CTLA-4 may be enrolled on the atezolizumab monotherapy arm, provided the following requirements and all other selection criteria are met: Minimum of 12 weeks from the first dose of anti-CTLA-4 and >6 weeks from the last dose and no history of severe immune-related adverse effects from anti-CTLA-4 (NCI CTCAE Grade 3 and 4). Patients who have progressed on P10005 atezolizumab monotherapy may be eligible for the atezolizumab plus bevacizumab arm, provided the following requirements, and all other selection criteria, are met: no prior disease progression on an immune checkpoint inhibitor plus tyrosine kinase inhibitor combination therapy, any prior toxicities have resolved to eligibility levels, and no contraindications.
3) Treatment with any other investigational agent within 4 weeks (or within five half-lives of the investigational product, whichever is shorter) prior to Cycle 1, Day 1 (minimum of 1 week between prior therapy and study enrollment). Patients must be ≥2 weeks since any investigational agent administered as part of a Phase 0 study (also referred to as an “early Phase I study” or “pre-Phase I study” where a sub-therapeutic dose of drug is administered) at the Coordinating Center PI's discretion, and should have recovered to eligibility levels from any toxicities.
4) Treatment with systemic immunostimulatory agents (including, but not limited to, interferon-α or interleukin-2 [aldesleukin]) within 6 weeks prior to Cycle 1, Day 1.
5) Treatment with systemic immunosuppressive medications (including, but not limited to, prednisone, cyclophosphamide, azathioprine, methotrexate, thalidomide, and anti-tumor necrosis factor [anti-TNF]agents) within 2 weeks prior to Cycle 1, Day 1. Patients who have received acute, low dose, systemic immunosuppressant medications (e.g., a one-time dose of dexamethasone for nausea) may be enrolled. The use of inhaled corticosteroids and systemic mineralocorticoids (e.g., fludrocortisone) for patients with orthostatic hypotension or adrenocortical insufficiency is allowed.
6) Patients taking bisphosphonate therapy for symptomatic hypercalcemia. Use of bisphosphonate therapy for other reasons (e.g., bone metastasis or osteoporosis) is allowed.
7) Patients with known primary central nervous system (CNS) malignancy or symptomatic CNS metastases are excluded, with the two following exceptions. Patients with asymptomatic untreated CNS disease may be enrolled, provided all of the following criteria are met (Evaluable or measurable disease outside the CNS, no metastases to brain stem, midbrain, pons, medulla, or cerebellum, no history of intracranial hemorrhage or spinal cord hemorrhage, no ongoing requirement for dexamethasone for CNS disease; patients on a stable dose of anticonvulsants are permitted, and no neurosurgical resection or brain biopsy within 28 days prior to Cycle 1, Day 1. Second, patients with asymptomatic treated CNS metastases may be enrolled, provided all the criteria listed above are met as well as the following: Radiographic demonstration of improvement upon the completion of CNS-directed therapy and no evidence of interim progression between the completion of CNS-directed therapy and radiographic screening for the current study; No stereotactic radiation or whole-brain radiation within 28 days prior to Cycle 1, Day 1; Screening CNS radiographic study ≥4 weeks from completion of radiotherapy and >2 weeks from discontinuation of corticosteroids.
8) Known hypersensitivity to Chinese hamster ovary cell products or other recombinant human antibodies.
9) History of severe allergic, anaphylactic, or other hypersensitivity reactions to chimeric or humanized antibodies (i.e., antibodies with generic names ending in “ximab” or “zumab”, respectively) or fusion proteins.
10) Uncontrolled intercurrent illness including, but not limited to, ongoing or active infection, symptomatic congestive heart failure, unstable angina pectoris, cardiac arrhythmia, or psychiatric illness/social situations that would limit compliance with study requirements.
11) Pregnant women are excluded from this study because atezolizumab is an investigational agent with the unknown potential for teratogenic or abortifacient effects. Because there is an unknown but potential risk for adverse events in nursing infants secondary to treatment of the mother with atezolizumab, breastfeeding should be discontinued if the mother is treated with atezolizumab.
12) HIV-positive patients may be on combination antiretroviral therapy but must have a CD4 count >350 cells/mm³ with an undetectable viral load.
13) Patients on supraphysiologic doses of steroids or use of such within the previous six weeks.
14) Known clinically significant liver disease, including active viral, alcoholic, or other hepatitis; cirrhosis; fatty liver; and inherited liver disease. Patients with past or resolved hepatitis B infection (defined as having a negative hepatitis B surface antigen [HBsAg] test and a positive anti-HBc [antibody to hepatitis B core antigen] antibody test) are eligible. Patients positive for hepatitis C virus (HCV) antibody are eligible only if polymerase chain reaction (PCR) is negative for HCV RNA.
15) History or risk of autoimmune disease, including, but not limited to, systemic lupus erythematosus, rheumatoid arthritis, inflammatory bowel disease, vascular thrombosis associated with antiphospholipid syndrome, Wegener's granulomatosis, Sjögren's syndrome, Bell's palsy, Guillain-Barré syndrome, multiple sclerosis, autoimmune thyroid disease, vasculitis, or glomerulonephritis.
16) Patients with a history of autoimmune hypothyroidism on a stable dose of thyroid replacement hormone may be eligible. Patients with controlled Type 1 diabetes mellitus on a stable insulin regimen may be eligible. Patients with eczema, psoriasis, lichen simplex chronicus, or vitiligo with dermatologic manifestations only (e.g., patients with psoriatic arthritis would be excluded) are permitted provided that they meet the following conditions: patients with psoriasis must have a baseline ophthalmologic exam to rule out ocular manifestations, rash must cover less than 10% of body surface area (BSA), disease is well controlled at baseline and only requiring low potency topical steroids (e.g., hydrocortisone 2.5%, hydrocortisone butyrate 0.1%, flucinolone 0.01%, desonide 0.05%, aclometasone dipropionate 0.05%), and no acute exacerbations of underlying condition within the last 12 months (not requiring psoralen plus ultraviolet A radiation [PUVA], methotrexate, retinoids, biologic agents, oral calcineurin inhibitors; high potency or oral steroids).
17) History of idiopathic pulmonary fibrosis, pneumonitis (including drug induced), organizing pneumonia (i.e., bronchiolitis obliterans, cryptogenic organizing pneumonia, etc.), or evidence of active pneumonitis on screening chest computed tomography (CT) scan. History of radiation pneumonitis in the radiation field (fibrosis) is permitted.
18) Patients with active tuberculosis (TB) are excluded.
19) Severe infections within 4 weeks prior to Cycle 1, Day 1, including, but not limited to, hospitalization for complications of infection, bacteremia, or severe pneumonia.
20) Signs or symptoms of infection within 2 weeks prior to Cycle 1, Day 1.
21) Received oral or intravenous (IV) antibiotics within 2 weeks prior to Cycle 1, Day 1. Patients receiving prophylactic antibiotics (e.g., for prevention of a urinary tract infection or chronic obstructive pulmonary disease) are eligible.
22) Major surgical procedure within 28 days prior to Cycle 1, Day 1 or anticipation of need for a major surgical procedure during the course of the study. Note: Patients with a surgical procedure (excluding biopsy procedures described in Biopsy collection and shipment) within 28 days prior to Cycle 1 Day 1, or any unhealed surgical wound, are not eligible for the atezolizumab+bevacizumab combination arm.
23) Administration of a live, attenuated vaccine within 4 weeks before Cycle 1, Day 1 or anticipation that such a live, attenuated vaccine will be required during the study and up to 5 months after the last dose of atezolizumab. Influenza vaccination should be given during influenza season only (approximately October to March). Patients must not receive live, attenuated influenza vaccine within 4 weeks prior to Cycle 1, Day 1 or at any time during the study.
24) Additional exclusion criteria include: history of leptomeningeal disease, uncontrolled tumor-related pain, patients requiring pain medication must be on a stable regimen at study entry, symptomatic lesions (e.g., bone metastases or metastases causing nerve impingement) amenable to palliative radiotherapy should be treated prior to enrollment (Patients should be recovered from the effects of radiation. There is no required minimum recovery period.), asymptomatic metastatic lesions that would likely cause functional deficits or intractable pain with further growth (e.g., epidural metastasis that is not currently associated with spinal cord compression) should be considered for loco-regional therapy if appropriate prior to enrollment, uncontrolled pleural effusion, pericardial effusion, or ascites requiring recurrent drainage procedures (once monthly or more frequently), patients with indwelling catheters (e.g., PleurX®) are allowed, uncontrolled or symptomatic hypercalcemia (ionized calcium >1.5 mmol/L, calcium >12 mg/dL or corrected serum calcium >ULN), history of malignancy within 3 years prior to screening, with the exception of malignancies with a negligible risk of metastasis or death (e.g., 5-year OS rate >90%), such as adequately treated carcinoma in situ of the cervix, non-melanoma skin carcinoma, localized prostate cancer, ductal carcinoma in situ, or Stage I uterine cancer, prior allogeneic stem cell or solid organ transplantation, and any other disease, metabolic dysfunction, physical examination finding, or clinical laboratory finding that contraindicates the use of an investigational drug, may affect the interpretation of the results, or may render the patient at high risk from treatment complications.

### C. Bevacizumab-Specific Exclusion Criteria

The following exclusion criteria, in addition to those listed above, apply to the atezolizumab+bevacizumab combination arm:
1. Age <18 years.
2. Prior disease progression on an immune checkpoint inhibitor plus tyrosine kinase inhibitor combination therapy.
3. Major surgery within 6 weeks of enrollment. Procedures (e.g. port placement, endoscopy with intervention) within 7 days of enrollment (excluding biopsy procedures described in Biopsy Collection and shipment).
4. Chronic daily treatment with a non-steroidal anti-inflammatory drug (NSAID), clopidogrel, dypiridamole, or aspirin therapy >81 mg/day. Treatment with any such agents within 7 days prior to Cycle 1 Day 1.
5. 2+ protein on urinalysis, followed by 24-hour protein of >1 g.
6. Patients with a history of bleeding varices within 1 year of enrollment.
7. Thromboembolic event within 6 months of enrollment (including cerebrovascular accident (CVA) and myocardial infarction (MI).
8. Evidence of bleeding diathesis or significant coagulopathy (with or without current therapeutic anticoagulation).
9. International normalized ratio (INR) ≥1.5 (or >therapeutic range if patient is receiving warfarin). PTT ≥1.5×ULN.
10. History of hemoptysis (>½ teaspoon of bright red blood per episode) within 1 month of enrollment.
11. Serious, non-healing wound, active ulcer, or untreated bone fracture.
12. Congestive heart failure, baseline LVEF <50%, transmural myocardial infarction, uncontrolled hypertension (defined as systolic blood pressure >140 mmHg and/or diastolic blood pressure >90 mmHg in patients ≥18 years of age), angina pectoris requiring medication, clinically significant valvular disease, high-risk arrhythmia within 12 months of enrollment. Prior history of hypertensive crisis or hypertensive encephalopathy.
13. History of abdominal fistula or gastrointestinal perforation within 6 months prior to enrollment.
14. History of allergic reactions attributed to compounds of similar chemical or biologic composition to bevacizumab.
15. For patients not receiving therapeutic anticoagulation: INR or aPTT ≤1.5×; for patients receiving therapeutic anticoagulation: stable anticoagulant regimen.
16. Significant vascular disease (e.g., aortic aneurysm requiring surgical repair or recent arterial thrombosis) within 6 months prior to randomization.
17. Core biopsy or other minor surgical procedure, excluding placement of a vascular access device, within 7 days prior to initiation of study treatment. Placement of a vascular access device should be at least 2 days prior to initiation of study treatment.
18. Active infection requiring IV antibiotics at the time of initiation of study treatment.
19. Current or recent (<10 days prior to initiation of study treatment) use of aspirin (>325 mg/day), or clopidogrel (>75 mg/day).
20. The use of full-dose oral or parenteral anticoagulants for therapeutic purpose is permitted as long as the INR and/or aPTT is within therapeutic limits (according to institution standards) within 7 days prior to initiation of study treatment and the patient has been on a stable dose of anticoagulants for ≥2 weeks prior to initiation of study treatment. Prophylactic use of anticoagulants is allowed. However, the use of direct oral anticoagulant therapies such as dabigatran (Pradaxa®) and rivaroxaban (Xarelto®) is not recommended due to bleeding risk.

### D. Eligibility Screening Evaluation

Patient eligibility screening tests will be performed as described below. Eligibility history, physical examination, laboratory evaluations, urinalysis, and EKG are to be conducted within 8 days prior to enrollment (or crossover) to either treatment arm. If protocol therapy is started within 8 days of these eligibility screening evaluations, values from the screening evaluations may be used as baseline measurements; if >8 days have passed since the screening evaluations, the medical history, physical examination, laboratory evaluations, urinalysis, and EKG must be repeated prior to starting protocol therapy. Eligibility and baseline imaging scans and ECHO must be done within 28 days prior to enrollment and prior to the start of protocol therapy, respectively.
1. Histologic confirmation: A block or stained slides of primary tumor tissue or of known recurrence will be required from each participant to confirm diagnosis. Pathology confirmation will be performed by the Laboratory of Pathology, NIH for patients enrolled at the NCI or by the department of pathology at the site where the patient is enrolled.
2. History and physical examination: Complete history and physical examination (including height, weight, vital signs, and ECOG performance score) will be conducted within 8 days prior to both patient enrollment and the start of protocol therapy (monotherapy or the combination).
3. Imaging Studies: Every participant should have an evaluation of known sites of disease. Scans for tumor measurements must be done within 28 days prior to both patient enrollment and the start of protocol therapy (monotherapy or the combination).
4. Laboratory Evaluation: Laboratory tests to establish eligibility are to be performed within 8 days prior to both patient enrollment and the start of protocol therapy (monotherapy or the combination):
   Hematological Profile: CBC with differential, platelets
   Biochemical Profile: ACTH and AM cortisol*, albumin, alkaline phosphatase, amylase, total bilirubin, BUN, bicarbonate, calcium, chloride, creatinine, glucose, LDH, lipase, phosphorus, potassium, total protein, SGOT (AST), SGPT (ALT), sodium, TSH
      The requirements for ACTH and AM cortisol monitoring are waived while a patient is on exogenous or replacement steroid therapy; ACTH and AM cortisol may be measured at the PI's discretion.
   Urinalysis
   Serum or urine pregnancy test (B-HCG) for female participants of childbearing potential
   Prothrombin time (PT), partial thromboplastin time (PTT), or international normalized ratio (INR) test for participants on the combination arm only.
5. Cardiac Evaluation to establish eligibility. (a) EKG within 21 days prior to both patient enrollment and start of protocol therapy (monotherapy or the combination), (b) Echocardiogram (ECHO) within 28 days prior to both patient enrollment and the start of protocol therapy (monotherapy or the combination)

Patients who crossover or re-enroll to the combination treatment arm must be screened within 28 days of initiating the combination and must meet all of the patient selection criteria outlined in the Patient Selection Section, with the exception of the prohibition against prior anti-PD-L1 therapy. Currently enrolled patients who meet all of the selection criteria will crossover to the combination arm without being re-registered; they will retain their study ID number. Previously enrolled patients who re-enroll and meet all selection criteria will be registered for the combination arm as new patients with new ID numbers.

### IV. Treatment Plan

This is an open-label phase 2 trial of atezolizumab in patients with advanced ASPS in adult subjects ≥18 years of age and in pediatric/adolescent subjects ≥2 years of age. This trial is also evaluating the combination of atezolizumab plus bevacizumab in adult subjects with advanced ASPS that progressed (definitive clinical progression or iCPD) on P10005 atezolizumab monotherapy.

Reported adverse events and potential risks for atezolizumab are described in the Adverse Events Section. Doses and drug administration are defined in the Treatment Plan section. There are no dose modifications for this agent. Each cycle is 21 days (±3 days due to scheduling conflicts). The start of a new cycle may be delayed up to 2 weeks to accommodate scheduling conflicts and other unexpected events.

Patient evaluations will be performed throughout the study as described below. Baseline history, physical examination, laboratory evaluations, urinalysis, and EKG are to be conducted within 8 days prior to the start of protocol therapy. If protocol therapy is started within 8 days of the eligibility screening evaluations, values from the screening evaluations may be used as baseline measurements; if >8 days have passed since the screening evaluations, the medical history, physical examination, laboratory evaluations, and urinalysis, must be repeated prior to starting protocol therapy (21 days are allowed for EKG). Baseline imaging scans and ECHO must be done within 28 days prior to the start of protocol therapy.

Baseline history and physical examination will be done within 8 days of cycle 1 day 1. History and physical examination will then be done within 8 days of day 1 of all subsequent cycles. Labs (CBC with differential; serum chemistries) will be performed at baseline (within 8 days of cycle 1 day 1) and then up to 3 days before the start of each subsequent cycle (LDH and protein do not need to be checked again after baseline). TSH will be checked at baseline (within 8 days of cycle 1 day 1) and then up to 3 days before the start of each subsequent cycle. Baseline EKG (within 21 days prior to start of protocol therapy) and baseline ECHO (within 28 days prior to start of protocol therapy) will be performed, and EKG and ECHO will be repeated as clinically indicated thereafter. Urinalysis will be done at baseline (within 8 days of cycle 1 day 1); urinalysis will be repeated up to 3 days before the start of each subsequent cycle for the atezolizumab+bevacizumab arm only.

Patients will have a CT scan (or other imaging test) at baseline (within 28 days prior to start of protocol therapy in either arm). Patients in the monotherapy arm will have a CT scan at the end of cycle 1, which will be used to assess for early response only; no treatment decisions will be made unless clinically warranted.

All patients will undergo a CT scan at the end of cycle 3 and every two cycles thereafter (every 3 cycles for patients on study for >1 year; every 4 cycles for patients on study for >2 years). Given the concern of pseudoprogression (“tumor flare”), patients will only be taken off treatment if two scans at normal intervals show progression along with worsening clinical picture.

Patients have the option to receive protocol therapy between restaging visits at an ETCTN or EDDOP study site other than their enrolling site if the protocol is open at that site and provided the following conditions are met:
They have completed 8 cycles of protocol therapy (or they have the PI's approval to exercise this option sooner).
There is an investigator at the non-enrolling study site who is willing to assume responsibility for periodically treating the patient and maintaining communication with the site where the patient is enrolled.
They have the approval of the Clinical Investigator at their site of enrollment.

Patients will need to visit their enrolling site for restaging visits (e.g., end of cycle 9, end of cycle 11, etc.) and will start the subsequent treatment cycle at their enrolling site (e.g., start of cycle 10, start of cycle 12, etc.). Research blood collections that coincide with a patient's treatment at a non-enrolling site may be waived (CTCs) or postponed to the next restaging visit (PMBCs and PK+ADA collections).

Research biopsies for adult patients can be performed, provided the biopsy is deemed safe and feasible. Patients that cannot be safely biopsied may be considered for the study. Blood samples can be collected for PD studies from patients ≥14 years of age (some mandatory and some optional) and blood samples for PK+ADA studies (mandatory) will be collected from patients who are <12 years of age at enrollment.

### A. Agent Administration

Treatment will be generally administered on an outpatient basis. Reported adverse events and potential risks are described in the Adverse Events Section. Administration of atezolizumab will be performed in a setting with emergency management capabilities and staff who are trained to monitor for and respond to medical emergencies.

No investigational or commercial agents or therapies other than those described below may be administered with the intent to treat the patient's malignancy. Patients on study may be eligible for palliative radiotherapy to non-targeted lesions after completing 2 cycles of therapy and achieving stable or responding disease at a physician's discretion.

Patients on treatment for >2 years could be given the option of going on a treatment holiday for up to 2 years. Imaging scans will be required every 3 months during the holiday. If scans show signs of disease progression, the patient will be required to resume treatment within 21 days of the scan or go off-study. For disease evaluation purposes, the new scan will be the new baseline scan for all future RECIST comparisons. The post-progression response rate among patients who progress while on holiday and then resume treatment will be assessed as an exploratory endpoint. This endpoint will not include patients who resume therapy after a drug holiday (≤2 years) without progression.

A physician can decide to end the holiday early (before 2 years) with documentation of the rationale. Patients resuming treatment must be reassessed and shown to meet study eligibility criteria 5 through 10 and none of the exclusion criteria (with the exception of the Bavacizumab-specific exclusion criteria 2, prior treatment with an anti-PD-L1 therapeutic antibody); if they do not meet these selection criteria within 3 months, they must go off study.

Patients who continue the treatment holiday for 2 full years without disease progression will be required to go off treatment and will undergo follow up every 6 months. Monotherapy and combination regiment descriptions are included in Tables 4 and 5.

TABLE 4
Monotherapy Regimen Description

| | | | | Cycle |
|---|---|---|---|---|
| Atezolizumab | Dose | Route | Schedule | Length |
| Adults ≥18 yrs | 1200 mg | IV | Day 1, week 1 | 21 days |
| Pediatrics ≥2 yrs | 15 mg/kg | IV | Day 1, week 1 | 21 days |
| | (1200 mg max) | | | |

TABLE 5
Combination Regiment Description

| Atezolizumab | Dose | Route | Schedule | Cycle Length |
|---|---|---|---|---|
| Adults ≥18 yrs | 1200 mg | IV | Day 1, week 1 | 21 days |
| Bevacizumab* | Dose | Route | Schedule | Cycle Length |
| Adults ≥18 yrs | 15 mg/kg | IV | Day 1, week 1 | 21 days |
| Bevacizumab* | Reduced Dose [symbol] | Route | Schedule | Cycle Length |
| Adults ≥18 yrs | 10 mg/kg [symbol] | IV | Day 1, week 1 | 21 days |

*Bevacizumab will be administered after atezolizumab. [symbol]
Dose reduction is prescribed for the management of select AEs.

For adult and pediatric patients, the initial dose of atezolizumab will be delivered as defined in Agent Administration. On the combination arm, bevacizumab will be administered after atezolizumab. On Cycle 1 Day 1 and Cycle 2 Day 1, bevacizumab will be administered 30 (±10) minutes after atezolizumab. If no atezolizumab-infusion-related reactions occur, the drugs can be administered sequentially in subsequent cycles.

The initial dose of bevacizumab will be delivered over 90 (±15) minutes. If the first infusion is tolerated without infusion-associated AEs, the second infusion may be delivered over 60 (±10) minutes. If the 60-minute infusion is well tolerated, all subsequent infusions may be delivered over 30 (±10) minutes.

For the first infusion of either agent, the patient's vital signs (heart rate, respiratory rate, blood pressure, and temperature) should be determined within 60 minutes before the first infusion. If clinically indicated, the patient's vital signs should be measured every 15 [±5] minutes during the first infusion and 30 (±10) minutes after the infusion. For subsequent infusions, vital signs will be collected within 60 minutes before the infusion. If the patient experienced an infusion-related reaction with the previous infusion or if clinically indicated, vital signs should be measured during the infusion and at 30 (±10) minutes after the infusion. Patients will be informed about the possibility of delayed post-infusion symptoms and instructed to contact their study physician if they develop such symptoms.

### B. Atezolizumab Infusion Related Reactions

For anaphylaxis precautions, see the management guidelines. Atezolizumab infusions will be administered per the instructions outlined in Table 6.

TABLE 6
Administration of First and Subsequent Atezolizumab Infusions

| First Infusion | Subsequent Infusions |
|---|---|
| No premedication is permitted prior to the | If the patient experienced an infusion-related |
| atezolizumab infusion. | reaction with any previous infusion, premedication |
| Vital signs (pulse rate, respiratory rate, blood | with antihistamines, antipyretics, and/or analgesics |
| pressure, and temperature) should be | may be administered for subsequent doses at the |
| measured within 60 minutes prior to the | discretion of the investigator. |
| infusion. | Vital signs should be measured within 60 |
| Atezolizumab should be infused over 60 | minutes prior to the infusion. |
| (±15) minutes. | Atezolizumab should be infused over 30 (±10) |
| If clinically indicated, vital signs should be | minutes if the previous infusion was tolerated |
| measured every 15 (±5) minutes during the | without an infusion-related reaction, or 60 (±15) |
| infusion and at 30 (±10) minutes after the | minutes if the patient experienced an infusion- |
| infusion. | related reaction with the previous infusion. |
| Patients should be informed about the | If the patient experienced an infusion-related |
| possibility of delayed post-infusion | reaction with the previous infusion or if |
| symptoms and instructed to contact their | clinically indicated, vital signs should be |
| study physician if they develop such | measured during the infusion and at 30 (±10) |
| symptoms. | minutes after the infusion. |

For anaphylaxis precautions, use the following procedure:

### Equipment Needed:

Monitoring devices: ECG monitor, blood pressure monitor, oxygen saturation monitor, and thermometer
Oxygen
Epinephrine for subcutaneous, intravenous, and/or endotracheal use in accordance with standard practice
Antihistamines
Corticosteroids

Procedures: In the event of a suspected anaphylactic reaction during atezolizumab infusion, the following procedures should be performed:
1. Stop the study drug infusion.
2. Call for additional medical assistance.
3. Ensure that appropriate monitoring is in place, with continuous ECG and pulse oximetry monitoring, if possible.
4. Administer antihistamines, epinephrine, or other medications as required by patient status and directed by the physician in charge.
5. Continue to observe the patient and document observation.
6. Draw serum/plasma samples for immunogenicity testing.
7. Ask participant to return for washout immunogenicity sample if appropriate.

### C. Bevacizumab Infusion Related Reactions

If a subject experiences an infusion-associated adverse event, he or she may be pre-medicated for the next study drug infusion; however, the infusion time may not be decreased for the subsequent infusion. If the next infusion is well tolerated with premedication, the subsequent infusion time may then be decreased by 30±10 minutes as long as the subject continues to be pre-medicated. If a subject experience an infusion-associated adverse event with the 60-minute infusion, all subsequent doses should be given over 90±15 minutes. Similarly, if a subject experience an infusion-associated adverse event with the 30-minute infusion, all subsequent doses should be given over 60±10 minutes.

### D. Duration of Therapy

In the absence of treatment delays due to adverse event(s) or treatment holiday, treatment may continue for until one of the following criteria applies:
Disease progression,
Intercurrent illness that prevents further administration of treatment,
Unacceptable adverse event(s),
Patient decides to withdraw from the study, or
General or specific changes in the patient's condition render the patient unacceptable for further treatment in the judgment of the investigator.
   Patients who show evidence of clinical benefit may be permitted to continue study treatment after disease progression, if they meet the criteria outlined in the Antitumor Effect—Solid Tumors,

### E. Duration of Follow Up

Safety Follow Up: Patients will be followed for 30 days after the last dose is administered or until the patient enrolls on another protocol or death, whichever comes first. The follow up will consist of a phone call between Days 27-30 after the last dose to evaluate adverse events that were ongoing and any new events that might be deemed related to the therapy. Toxicities felt to be possibly, probably, or definitely related to the study drugs that have not resolved or stabilized by Day 30 post-treatment will be followed until stabilization or resolution via biweekly phone calls.

Response Follow Up: Patients who reach the end of a 2-year treatment holiday will undergo a CT scan (or other imaging test; see Methods for Evaluation of Measurable Disease below). Those who remain progression-free at the end of the 2-year treatment holiday will be formally taken off treatment and will be ineligible to resume that treatment on this study. These patients will be followed for response evaluation every 6 months (±1 month) until disease progression, death, patient withdrawal, or enrollment in another protocol, whichever comes first. Follow up will consist of a scan every 6 months (±1 month).

Criteria for Removal from Treatment and/or Study: Patients will be removed from treatment when any of the criteria listed above applies.

### V. Dosing Delays/Dose Modifications

### A. General AE Management and Dose Modification Guidelines

Atezolizumab: There will be no dose reduction for atezolizumab in this study. Patients may temporarily suspend study treatment for up to 84 days (12 weeks) beyond the scheduled date of delayed infusion if study drug-related toxicity requiring dose suspension is experienced. If atezolizumab is held because of AEs for >84 days beyond the scheduled date of infusion, the patient will be discontinued from atezolizumab and will be followed for safety and efficacy as specified in this protocol. If the AE resolves within 84 days and the patient is receiving corticosteroid therapy for the event, atezolizumab may be held for longer than 84 days (up to 4 weeks) in order to allow tapering of the steroid dose to ≤10 mg oral prednisone or equivalent.

Dose interruptions for reasons other than toxicity, such as surgical procedures, may be allowed. The acceptable length of interruption will be at the discretion of the study PI in consultation with CTEP.

The primary approach to grade 1 to 2 irAEs is supportive and symptomatic care with continued treatment with atezolizumab; for higher-grade irAEs, atezolizumab should be withheld and oral and/or parenteral steroids administered. Recurrent grade 2 irAEs may also mandate withholding atezolizumab or the use of steroids. Assessment of the benefit risk balance should be made by the investigator, with consideration of the totality of information as it pertains to the nature of the toxicity and the degree of clinical benefit a given patient may be experiencing prior to further administration of atezolizumab. Atezolizumab should be permanently discontinued in patients with life threatening irAEs.

Systemic Immune Activation: Systemic immune activation is a rare condition characterized by an excessive immune response. Given the mechanism of action of atezolizumab, systemic immune activation is considered a potential risk when given in combination with other immunomodulating agents. Systemic immune activation should be included in the differential diagnosis for patients who, in the absence of an alternative etiology, develop a sepsis-like syndrome after administration of atezolizumab, and the initial evaluation should include the following:
CBC with peripheral smear
PT, PTT, fibrinogen, and D-dimer
Ferritin
Triglycerides
AST, ALT, and total bilirubin
LDH
Complete neurologic and abdominal examination (assess for hepatosplenomegaly)

If systemic immune activation is still suspected after the initial evaluation, additional recommendations can be considered.

Bevacizumab: For detailed information regarding management of adverse events associated with bevacizumab, please refer to management of specific adverse events.

Bevacizumab must be discontinued, and may NOT be resumed, if the patient experiences any of the following events, regardless of benefit:
gastrointestinal perforation, tracheoesophageal fistula, or any Grade 4 fistula
fistula formation involving any internal organ
wound healing complications requiring medical intervention
necrotizing fasciitis
Grades 3-4 hemorrhage
arterial thromboembolic events (ATE)
Grade 4 venous thromboembolic events (VTE), including pulmonary embolism
hypertensive crisis or hypertensive encephalopathy
posterior reversible encephalopathy syndrome (PRES)
nephrotic syndrome
severe infusion-related reaction
congestive heart failure (CHF)
   If treatment with bevacizumab is discontinued, patients may continue to receive atezolizumab after discussion between the patient and PI.

### B. Management of Specific AEs

Atezolizumab: Management of certain AEs of concern, including immune-related pneumonitis, hepatitis, colitis, endocrinopathies, pancreatitis, neuropathies, meningoencephalitis, and potential ocular toxicities are presented in the Atezolizumab Investigator's Brochure. See Agent Administration for guidelines for the management of infusion-related reactions (IRRs) and anaphylaxis.

Atezolizumab has been associated with risks such as the following: IRRs and immune-related hepatitis, pneumonitis, colitis, pancreatitis, diabetes mellitus, hypothyroidism, hyperthyroidism, adrenal insufficiency, hypophysitis, Guillain-Barré syndrome, myasthenic syndrome or myasthenia gravis, meningoencephalitis, myocarditis, nephritis, myositis, and severe cutaneous adverse reactions. Immune-mediated reactions may involve any organ system and may lead to hemophagocytic lymphohistiocytosis and macrophage activation syndrome.

Pleural and pericardial effusion: Patients experiencing dyspnea, chest pain, or unexplained tachycardia should be evaluated for the presence of a pericardial effusion. Patients with pre-existing pericardial effusion should be followed closely for pericardial fluid volume measurements and impact on cardiac function. When intervention is required for pericardial or pleural effusions, atezolizumab should be held, and appropriate workup includes cytology, lactate dehydrogenase (LDH), glucose, cholesterol, protein concentrations (with pleural effusions), and cell count.

Pulmonary events: Dyspnea, cough, fatigue, hypoxia, pneumonitis, and pulmonary infiltrates have been associated with the administration of atezolizumab. Patients will be assessed for pulmonary signs and symptoms throughout the study and will have computed tomography (CT) scans of the chest performed at every tumor assessment. All pulmonary events should be thoroughly evaluated for other commonly reported etiologies such as pneumonia or other infection, lymphangitic carcinomatosis, pulmonary embolism, heart failure, chronic obstructive pulmonary disease, or pulmonary hypertension. Management guidelines for pulmonary events are provided in Table 7.

TABLE 7
Pulmonary Event Management Guidelines

| Event | Management |
|---|---|
| Pulmonary event, | Continue atezolizumab and monitor closely. |
| Grade 1 | Re-evaluate on serial imaging. |
| | Consider patient referral to pulmonary specialist. |
| Pulmonary event, | Withhold atezolizumab for up to 12 weeks after event onset. ^{a} |
| Grade 2 | Refer patient to pulmonary and infectious disease specialists and consider |
| | bronchoscopy or BAL. |
| | Initiate treatment with corticosteroids equivalent to 1-2 mg/kg/day oral |
| | prednisone. |
| | If event resolves to Grade 1 or better, resume atezolizumab. ^{b} |
| | If event does not resolve to Grade 1 or better while withholding |
| | atezolizumab, permanently discontinue atezolizumab. |
| | For recurrent events, treat as a Grade 3 or 4 event. |
| Pulmonary event, | Permanently discontinue atezolizumab. |
| Grade 3 or 4 | Bronchoscopy or BAL is recommended. |
| | Initiate treatment with corticosteroids equivalent to 1-2 mg/kg/day oral |
| | prednisone. |
| | If event does not improve within 48 hours after initiating corticosteroids, |
| | consider adding an immunosuppressive agent. |
| | If event resolves to Grade 1 or better, taper corticosteroids over ≥1 month. |

BAL = bronchoscopic alveolar lavage
^{a} Atezolizumab may be withheld for a longer period of time (i.e., >12 weeks after event onset) to allow for corticosteroids (if initiated) to be reduced to the equivalent of ≤10 mg/day oral prednisone. The acceptable length of the extended period of time must be determined by the investigator and the Medical Monitor.
^{b} If corticosteroids have been initiated, they must be tapered over ≥1 month to the equivalent of ≤10 mg/day oral prednisone before atezolizumab can be resumed.

Hepatic events: Immune-mediated hepatitis has been associated with the administration of atezolizumab. Eligible patients must have adequate liver function, as manifested by measurements of total bilirubin and hepatic transaminases, and liver function will be monitored throughout study treatment. Management guidelines for hepatic events are provided in Table 8.

Patients with right upper-quadrant abdominal pain and/or unexplained nausea or vomiting should have liver function tests (LFTs) performed immediately and reviewed before administration of the next dose of study drug.

For patients with elevated LFTs, concurrent medication, viral hepatitis, and toxic or neoplastic etiologies should be considered and addressed, as appropriate.

TABLE 8
Hepatic Event Management

| Event | Management |
|---|---|
| Hepatic event, | Continue atezolizumab. |
| Grade 1 | Monitor LFTs until values resolve to within normal limits or to baseline values. |
| Hepatic event, | All events: |
| Grade 2 | Monitor LFTs more frequently until return to baseline values. |
| | Events of >5 days' duration: |
| | Withhold atezolizumab for up to 12 weeks after event onset. ^{a} |
| | Initiate treatment with corticosteroids equivalent to 1-2 mg/kg/day oral |
| | prednisone. |
| | If event resolves to Grade 1 or better, resume atezolizumab. ^{b} |
| | If event does not resolve to Grade 1 or better while withholding atezolizumab, |
| | permanently discontinue atezolizumab. |
| Hepatic event, | Permanently discontinue atezolizumab. |
| Grade 3 or 4 | Consider patient referral to gastrointestinal specialist for evaluation and liver |
| | biopsy to establish etiology of hepatic injury. |
| | Initiate treatment with corticosteroids equivalent to 1-2 mg/kg/day oral |
| | prednisone. |
| | If event does not improve within 48 hours after initiating corticosteroids, |
| | consider adding an immunosuppressive agent. |
| | If event resolves to Grade 1 or better, taper corticosteroids over ≥1 month. |

LFT = liver function test;
^{a} Atezolizumab may be withheld for a longer period of time (i.e., >12 weeks after event onset) to allow for corticosteroids (if initiated) to be reduced to the equivalent of ≤10 mg/day oral prednisone. The acceptable length of the extended period of time must be determined by the investigator;
^{b} If corticosteroids have been initiated, they must be tapered over ≥1 month to the equivalent of ≤10 mg/day oral prednisone before atezolizumab can be resumed.

Gastrointestinal events: Immune-mediated colitis has been associated with the administration of atezolizumab. Management guidelines for diarrhea or colitis are provided in Table 9.

All events of diarrhea or colitis should be thoroughly evaluated for other more common etiologies. For events of significant duration or magnitude or associated with signs of systemic inflammation or acute-phase reactants (e.g., increased C-reactive protein, platelet count, or bandemia): Perform sigmoidoscopy (or colonoscopy, if appropriate) with colonic biopsy, with three to five specimens for standard paraffin block to check for inflammation and lymphocytic infiltrates to confirm colitis diagnosis.

TABLE 9
Management of Gastrointestinal Events

| Event | Management |
|---|---|
| Diarrhea or | Continue atezolizumab. |
| colitis, Grade 1 | Initiate symptomatic treatment. |
| | Endoscopy is recommended if symptoms persist for >7 days. |
| | Monitor closely. |
| Diarrhea or | Withhold atezolizumab for up to 12 weeks after event onset. ^{a} |
| colitis, Grade 2 | Initiate symptomatic treatment. |
| | Patient referral to GI specialist is recommended. |
| | For recurrent events or events that persist >5 days, initiate treatment with |
| | corticosteroids equivalent to 1-2 mg/kg/day oral prednisone. |
| | If event resolves to Grade 1 or better, resume atezolizumab. ^{b} |
| | If event does not resolve to Grade 1 or better while withholding |
| | atezolizumab, permanently discontinue atezolizumab. |
| Diarrhea or | Withhold atezolizumab for up to 12 weeks after event onset. ^{a} |
| colitis, Grade 3 | Refer patient to GI specialist for evaluation and confirmatory biopsy. |
| | Initiate treatment with corticosteroids equivalent to 1-2 mg/kg/day IV |
| | methylprednisolone and convert to 1-2 mg/kg/day oral prednisone or |
| | equivalent upon improvement. |
| | If event resolves to Grade 1 or better, resume atezolizumab. ^{b} |
| | If event does not resolve to Grade 1 or better while withholding |
| | atezolizumab, permanently discontinue atezolizumab. |
| Diarrhea or | Permanently discontinue atezolizumab. |
| colitis, Grade 4 | Refer patient to GI specialist for evaluation and confirmation biopsy. |
| | Initiate treatment with corticosteroids equivalent to 1-2 mg/kg/day IV |
| | methylprednisolone and convert to 1-2 mg/kg/day oral prednisone or |
| | equivalent upon improvement. |
| | If event does not improve within 48 hours after initiating corticosteroids, |
| | consider adding an immunosuppressive agent. |
| | If event resolves to Grade 1 or better, taper corticosteroids over ≥1 month. |

GI = gastrointestinal.
^{a} Atezolizumab may be withheld for a longer period of time (i.e., >12 weeks after event onset) to allow for corticosteroids (if initiated) to be reduced to the equivalent of ≤10 mg/day oral prednisone. The acceptable length of the extended period of time can be determined by the investigator.
^{b} If corticosteroids have been initiated, they must be tapered over ≥1 month to the equivalent of ≤10 mg/day oral prednisone before atezolizumab can be resumed.

Endocrine disorders: Thyroid disorders, adrenal insufficiency, diabetes mellitus, and pituitary disorders have been associated with the administration of atezolizumab. Management guidelines for endocrine events are provided in Table 10.

Patients experiencing one or more unexplained AEs possibly indicative of endocrine dysfunction (including headache, fatigue, myalgias, impotence, mental status changes, and constipation) should be investigated for the presence of thyroid, pituitary, or adrenal endocrinopathies. The patient should be referred to an endocrinologist if an endocrinopathy is suspected. Thyroid stimulating hormone (TSH) and free T3 and T4 levels should be measured to determine whether thyroid abnormalities are present. Pituitary hormone levels and function tests [e.g., TSH, growth hormone, luteinizing hormone, follicle-stimulating hormone, testosterone, prolactin, adrenocorticotropic hormone (ACTH) levels, and ACTH stimulation test] and MRI of the brain (with detailed pituitary sections) may help to differentiate primary pituitary insufficiency from primary adrenal insufficiency. Table 10 describes dose management guidelines for hyperthyroidism, hypothyroidism, symptomatic adrenal insufficiency, and hyperglycemia.

TABLE 10
Management of Endocrine Events

| Event | Management |
|---|---|
| Asymptomatic | Continue atezolizumab. |
| hypothyroidism | Initiate treatment with thyroid replacement hormone. |
| | Monitor TSH weekly. |
| Symptomatic | Withhold atezolizumab. |
| hypothyroidism | Initiate treatment with thyroid replacement hormone. |
| | Monitor TSH weekly. |
| | Consider patient referral to endocrinologist. |
| | Resume atezolizumab when symptoms are controlled and thyroid |
| | function is improving. |
| Asymptomatic | TSH ≥0.1 mU/L and <0.5 mU/L: |
| hyperthyroidism | Continue atezolizumab. |
| | Monitor TSH every 4 weeks. |
| | TSH <0.1 mU/L: |
| | Follow guidelines for symptomatic hyperthyroidism. |
| Symptomatic | Withhold atezolizumab. |
| hyperthyroidism | Initiate treatment with anti-thyroid drug such as methimazole or |
| | carbimazole as needed. |
| | Consider patient referral to endocrinologist. |
| | Resume atezolizumab when symptoms are controlled and thyroid |
| | function is improving. |
| | Permanently discontinue atezolizumab. |
| Symptomatic adrenal | Withhold atezolizumab for up to 12 weeks after event onset. ^{a} |
| insufficiency, | Refer patient to endocrinologist. |
| Grade 2-4 | Perform appropriate imaging. |
| | Initiate treatment with corticosteroids equivalent to 1-2 mg/kg/day |
| | IV methylprednisolone and convert to 1-2 mg/kg/day oral prednisone |
| | or equivalent upon improvement. |
| | If event resolves to Grade 1 or better and patient is stable on |
| | replacement therapy, resume atezolizumab. ^{b} |
| | If event does not resolve to Grade 1 or better or patient is not stable |
| | on replacement therapy while withholding atezolizumab, |
| | permanently discontinue atezolizumab. |
| Hyperglycemia, | Continue atezolizumab. |
| Grade 1 or 2 | Investigate for diabetes. If patient has Type 1 diabetes, treat as a |
| | Grade 3 event. If patient does not have Type 1 diabetes, treat as per |
| | institutional guidelines. |
| | Monitor for glucose control. |
| Hyperglycemia, | Withhold atezolizumab. |
| Grade 3 or 4 | Initiate treatment with insulin. |
| | Monitor for glucose control. |
| | Consider referral to endocrinologist, particularly if patient is deemed |
| | to have atezolizumab-induced diabetes; if so, obtain C-peptide level |
| | paired with glucose, autoantibody levels (e.g. GAD65, islet cell |
| | autoantibodies), and hemoglobin A1C level. |
| | If patient is found to have diabetic ketoacidosis or hyperglycemic |
| | hyperosmolar syndrome, treat as per institutional guidelines with |
| | appropriate management and laboratory values (e.g. anion gap, |
| | ketones, blood pH, etc.) reported. |
| | Resume atezolizumab when symptoms resolve and glucose levels are |
| | stable. |
| Hypophysitis (pan- | Withhold atezolizumab for up to 12 weeks after event onset. ^{a} |
| hypopituitarism), | Refer patient to endocrinologist. |
| Grade 2 or 3 | Perform brain MRI (pituitary protocol). |
| | Initiate treatment with corticosteroids equivalent to 1-2 mg/kg/day |
| | IV methylprednisolone and convert to 1-2 mg/kg/day oral prednisone |
| | or equivalent upon improvement. |
| | Initiate hormone replacement if clinically indicated. |
| | If event resolves to Grade 1 or better, resume atezolizumab. ^{b} |
| | If event does not resolve to Grade 1 or better while withholding |
| | atezolizumab, permanently discontinue atezolizumab. |
| | For recurrent hypophysitis, treat as a Grade 4 event. |
| Hypophysitis (pan- | Permanently discontinue atezolizumab. |
| hypopituitarism), | Refer patient to endocrinologist. |
| Grade 4 | Perform brain MRI (pituitary protocol). |
| | Initiate treatment with corticosteroids equivalent to 1-2 mg/kg/day |
| | IV methylprednisolone and convert to 1-2 mg/kg/day oral prednisone |
| | or equivalent upon improvement. |
| | Initiate hormone replacement if clinically indicated. |

MRI = magnetic resonance imaging; TSH = thyroid-stimulating hormone.
^{a} Atezolizumab may be withheld for a longer period of time (i.e., >12 weeks after event onset) to allow for corticosteroids (if initiated) to be reduced to the equivalent of ≤10 mg/day oral prednisone. The acceptable length of the extended period of time can be determined by the investigator.
^{b} If corticosteroids have been initiated, they must be tapered over ≥1 month to the equivalent of ≤10 mg/day oral prednisone before atezolizumab can be resumed.

Ocular events: An ophthalmologist should evaluate visual complaints (e.g., uveitis, retinal events). Management guidelines for ocular events are provided in Table 11.

TABLE 11
Management guidelines for ocular events

| Event | Management |
|---|---|
| Ocular event, | Continue atezolizumab. |
| Grade 1 | Patient referral to ophthalmologist is strongly recommended. |
| | Initiate treatment with topical corticosteroid eye drops and topical |
| | immunosuppressive therapy. |
| | If symptoms persist, treat as a Grade 2 event. |
| Ocular event, | Withhold atezolizumab for up to 12 weeks after event onset. ^{a} |
| Grade 2 | Patient referral to ophthalmologist is strongly recommended. |
| | Initiate treatment with topical corticosteroid eye drops and topical |
| | immunosuppressive therapy. |
| | If event resolves to Grade 1 or better, resume atezolizumab. ^{b} |
| | If event does not resolve to Grade 1 or better while withholding |
| | atezolizumab, permanently discontinue atezolizumab. |
| Ocular event, | Permanently discontinue atezolizumab. |
| Grade 3 or 4 | Refer patient to ophthalmologist. |
| | Initiate treatment with corticosteroids equivalent to 1-2 mg/kg/day oral |
| | prednisone. |
| | If event resolves to Grade 1 or better, taper corticosteroids over ≥1 month. |

^{a} Atezolizumab may be withheld for a longer period of time (i.e., >12 weeks after event onset) to allow for corticosteroids (if initiated) to be reduced to the equivalent of ≤10 mg/day oral prednisone. The acceptable length of the extended period of time can be determined by the investigator.
^{b} If corticosteroids have been initiated, they must be tapered over ≥1 month to the equivalent of ≤10 mg/day oral prednisone before atezolizumab can be resumed.

Immune-mediated Myocarditis: Immune-mediated myocarditis has been associated with the administration of atezolizumab. Immune-mediated myocarditis should be suspected in any patient presenting with signs or symptoms suggestive of myocarditis, including, but not limited to, laboratory (e.g., B-NP [B-Natriuretic Peptide]) or cardiac imaging abnormalities, dyspnea, chest pain, palpitations, fatigue, decreased exercise tolerance, or syncope.

Immune-mediated myocarditis needs to be distinguished from myocarditis resulting from infection (commonly viral, e.g., in a patient who reports a recent history of gastrointestinal illness), ischemic events, underlying arrhythmias, exacerbation of preexisting cardiac conditions, or progression of malignancy. All patients with possible myocarditis should be urgently evaluated by performing cardiac enzyme assessment, an electrocardiogram (ECG), a chest X-ray, an echocardiogram, and a cardiac MRI as appropriate per institutional guidelines. A cardiologist should be consulted. An endomyocardial biopsy may be considered to enable a definitive diagnosis and appropriate treatment, if clinically indicated.

Patients with signs and symptoms of myocarditis, in the absence of an identified alternate etiology, should be treated according to the guidelines in Table 12.

TABLE 12
Management of Immune-mediated Myocarditis Events

| Event | Management |
|---|---|
| Immune-related | Withhold atezolizumab for up to 12 weeks after event onset ^{a}. |
| myocarditis, Grade 2 | Refer patient to cardiologist. |
| | Initiate treatment as per institutional guidelines and consider antiarrhythmic |
| | drugs, temporary pacemaker, ECMO, or VAD as appropriate. |
| | Consider treatment with corticosteroids equivalent to 1-2 |
| | mg/kg/day IV methylprednisolone and convert to |
| | 1-2 mg/kg/day oral prednisone or equivalent upon improvement. |
| | If symptoms resolve to below Grade 2 (i.e. patient is completely |
| | asymptomatic), resume atezolizumab. ^{b} |
| | If symptoms do not resolve to below Grade 2 while withholding |
| | atezolizumab, permanently discontinue atezolizumab. |
| Immune-related | Permanently discontinue atezolizumab. |
| myocarditis, Grade | Refer patient to cardiologist. |
| 3-4 | Initiate treatment as per institutional guidelines and consider antiarrhythmic |
| | drugs, temporary pacemaker, ECMO, or VAD as appropriate. |
| | Initiate treatment with corticosteroids equivalent to 1-2 mg/kg/day IV |
| | methylprednisolone and convert to 1-2 mg/kg/day oral prednisone or |
| | equivalent upon improvement. |
| | If event does not improve within 48 hours after initiating corticosteroids, |
| | consider adding an immunosuppressive agent. |
| | If symptoms resolve to below Grade 2, taper corticosteroids over ≥1 |
| | month. |

ECMO = extracorporeal membrane oxygenation; VAD = ventricular assist device.
^{a} Atezolizumab may be withheld for a longer period of time (i.e., >12 weeks after event onset) to allow for corticosteroids (if initiated) to be reduced to the equivalent of ≤10 mg/day oral prednisone. The acceptable length of the extended period of time must be documented by the investigator.
^{b} If corticosteroids have been initiated, they must be tapered over ≥1 month to the equivalent of ≤10 mg/day oral prednisone before atezolizumab can be resumed.

Infusion-Related Reactions and Cytokine-Release Syndrome: No premedication is indicated for the administration of Cycle 1 of atezolizumab. However, patients who experience an infusion-related reaction (IRR) or cytokine-release syndrome (CRS) with atezolizumab may receive premedication with antihistamines, antipyretics, and/or analgesics (e.g., acetaminophen) for subsequent infusions. Metamizole (dipyrone) is prohibited in treating atezolizumab-associated IRRs because of its potential for causing agranulocytosis.

IRRs can occur with the administration of monoclonal antibodies and have been reported with atezolizumab. These reactions, which are thought to be due to release of cytokines and/or other chemical mediators, occur within 24 hours of atezolizumab administration and are generally mild to moderate in severity.

CRS is a supraphysiologic response following administration of any immune therapy that results in activation or engagement of endogenous or infused T cells and/or other immune effector cells. Symptoms can be progressive, always include fever at the onset, and may include hypotension, capillary leak (hypoxia), and end-organ dysfunction (Lee et al., 2019). CRS has been well documented with chimeric antigen receptor T-cell therapies and bispecific T-cell engager antibody therapies but has also been reported with immunotherapies that target PD-1 or PD-L1 (Rotz et al., 2017; Adashek and Feldman 2019) including atezolizumab.

There may be significant overlap in signs and symptoms of IRRs and CRS, and in recognition of the challenges in clinically distinguishing between the two, consolidated guidelines in for medical management of IRRs and CRS are provided in Table 13.

TABLE 13
Management Guidelines for Infusion-Related Reactions and Cytokine-Release Syndrome

| Event | Management |
|---|---|
| Grade 1^{a} | Immediately interrupt infusion. |
| Fever^{b} with or | Upon symptom resolution, wait 30 minutes and then restart infusion at half |
| without | the rate being given at the time of event onset. |
| constitutional | If the infusion is tolerated at the reduced rate for 30 minutes, the infusion |
| symptoms | rate may be increased to the original rate. |
| | If symptoms recur, discontinue infusion of this dose. |
| | Administer symptomatic treatment,^{c} including maintenance of IV fluids for |
| | hydration. |
| | In case of rapid decline or prolonged CRS (>2 days) or in patients with |
| | significant symptoms and/or comorbidities, consider managing as per Grade 2. |
| | For subsequent infusions, consider administration of oral premedication |
| | with antihistamines, anti-pyretics, and/or analgesics, and monitor closely for |
| | IRRs and/or CRS. |
| Grade 2^{a} | Immediately interrupt atezolizumab infusion. |
| Fever^{b} with | Upon symptom resolution, wait for 30 minutes and then restart infusion at |
| hypotension not | half the rate being given at the time of event onset. |
| requiring | If symptoms recur, discontinue infusion of this dose. |
| vasopressors | Administer symptomatic treatment.^{c} |
| and/or | For hypotension, administer IV fluid bolus as needed. |
| Hypoxia | Monitor cardiopulmonary and other organ function closely (in the ICU, if |
| requiring low- | appropriate). Administer IV fluids as clinically indicated and manage |
| flow oxygen^{d} | constitutional symptoms and organ toxicities as per institutional practice. |
| by nasal cannula | Rule out other inflammatory conditions that can mimic CRS (e.g., sepsis). |
| or blow-by | If no improvement within 24 hours, initiate workup and assess for signs and |
| | symptoms of HLH or MAS. |
| | Consider IV corticosteroids (e.g., methylprednisolone 2 mg/kg/day or |
| | dexamethasone 10 mg every 6 hours). |
| | Consider anti-cytokine therapy.^{e} |
| | Consider hospitalization until complete resolution of symptoms. If no |
| | improvement within 24 hours, manage as per Grade 3, that is, hospitalize |
| | patient (monitoring in the ICU is recommended), permanently discontinue |
| | atezolizumab. |
| | If symptoms resolve to Grade 1 or better for 3 consecutive days, the next |
| | dose of atezolizumab may be administered. For subsequent infusions, |
| | consider administration of oral premedication with antihistamines, anti- |
| | pyretics, and/or analgesics and monitor closely for IRRs and/or CRS. |
| | If symptoms do not resolve to Grade 1 or better for 3 consecutive days, |
| | contact a physician. |
| Grade 3^{a} | Permanently discontinue atezolizumab. |
| Fever^{b} with | Administer symptomatic treatment.^{c} |
| hypotension | For hypotension, administer IV fluid bolus and vasopressor as needed. |
| requiring a | Monitor cardiopulmonary and other organ function closely; monitoring in |
| vasopressor | the ICU is recommended. Administer IV fluids as clinically indicated and |
| (with or without | manage constitutional symptoms and organ toxicities as per institutional |
| vasopressin) | practice. |
| and/or | Rule out other inflammatory conditions that can mimic CRS (e.g., sepsis). |
| Hypoxia | If no improvement within 24 hours, initiate workup and assess for signs and |
| requiring high- | symptoms of HLH or MAS. |
| flow oxygen^{d} | Administer IV corticosteroids (e.g., methylprednisolone 2 mg/kg/day or |
| by nasal | dexamethasone 10 mg every 6 hours). |
| cannula, face | Consider anti-cytokine therapy.^{e} |
| mask, non- | Hospitalize patient until complete resolution of symptoms. If no |
| rebreather mask, | improvement within 24 hours, manage as per Grade 4, that is, admit patient |
| or venturi mask | to ICU and initiate hemodynamic monitoring, mechanical ventilation, and/or |
| | IV fluids and vasopressors as needed; for patients who are refractory to anti- |
| | cytokine therapy, experimental treatments may be considered at the |
| | discretion of the investigator. |
| Grade 4^{a} | Permanently discontinue atezolizumab. |
| Fever^{b} with | Administer symptomatic treatment.^{c} |
| hypotension | Admit patient to ICU and initiate hemodynamic monitoring, mechanical |
| requiring | ventilation, and/or IV fluids and vasopressors as needed. Monitor other |
| multiple | organ function closely. Manage constitutional symptoms and organ |
| vasopressors | toxicities as per institutional practice. |
| (excluding | Rule out other inflammatory conditions that can mimic CRS (e.g., sepsis). |
| vasopressin) | If no improvement within 24 hours, initiate workup and assess for signs and |
| and/or | symptoms of HLH or MAS. |
| Hypoxia | Administer IV corticosteroids (e.g., methylprednisolone 2 mg/kg/day or |
| requiring | dexamethasone 10 mg every 6 hours). |
| oxygen by | Consider anti-cytokine therapy.^{e} For patients who are refractory to anti- |
| positive pressure | cytokine therapy, experimental treatments^{f} may be considered at the |
| (e.g., CPAP, | discretion of the investigator. |
| BiPAP, | Hospitalize patient until complete resolution of symptoms. |
| intubation and | |
| mechanical | |
| ventilation) | |

ASTCT = American Society for Transplantation and Cellular Therapy; BiPAP = bi-level positive airway pressure; CAR = chimeric antigen receptor; CPAP = continuous positive airway pressure; CRS = cytokine-release syndrome; HLH = hemophagocytic lymphohistiocytosis; IRR = infusion-related reaction; MAS = macrophage activation syndrome.
Note:
The management guidelines have been adapted from NCCN guidelines for management of CAR T-cell-related toxicities (Version 2.2019).
Grading system for management guidelines is based on ASTCT consensus grading for CRS. NCI CTCAE (version as specified in the protocol) should be used when reporting severity of IRRs, CRS, or organ toxicities associated with CRS on the Adverse Event eCRF. Organ toxicities associated with CRS should not influence overall CRS grading.
Fever is defined as temperature ≥38° C. not attributable to any other cause. In patients who develop CRS and then receive anti-pyretic, anti-cytokine, or corticosteroid therapy, fever is no longer required when subsequently determining event severity (grade). In this case, the grade is driven by the presence of hypotension and/or hypoxia.
Symptomatic treatment may include oral or IV antihistamines, anti-pyretics, analgesics, bronchodilators, and/or oxygen. For bronchospasm, urticaria, or dyspnea, additional treatment may be administered as per institutional practice.
Low flow is defined as oxygen delivered at ≤6 L/min, and high flow is defined as oxygen delivered at >6 L/min.
There are case reports where anti-cytokine therapy has been used for treatment of CRS with immune checkpoint inhibitors (Rotz et al. 2017; Adashek and Feldman 2019), but data are limited, and the role of such treatment in the setting of antibody-associated CRS has not been established.
Refer to Riegler et al. for information on experimental treatments for CRS.

Pancreatic events: Symptoms of abdominal pain associated with elevations of amylase and lipase, suggestive of pancreatitis, have been associated with the administration of atezolizumab. The differential diagnosis of acute abdominal pain should include pancreatitis. Appropriate workup should include an evaluation for ductal obstruction, as well as serum amylase and lipase tests. Management guidelines for pancreatic events, including pancreatitis, are provided in Table 14.

TABLE 14
Management guidelines for Pancreatic Events

| Event | Management |
|---|---|
| Amylase and/or lipase | Amylase and/or lipase >1.5-2.0 × ULN: |
| elevation, Grade 2 | Continue atezolizumab. |
| | Monitor amylase and lipase weekly. |
| | For prolonged elevation (e.g., >3 weeks), consider treatment with |
| | corticosteroids equivalent to 10 mg/day oral prednisone. |
| | Asymptomatic with amylase and/or lipase >2.0-5.0 × ULN: |
| | Treat as a Grade 3 event. |
| Amylase and/or lipase | Withhold atezolizumab for up to 12 weeks after event onset. ^{a} |
| elevation, Grade 3 or 4 | Refer patient to GI specialist. |
| | Monitor amylase and lipase every other day. |
| | If no improvement, consider treatment with corticosteroids |
| | equivalent to 1-2 mg/kg/day oral prednisone. |
| | If event resolves to Grade 1 or better, resume atezolizumab. ^{b} |
| | If event does not resolve to Grade 1 or better while withholding |
| | atezolizumab, permanently discontinue atezolizumab. ^{c} |
| | For recurrent events, permanently discontinue atezolizumab. |
| Immune-related pancreatitis, | Withhold atezolizumab for up to 12 weeks after event onset. ^{a} |
| Grade 2 or 3 | Refer patient to GI specialist. |
| | Initiate treatment with corticosteroids equivalent to |
| | 1-2 mg/kg/day IV methylprednisolone and convert to |
| | 1-2 mg/kg/day oral prednisone or equivalent upon improvement. |
| | If event resolves to Grade 1 or better, resume atezolizumab. ^{b} |
| | If event does not resolve to Grade 1 or better while withholding |
| | atezolizumab, permanently discontinue atezolizumab. |
| | For recurrent events, permanently discontinue atezolizumab. |
| Immune-related pancreatitis, | Permanently discontinue atezolizumab. |
| Grade 4 | Refer patient to GI specialist. |
| | Initiate treatment with corticosteroids equivalent to |
| | 1-2 mg/kg/day IV methylprednisolone and convert to |
| | 1-2 mg/kg/day oral prednisone or equivalent upon improvement. |
| | If event does not improve within 48 hours after initiating |
| | corticosteroids, consider adding an immunosuppressive agent. |
| | If event resolves to Grade 1 or better, taper corticosteroids |
| | over ≥1 month. |

GI = gastrointestinal.
^{a} Atezolizumab may be withheld for a longer period of time (i.e., >12 weeks after event onset) to allow for corticosteroids (if initiated) to be reduced to the equivalent of ≤10 mg/day oral prednisone. The acceptable length of the extended period of time can be determined by the investigator.
^{b} If corticosteroids have been initiated, they must be tapered over ≥1 month to the equivalent of ≤10 mg/day oral prednisone before atezolizumab can be resumed.

Dermatologic events Treatment-emergent rash has been associated with atezolizumab. The majority of cases of rash were mild in severity and self-limited, with or without pruritus. A dermatologist should evaluate persistent and/or severe rash or pruritus. Although uncommon, cases of severe cutaneous adverse reactions such as Stevens-Johnson syndrome and toxic epidermal necrolysis have been reported with atezolizumab. A biopsy should be considered unless contraindicated. Management guidelines for dermatologic events are provided in Table 15.

TABLE 15
Management Guidelines for Dermatologic Events

| Event | Management |
|---|---|
| Dermatologic | Continue atezolizumab. |
| event, Grade 1 | Consider treatment with topical corticosteroids and/or other symptomatic |
| | therapy (e.g., antihistamines). |
| Dermatologic | Continue atezolizumab. |
| event, Grade 2 | Consider patient referral to dermatologist for evaluation and if indicated, |
| | biopsy. |
| | Initiate treatment with topical corticosteroids. |
| | Consider treatment with higher-potency topical corticosteroids if event |
| | does not improve. |
| Dermatologic | Withhold atezolizumab for up to 12 weeks after event onset. ^{a} |
| event, Grade 3 | Refer patient to dermatologist for evaluation and if indicated, biopsy. |
| | Initiate treatment with corticosteroids equivalent to 10 mg/day oral |
| | prednisone, increasing dose to 1-2 mg/kg/day if event does not improve |
| | within 48-72 hours. |
| | If event resolves to Grade 1 or better, resume atezolizumab. ^{b} |
| | If event does not resolve to Grade 1 or better while withholding |
| | atezolizumab, permanently discontinue atezolizumab. |
| Dermatologic | Permanently discontinue atezolizumab. |
| event, Grade 4 | |
| Stevens-Johnson | Additional guidance for Stevens-Johnson syndrome or toxic |
| syndrome or toxic | epidermal necrolysis: |
| epidermal | Withhold atezolizumab for suspected Stevens-Johnson syndrome or toxic |
| necrolysis (any | epidermal necrolysis. |
| grade) | Confirm diagnosis by referring patient to a specialist (dermatologist, |
| | ophthalmologist, or urologist as relevant) for evaluation and, if indicated, |
| | biopsy. |
| | Follow the applicable treatment and management guidelines above. |
| | If Stevens-Johnson syndrome or toxic epidermal necrolysis is confirmed, |
| | permanently discontinue atezolizumab. |

Atezolizumab may be withheld for a longer period of time (i.e., >12 weeks after event onset) to allow for corticosteroids (if initiated) to be reduced to the equivalent of ≤10 mg/day oral prednisone. The acceptable length of the extended period of time can be determined by the investigator.
^{b} If corticosteroids have been initiated, they must be tapered over ≥1 month to the equivalent of ≤10 mg/day oral prednisone before atezolizumab can be resumed.

Neurologic disorders: Myasthenia gravis and Guillain-Barré syndrome have been observed with single agent atezolizumab. Patients may present with signs and symptoms of sensory and/or motor neuropathy. Diagnostic work-up is essential for an accurate characterization to differentiate between alternative etiologies. Management guidelines for neurologic disorders are provided in Table 16.

TABLE 16
Management Guidelines for Neurologic Disorders

| Event | Management |
|---|---|
| Immune-related | Continue atezolizumab. |
| neuropathy, | Investigate etiology. |
| Grade 1 | |
| Immune-related | Withhold atezolizumab for up to 12 weeks after event |
| neuropathy, | onset. ^{a} |
| Grade 2 | Investigate etiology. |
| | Initiate treatment as per institutional guidelines. |
| | If symptoms resolve to below Grade 2, resume |
| | atezolizumab. ^{b} |
| | If symptoms do not resolve to below Grade 2 while |
| | withholding atezolizumab, permanently discontinue |
| | atezolizumab. |
| Immune-related | Permanently discontinue atezolizumab. |
| neuropathy, | Initiate treatment as per institutional guidelines. |
| Grade 3 or 4 | |
| Myasthenia gravis | Permanently discontinue atezolizumab. |
| and Guillain- | Refer patient to neurologist. |
| Barré syndrome | Initiate treatment as per institutional guidelines. |
| (any grade) | Consider initiation of corticosteroids equivalent to 1-2 |
| | mg/kg/day oral or IV prednisone. |

^{a} Atezolizumab may be withheld for a longer period of time (i.e., >12 weeks after event onset) to allow for corticosteroids (if initiated) to be reduced to the equivalent of ≤10 mg/day oral prednisone. The acceptable length of the extended period of time must be determined by the investigator.
^{b} If corticosteroids have been initiated, they must be tapered over ≥1 month to the equivalent of ≤10 mg/day oral prednisone before atezolizumab can be resumed.

Immune-Mediated Meningoencephalitis: Immune-mediated meningoencephalitis is an identified risk associated with the administration of atezolizumab. Immune-mediated meningoencephalitis should be suspected in any patient presenting with signs or symptoms suggestive of meningitis or encephalitis, including, but not limited to, headache, neck pain, confusion, seizure, motor or sensory dysfunction, and altered or depressed level of consciousness. Encephalopathy from metabolic or electrolyte imbalances needs to be distinguished from potential meningoencephalitis resulting from infection (bacterial, viral, or fungal) or progression of malignancy, or secondary to a paraneoplastic process.

All patients being considered for meningoencephalitis should be urgently evaluated with a CT scan and/or MRI scan of the brain to evaluate for metastasis, inflammation, or edema. If deemed safe by the treating physician, a lumbar puncture should be performed, and a neurologist should be consulted.

Patients with signs and symptoms of meningoencephalitis, in the absence of an identified alternate etiology, should be treated according to the guidelines in Table 17.

TABLE 17
Management Guidelines for Immune-
related Meningoencephalitis Events

| Event | Management |
|---|---|
| Immune-related | Permanently discontinue atezolizumab. |
| meningoencephalitis, | Refer patient to neurologist. |
| all grades | Initiate treatment with corticosteroids |
| | equivalent to 1-2 mg/kg/day IV |
| | methylprednisolone and convert to 1-2 |
| | mg/kg/day oral prednisone or equivalent |
| | upon improvement. |
| | If event does not improve within 48 hours after |
| | initiating corticosteroids, consider adding an |
| | immunosuppressive agent. |
| | If event resolves to Grade 1 or better, taper |
| | corticosteroids over ≥1 month. |

Renal events: Immune-mediated nephritis has been associated with the administration of atezolizumab. Eligible patients must have adequate renal function. Renal function, including serum creatinine, should be monitored throughout study treatment. Patients with abnormal renal function should be evaluated and treated for other more common etiologies (including prerenal and postrenal causes, and concomitant medications such as non-steroidal anti-inflammatory drugs). Refer the patient to a renal specialist if clinically indicated. A renal biopsy may be required to enable a definitive diagnosis and appropriate treatment. Patients with signs and symptoms of nephritis, in the absence of an identified alternate etiology, should be treated according to the guidelines in Table 18 below.

TABLE 18
Management Guidelines for Renal Events

| Event | Management |
|---|---|
| Renal event, | Continue atezolizumab. |
| Grade 1 | Monitor kidney function, including creatinine, closely until values resolve to |
| | within normal limits or to baseline values. |
| Renal event, | Withhold atezolizumab for up to 12 weeks after event onset. ^{a} |
| Grade 2 | Refer patient to renal specialist. |
| | Initiate treatment with corticosteroids equivalent to 1-2 mg/kg/day oral |
| | prednisone. |
| | If event resolves to Grade 1 or better, resume atezolizumab. ^{b} |
| | If event does not resolve to Grade 1 or better while withholding atezolizumab, |
| | permanently discontinue atezolizumab. |
| Renal event, | Permanently discontinue atezolizumab. |
| Grade 3 or 4 | Refer patient to renal specialist and consider renal biopsy. |
| | Initiate treatment with corticosteroids equivalent to 1-2 mg/kg/day oral |
| | prednisone. |
| | If event does not improve within 48 hours after initiating corticosteroids, |
| | consider adding an immunosuppressive agent. |
| | If event resolves to Grade 1 or better, taper corticosteroids over ≥1 month. |

^{a} Atezolizumab may be withheld for a longer period of time (i.e., >12 weeks after event onset) to allow for corticosteroids (if initiated) to be reduced to the equivalent of ≤10 mg/day oral prednisone. The acceptable length of the extended period of time can be determined by the investigator.
^{b} If corticosteroids have been initiated, they must be tapered over ≥1 month to the equivalent of ≤10 mg/day oral prednisone before atezolizumab can be resumed.

Immune-Mediated Myositis: Immune-mediated myositis has been associated with the administration of atezolizumab. Myositis or inflammatory myopathies are a group of disorders sharing the common feature of inflammatory muscle injury; dermatomyositis and polymyositis are among the most common disorders. Initial diagnosis is based on clinical (muscle weakness, muscle pain, skin rash in dermatomyositis), biochemical (serum creatine kinase increase), and imaging (electromyography/MRI) features, and is confirmed with a muscle biopsy. Patients with signs and symptoms of myositis, in the absence of an identified alternate etiology, should be treated according to the guidelines in Table 19.

TABLE 19
Management Guidelines for Immune-Mediated Myositis

| Event | Management |
|---|---|
| Immune-mediated | Continue atezolizumab. |
| myositis, Grade 1 | Refer patient to rheumatologist or neurologist. |
| | Initiate treatment as per institutional guidelines. |
| Immune-mediated | Withhold atezolizumab for up to 12 weeks after event onset ^{a} and contact |
| myositis, Grade 2 | Medical Monitor. |
| | Refer patient to rheumatologist or neurologist. |
| | Initiate treatment as per institutional guidelines. |
| | Consider treatment with corticosteroids equivalent to 1-2 mg/kg/day IV |
| | methylprednisolone and convert to 1-2 mg/kg/day oral prednisone or |
| | equivalent upon improvement. |
| | If corticosteroids are initiated and event does not improve within 48 hours |
| | after initiating corticosteroids, consider adding an immunosuppressive |
| | agent. |
| | If event resolves to Grade 1 or better, resume atezolizumab. ^{b} |
| | If event does not resolve to Grade 1 or better while withholding |
| | atezolizumab, permanently discontinue atezolizumab and contact Medical |
| | Monitor. |
| Immune-mediated | Withhold atezolizumab for up to 12 weeks after event onset ^{a} and contact |
| myositis, Grade 3 | Medical Monitor. |
| | Refer patient to rheumatologist or neurologist. |
| | Initiate treatment as per institutional guidelines. |
| | Respiratory support may be required in more severe cases. |
| | Initiate treatment with corticosteroids equivalent to 1-2 mg/kg/day IV |
| | methylprednisolone, or higher-dose bolus if patient is severely |
| | compromised (e.g., cardiac or respiratory symptoms, dysphagia, or |
| | weakness that severely limits mobility); convert to 1-2 mg/kg/day oral |
| | prednisone or equivalent upon improvement. |

^{a} Atezolizumab may be withheld for a longer period of time (i.e., >12 weeks after event onset) to allow for corticosteroids (if initiated) to be reduced to the equivalent of ≤10 mg/day oral prednisone. The acceptable length of the extended period of time must be agreed upon by the investigator and the Medical Monitor.

Hemophagocytic Lymphohistiocytosis and Macrophage Activation Syndrome: Immune-mediated reactions may involve any organ system and may lead to hemophagocytic lymphohistiocytosis (HLH) and macrophage activation syndrome (MAS). Patients with suspected HLH should be diagnosed according to published criteria by McClain and Eckstein (2017). A patient should be classified as having HLH if five of the following eight criteria are met:
Fever ≥38.5° C.
Splenomegaly
Peripheral blood cytopenia consisting of at least two of the following:
Hemoglobin <90 g/L (9 g/dL) (<100 g/L [10 g/dL] for infants <4 weeks old)
Platelet count <100×10⁹/L (100,000/mcL)
ANC <1.0×10⁹/L (1000/mcL)
Fasting triglycerides >2.992 mmol/L (265 mg/dL) and/or fibrinogen <1.5 g/L (150 mg/dL)
Hemophagocytosis in bone marrow, spleen, lymph node, or liver
Low or absent natural killer cell activity
Ferritin >500 mg/L (500 ng/mL)
Soluble interleukin 2 (IL-2) receptor (soluble CD25) elevated ≥2 standard deviations above age-adjusted laboratory-specific norms
   Patients with suspected MAS should be diagnosed according to published criteria for systemic juvenile idiopathic arthritis by Ravelli et al. (2016). A febrile patient should be classified as having MAS if the following criteria are met:
Ferritin >684 mg/L (684 ng/mL)
At least two of the following:
Platelet count ≤181×10⁹/L (181,000/mcL)
AST ≥48 U/L
Triglycerides >1.761 mmol/L (156 mg/dL)
Fibrinogen ≤3.6 g/L (360 mg/dL)
   Patients with suspected HLH or MAS should be treated according to the guidelines in Table 20.

TABLE 20
Management Guidelines for suspected HLH or MAS

| Event | Management |
|---|---|
| Suspected | Permanently discontinue atezolizumab and contact Medical |
| HLH | Monitor. |
| or MAS | Consider patient referral to hematologist. |
| | Initiate supportive care, including intensive care monitoring |
| | if indicated per institutional guidelines. |
| | Consider initiation of IV corticosteroids and/or an |
| | immunosuppressive agent. |
| | If event does not improve within 48 hours after initiating |
| | corticosteroids, consider adding an immunosuppressive agent. |
| | If event resolves to Grade 1 or better, taper corticosteroids |
| | over ≥1 month. |

HLH = hemophagocytic lymphohistiocytosis; MAS = macrophage activation syndrome.

### Bevacizumab

Surgery and wound healing complications: Discontinue bevacizumab in patients with wound healing complications requiring medical intervention. Withhold for at least 28 days prior to elective surgery. Do not administer for at least 28 days following surgery and until the wound is fully healed. Necrotizing fasciitis including fatal cases, has been reported in patients receiving bevacizumab, usually secondary to wound healing complications, gastrointestinal perforation or fistula formation. Discontinue bevacizumab in patients who develop necrotizing fasciitis.

Hypertension: Monitor blood pressure every two to three weeks during treatment with bevacizumab. Repeat blood pressure measurements, treat with appropriate anti-hypertensive therapy, and continue, reduce, or discontinue protocol therapy according to the guidelines specified in FIG. 19. Discontinue bevacizumab in patients who develop hypertensive crisis or hypertensive encephalopathy. Continue to monitor blood pressure at regular intervals in patients with bevacizumab-induced or -exacerbated hypertension after discontinuing bevacizumab.

Renal injury and proteinuria: At the start of each cycle, monitor proteinuria by urinalysis for the development or worsening of proteinuria with serial urinalyses during bevacizumab therapy. Patients with 2+ proteinuria on urinalysis should undergo further assessment with a 24-hour urine collection. Withhold for proteinuria greater than or equal to 2 grams per 24 hours and resume when less than 2 grams per 24 hours. Discontinue in patients who develop nephrotic syndrome.

Data from a postmarketing safety study showed poor correlation between UPCR (Urine Protein/Creatinine Ratio) and 24-hour urine protein [Pearson Correlation 0.39 (95% CI: 0.17, 0.57)].

Infusion-related reactions: Decrease the rate of infusion for mild, clinically insignificant infusion-related reactions. For subsequent infusions, see the premedication and infusion time guidance in the Agent Administration Section. Interrupt the infusion in patients with clinically significant infusion-related reactions and consider resuming at a slower rate following resolution. Discontinue in patients who develop a severe infusion-related reaction and administer appropriate medical therapy (e.g., epinephrine, corticosteroids, intravenous antihistamines, bronchodilators and/or oxygen). Management Guidelines for Bevacizumab are shown in Table 21.

TABLE 21
Management Guidelines for Bevacizumab

| | CTCAE Version | |
|---|---|---|
| Event | 5.0 Grade | Action to be Taken |
| Allergic reactions | Grade 1 | Systemic intervention not indicated - continue |
| or | | bevacizumab |
| Infusion-related | Grade 2 | Oral intervention indicated - slow infusion to 50% |
| reactions | | or interrupt if clinically indicated (re-start infusion |
| Or | | at 50% and increase in 50% increments if well |
| Anaphylaxis | | tolerated). Infusion can be re-started at the full rate |
| | | for subsequent infusions. |
| | Grade 3 | Bronchospasm (allergy-related |
| | | oedema/angioedema; hypotension); hospitalization |
| | | for clinical sequelae; intravenous intervention |
| | | indicated - discontinue bevacizumab |
| | Grade 4 | Life-threatening consequences; urgent intervention |
| | | indicated - discontinue bevacizumab |
| Thromboembolic | Any Grade | Discontinue bevacizumab |
| Event (arterial) | | |
| Thromboembolic | Grade 3 | Hold bevacizumab treatment. If the planned |
| Event (Venous) | | duration of full-dose anticoagulation is <2 weeks, |
| | | bevacizumab should be held until the full-dose |
| | | anticoagulation period is over. The use of direct |
| | | oral anticoagulants is not recommended. |
| | | If the planned duration of full-dose anticoagulation |
| | | is >2 weeks, bevacizumab may be resumed during |
| | | full-dose anticoagulation IF all of the criteria below |
| | | are met: |
| | | The patient must not have pathological conditions |
| | | that carry high risk of bleeding (e.g. tumor |
| | | involving major vessels or other conditions) |
| | | The patient must not have had hemorrhagic |
| | | events > grade 2 while on study |
| | | The patient must be on stable dose of heparin, low |
| | | molecular weight heparin, or have an in-range INR |
| | | (usually 2-3) on a stable dose of warfarin prior to |
| | | restarting bevacizumab. |
| | | If thromboemboli worsen/recur upon resumption of |
| | | study therapy, discontinue bevacizumab |
| | Grade 4 | Discontinue bevacizumab |
| Hypertension | [Treat with anti-hypertensive medication as needed. The goal of BP | |
| | control should be consistent with general medical practice] | |
| | Grade 1 | Consider increased BP monitoring; start anti- |
| | (SBP 120-139 mmHg | hypertensive medication if appropriate |
| | or DBP80-89 mm Hg) | |
| | Grade 2 asymptomatic | Begin (or modify baseline anti-HTN therapy) |
| | (SBP 140-159 mmHg | anti-hypertensive therapy and continue |
| | or DBP 90-99 mm Hg) | bevacizumab |
| | Grade 2 symptomatic | Start or adjust anti-hypertensive medication |
| | (SBP 140-159 mmHg | |
| | or DBP 90-99 mm Hg) | |
| | Grade 3 | Modify existing anti-HTN therapy (more than |
| | (≥SBP 160 mmHg | one drug or more intensive therapy than |
| | or ≥DBP 100 mmHg | previously indicated. |
| | | Hold bevacizumab until symptoms resolve |
| | | AND BP <160/90 mmHg |
| | Grade 4 | Discontinue bevacizumab |
| | (e.g.. Hypertensive | |
| | crisis or malignant | |
| | hypertension) | |
| Heart Failure or left | Grade 3 | Discontinue bevacizumab |
| ventricular dysfunction | Grade 4 | Discontinue bevacizumab |
| Proteinuria* | 1+ proteinuria | Continue bevacizumab |
| | (≥ULN - <1.0 g/24 h) | |
| | 2+ and 3+ proteinuria | 2+ - administer bevacizumab and obtain 24- |
| | (1.0 - <3.5 g/24 h) | hour urine protein before next administration |
| | | 3+ - obtain 24-hour urine protein and |
| | | administer bevacizumab if <2.0 g/24 h |
| | 4+ proteinuria | Obtain 24-hour urine protein and administer |
| | (≥3.5 g/24 h) | bevacizumab only when <2.0 g/24 h |
| Nephrotic syndrome | | Grade 3 or 4 Discontinue bevacizumab |
| Haemorrhage (CNS) | Any grade | Discontinue bevacizumab |
| | Grade 1 | Trace haemoptysis; continue bevacizumab |
| Haemorrhage | Grade 2-4 | ≥2.5 mL bright red blood per episode; |
| (haemoptysis) | | discontinue bevacizumab |
| Haemorrhage (other) | Grade 3-4 | Discontinue bevacizumab |
| RPLS (Reversible Posterior | | Discontinue bevacizumab |
| Leukoencephalopathy syndrome or PRES | | |
| (Posterior Reversible Encephalopathy | | |
| Syndrome) | | |
| Wound dehiscence requiring medical or | | Discontinue bevacizumab |
| surgical intervention | | |
| Perforation (GI, or any other organ) | | Discontinue bevacizumab |
| Fistula (GI, pulmonary or any other organ) | | Discontinue bevacizumab |
| Obstruction of GI tract | G2 requiring medical | Hold bevacizumab until complete resolution |
| | intervention | |
| | G3-4 | Hold bevacizumab until complete resolution |
| | | If surgery is required, patient may restart |
| | | bevacizumab after full recovery from surgery, |
| | | and at investigator's discretion |
| Febrile neutropenia | Grade 3 | Continue bevacizumab |
| | Grade 4 | Hold bevacizumab until resolution or return to |
| | | baseline |
| Platelet count | Grades 1-3 | Continue bevacizumab |
| decreased | Grade 4 | Hold bevacizumab until resolution or return to |
| | | baseline |
| Other Unspecified | Grade 3 | Hold bevacizumab until symptoms resolve |
| bevacizumab-related | | to ≤ grade 1 or baseline |
| AEs (except controlled | Grade 4 | Discontinue bevacizumab |
| nausea/vomiting). | | Upon consultation with the study |
| | | chair/medical monitor, resumption of |
| | | bevacizumab may be considered if a patient is |
| | | benefiting from therapy, and the G4 toxicity is |
| | | transient, has recovered to ≤ |
| | | grade 1 (or baseline) and unlikely to recur |
| | | with retreatment. |

### VI. Pharmaceutical Information

### A. Atezolizumab (NSC #783608): Other Names: Tecentriq™, MPDL3280A; Classification: Monoclonal Antibody; M.W.: 150 KD, Ode of Action: Anti-PD-L1.

Description: Atezolizumab is a humanized IgG1 monoclonal antibody consisting of two heavy chains (448 amino acids) and two light chains (214 amino acids). Atezolizumab targets human PD-L1 and inhibits its interaction with its receptor PD-1. Atezolizumab also blocks the binding of PD-L1 to B7.1, an interaction that is reported to provide additional inhibitory signals to T cells (Butte et al. 2007).

How Supplied: Atezolizumab is provided by Genentech/F.Hoffmann-La Roche LTD and distributed by the Pharmaceutical Management Branch, CTEP, NCI. The agent is supplied in a single-use, 20-mL glass vial as a colorless-to-slightly-yellow, sterile, preservative-free clear liquid solution intended for IV administration. Each 20 mL vial contains 1200 mg of atezolizumab and is formulated in glacial acetic acid (16.5 mg), L-histidine (62 mg), polysorbate 20 (8 mg), and sucrose (821.6 mg), with a pH of 5.8. The vial is designed to deliver 20 mL (1200 mg) of atezolizumab solution but may contain more than the stated volume to enable delivery of the entire 20 mL volume.

Preparation: The prescribed dose of atezolizumab should be diluted in 0.9% NaCl to a concentration between 3.2 mg/mL and 16.8 mg/mL and infused with or without a low-protein binding 0.2 or 0.22 micrometer in-line filter. The IV bag may be constructed of polyvinyl chloride (PVC), polyolefin (PO), or polyethylene (PE). The prepared solution may be stored at 2° C.-8° C. for up to 24 hours or at ambient <25° C. (77° F.) for 6 hours from the time of preparation. If the dose solution is stored at 2° C.-8° C. (36° F.-46° F.), it should be removed from refrigeration and allowed to reach room temperature prior to administration. These times include the storage and administration times for the infusion. Do not shake or freeze infusion bags containing the dose solution.

Storage: 2° C.-8° C. (36° F.-46° F.) Vial contents should not be frozen or shaken and should be protected from direct sunlight. If a storage temperature excursion is identified, promptly return atezolizumab to 2° C.-8° C. (36° F.-46° F.) and quarantine the supplies. Provide a detailed report of the excursion (including documentation of temperature monitoring and duration of the excursion) to PMBAfterHours@mail.nih.gov for determination of suitability.

Stability: Stability studies are ongoing.

CAUTION: No preservative is used in atezolizumab; therefore, the vial is intended for single use only. Discard any unused portion of drug remaining in a vial.

Route of Administration: IV infusion

Method of Administration: Atezolizumab is administered as an intravenous infusion over 60 minutes. If the first infusion is tolerated, all subsequent infusions may be delivered over 30 minutes. Do not administer atezolizumab as an intravenous push or bolus. No premedication is indicated for administration of Cycle 1 of atezolizumab. Patients who experience an infusion related reaction with Cycle 1 of atezolizumab may receive premedications with subsequent infusions.

Potential Drug Interactions: Cytochrome P450 enzymes as well as conjugation/glucuronidation reactions are not involved in the metabolism of atezolizumab. No drug interaction studies for atezolizumab have been conducted or are planned. There are no known interactions with other medicinal products or other form of interactions.

Patient Care Implications: Female patients of childbearing potential should utilize contraception and take active measures to avoid pregnancy while undergoing atezolizumab treatment and for at least 150 days after the last dose of atezolizumab.

B. Bevacizumab (NSC #704865): Other Names: rhuMAb VEGF, Avastin® Classification: Recombinant Humanized Monoclonal Antibody, Molecular Weight: Approximate Molecular Weight is 149,000 Daltons.

Mode of Action: Bevacizumab blocks the binding of vascular endothelial growth factor (VEGF) to its receptors resulting in inhibition of angiogenesis.

Description: Bevacizumab is a recombinant humanized anti-VEGF monoclonal antibody consisting of 93% human and 7% murine amino acid sequences. The agent is composed of human IgG framework and murine antigen-binding complementarity-determining regions.

How Supplied: Bevacizumab is supplied as a clear to slightly opalescent, sterile liquid for parenteral administration. Each 400 mg (25 mg/ml-16 mL fill) glass vial contains bevacizumab with phosphate, trehalose, polysorbate 20, and Sterile Water for Injection, USP.

Preparation: Vials contain no preservatives and are intended for single use only. The calculated dose should be diluted in 0.9% sodium chloride for injection to a final concentration of bevacizumab between 1.4 and 16.5 mg/ml.

Storage: Upon receipt, refrigerate bevacizumab (2° to 8° C.). Keep vial in the outer carton due to light sensitivity. Do not freeze. Do not shake.

If a storage temperature excursion is identified, promptly return bevacizumab to 2° C.-8° C. (36° F.-46° F.) and quarantine the supplies. Provide a detailed report of the excursion (including documentation of temperature monitoring and duration of the excursion) to PMBAfterHours@mail.nih.gov for determination of suitability.

Stability: Please see the product label for expiration information. The sterile single use vials contain no antibacterial preservatives. Discard vials 8 hours after initial entry.

Once diluted in 0.9% sodium chloride, the product should be used immediately. If not used immediately, in-use storage times are the responsibility of the user and should not be longer than 24 hours at 2° C.-8° C.

Route of Administration: Intravenous

Method of Administration: Administer the initial dose over a minimum of 90 minutes. If no adverse reactions occur, administer the second dose over a minimum of 60 minutes. If no adverse reactions occur after the second dose, administer subsequent doses over a minimum of 30 minutes. If infusion-related adverse reactions occur, all subsequent infusions should be administered over the shortest period that was well tolerated.

Patient Care Implications: Male patients and female patients of childbearing potential should utilize contraception and take active measures to avoid pregnancy prior to study entry, while undergoing bevacizumab treatment, and for at least 6 months (180 days) after the last dose of bevacizumab.

### VII. Correlative Studies

The effect of the study drugs on immune cell components of the tumor and microenvironment will also be studied. The correlative studies that will be carried out on the paired (pre- and post-treatment) biopsies will help elucidate the mechanism of action of atezolizumab monotherapy and the atezolizumab plus bevacizumab combination on signaling pathways mediating the immune response, focusing on subsets of effector cells. Additionally, the samples will be used to evaluate the tumor microenvironment to see if any changes or lack thereof are associated with either response or resistance to therapy. For patients who cross over directly from the monotherapy arm to the combination therapy arm, the baseline biopsy collected for the combination arm will provide insight into the biochemical signaling and genetic composition of their tumor at the time of disease progression on atezolizumab monotherapy. If additional tissue is available from any patient and its collection poses no new risks to the patient, the tissue may be collected by NCI DCTD SOPs for analysis by any of the correlative study assays described in the Correlative Studies section, provided appropriate consent is obtained.

An optional tumor biopsy may be collected from adult patients (≥18 years of age), on either treatment arm, at the time of disease progression (or pre-progression, following any restaging at which a 10-19% increase in tumor volume is observed) for analysis by any of the correlative study assays described in the Correlative Studies section, provided appropriate consent is obtained. At the PI's discretion, a progression biopsy collected while a patient is on the monotherapy arm may be utilized as the baseline biopsy for the combination arm, provided the patient does not receive any anti-cancer agents or immunotherapy in the interim.

Blood samples will also be collected for the purification and analysis of circulating tumor cells (CTCs). One additional optional blood sample will be collected at baseline (or any other time on study) from consenting patients ≥18 years of age and the nucleic acid from the mononuclear cells will be sequenced to serve as a germline control for calling somatic alterations in WES and RNA-Seq results from the patient's tumor specimen, or to enable the assessment of somatic alterations in the blood that may arise from clonal hematopoiesis of indeterminant potential.

For patients who consent to genetic sequencing of blood samples, plasma from CTC blood samples correlating to clinically informative time points (minimally, before treatment and at restaging scans suggesting changes in tumor burden) will be analyzed for circulating tumor DNA (ctDNA) by the TSO500 panel in order to identify potential mechanisms of response and resistance in cell free DNA (cfDNA) and to identify TFE3 fusions in plasma. If a sufficient proportion of the circulating cfDNA detected is ctDNA, then WES of cfDNA will also be considered to uncover additional relevant changes in the tumor genome. The results of these genomic analyses of blood samples will not be returned to patients.

In some aspects, peripheral blood mononuclear cells (PBMCs) will be collected from patients ≥14 years of age who consent to the PBMC collection and genetic sequencing component.

Blood (mandatory) will be collected from pediatric patients <12 years of age at multiple time points for measurement of PK and ADA, as described in Table 22.

TABLE 22
Tissue Specimen Prioritization Table

| | Baseline and C3D1 Biopsies |
|---|---|
| Collection Information | All cores flash frozen |
| First Priority Assay(s) | First 2 cores |
| | Immuno-PD, Apoptosis IFA, IHC |
| | (PD-1, PD-L1) |
| Second Priority Assay(s) | 3^{rd} core (and 4^{th} if needed) |
| | WES*, RNA-Seq*, targeted sequencing* |
| | (e.g., Archer panel) |
| Third Priority Assay(s) - | 5^{th} (or other remaining) core |
| hold core until | Luminex multiplex immunoassay for |
| sequencing results | apoptosis/cell death |
| obtained | |
| Reporting to Patients | None |

*Provided informed consent for genetic sequencing is obtained from the patient.

### A. Pharmacodynamic Assays in Tumor Tissue

Tumor biopsies (mandatory at baseline and cycle 3 day 1 for patients in whom biopsies are deemed safe and feasible, and optional at time of disease progression) will be collected for research from adult patients (≥18 years of age). Pharmacodynamic assays will be performed on these tumor specimens to assess the number of activated CD8+ T cells infiltrating the tumor before and after atezolizumab treatment. Efforts will be made to correlate any observed treatment-induced changes with clinical response.

Activated CD8+ T cells will be defined by the expression of phosphorylated Zap70 (pY493) and SHP2 (pY580); CD8+ cells present within the tumor section that are positive for these markers will be quantified. Other targets of PD assays may include PD-L1 and PD-1 levels in cells present in the biopsy, TCR activation (Zeta chain phosphorylation), T-cell Ki67 expression, the presence of T-reg cells (FOXP3+) and MDSCs, and other potential markers of response or resistance.

These biomarkers will be measured through multiplex immunofluorescence assays designed and validated by PADIS. Measurements on the Nikon Ai/Definiens platform will include cell enumeration and proximity of tumor cells (by pathology analysis and ASPL-TFE3 fusion-protein IFA) to T-cells. Monoclonal antibodies to all markers have been validated by the standard PADIS approach, and PADIS has previously validated and reported on the tumor cell marker set.

Mandatory biopsies will be collected from adult patients on both treatment arms at: Baseline (prior to the first dose of study agent(s) on that arm)

Prior to cycle 3 day 1 (±3 days) or at any point where there is clinical evidence that the patient is responding to drug

An optional tumor biopsy may be collected from adult patients (≥18 years of age), enrolled to either arm, at the time of disease progression (or pre-progression, following any restaging at which a 10-19% increase in tumor volume is observed).

For adult patients who progress on atezolizumab monotherapy and elect to cross over to the combination arm, the baseline biopsy for the combination arm should be collected after coming off monotherapy treatment due to progressive disease (see Measurement of Effect Section) but before initiating treatment on the combination arm. However, if the patient progressed on atezolizumab monotherapy after <4 cycles and had a biopsy collected on cycle 3 day 1 (±3 days) of atezolizumab monotherapy treatment, that cycle 3 day 1 biopsy will be used as their baseline biopsy for the combination treatment arm; a biopsy will not be collected between disease progression and initiation of the combination treatment. If a patient on the monotherapy arm elects to undergo an optional progression biopsy, that biopsy may, at the PI's discretion, serve as the baseline biopsy for the combination arm, provided the patient does not receive any anti-cancer agents or immunotherapy in the interim.

The post-treatment biopsy on the atezolizumab+bevacizumab arm will be collected prior to cycle 3 day 1 (±3 days) of the combination treatment (or at any point after initiating bevacizumab where there is clinical evidence that the patient is responding to drug).

### B. Genetic Assays in Tumor Tissue

Exploratory genetic sequencing will be performed on biopsy tissue from patients who consent to this optional component, with the goals of 1) classifying tumors by ASPL-TFE3 fusion type (Type 1 or Type 2), 2) characterizing genomic features associated with each fusion type, and 3) identifying potential markers of atezolizumab response and resistance. Tumor specimens that remain after the completion of the pharmacodynamic analysis described in the Correlative Studies section will be analyzed by whole exome and whole RNA transcriptome sequencing. Germline DNA obtained from a whole blood sample will be used as a point of comparison to identify somatic mutations. Targeted sequencing may also be performed (e.g., Archer panel to identify ASPL-TFE3 fusion types if they cannot be called from RNA-Seq data), as well as spatial profiling of RNA and protein expression. Single cell RNA sequencing may also be performed if appropriately preserved tissue is available.

### C. Biopsy Collection and Shipment

Serial tumor biopsies will be obtained by the Interventional Radiology team by a percutaneous approach, a dermatologist for skin lesions, or an ENT for lesions that are easily biopsiable through ENT exam. If a site is deemed appropriate for biopsy with minimal risk to the participant by agreement between the investigators and the biopsy team, an attempt for biopsy will be made. Because approximately 20% of tumor biopsies collected on research trials are not usable due to the presence of stroma or normal and/or necrotic tissue and paired biopsies are necessary for analysis, up to 5 core biopsies ≥18-gauge in diameter and >1 cm in length, or the equivalent, will be obtained during each procedure to try and ensure adequate tumor content and quality. If possible, the lesion from which each biopsy is taken will be documented. Acceptable biopsy procedures are: percutaneous biopsy with local anesthetic, excisional cutaneous biopsy with local anesthetic, and other biopsy with local anesthetic and/or sedation that has been shown to have a risk of severe complications <2%

The use of imaging to facilitate biopsies will be decided by members of the biopsy team and may include ultrasound, CT scan, or MRI. Should a CT scan be needed for biopsy, the number of scans for each procedure will be limited to the minimum number needed to safely obtain a biopsy. Tumor biopsies and local anesthesia will be administered only if they are considered to be of low risk to the participant, as determined by the investigators and the biopsy team. The clinical, radiologic, dermatologic, ENT, and pharmacodynamic members of the research team will meet monthly to review the adequacy of the biopsy specimens for analysis.

Tumor biopsies are mandatory at all participating sites. Baseline biopsies will be performed following patient enrolling on study, prior to initiating study treatment (on the monotherapy arm and the combination arm). If an initial attempt at biopsy is unsuccessful, the patient will be given an option to proceed with a repeated attempt. A separate consent form must be signed for each biopsy procedure, so patients may choose not to undergo subsequent biopsies. If the baseline biopsy is unsuccessful or the patient refuses to undergo subsequent biopsies, no further biopsies will be performed but the patient will remain on study, receive study medication, and other correlative studies will be performed.

Solid Tumor Biopsy Processing: Biopsy cores will be processed as described in NCI DCTD SOP340507. All external sites are required to follow NCI DCTD SOP340507 (found at dctd.cancer.gov/ResearchResources/biomarkers/docs/par/SOP340507_Biopsy_Froz en.pdf) for biopsy collection, processing, and shipment.

Briefly, the biopsy cores or, equivalent tissue, will be transferred into 1.5-mL pre-chilled cryovials and then flash frozen in liquid nitrogen. Biopsies should be collected, placed in pre-chilled cryogenic vials, and frozen within 2 minutes of collection. The frozen biopsy specimens are transferred to PADIS in pairs on dry ice, where they are stored at −80° C. or colder, and subsequently processed within 7-10 days for analysis or as directed by a physician. Biopsy samples will be analyzed as described above; any additional samples will be kept for future analysis in liquid nitrogen freezers. Additional studies, if performed, will be conducted following an amendment to the current protocol.

Baseline and post-treatment biopsy samples should be held and shipped to PADIS together upon completion of the second biopsy. If a patient crosses over from the monotherapy arm to the combination arm, the pre-treatment biopsy for the combination arm (collected after disease progression on monotherapy) should be held and shipped with the post-combination treatment biopsy.

### D. Blood Collection and Shipment

No more than 5 mL/kg may be drawn for research purposes in a single day, and no more than 9.5 mL/kg may be drawn over any eight-week period.

Circulating Tumor Cells (CTCs): Collection of blood samples for PD studies in CTCs will be mandatory. Blood specimens will be obtained from patients ≥14 years of age. CTCs will be isolated from specimens associated with clinically significant time points and analyzed for various biomarkers, such as ASPL-TFE3 fusion protein, beta-Catenin, cytokeratin, and vimentin, as well as PD-L1 and apoptosis markers γH2AX and activated caspase 3 and 8. Plasma obtained during the isolation of CTCs at the PADIS laboratory will be retained for genetic analysis of cell-free DNA (see Section 9.4.6). This does not involve any changes to CTC collection or processing procedures at clinical sites. CTC specimens may also be used for identification of ASPL-TFE3 fusions via RT-PCR.

Collection: Blood samples (7.5 mL) will be collected at the following times on both treatment arms:
at baseline (on cycle 1 day 1 prior to drug administration)
on cycle 2 day 1
on the first day of every subsequent cycle prior to drug administration
at disease progression
at the time of each response assessment during a treatment holiday
at the time of each response assessment during the response follow-up period after completion of a treatment holiday
   Note that while CTC collection is required at these timepoints, the requirement is waived if a collection time point falls on a Friday or too late on a Thursday to ensure same-day FedEx pickup, due to shipment and processing requirements.

CTC collections will be repeated if a patient crosses over or re-enrolls to the combination arm.

At participating ETCTN sites, blood will be collected aseptically by venipuncture or from a venous port into one 10 mL Streck Cell-Free DNA BCT blood collection tube (Streck, Cat #218962). The collected blood samples are stable for up to 48 hours at room temperature (15° C. to 30° C.) prior to processing. Refer to NCI DCTD SOP LHTP003.08.19 for additional guidance regarding blood collection in a Streck tube: dctd.cancer.gov/ResearchResources/biomarkers/emt/LHTP003.08.19_Streck.pdf

At the NCI Clinical Center, each blood specimen will be collected into one 10 mL Streck tube (catalog number 218962). Tubes must be inverted 8 times to ensure adequate mixing of the additive.

### Blood for Germline Sequencing

Collection: Beginning with the 01/07/21 protocol amendment, one whole blood sample (at least 7.5 mL in a 10-mL Streck tube) will be collected at baseline (or at any point while the patient is on study) from patients who consent to genomic analyses, and mononuclear cells will be isolated from whole blood by the MoCha Laboratory for nucleic acid extraction and exploratory WES to allow for germline and somatic variants to be accurately identified, and to enable the accurate assessment of mutational signatures in tumor specimens. The results of these studies will not be returned to patients.

### Cryopreserved Peripheral Blood Mononuclear Cells (PBMCs)

In some aspects, PBMCs will be isolated and cryopreserved for genomic analysis of T cell receptor repertoire. These samples should not be collected at participating (non-NCI) sites.

PBMCs will be collected from patients ≥14 years of age who consent to this component. Whole blood samples (at least 4.5 mL in a 6-mL EDTA tube) will be collected at the following times on both treatment arms:
at baseline
on cycle 3 day 1 (or the next restaging visit if C3D1 has already passed)
12 weeks (±1 week) after the C3D1 or restaging collection*
another 12 weeks (±1 week) later (e.g., C11D1 if collected on C3D1)*
at disease progression or response.
In the event that the collection coincides with a patient's treatment at a non-NCI site, the collection will be postponed to the next restaging visit at the NCI.

PBMC collections will be repeated if a patient crosses over or re-enrolls to the combination ann.

Each whole blood specimen will be collected into one standard 6 mL EDTA tube. Within 60 minutes of collection, PBMCs will be isolated using SepMate tubes (kits provided) according to instructions in “Cryopreservation of PBMCs” below. Briefly, add density gradient medium through SepMate insert, dilute whole blood sample with an equal volume PBS+2% FBS and pipette slowly down the side of the SepMate tube, centrifuge for 10 minutes with the brake on, pipette off some supernatant above the PBMC layer, pour the remaining supernatant and isolated PBMCs into a 15 mL or 50 mL tube (inverting for ≤2 seconds), wash the PBMCs twice with 10 mL PBS+2% FBS, and resuspend in 2.5 mL of cryopreservation Component #1. Within 60 minutes of isolation, the PBMCs must be aliquoted into five cryovials (0.5 mL per cryovial) containing cryopreservation Component #2 and frozen slowly in a CoolCell container to −80° C.

### Pharmacokinetics and Anti-Drug Antibodies (Ages 2-11 Only)

Serum samples (mandatory) will be collected at multiple time points from pediatric patients who are 2-11 years old at enrollment, in order to measure pharmacokinetics (PK) and the presence of anti-drug (i.e., atezolizumab) antibodies (ADA) in this sparse patient population. If a patient participating in this PK+ADA study turns 12 years old while still receiving study treatment, serum will continue to be collected past age 11. All PK and ADA analyses for this study will be performed by ICON.

### Collection:

Please see “Procedure for pediatric PK+ADA blood collection” below for sample collection and labeling instructions. The blood will be collected at the following time points as described in Table 23:

TABLE 23
Blood Collection Timepoints for Pediatric PK + ADA

| Timepoint | PK | ADA |
|---|---|---|
| C 1 D 1 | Baseline (predose) | predose |
| C 1 D 1 | 30 min after the end of infusion | — |
| C 2 D 1 | predose | predose |
| C 3 D 1 | predose | predose |
| C 4 D 1 | predose | predose |
| C 4 D 1 | 30 min after the end of infusion | — |
| C 8 D 1* | predose | predose |
| C 12 D 1* | predose | predose |
| C 16 D 1* | predose | predose |
| Progression/Study drug | Any time during visit | Any time |
| discontinuation | | during visit |

*In the event that the collection coincides with a patient's treatment at their local, non-enrolling site, the collection will be postponed to the next restaging visit at their enrolling site.

Kits for sample collection will be shipped to the clinical site and will include specimen labels and a sample collection worksheet/manifest. The sample worksheet/manifest should be completed and included with the return shipment of each serum sample. Serum should be shipped back to Labcorp on the day of collection (or the following day when necessary); samples should be stored at or below −20° C. until shipment to Labcorp.

### Pharmacokinetics and Anti-Drug, Antibodies (Monotherapy Arm, Ages 12 Only)

Serum samples (optional) will be collected from patients who were 12 years old at enrollment and are enrolled to the monotherapy arm. These samples will be collected on Day 1 of one to a maximum of three cycles from patients, in order to measure steady-state PK in this ASPS patient population. All PK analyses for this study will be performed by ICON.

Collection: The blood will be collected at the following time points as described in Table 24.

TABLE 24
Blood Collection Timepoints for Pediatric PK (≥12 Only)

| Time point | PK |
|---|---|
| Cx D 1 | predose |
| Cx D 1 | 30 min after the end of atezolizumab infusion |
| Cx + 1 D 1 | predose |
| Cx + 1 D 1 | 30 min after the end of atezolizumab infusion |
| Cx + 2 D 1 | predose |
| Cx + 2 D 1 | 30 min after the end of atezolizumab infusion |

Cx = the cycle a patient begins once collection kits are available and the patient’s consent for this optional collection is obtained
*In the event that the collection coincides with a patient’s treatment at their local, non-enrolling site, the collection will be postponed to the next restaging visit at their enrolling site.

Kits for sample collection will be shipped to the clinical site and will include specimen labels and a sample collection worksheet/manifest. The sample worksheet/manifest should be completed and included with the return shipment of each serum sample. Serum should be shipped back to Labcorp on the day of collection (or the following day when necessary); samples should be stored at or below −20° C. until shipment to Labcorp.

Cell-Free DNA Sequencing: For patients who consent to genetic sequencing of blood samples, plasma from CTC blood samples correlating to clinically informative time points (minimally, before treatment and at restaging scans suggesting changes in tumor burden) will be analyzed for circulating tumor DNA (ctDNA) by the TSO500 panel. If a sufficient proportion of the circulating cfDNA detected is ctDNA, then WES of cfDNA will also be considered to uncover additional relevant changes in the tumor genome. The results of these studies will not be returned to patients.

### E. Sample Collection and Processing

Biospecimens will be collected and processed using validated SOPs that will ensure both specimen quality and patient confidentiality pursuant to informed consent provisions. Information about each specimen (e.g., blood, tumor biopsy, per specific protocol) will be recorded on a PK/PD collection worksheet.

Using a computerized inventory system and a backup hardcopy process, all specimen collection and processing steps will be documented and the specific location of each specimen will be tracked. Each new specimen collected will be assigned a unique barcode identifier that can be linked to the original specimen collected and other relevant information within the inventory system. To ensure patient confidentiality, only containers used for the initial specimen collections will be labeled with patient identifiers.

Only the barcode identifier will be applied to all subsequent specimen containers. When specimens are processed and aliquoted, no patient information will be included on the new containers. Original specimen containers will be discarded. Only barcode-labeled specimens without patient identifiers will be shipped for analysis and/or storage.

Specimen labels will indicate: CTEP protocol number, unique patient accession number, 3-digit sample number (see list below), collection time, and total volume collected, as appropriate. Samples from sets of at least three patients will be grouped for scientific analysis. Standardized 3-digit sample collection numbers:
300 series: PBMCs for T cell receptor sequencing 400 series: blood for circulating tumor cells
500 series: tumor biopsies
800 series: blood for genetic/genomic analyses

The inventory process contains other security provisions sufficient to safeguard patient privacy and confidentiality. Access to the inventory system and associated documents will be restricted to appropriate individuals. Requests to use specimens stored in the repository must be approved. The only patient information available in the inventory system will be the patient sex, diagnosis, and level of informed consent given. SOPs ensure that any changes in informed consent made by a patient and relayed to the PI will be reflected in the inventory system to ensure that specimens are destroyed as appropriate. All laboratory personnel will be trained to adhere to SOPs and will be monitored for high-quality performance.

Any new use of these samples will require prospective IRB review and approval. Access to these samples will only be granted following IRB approval of an additional protocol, granting the rights to use the material.

If at any time, a patient withdraws from the study and does not wish for their existing samples to be utilized, the individual must provide a written request. Following receipt of this request, the samples will be destroyed (or returned to the patient, if so requested), and reported as such to the IRB. Any samples lost (in transit or by a researcher) or destroyed due to unknown sample integrity (i.e., broken freezer allows for extensive sample thawing, etc.) will be reported as such to the IRB.

### F. Management of Genomic Results

This trial will collect identifiable genetic data from patients for exploratory studies to classify tumors by ASPL-TFE3 fusion type and associated genomic differences, and to identify potential markers of atezolizumab response and resistance. Designing the study poses challenging questions about informed consent, the privacy of the patient and the patient's family, the researchers' obligation to disclose genetic information to the patient, and the use and storage of research data [Dressler et al., Clin Pharmacol Ther, 2009. 86(5): p. 472-5; Kass et al., Am J Med Genet A, 2004. 128a(3): p. 261-70; Tabor et al., Am J Med Genet A, 2011. 155a(12): p. 2916-24]. In the vast majority of cases, we do not know the medical significance of genetic variants [Berg et al., Genet Med, 2011. 13(6): p. 499-504; Caulfield et al., PLoS Biol, 2008. 6(3): p. e73]. These challenges will continue to be evaluated.

### VIII. Study Calendar

Eligibility screening evaluations are to be conducted within 8 days prior to patient enrollment to either treatment arm, with the exception of informed consent, echocardiogram (ECHO), and imaging, which must be done within 28 days prior to patient enrollment. Baseline history, physical examination, laboratory evaluations, and urinalysis are to be conducted within 8 days prior to the start of protocol therapy (21 days allowed for EKG). If protocol therapy is started within 8 days of the eligibility screening evaluations, values from the screening evaluations may be used as baseline measurements; if >8 days have passed since the screening evaluations, the medical history, physical examination, laboratory evaluations, urinalysis, and EKG must be repeated prior to starting protocol therapy. Baseline imaging scans and ECHO must be done within 28 days prior to the start of protocol therapy.

Each cycle is 21 days (±3 days due to scheduling conflicts). The start of a new cycle may be delayed up to 2 weeks to accommodate scheduling conflicts and other unexpected events. In the event that the patient's condition is deteriorating, laboratory evaluations should be repeated within 48 hours prior to initiation of the next cycle of therapy.

### IX. Measurement of Effect

Antitumor Effect—Solid Tumors: All patients will have a CT scan (or other imaging test; see Methods for Evaluation of Measurable Disease below) at baseline, at the end of cycle 3, and every two cycles (6 weeks) thereafter (every 3 cycles for patients on study for >1 year; every 4 cycles for patients on study for >2 years). Confirmatory scans should also be obtained at ≥4 weeks following initial documentation of objective response. Patients on the monotherapy arm will have a CT scan at the end of cycle 1 which will be used to assess for early response only; no treatment decisions will be made unless clinically warranted.

Given the concern of pseudoprogression (“tumor flare”), patients who show evidence of clinical benefit will be permitted to continue their monotherapy or combination treatment following RECIST progression, as long as the increase in disease burden does not meet the definition of confirmed PD by immune response criteria and if they meet all of the following additional criteria:
1. Evidence of clinical benefit as assessed by the investigator and agreed upon by the Medical Monitor
2. Absence of symptoms or signs (including worsening laboratory values) indicating unequivocal progression of disease
3. No significant decline in performance status that can be attributed to disease progression
4. Absence of tumor progression at critical anatomical sites (e.g., leptomeningeal disease) that cannot be managed by protocol-allowed medical interventions
5. The investigator must discuss with the patient the risks and benefits of treatment continuation after possible worsening disease and must document the patient's agreement to continue receiving treatment (atezolizumab monotherapy or atezolizumab+bevacizumab, as enrolled) in the medical record.

Adult patients who do not meet the criteria for continuing atezolizumab monotherapy may cross over to the atezolizumab+bevacizumab combination arm, provided they meet the patient selection criteria.

Response and progression will be evaluated in this study using the new international criteria proposed by the revised Response Evaluation Criteria in Solid Tumors (RECIST) guideline (version 1.1) [Eisenhauer et al., Eur J Cancer, 2009. 45(2): p. 228-47]. Immune RECIST (iRECIST) [Seymour et al., The Lancet Oncology, 2017. 18(3): p. e143-e152] will be used only as an exploratory endpoint, for comparison to RECIST 1.1 (see Immune RECIST Criteria). Changes in the largest diameter (unidimensional measurement) of the tumor lesions and the shortest diameter in the case of malignant lymph nodes are used in the RECIST criteria.

### Definitions

Evaluable for toxicity. All patients will be evaluable for toxicity from the time of their first treatment with atezolizumab

Evaluable for objective response. All patients who met the eligibility criteria (with the possible exception of those who received no study medication) will be considered evaluable for response. These patients will have their response classified according to the definitions stated below.

Evaluable Non-Target Disease Response. Patients who have lesions present at baseline that are evaluable but do not meet the definitions of measurable disease, have received at least one cycle of therapy, and have had their disease re-evaluated will be considered evaluable for non-target disease. The response assessment is based on the presence, absence, or unequivocal progression of the lesions.

### Disease Parameters

Measurable disease. Measurable lesions are defined as those that can be accurately measured in at least one dimension (longest diameter to be recorded) as ≥20 mm (≥2 cm) by chest x-ray or as ≥10 mm (≥1 cm) with CT scan, MRI, or calipers by clinical exam.

All tumor measurements must be recorded in millimeters (or decimal fractions of centimeters).

Malignant lymph nodes. To be considered pathologically enlarged and measurable, a lymph node must be ≥15 mm (≥1.5 cm) in short axis when assessed by CT scan (CT scan slice thickness recommended to be no greater than 5 mm [0.5 cm]). At baseline and in follow-up, only the short axis will be measured and followed.

Non-measurable disease. All other lesions (or sites of disease), including small lesions (longest diameter <10 mm [<1 cm] or pathological lymph nodes with ≥10 to <15 mm [>1 to ≤1.5 cm] short axis), are considered non-measurable disease. Bone lesions, leptomeningeal disease, ascites, pleural/pericardial effusions, lymphangitis cutis/pulmonitis, inflammatory breast disease, and abdominal masses (not followed by CT or MRI), are considered as non-measurable.

Note: Cystic lesions that meet the criteria for radiographically defined simple cysts should not be considered as malignant lesions (neither measurable nor non-measurable) since they are, by definition, simple cysts.

‘Cystic lesions’ thought to represent cystic metastases can be considered as measurable lesions, if they meet the definition of measurability described above. However, if non-cystic lesions are present in the same patient, these are preferred for selection as target lesions.

Target lesions. All measurable lesions up to a maximum of 2 lesions per organ and 5 lesions in total, representative of all involved organs, should be identified as target lesions and recorded and measured at baseline. Target lesions should be selected on the basis of their size (lesions with the longest diameter), be representative of all involved organs, but in addition should be those that lend themselves to reproducible repeated measurements. It may be the case that, on occasion, the largest lesion does not lend itself to reproducible measurement in which circumstance the next largest lesion which can be measured reproducibly should be selected. A sum of the diameters (longest for non-nodal lesions, short axis for nodal lesions) for all target lesions will be calculated and reported as the baseline sum diameters. If lymph nodes are to be included in the sum, then only the short axis is added into the sum. The baseline sum diameters will be used as reference to further characterize any objective tumor regression in the measurable dimension of the disease.

Non-target lesions. All other lesions (or sites of disease) including any measurable lesions over and above the 5 target lesions should be identified as non-target lesions and should also be recorded at baseline. Measurements of these lesions are not required, but the presence, absence, or in rare cases unequivocal progression of each should be noted throughout follow-up.

Methods for Evaluation of Measurable Disease: All measurements should be taken and recorded in metric notation using a ruler or calipers. All baseline evaluations should be performed as closely as possible to the beginning of treatment and never more than 4 weeks before the beginning of the treatment.

The same method of assessment and the same technique should be used to characterize each identified and reported lesion at baseline and during follow-up. Imaging-based evaluation is preferred to evaluation by clinical examination unless the lesion(s) being followed cannot be imaged but are assessable by clinical exam.

Clinical lesions: Clinical lesions will only be considered measurable when they are superficial (e.g., skin nodules and palpable lymph nodes) and >10 mm (≥1 cm) diameter as assessed using calipers (e.g., skin nodules). In the case of skin lesions, documentation by color photography, including a ruler to estimate the size of the lesion, is recommended.

Chest x-ray: Lesions on chest x-ray are acceptable as measurable lesions when they are clearly defined and surrounded by aerated lung. However, CT is preferable.

Conventional CT and MRI: This guideline has defined measurability of lesions on CT scan based on the assumption that CT slice thickness is 5 mm (0.5 cm) or less. If CT scans have slice thickness greater than 5 mm (0.5 cm), the minimum size for a measurable lesion should be twice the slice thickness. MRI is also acceptable in certain situations (e.g. for body scans).

Use of MRI remains a complex issue. MRI has excellent contrast, spatial, and temporal resolution; however, there are many image acquisition variables involved in MRI, which greatly impact image quality, lesion conspicuity, and measurement. Furthermore, the availability of MRI is variable globally. As with CT, if an MRI is performed, the technical specifications of the scanning sequences used should be optimized for the evaluation of the type and site of disease. Furthermore, as with CT, the modality used at follow-up should be the same as was used at baseline and the lesions should be measured/assessed on the same pulse sequence. It is beyond the scope of the RECIST guidelines to prescribe specific MRI pulse sequence parameters for all scanners, body parts, and diseases. Ideally, the same type of scanner should be used and the image acquisition protocol should be followed as closely as possible to prior scans. Body scans should be performed with breath-hold scanning techniques, if possible.

PET-CT: At present, the low dose or attenuation correction CT portion of a combined PET-CT is not always of optimal diagnostic CT quality for use with RECIST measurements. However, if the site can document that the CT performed as part of a PET-CT is of identical diagnostic quality to a diagnostic CT (with IV and oral contrast), then the CT portion of the PET-CT can be used for RECIST measurements and can be used interchangeably with conventional CT in accurately measuring cancer lesions over time. Note, however, that the PET portion of the CT introduces additional data which may bias an investigator if it is not routinely or serially performed.

Ultrasound: Ultrasound is not useful in assessment of lesion size and should not be used as a method of measurement. Ultrasound examinations cannot be reproduced in their entirety for independent review at a later date and, because they are operator dependent, it cannot be guaranteed that the same technique and measurements will be taken from one assessment to the next. If new lesions are identified by ultrasound in the course of the study, confirmation by CT or MRI is advised. If there is concern about radiation exposure at CT, MRI may be used instead of CT in selected instances.

Endoscopy, Laparoscopy: The utilization of these techniques for objective tumor evaluation is not advised. However, such techniques may be useful to confirm complete pathological response when biopsies are obtained or to determine relapse in trials where recurrence following complete response (CR) or surgical resection is an endpoint.

Tumor markers: Tumor markers alone cannot be used to assess response. If markers are initially above the upper normal limit, they must normalize for a patient to be considered in complete clinical response. Specific guidelines for both CA-125 response (in recurrent ovarian cancer) and PSA response (in recurrent prostate cancer) have been published [JNCI 96:487-488, 2004; J Clin Oncol 17, 3461-3467, 1999; J Clin Oncol 26:1148-1159, 2008]. In addition, the Gynecologic Cancer Intergroup has developed CA-125 progression criteria which are to be integrated with objective tumor assessment for use in first-line trials in ovarian cancer [JNCI 92:1534-1535, 2000].

Cytology, Histology: These techniques can be used to differentiate between partial responses (PR) and complete responses (CR) in rare cases (e.g., residual lesions in tumor types, such as germ cell tumors, where known residual benign tumors can remain).

The cytological confirmation of the neoplastic origin of any effusion that appears or worsens during treatment when the measurable tumor has met criteria for response or stable disease is mandatory to differentiate between response or stable disease (an effusion may be a side effect of the treatment) and progressive disease.

FDG-PET: While FDG-PET response assessments need additional study, it is sometimes reasonable to incorporate the use of FDG-PET scanning to complement CT scanning in assessment of progression (particularly possible ‘new’ disease). New lesions on the basis of FDG-PET imaging can be identified according to the following algorithm:
1. Negative FDG-PET at baseline, with a positive FDG-PET at follow-up is a sign of PD based on a new lesion.
2. No FDG-PET at baseline and a positive FDG-PET at follow-up: If the positive FDG-
3. PET at follow-up corresponds to a new site of disease confirmed by CT, this is PD. If the positive FDG-PET at follow-up is not confirmed as a new site of disease on CT, additional follow-up CT scans are needed to determine if there is truly progression occurring at that site (if so, the date of PD will be the date of the initial abnormal FDG-PET scan). If the positive FDG-PET at follow-up corresponds to a pre-existing site of disease on CT that is not progressing on the basis of the anatomic images, this is not PD.
4. FDG-PET may be used to upgrade a response to a CR in a manner similar to a biopsy in cases where a residual radiographic abnormality is thought to represent fibrosis or scarring. The use of FDG-PET in this circumstance should be prospectively described in the protocol and supported by disease-specific medical literature for the indication. However, it must be acknowledged that both approaches may lead to false positive CR due to limitations of FDG-PET and biopsy resolution/sensitivity.
   Note: A ‘positive’ FDG-PET scan lesion means one which is FDG avid with an uptake greater than twice that of the surrounding tissue on the attenuation corrected image.

### Response Criteria

### Evaluation of Target Lesions

Complete Response (CR): Disappearance of all target lesions. Any pathological lymph nodes (whether target or non-target) must have reduction in short axis to <10 mm (<1 cm).

Partial Response (PR): At least a 30% decrease in the sum of the diameters of target lesions, taking as reference the baseline sum diameters.

Progressive Disease (PD): At least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm (0.5 cm). (Note: the appearance of one or more new lesions is also considered progressions).

Stable Disease (SD): Neither sufficient shrinkage to qualify for PR nor sufficient increase to qualify for PD, taking as reference the smallest sum diameters while on study.

### Evaluation of Non-Target Lesions

Complete Response (CR): Disappearance of all non-target lesions and normalization of tumor marker level. All lymph nodes must be non-pathological in size (<10 mm [<1 cm] short axis). Note: If tumor markers are initially above the upper normal limit, they must normalize for a patient to be considered in complete clinical response.

Non-CR/Non-PD: Persistence of one or more non-target lesion(s) and/or maintenance of tumor marker level above the normal limits.

Progressive Disease (PD): Appearance of one or more new lesions and/or unequivocal progression of existing non-target lesions. Unequivocal progression should not normally trump target lesion status. It must be representative of overall disease status change, not a single lesion increase.

Although a clear progression of “non-target” lesions only is exceptional, the opinion of the treating physician should prevail in such circumstances, and the progression status can be confirmed at a later time.

### Evaluation of Non-Measurable Lesions

Complete Response (CR): Complete disappearance of disease.

Non-CR/Non-PD: Persistence of evidence of disease.

Progressive Disease (PD): Unequivocal disease progression.

Evaluation of Best Overall Response: The best overall response is the best response recorded from the start of the treatment until disease progression/recurrence (taking as reference for progressive disease the smallest measurements recorded since the treatment started). The patient's best response assignment will depend on the achievement of both measurement and confirmation criteria. Best overall response confirmation timeline for patients with measurable and non-measurable disease are described in Table 25 and 26.

TABLE 25
For Patients with Measurable Disease (i.e., Target Disease)

| Target | Non-Target | New | Overall | Best Overall Response when |
|---|---|---|---|---|
| Lesions | Lesions | Lesions | Response | Confirmation is Required* |
| CR | CR | No | CR | ≥4 wks. Confirmation** |
| CR | Non-CR/Non-PD | No | PR | ≥4 wks. Confirmation** |
| CR | Not evaluated | No | PR | |
| PR | Non-CR/Non- | No | PR | |
| | PD/not evaluated | | | |
| SD | Non-CR/Non- | No | SD | Documented at least once ≥4 wks. |
| | PD/not evaluated | | | from baseline** |
| PD | Any | Yes or No | PD | no prior SD, PR or CR |
| Any | PD*** | Yes or No | PD | |
| Any | Any | Yes | PD | |

See RECIST 1.1 manuscript for further details on what is evidence of a new lesion.
**Required in this study.
***In exceptional circumstances, unequivocal progression in non-target lesions may be accepted as disease progression.
Note:
Patients with a global deterioration of health status requiring discontinuation of treatment without objective evidence of disease progression at that time should be reported as “symptomatic deterioration.” Every effort should be made to document the objective progression even after discontinuation of treatment.

TABLE 26
For Patients with Non-Measurable Disease
(i.e., Non-Target Disease)

| Non-Target Lesions | New Lesions | Overall Response |
|---|---|---|
| CR | No | CR |
| Non-CR/non-PD | No | Non-CR/non-PD* |
| Not all evaluated | No | not evaluated |
| Unequivocal PD | Yes or No | PD |
| Any | Yes | PD |

‘Non-CR/non-PD’ is preferred over ‘stable disease’ for non-target disease since SD is increasingly used as an endpoint for assessment of efficacy in some trials so to assign this category when no lesions can be measured is not advised

For those patients that go on treatment holiday, tumor response and progression will continue to be assessed among those patients who have not yet progressed. In addition, the post-progression response rate among patients who progress while on holiday and then resume treatment will be assessed as an exploratory endpoint. This endpoint will not include patients who resume therapy after a drug holiday (≤2 years) without progression.

### Duration of Response

Duration of overall response: The duration of overall response is measured from the time measurement criteria are met for CR or PR (whichever is first recorded) until the first date that recurrent or progressive disease is objectively documented (taking as reference for progressive disease the smallest measurements recorded since the treatment started).

The duration of overall CR is measured from the time measurement criteria are first met for CR until the first date that progressive disease is objectively documented.

Duration of stable disease: Stable disease is measured from the start of the treatment until the criteria for progression are met, taking as reference the smallest measurements recorded since the treatment started, including the baseline measurements.

Data Reporting: Data collection for this study will be done exclusively through Medidata Rave. Access to the trial in Rave is granted through the iMedidata application to all persons with the appropriate roles assigned in the Regulatory Support System (RSS). To access Rave via iMedidata, the site user must have an active CTEP IAM account ctepcore.nci.nih.gov/iam) and the appropriate Rave role (Rave CRA, Rave Read-Only, Rave CRA (Lab Admin), Rave SLA, or Rave Investigator) on either the LPO or participating organization roster at the enrolling site. To the hold Rave CRA role or Rave CRA (Lab Admin) role, the user must hold a minimum of an AP registration type. To hold the Rave Investigator role, the individual must be registered as an NPIVR or IVR. Associates can hold read-only roles in Rave.

Method: This study will be monitored by the Clinical Trials Monitoring Service (CTMS). Data will be: submitted to CTMS at least once every two weeks via Medidata Rave (or other modality if approved by CTEP). Information on CTMS reporting is available at theradex.com/clinicalTechnologies/?National-Cancer-Institute-NCI-11. On-site audits will be conducted three times annually (one annual site visit and two data audits).

Data Monitoring for Pediatric Patients: Biweekly meetings with research staff, including pediatric oncologists, can be conducted to discuss pediatric patient progress and toxicities. Any toxicities identified can be reported and according to IRB guidelines.

### X. Statistical Considerations

### A. Study Design/Endpoints

For the atezolizumab monotherapy arm, a Phase 2 Simon two stage design will be employed. Accrual to the monotherapy arm will be halted at 9 patients to evaluate for response before further enrollment. If no responses are observed among the initial 9 patients, the study will be terminated early and declared negative; if at least one response is observed, the study will continue. If at least 3 responses (at least 12.5%) are observed among the initial 24 evaluable patients, this regimen will be considered worthy of further testing in this disease. If the study does not meet its primary objective with the initial 24 evaluable patients, accrual will stop and no further patients will be added to the study. This design yields at least 90% power to detect a true response rate of at least 25%. It yields at least 0.91 probability of a negative result if the true response rate is no more than 5%, with at least 0.63 probability of early negative stopping.

A partial response was observed in the initial 9 patients treated in the monotherapy arm. The study continued to enroll to meet accrual goals.

Eight partial responses had been observed among the initial 19 evaluable patients in the monotherapy arm, indicating that the regimen is worthy of further testing. The accrual ceiling was raised to allow for the collection of research biopsies for pharmacodynamic analysis.

For the atezolizumab+bevacizumab arm, nine patients will be enrolled (via crossover or re-enrollment). If zero responses are observed among these nine patients, the combination arm will not be pursued further on this trial. If at least one response is observed among the first nine patients, an amendment will be submitted to CTEP to request that an additional 15 patients be enrolled to this arm, with 3 responses (out of 24 total patients) being required for a positive trial in this arm. Note that this is the same design employed for the atezolizumab monotherapy cohort. It should also be noted that, regardless of the number of responses observed in the first nine patients, no additional patients will be enrolled to this cohort unless our request (via an amendment) for additional patients is approved by CTEP. Interim and final analyses for this arm will be modified ITT, i.e. all eligible patients who receive at least one dose of treatment will be included.

### B. Sample Size/Accrual Rate

To allow for the collection of research biopsies for pharmacodynamic studies, additional patients may be enrolled to the monotherapy arm beyond the initial 24 evaluable patients (initial accrual ceiling of 26 patients).

Mandatory biopsies will be collected for pharmacodynamic analysis from adult patients only. With biopsies collected from 15 adult patients, there will be 95% likelihood of getting at least 10 evaluable biopsy pairs. For a given PD endpoint, this will yield 82% power to detect a difference in change from baseline to post-treatment, between the clinical responders and the non-responders, corresponding to at least 2 SD's (with respect to the baseline measures), at the 1-sided 0.01 significance level (to accommodate multiple comparisons).

The monotherapy arm will remain open to pediatric patients until 5 pediatric patients have contributed Cycle 3 Day 1 blood samples for the optional PK+ADA study, 13 pediatric patients have enrolled (with or without blood sample collections), or the atezolizumab plus bevacizumab arm is closed to accrual, whichever happens sooner. Pediatric patients will not provide research biopsies, but will have the opportunity to be treated on the monotherapy arm due to the possibility of therapeutic benefit; patients <12 years of age may choose to participate in the optional PK+ADA study.

To successfully obtain biopsies from 15 adult participants, and to allow patient slots for the enrollment of pediatric participants, the accrual ceiling for the monotherapy arm was increased from 26 patients to 53 patients. It was subsequently raised to 63 to allow enrollment of an additional 10 pediatric patients, thus offering pediatric patients the possibility of therapeutic benefit and facilitating the collection of blood samples for the pediatric PK+ADA study. In addition to the monotherapy arm, a maximum of 9 adult patients will be enrolled to the combination arm (all of whom were previously enrolled to the monotherapy arm). Thus, the anticipated maximum combined sample size is 72 enrollments, comprised of a maximum of 63 unique patients.

With an anticipated accrual of 12-24 monotherapy patients per year, it was expected that 2.5-4.5 year would be required to enroll 53 adult and pediatric patients to the monotherapy arm. With an anticipated accrual of 3 pediatric patients per year, it is expected that an additional 2-4 years will be required to enroll the 5-10 additional pediatric patients to the monotherapy arm. Assuming an accrual rate of 18 combination arm patients per year, enrollment to the combination arm is expected to take approximately 0.5 years.

### C. Analysis of Secondary and Exploratory Endpoints

As secondary analyses, response analyses will be stratified by prior tyrosine kinase inhibitor/anti-angiogenic agent-naïve patients versus those with prior tyrosine kinase inhibitor or anti-angiogenic agent exposure.

The analysis of pediatric PK+ADA data from this trial will be descriptive in nature; the anticipated sample size is 5 patients. The PK+ADA data may also be included with data from the G029664 trial (from 25 and 24 patients, respectively) for pooled population PK modeling efforts, in which ADA is a covariate. The data from this trial in ASPS, where responses are likely, is expected to augment the limited exposure-response data from G029664.

The analysis of adolescent and adult (≥12 years old) PK data from this trial will be descriptive in nature; the anticipated sample size is 8 patients. Serum concentrations of atezolizumab may be reported as individual values and summarized (mean, standard deviation, coefficient of variation, median, range, geometric mean, and geometric mean coefficient of variation) by treatment arm and cycle, when appropriate and as data allow. Individual and mean serum atezolizumab concentrations may be plotted. Atezolizumab concentration data may be compared with data from other studies as warranted by the data.

Exploratory evaluations will be performed, with results reported with appropriate caveats about the exploratory nature of the analysis, and without formal adjustment for multiple comparisons.

### D. Reporting and Exclusions

Evaluation of Toxicity: All patients will be evaluable for toxicity from the time of their first treatment with atezolizumab.

Evaluation of Response: All patients included in the study must be assessed for response to treatment, even if there are major protocol treatment deviations or if they are ineligible. Each patient will be assigned one of the following categories: 1) complete response, 2) partial response, 3) stable disease, 4) progressive disease, 5) early death from malignant disease, 6) early death from toxicity, 7) early death because of other cause, or 9) unknown (not assessable, insufficient data). [Note: By arbitrary convention, category 9 usually designates the “unknown” status of any type of data in a clinical database.]

All of the patients who met the eligibility criteria (with the possible exception of those who received no study medication) should be included in the main analysis of the response rate. Patients in response categories 4-9 should be considered to have a treatment failure (disease progression). Thus, an incorrect treatment schedule or drug administration does not result in exclusion from the analysis of the response rate. Precise definitions for categories 4-9 will be protocol specific.

All conclusions should be based on all eligible patients. Subanalyses may then be performed on the basis of a subset of patients, excluding those for whom major protocol deviations have been identified (e.g., early death due to other reasons, early discontinuation of treatment, major protocol violations, etc.). However, these subanalyses may not serve as the basis for drawing conclusions concerning treatment efficacy, and the reasons for excluding patients from the analysis should be clearly reported. The 95% confidence intervals should also be provided.

### XI. Immune RECIST Criteria

Increasing clinical experience indicates that traditional response criteria (e.g., Response Evaluation Criteria in Solid Tumors, Version 1.1 [RECIST v1.1] and World Health Organization [WHO]) may not be sufficient to fully characterize activity in the new era of target therapies and/or biologics. In studies with cytokines, cancer vaccines, and monoclonal antibodies, complete response, partial response, or stable disease has been shown to occur after an increase in tumor burden as characterized by progressive disease by traditional response criteria.

Therefore, conventional response criteria may not adequately assess the activity of immunotherapeutic agents because progressive disease (by initial radiographic evaluation) does not necessarily reflect therapeutic failure. Long-term effect on the target disease must also be captured.

The immune RECIST (iRECIST) criteria, by incorporating specific response patterns that have been observed with immunotherapeutic agents, have been developed to address these issues and provide standardized response criteria for use with immunotherapy [Seymour et al., The Lancet Oncology, 2017. 18(3): p. e143-e152].

iRECIST Assessment of Response

All patients will have their BEST RESPONSE from the start of study treatment until the end of treatment classified as outlined below:

Complete Response (CR): disappearance of target and non-target lesions and normalization of tumor markers. Pathological lymph nodes must have short axis measures <10 mm (Note: continue to record the measurement even if <10 mm and considered CR). Residual lesions (other than nodes <10 mm) thought to be non-malignant should be further investigated (by cytology specialized imaging or other techniques as appropriate for individual cases [Eisnhauer et al., Eur J Cancer, 2009. 45(2): p. 228-47] before CR can be accepted.

Partial Response (PR): at least a 30% decrease in the sum of measures (longest diameter for tumor lesions and short axis measure for nodes) of target lesions, taking as reference the baseline sum of diameters. Non target lesions must be non-PD.

Stable Disease (SD): Neither sufficient shrinkage to qualify for PR nor sufficient increase to qualify for PD taking as reference the smallest sum of diameters on study.

Progressive Disease (PD): at least a 20% increase in the sum of diameters of measured lesions taking as references the smallest sum of diameters recorded on study (including baseline) AND an absolute increase of ≥5 mm. Appearance of new lesions will also constitute progressive disease (including lesions in previously unassessed areas). In exceptional circumstances, unequivocal progression of non-target disease may be accepted as evidence of disease progression, where the overall tumor burden has increased sufficiently to merit discontinuation of treatment or where the tumor burden appears to have increased by at least 73% in volume. Modest increases in the size of one or more non-target lesions are NOT considered unequivocal progression. If the evidence of PD is equivocal (target or non-target), treatment may continue until the next assessment, but if confirmed, the earlier date must be used.

Patients that are clinically well may continue on therapy following progression with new lesions or increase in target lesions if the increase in disease burden does not meet the definition of PD by immune response criteria [Seymour et al., The Lancet Oncology, 2017. 18(3): p. e143-e152]. In this situation, patients do not have unequivocal progression until immune response criteria are met (see Table 27).

TABLE 27
Integration of target, non-target and new lesions into response assessment

| | | | | |
|---|---|---|---|---|
| Target | Non-Target | New | Overall | Best Response for this |
| Lesions | Lesions | Lesions | Response | Category also Requires |

Target lesions ± non target lesions

| | | | | |
|---|---|---|---|---|
| CR | CR | No | CR | Normalization of tumor markers, |
| | | | | tumor nodes <10 mm |
| CR | Non-CR/Non-PD | No | PR | |
| CR | Not all evaluated | No | PR | |
| PR | Non-PD/not all | No | PR | |
| | evaluated | | | |
| SD | Non-PD/not all | No | SD | Documented at least once ≥4 wks. |
| | evaluated | | | from baseline |
| Not all evaluated | Non-PD | No | NE | |
| PD | Any | Any | PD | |
| Any | PD | Any | PD | |
| Any | Any | Yes | PD | |

Non target lesions ONLY

| | | | | |
|---|---|---|---|---|
| No Target | CR | No | CR | Normalization of tumor markers, |
| | | | | tumor nodes <10 mm |
| No Target | Non-CR/non-PD | No | Non-CR/ | |
| | | | non-PD | |
| No Target | Not all evaluated | No | NE | |
| No Target | Unequivocal PD | Any | PD | |
| No Target | Any | Yes | PD | |

Note:
Patients with a global deterioration of health status requiring discontinuation of treatment without objective evidence of disease progression at that time should be reported as “symptomatic deterioration”. This is a reason for stopping therapy, but is NOT objective PD. Every effort should be made to document the objective progression even after discontinuation of treatment.

iRECIST Assessment of Progression

Immunotherapeutics may result in infiltration of immune cells leading to transient increase in the size in malignant lesions, or undetectable lesions becoming detectable. Investigators should continue treatment, as appropriate, in the absence of unacceptable toxicity, until unequivocal disease progression. This is particularly important for patients in whom pseudoprogression may have occurred. Follow up response assessments must be continued until unequivocal disease progression has occurred.

The iRECIST criteria are identical to those of RECIST 1.1 in many respects but have been adapted to account for instances where an increase in tumor burden, or the appearance of new lesions, does not reflect true tumor progression.

Key differences are described below. All responses defined using iRECIST criteria are designated with a prefix. iRECIST time-point and best overall responses will be recorded separately. Unlike RECIST 1. 1, iRECIST requires the confirmation of progression and uses the terms iUPD (unconfirmed progression) and iCPD (confirmed progression). Confirmatory scans should be performed at least 4 weeks, but no longer than 8 weeks after iUPD.

### Confirming Progression

iCPD is confirmed if further increase in tumor burden, compared to the last assessment, is seen as evidenced by one or more of the following:

Continued increase in tumor burden (from iUPD) where RECIST 1.1 definitions of progression had been met (from nadir) in target, non-target disease or new lesions.

Progression in target disease worsens with an increase of at least 5 mm in the absolute value of the sum.

Continued unequivocal progression in non-target disease with an increase in tumor burden.

Increase in size of previously identified new lesion (s) (an increase of at least 5 mm in the absolute value of the sum of those considered to be target new lesions) or additional new lesions. RECIT 1.1 criteria are met in lesions types (target or non-target or new lesions) where progression was not previously identified, including the appearance of additional new lesions.

If the criteria are still met, but no worsening, or iSD, iPR or iCR if those criteria are met compared to baseline). As can be seen in Table 28, prior documentation of iUPD does not preclude assigning iCR, iPR, or iSD in subsequent time-point assessments or as best overall response (BOR) providing that iCPD is not documented at the next assessment after iUPD.

New lesions: New lesions should be assessed and measured as they appear using RECIST 1.1 criteria (maximum of 5 lesions, no more than 2 per site, at least 10 mm in long axis (or 15 mm in short axis for nodal lesions), and recorded as New Lesions-Target (NLT) and New Lesion-Non-Target (NLNT) to allow clear differentiation from baseline target and non-target lesions.

New lesions may either meet the criteria of NLT or NLNT to drive iUPD (or iCPD). However, the measurements of target lesions should NOT be included in the sum of measures of original target lesions identified at baseline. Rather, these measurements will be collected on a separate table in the case record form.

PD is confirmed in the New Lesion category if the next imaging assessment, conducted at least 4 weeks (but not more than 8 weeks) after iUPD confirms further progression from iUPD with either an increase of at least 5 mm in the absolute value of the sum of NLT OR an increase (but not necessarily unequivocal increase) in the size of NLNT lesions OR the appearance of additional new lesions.

TABLE 28
Time-point (TP) iResponse

| | | | Time Point Response | |
|---|---|---|---|---|
| Target | Non-Target | New | No prior | |
| Lesions* | Lesions* | Lesions* | IUPD** | Prior iUPD** |
| CR/iCR | CR/ | No | CR | iCR |
| CR/iCR | Non-CR/Non-PD | No | PR | iPR |
| | Non-iCR/Non- | | | |
| | iUPD | | | |
| PR/iPR | Non-CR/Non-PD | No | PR | iPR |
| | Non-iCR/Non- | | | |
| | iUPD | | | |
| SD/ISD | Non-CR/Non-PD | No | SD | iSD |
| | Non-iCR/Non- | | | |
| | iUPD | | | |
| iUPD with no | iUPD with no | Yes | NA | The appearance NLs confirms PD if Additional |
| change OR | decrease from last | | | NLs or iUPD in last TP based on NLs and |
| decrease from | change OR | | | increase in size (≥5 mm for NLT or any increase |
| last TP | TP | | | for NLNT) |
| | | | | If no change in NLs from last TP, remains iUPD |
| PD | Non-CR/Non-PD | No | iUPD | Remains iUPD unless iCPD confirmed based on |
| | Non-iCR/Non- | | | further increase in sum of at least 5 mm, |
| | IUPD | | | otherwise remains iUPD |
| PD | PD | No | iUPD | iCPD if further increase in previously |
| | | | | identified*** |
| | | | | T lesion iUPD ≥5 mm and/or NT lesion iUPD |
| PD | PD | Yes | iUPD | iCPD if further increase in previously identified |
| | | | | T lesion iUPD ≥5 mm and/or NT lesion iUPD |
| | | | | and/or size or number of new lesion |
| Non-iUPD | Non-iUPD | Yes | iUPD | iCPD if increase in size of previously identified |
| | | | | new lesions of increased number of new lesion |

*Using RECIST 1.1 principles. If no pseudoprogression occurs, RECIST 1.1 and iRECIST categories for CR, PR and SD would be the same.
**In any category.
***Previously identified in assessment immediately prior to this time-point (TP)
NA = not applicable.

All patients will have their iBOR from the start of study treatment until the end of treatment classified as outlined in Table 29.

TABLE 29
iRECIST Best Overall Response (iBOR)

| TPR1 | TPR2 | TPR3 | TPR4 | TPR5 | iBOR |
|---|---|---|---|---|---|
| CR | CR, PR, | CR/iCR, PR/ | iUPD | iCPD | iCR |
| | iUPD, NE | iPR, iUPD, | | | |
| iUPD | PR, SD, | iCPD, NE | CR, PR, SD, | CR, PR, SD, | iCR |
| | NE | CR | iUPD, NE | iUPD, iCPD, | |
| | | | | NE | |
| iUPD | PR | PR, SD, | PR, SD, iUPD, | PR, SD, iUPD, | iPR |
| | | iUPD, NE | NE, cPD | NE, iCPD | |
| iUPD | SD, NE | PR | PR, SD, | PR, SD, iUPD, | iPR |
| | | | iUPD, NE | iCPD, NE | |
| iUPD | SD | SD, iUPD, | SD, iUPD, | SD, iUPD, | iSD |
| | | NE | iCPD, NE | iCPD, NE | |
| iUPD | iUPD | Anything | Anything | Anything | iCPD |
| iUPD | iUPD | iCPD | Anything | Anything | iCPD |
| iUPD | NE | NE | NE | NE | iUPD |

1. Table assumes a randomized study where confirmation of CR or PR is not required.
2. NE = not evaluable that cycle.
3. Designation “I” for BOR can be used to indicate prior uPD to aid in data interpretation.
4. For patients with non-target disease only at baseline, only CR or non-CR/non-PD can be assigned at each TPR but is not shown in the table for ease of presentation.

Response and Stable Disease Duration (RECIST 1.1 and iRECIST)

Response duration will be measured from the time measurement criteria for CR/PR or iCR/iPR (whichever is first recorded) are first met until the first date that recurrent or progressive disease is objectively documented, taking as reference the smallest measurements recorded on study (including baseline).

Stable disease duration will be measured from the time of start of treatment until the criteria for progression are met, taking as reference the smallest sum on study (including baseline).

### XII. Procedure for Isolation and Cryopreservation of PBMCs from Whole Blood

### A. Isolation of PBMCs

Equipment: 10 mL EDTA tube, SepMate 50 mL tube, PipetAid, centrifuge

Reagents/Materials: LymphoPrep (500 mL), Dulbecco's PBS with 2% FBS (500 mL), Gibco™ Advanced DMEM/F12 media, 25 mL disposable serological pipets, 10 mL disposable serological pipets, 50 mL conical tubes, sterile polystyrene aspirating pipets

### Notes:

Lab coats, safety glasses, and gloves must be worn at all times when handling hazardous or sensitive equipment, samples, reagents, and materials. These safety measures must also be followed when in close proximity to those who are working with these items. Ensure that the whole blood, PBS+2% FBS, Lymphoprep density gradient medium, and centrifuge are all at room temperature (15-25° C.).

Before isolating PBMCs, prepare cryopreservation Component #1, as described in the Cryopreservation section below, for resuspending PBMCs after isolation.

### Procedure:

Blood will be collected aseptically by venipuncture or from a venous port into one 6 mL EDTA tube; the collected blood samples are stable for up to 60 minutes at room temperature (15° C. to 30° C.) prior to PMBC isolation. Obtain correct size SepMate tubes for volume of blood needing to be isolated as described in Table 30.

TABLE 30
Sample and Density Gradient Medium Volumes:

| | Initial | Density Gradient |
|---|---|---|
| Sepmate ™ Tube | Sample (mL) | Medium (mL) |
| 15 | 0.5-4.0 | 4.5 |
| 15 | >4-5 | 3.5 |
| 50 | 4-17 | 15 |

Keeping the SepMate™ tube completely upright, add Lymphoprep density gradient media by carefully pipetting it through the central hole of the SepMate™ insert. Refer to Table 1 for required volumes of Lymphoprep density gradient medium per sample; for 6 mL whole blood, use a 50 mL SepMate tube and 15 mL of density gradient. The top of the Lymphoprep density gradient medium will be above the insert. NOTE: Small bubbles may be present in the density gradient medium after pipetting. These bubbles will not affect performance.

Dilute all whole blood samples with an equal volume of PBS+2% FBS in new 50 mL conical tube and mix gently by pipetting up and down. Keeping the SepMate™ tube vertical, add the diluted sample by pipetting it slowly down the side of the tube. Repeat for each sample to be processed. The sample will mix with the density gradient medium above the insert. Centrifuge at 1200×g for 10 minutes at room temperature, with the brake on. Note: For samples older than 24 hours, a centrifugation time of 20 minutes is recommended. To reduce platelet contamination in the enriched PBMCs, pipette off some of the supernatant above the PBMC layer, leaving 1 inch above the insert or RBC pack. Pour off the rest of the supernatant which contains the enriched PBMCs, into a new 50 mL tube. Do not hold the SepMate™ tube in the inverted position for longer than 2 seconds. If there is substantial carryover of red blood cells (RBCs) in the PBMC supernatant repeat steps 8.1 to 8.6 using another SepMate tube with Lymphoprep density gradient media. Add 10 mL PBS+2% FBS to the 50 mL tube containing PBMCs. Centrifuge at 120×g for 10 minutes at room temperature, with the brake off. Aspirate supernatant without aspirating the PBMC pellet. This will remove platelets that are present.

Wash enriched PBMCs by adding another 10 mL PBS+2% FBS to the 50 mL tube. Centrifuge at 300×g for 8 minutes at room temperature, with the brake on. Aspirate supernatant without aspirating the PBMC pellet. Resuspend PBMCs in 2.5 mL of cryopreservation Component #1.

### B. Cryopreservation of PBMCs

Equipment: 1000 μL single channel pipet, CoolCell® LX, cryo-resistant label supplies, PipetAid, Nalgene™ Rapid-Flow™ Sterile Disposable Filter Units with CN Membrane, 2 microns

Reagents/Materials: FBS, DMSO, Gibco™ Advanced DMEM/F12 media, Thermo Scientific Nunc 368632 CryoTube, free standing round bottom; 1.8 mL, 50/bag, 25 mL disposable serological pipets, 10 mL disposable serological pipets, 3′ Core Polyester Chemical Resistant labels, 1000 μL filtered pipet tips (RTL-1000F)

Notes: Lab coats, safety glasses, and gloves must be worn at all times when handling hazardous or sensitive equipment, samples, reagents, and materials. These safety measures must also be followed when in close proximity to those who are working with these items. Ensure that all reagents and cryopods are all at room temperature (15-25° C.).

Procedure: Prepare tumor derived cell cryopreservation media, Component #1 and Component #2 as described in Table 31.

TABLE 31
Preparation of tumor derived cell cryopreservation media

| | | |
|---|---|---|
| | | Final concentration |
| Reagent | For 100 mL | in 100 mL |

Component #1

| | | |
|---|---|---|
| Advanced DMEM/F12 media | 80 mL | 80% |
| FBS | 20 mL | 20% |

Component #2

| | | |
|---|---|---|
| Advanced DMEM/F12 media | 60 mL | 60% |
| FBS | 20 mL | 20% |
| DMSO | 20 mL | 20% |

Create Component #1 and #2 separately. Filter the various volumes per component of Advanced DMEM/F12 media and FBS, Do not filter DMSO. Add DMSO directly to filtered Component #2 and mix. Store at both Components at 4° C.

After Components #1 and #2 are combined, Advanced DMEM/F12 will have a final concentration of 70%, FBS 20% and DMSO 10%.

Combine cryopreservation media with PBMCs. Label cryotubes using labels compatible with cryogenic storage for each sample. Add 500 μL of Component #2 to each cryotube. Slowly add 500 μL of PBMCs resuspended in Component #1 to a cryotube containing 500 uL Component #2. Do not mix. Place all cryotubes in a cryopod, Place cryopod in a −80° C. freezer for 1 to 3 days. Note: Longer incubation at −80 C results in progressive loss of viability. For longer storage, place cryopods on dry ice and quickly transfer cryotubes from the cryopod to a box in liquid nitrogen storage. For shipment to MoCha, transfer cryotubes to a box or plastic bag prechilled on dry ice in a styrofoam container, cover with a sufficient amount of dry ice for an overnight shipment and close and tape container tightly.

### XIII. Procedure for Pediatric PK+ADA Blood Collection

Overview: Blood will be collected from pediatric patients <12 years of age for assessment of pediatric PK+ADA response (Table 32). Blood samples will be collected at the time points found in Table 32. Kits for sample collection will be provided to study sites by Labcorp; kits will include red top serum separator tubes for blood collection and cryovial specimen transport tubes for the PK and ADA serum samples. Serum samples will be shipped back to Labcorp on the day of collection or, if necessary, on the following day. Labcorp will then ship batches of samples to ICON, who will complete the PK and ADA analyses.

TABLE 32
Blood Collection Time Points for Pediatric PK + ADA

| Timepoint | PK | ADA |
|---|---|---|
| C 1 D 1 | Baseline (predose) | predose |
| C 1 D 1 | 30 min after the end of infusion | — |
| C 2 D 1 | predose | predose |
| C 3 D 1 | predose | predose |
| C 4 D 1 | predose | predose |
| C 4 D 1 | 30 min after the end of infusion | — |
| C 8 D 1 | predose | predose |
| C 12 D 1 | predose | predose |
| C 16 D 1 | predose | predose |
| Progression/Study drug | Any time during visit | Any time |
| discontinuation | | during visit |

Blood collection will be waived if a time point falls on a Friday or too late on a Thursday to ensure same-day FedEx pickup, due to shipment and processing requirements.

Collection and Processing: Obtain one 2.5 mL red top serum separator tube per time point. Draw blood into a 2.5 mL red top serum separator tube using standard venipuncture techniques. Document the blood volume and actual time of collection for each sample. Mix the blood with the clotting activation agent by gently inverting the tube 10 times. Place the serum separator tube upright to clot at room temperature for 30 minutes until centrifugation. Centrifuge for 15 minutes at 1500-2000×g (or minimum of 3000 rpm). Separate serum from blood sample within 20 minutes of the end of centrifugation. Use pipette provided to transfer approximately half of the serum (at least 150 μL) into an appropriately labeled PK transport tube. For all predose time points, use the pipette provided to transfer at least 550 μL of the remaining serum into an appropriately labeled ADA transport tube. Ensure that the patient ID is recorded on the label of the transport tube(s). Immediately freeze and store the sample(s) upright at or below −20° C. until shipment to Labcorp. Ship FROZEN to Labcorp on dry ice the day of collection (DOC) or the following day if DOC is not possible.

### XIV. Procedure for PK Blood Collection from Patients Ages 12 Years and Older

Overview: Blood will be collected from patients ≥12 years of age who are enrolled to the monotherapy arm and who consent to this optional PK study. Kits for sample collection will be provided to study sites by Labcorp; kits will include red top serum separator tubes for blood collection and cryovial specimen transport tubes for the PK serum samples. Serum samples will be shipped back to Labcorp on the day of collection or, if necessary, on the following day. Labcorp will then ship batches of samples to ICON, who will complete the PK analyses. These optional blood collections will begin on Day 1 of the next cycle that a patient begins once collection kits are available and the patient's consent for this optional collection is obtained. The collections may continue for up to three cycles, total, as detailed in Table 33.

TABLE 33
Blood Collection Time Points for PK
Samples from Patients ≥12 Years Old

| Time point | PK |
|---|---|
| Cx D 1 | predose |
| Cx D 1 | 30 min after the end of atezolizumab infusion |
| Cx + 1 D 1 | predose |
| Cx + 1 D 1 | 30 min after the end of atezolizumab infusion |
| Cx + 2 D 1 | predose |
| Cx + 2 D 1 | 30 min after the end of atezolizumab infusion |

Cx = the cycle a patient begins once collection kits are available and the patient’s consent for this optional collection is obtained. Blood collection will be waived if a time point falls on a Friday or too late on a Thursday to ensure same-day FedEx pickup, due to shipment and processing requirements.

Collection and Processing: Obtain one 2.5 mL red top serum separator tube per time point. Draw blood into a 2.5 mL red top serum separator tube using standard venipuncture techniques. Document the blood volume and actual time of collection for each sample. Mix the blood with the clotting activation agent by gently inverting the tube 10 times. Place the serum separator tube upright to clot at room temperature for 30 minutes until centrifugation. Centrifuge for 15 minutes at 1500-2000×g (or minimum of 3000 rpm). Separate serum from blood sample within 20 minutes of the end of centrifugation. Use pipette provided to transfer the serum (at least 150 μL) into an appropriately labeled PK transport tube. Ensure that the patient ID is recorded on the label of the transport tube(s). Immediately freeze and store the sample(s) upright at or below −20° C. until shipment to Labcorp. Ship FROZEN to Labcorp on dry ice the day of collection (DOC) or the following day if DOC is not possible.

### Example 4: Protocol for NCT03141684. Version 4/19/2024

### I. Overview

### A. Background

Alveolar soft part sarcoma (ASPS) is a rare, indolent disease with a poor prognosis that occurs most frequently in patients between 15 and 35 years of age. The tumors are slow-growing, painless masses involving almost every part of the body but predominantly in the trunk and proximal extremities; metastatic disease is frequently observed at presentation. An adolescent with ASPS who received atezolizumab under an individual-patient IND had a marked response, including complete resolution of a large brain lesion.

### B. Objectives

Determine the objective response rate (ORR) of atezolizumab in patients with advanced ASPS in adult subjects ≥18 years and in pediatric/adolescent subjects ≥2 years. Determine the ORR using RECIST v 1.1 of atezolizumab and bevacizumab in subjects ≥18 years old with advanced ASPS that has progressed atezolizumab monotherapy. Determine duration of response (DOR) to atezolizumab monotherapy or atezolizumab plus bevacizumab using RECIST v 1.1 and/or change in clinical symptoms. Measure PFS time for patients receiving atezolizumab monotherapy or atezolizumab plus bevacizumab as determined by investigator using RECIST v 1.1 Assess the number of activated CD8+ T cells infiltrating the tumor before and after treatment with atezolizumab monotherapy or atezolizumab plus bevacizumab, and correlate treatment-induced changes with clinical response. Measure and describe the atezolizumab pharmacokinetics (PK) and anti-drug antibodies (ADA) in patients ages 2-11 years old. Compare RECIST v 1.1 vs immune RECIST (iRECIST) in patients with ASPS on atezolizumab and atezolizumab+bevacizumab. Assess the post-progression response rate among patients who progress while on treatment holiday and then resume treatment. Analyze the genomic and immune profiles of tumors expressing Type 1 or Type 2 ASPL-TFE3 fusions and correlate findings with response to atezolizumab or atezolizumab in combination with bevacizumab. Measure and describe the atezolizumab pharmacokinetics (PK) in patients ≥12 years old.

### C. Eligibility

Patients with histologically or cytologically confirmed ASPS that is not curable by surgery. To be eligible for atezolizumab monotherapy, subjects must be ≥2 years of age at the NCI clinical center (≥14 years at other participating sites). To be eligible for atezolizumab in combination with bevacizumab, subjects must be ≥18 years of age and must have progressed on P10005 atezolizumab monotherapy.

### D. Study Design

Patients will receive atezolizumab at a fixed dose of 1200 mg IV for adults and 15 mg/kg for pediatric patients once every 21 days, in 21-day cycles. Patients in the atezolizumab plus bevacizumab arm will receive bevacizumab at 15 mg/kg IV after each dose of atezolizumab.

Tumor biopsies (mandatory at all sites) will be collected from adult patients (18 years of age) at baseline and prior to cycle 3 day 1 (±3 days), or at any point where there is clinical evidence that the patient is responding to drug, to evaluate any changes in either tumor or blood targets of immune checkpoint blockade.

An optional tumor biopsy may be collected from adult patients (18 years of age), on either treatment arm, at the time of disease progression (or pre-progression, following any restaging at which a 10-19% increase in tumor volume is observed). At the PI's discretion, a progression biopsy collected while a patient is on the monotherapy arm may be utilized as the baseline biopsy for the combination arm, provided the patient does not receive any anti-cancer agents or immunotherapy in the interim.

The trial will be halted unless there is at least one response in the initial 9 patients accrued to the monotherapy arm.

Adult patients who progress on atezolizumab monotherapy may cross over to receive atezolizumab in combination with bevacizumab.

The monotherapy arm was to remain open to pediatric patients until 5 pediatric patients contributed Cycle 3 Day 1 blood samples for the optional PK+ADA study, 13 pediatric patients enrolled (with or without blood sample collections), or the atezolizumab plus bevacizumab arm was closed to accrual, whichever happened first. The monotherapy arm was closed to all pediatric and adult patient accrual because enrollment was sufficient to meet study endpoints and atezolizumab is now approved for those patient populations.

### II. Summary of Collections and Measurements

A schema of the Atezolizumab Monotherapy Arm and Atezolizumab and Bevacizumab Combination Arm are provided in FIGS. 15 and 16. Tumor biopsies (mandatory for patients in which biopsies are deemed safe and feasible) will be collected from adult patients (≥18 years of age) at baseline and prior to cycle 3 day 1 (±3 days), or at any point where there is clinical evidence that the patient is responding to drug. If a patient is crossing over directly from monotherapy to the combination after <4 monotherapy cycles, that patient's monotherapy cycle 3 day 1 biopsy will be used as their baseline biopsy for the combination treatment arm.

An optional tumor biopsy may be collected from adult patients (≥18 years of age), on either treatment arm, at the time of disease at progression (or pre-progression, following any restaging at which a 10-19% increase in tumor volume is observed). A progression biopsy can be collected while a patient is on the monotherapy arm may be utilized as the baseline biopsy for the combination arm, provided the patient does not receive any anti-cancer agents or immunotherapy in the interim.

Mandatory collection of CTCs from patients ≥14 years of age at baseline, on cycle 2 day 1, at the start of every subsequent cycle (before drug administration), at disease progression, and at the time of each response assessment during treatment holidays and the subsequent follow-up period. While CTC collection is required at all of these timepoints, the requirement is waived if a collection time point falls on a Friday or too late on a Thursday to ensure same-day FedEx pickup, due to shipment and processing requirements. CTC collections will be repeated if a patient crosses over or re-enrolls to the combination arm. Plasma obtained during the isolation of CTCs will be retained to enable genetic analysis of cell-free DNA.

One additional blood sample will be collected for germline analysis at baseline (or any other time on study) from patients ≥18 years of age who consent to the optional sequencing component. In some aspects, PBMCs (optional) will be collected from patients ≥14 years of age at baseline, on cycle 3 day 1 (or the next restaging visit if cycle 3 day 1 has already passed), 12 weeks (±1 week) and 24 weeks (±1 week) later, and at disease progression or response.

Blood can be collected from pediatric patients <12 years of age* at the following time points for measurement of pharmacokinetics (PK) and anti-drug antibodies (ADA) (Table 2).

Blood can be collected from patients on the monotherapy arm who are ≥12 years of age at the time of enrollment for measurement of PK. These optional blood collections will begin on Day 1 of the next cycle that a patient begins once collection kits are available and the patient's consent for this optional collection is obtained. The collections may continue for up to three cycles, total, as detailed in Table 3.

Patients will undergo a CT scan at the end of cycle 3 and every two cycles thereafter (every 3 cycles for patients on study for >1 year; every 4 cycles for patients on study for >2 years). Patients on the atezolizumab monotherapy arm will have a CT scan at the end of cycle 1 which will be used to assess for early response only; no treatment decisions will be made unless clinically warranted. A

Adult patients who progress on atezolizumab monotherapy may cross over to receive atezolizumab in combination with bevacizumab.

### III. Objectives

A. Primary Objectives: Determine the objective response rate (ORR) using RECIST v 1.1 of atezolizumab in patients with advanced Alveolar Soft Part Sarcoma (ASPS) in adult subjects ≥18 years and in pediatric/adolescent subjects ≥2 years. Determine the ORR using RECIST v 1.1 of atezolizumab and bevacizumab in subjects ≥18 years old with advanced ASPS that has progressed on atezolizumab monotherapy.

B. Secondary Objectives. Determine duration of response (DOR) to atezolizumab monotherapy or atezolizumab plus bevacizumab using RECIST v 1.1 and/or change in clinical symptoms (Time frame: baseline until disease progression, death, loss to follow-up, initiation of another anti-cancer treatment, withdrawal of consent, or study termination). Measure PFS time for patients receiving atezolizumab monotherapy or atezolizumab plus bevacizumab as determined by investigator using RECIST v 1.1 (Time frame: baseline until disease progression, death, loss to follow-up, initiation of another anti-cancer treatment, withdrawal of consent, or study). Assess the number of activated CD8+ T cells infiltrating the tumor before and after treatment with atezolizumab monotherapy or atezolizumab plus bevacizumab, and correlate treatment-induced changes with clinical response. Measure and describe the atezolizumab pharmacokinetics (PK) and anti-drug antibodies (ADA) in patients ages 2-11 years old.

C. Exploratory Objectives: Compare RECIST v 1.1 vs immune RECIST (iRECIST) in patients with ASPS on atezolizumab and atezolizumab+bevacizumab. Assess the post-progression response rate among patients who progress while on treatment holiday and then resume treatment. Analyze the genomic and immune profiles of tumors expressing Type 1 or Type 2 ASPL-TFE3 fusions and correlate findings with response to atezolizumab or atezolizumab in combination with bevacizumab. Measure and describe the atezolizumab pharmacokinetics (PK) in patients ≥12 years old.

### IV. Patient-Selection

### A. Eligibility Criteria

Patients must have histologically or cytologically confirmed Alveolar Soft Part Sarcoma that is not curable by surgery. Diagnosis of malignancy must be confirmed by the department of pathology at the institution where the patient is enrolled prior to patient enrollment. To be eligible for atezolizumab in combination with bevacizumab, the patient must have atezolizumab-refractory/resistant disease that has progressed (definitive clinical progression or iCPD) on prior P10005 atezolizumab monotherapy.

Patients must have measurable disease, defined as at least one lesion that can be accurately measured in at least one dimension (longest diameter to be recorded for non-nodal lesions and short axis for nodal lesions) as ≥20 mm (≥2 cm) by chest x-ray or as ≥10 mm (1 cm) with CT scan, MRI, or calipers by clinical exam. See the Measurement of Effect section for the evaluation of measurable disease. Note: Once the primary endpoint of the trial has been met, pediatric patients may enroll with evaluable non-measurable disease and will not be required to have measurable disease. Evaluable non-measurable disease is that which is not measurable by RECIST1.1 (see the Measurement of Effect section) but can be evaluated by imaging (e.g., CT, bone scan, or ultrasound) or other methods.

Patients with newly diagnosed, unresectable, metastatic and measurable ASPS will also be eligible for atezolizumab monotherapy if they show clinical evidence of disease progression (including history and increasing physical symptoms). On-study documentation will include a physician's rationale that supports evidence of clinical disease progression (i.e., increasing tumor pain).

To be eligible for atezolizumab monotherapy, subjects must be ≥2 years of age. To be eligible for atezolizumab in combination with bevacizumab, subjects must be ≥18 years of age.

ECOG performance status ≤2 (Karnofsky or Lansky ≥70%, FIG. 17).

Life expectancy of greater than 3 months.

Patients must have normal organ and marrow function as defined below: absolute neutrophil count≥1,500/mcL platelets≥100,000/mcL hemoglobin≥8 g/dL total bilirubin≤1.5×institutional upper limit of normal (ULN) (however, patients with known Gilbert disease who have serum bilirubin level 3×ULN may be enrolled) AST(SGOT)/ALT(SGPT)≤3×ULN (AST and/or ALT 5×ULN for patients with liver involvement) alkaline phosphatase ≤2.5×ULN (5×ULN for patients with documented liver involvement or bone metastases) creatinine clearance. For adult patients (≥18 years of age): ≥30 mL/min/1.73 m² by Cockroft-Gault Creatine clearance is determined by the following formula ((140−age)×(weight in kg))/(72×(serum creatine in mg/dL) (×0.85 if female). For pediatric patients (<18 years of age), serum creatinine based on age and gender are shown in FIG. 18.)

Administration of atezolizumab or bevacizumab may have an adverse effect on pregnancy and poses a risk to the human fetus, including embryo-lethality. Female patients of child-bearing potential and male patients must agree to use adequate contraception (hormonal or barrier method of birth control; abstinence) prior to study entry, for the duration of study participation, and for 5 months (150 days) after the last dose of atezolizumab or 6 months (180 days) after the last dose of bevacizumab. Should a woman become pregnant or suspect she is pregnant while she or her partner is participating in this study, she should inform her treating physician immediately. Ability to understand and the willingness to sign a written informed consent document or a parent/guardian able to do the same.

Willingness to provide biopsy samples for research purposes (patients 18 years of age only). At the PI's discretion, archival tissue that was collected and preserved in a manner consistent with the tissue processing details in the Biopsy Collection and Shipment section may be used in lieu of a baseline biopsy, provided no anti-cancer agents or immunotherapies were received since collection of the archival tissue.

Patients who have received live attenuated vaccines within 30 days of the first dose of trial treatment are eligible at the discretion of the investigator. All seasonal influenza vaccines and vaccines intended to prevent SARS-CoV-2 and coronavirus disease 2019 (COVID-19) are allowed.

For patients not receiving therapeutic anticoagulation: INR or aPTT ≤1.5×ULN for bevacizumab patients.

For patients receiving therapeutic anticoagulation: stable anticoagulant regimen for bevacizumab patients.

### B. Exclusion Criteria

1) Any prior therapy must have been completed ≥4 weeks or, if known, ≥5 half-lives of the prior agent (whichever is shorter) prior to enrollment on protocol (minimum of 1 week between prior therapy and study enrollment), and the participant must have recovered to eligibility levels from prior toxicity. Note: For patients being treated on the atezolizumab plus bevacizumab arm, there is no washout requirement for prior anti-PD-1 or anti-PD-L1 agents. Patients should be at least 6 weeks out from nitrosoureas and mitomycin C. Prior definitive radiation should have been completed ≥4 weeks or palliative radiation should have been completed ≥2 weeks prior to study enrollment or crossover and all associated toxicities resolved to eligibility levels (patients on study may be eligible for palliative radiotherapy to non-targeted lesions after 2 cycles of therapy at the PI's discretion). Patients who have had prior monoclonal antibody therapy must have completed that therapy ≥6 weeks (or 3 half-lives of the antibody, whichever is shorter) prior to enrollment on protocol (minimum of 1 week between prior therapy and study enrollment). A patient who has received a cumulative dose of >350 mg/m² of anthracycline (regardless of cardioprotectant) may only be enrolled if their ejection fraction measured by an echocardiogram is within normal institutional limits.
2) Prior treatment with anti-PD-1, or anti-PD-L1 therapeutic antibody or pathway-targeting agents. Patients who have received prior treatment with anti-CTLA-4 may be enrolled on the atezolizumab monotherapy arm, provided the following requirements and all other selection criteria are met: Minimum of 12 weeks from the first dose of anti-CTLA-4 and >6 weeks from the last dose and no history of severe immune-related adverse effects from anti-CTLA-4 (NCI CTCAE Grade 3 and 4). Patients who have progressed on P10005 atezolizumab monotherapy may be eligible for the atezolizumab plus bevacizumab arm, provided the following requirements, and all other selection criteria, are met: no prior disease progression on an immune checkpoint inhibitor plus tyrosine kinase inhibitor combination therapy, any prior toxicities have resolved to eligibility levels, and no contraindications.
3) Treatment with any other investigational agent within 4 weeks (or within five half-lives of the investigational product, whichever is shorter) prior to Cycle 1, Day 1 (minimum of 1 week between prior therapy and study enrollment). Patients must be ≥2 weeks since any investigational agent administered as part of a Phase 0 study (also referred to as an “early Phase I study” or “pre-Phase I study” where a sub-therapeutic dose of drug is administered) at the Coordinating Center PI's discretion, and should have recovered to eligibility levels from any toxicities.
4) Treatment with systemic immunostimulatory agents (including, but not limited to, interferon-α or interleukin-2 [aldesleukin]) within 6 weeks prior to Cycle 1, Day 1.
5) Treatment with systemic immunosuppressive medications (including, but not limited to, prednisone, cyclophosphamide, azathioprine, methotrexate, thalidomide, and anti-tumor necrosis factor [anti-TNF]agents) within 2 weeks prior to Cycle 1, Day 1. Patients who have received acute, low dose, systemic immunosuppressant medications (e.g., a one-time dose of dexamethasone for nausea) may be enrolled. The use of inhaled corticosteroids and systemic mineralocorticoids (e.g., fludrocortisone) for patients with orthostatic hypotension or adrenocortical insufficiency is allowed.
6) Patients taking bisphosphonate therapy for symptomatic hypercalcemia. Use of bisphosphonate therapy for other reasons (e.g., bone metastasis or osteoporosis) is allowed.
7) Patients with known primary central nervous system (CNS) malignancy or symptomatic CNS metastases are excluded, with the two following exceptions. Patients with asymptomatic untreated CNS disease may be enrolled, provided all of the following criteria are met (Evaluable or measurable disease outside the CNS, no metastases to brain stem, midbrain, pons, medulla, or cerebellum, no history of intracranial hemorrhage or spinal cord hemorrhage, no ongoing requirement for dexamethasone for CNS disease; patients on a stable dose of anticonvulsants are permitted, and no neurosurgical resection or brain biopsy within 28 days prior to Cycle 1, Day 1. Second, patients with asymptomatic treated CNS metastases may be enrolled, provided all the criteria listed above are met as well as the following: Radiographic demonstration of improvement upon the completion of CNS-directed therapy and no evidence of interim progression between the completion of CNS-directed therapy and radiographic screening for the current study; No stereotactic radiation or whole-brain radiation within 28 days prior to Cycle 1, Day 1; Screening CNS radiographic study ≥4 weeks from completion of radiotherapy and >2 weeks from discontinuation of corticosteroids.
8) Known hypersensitivity to Chinese hamster ovary cell products or other recombinant human antibodies.
9) History of severe allergic, anaphylactic, or other hypersensitivity reactions to chimeric or humanized antibodies (i.e., antibodies with generic names ending in “ximab” or “zumab”, respectively) or fusion proteins.
10) Uncontrolled intercurrent illness including, but not limited to, ongoing or active infection, symptomatic congestive heart failure, unstable angina pectoris, cardiac arrhythmia, or psychiatric illness/social situations that would limit compliance with study requirements.
11) Pregnant women are excluded from this study because atezolizumab is an investigational agent with the unknown potential for teratogenic or abortifacient effects. Because there is an unknown but potential risk for adverse events in nursing infants secondary to treatment of the mother with atezolizumab, breastfeeding should be discontinued if the mother is treated with atezolizumab.
12) HIV-positive patients may be on combination antiretroviral therapy but must have a CD4 count >350 cells/mm³ with an undetectable viral load.
13) Patients on supraphysiologic doses of steroids or use of such within the previous six weeks.
14) Known clinically significant liver disease, including active viral, alcoholic, or other hepatitis; cirrhosis; fatty liver; and inherited liver disease. Patients with past or resolved hepatitis B infection (defined as having a negative hepatitis B surface antigen [HBsAg] test and a positive anti-HBc [antibody to hepatitis B core antigen] antibody test) are eligible. Patients positive for hepatitis C virus (HCV) antibody are eligible only if polymerase chain reaction (PCR) is negative for HCV RNA.
15) History or risk of autoimmune disease, including, but not limited to, systemic lupus erythematosus, rheumatoid arthritis, inflammatory bowel disease, vascular thrombosis associated with antiphospholipid syndrome, Wegener's granulomatosis, Sjögren's syndrome, Bell's palsy, Guillain-Barré syndrome, multiple sclerosis, autoimmune thyroid disease, vasculitis, or glomerulonephritis.
16) Patients with a history of autoimmune hypothyroidism on a stable dose of thyroid replacement hormone may be eligible. Patients with controlled Type 1 diabetes mellitus on a stable insulin regimen may be eligible. Patients with eczema, psoriasis, lichen simplex chronicus, or vitiligo with dermatologic manifestations only (e.g., patients with psoriatic arthritis would be excluded) are permitted provided that they meet the following conditions: patients with psoriasis must have a baseline ophthalmologic exam to rule out ocular manifestations, rash must cover less than 10% of body surface area (BSA), disease is well controlled at baseline and only requiring low potency topical steroids (e.g., hydrocortisone 2.5%, hydrocortisone butyrate 0.1%, flucinolone 0.01%, desonide 0.05%, aclometasone dipropionate 0.05%), and no acute exacerbations of underlying condition within the last 12 months (not requiring psoralen plus ultraviolet A radiation [PUVA], methotrexate, retinoids, biologic agents, oral calcineurin inhibitors; high potency or oral steroids).
17) History of idiopathic pulmonary fibrosis, pneumonitis (including drug induced), organizing pneumonia (i.e., bronchiolitis obliterans, cryptogenic organizing pneumonia, etc.), or evidence of active pneumonitis on screening chest computed tomography (CT) scan. History of radiation pneumonitis in the radiation field (fibrosis) is permitted.
18) Patients with active tuberculosis (TB) are excluded.
19) Severe infections within 4 weeks prior to Cycle 1, Day 1, including, but not limited to, hospitalization for complications of infection, bacteremia, or severe pneumonia.
20) Signs or symptoms of infection within 2 weeks prior to Cycle 1, Day 1.
21) Received oral or intravenous (IV) antibiotics within 2 weeks prior to Cycle 1, Day 1. Patients receiving prophylactic antibiotics (e.g., for prevention of a urinary tract infection or chronic obstructive pulmonary disease) are eligible.
22) Major surgical procedure within 28 days prior to Cycle 1, Day 1 or anticipation of need for a major surgical procedure during the course of the study. Note: Patients with a surgical procedure (excluding biopsy procedures described in Biopsy collection and shipment) within 28 days prior to Cycle 1 Day 1, or any unhealed surgical wound, are not eligible for the atezolizumab+bevacizumab combination arm.
23) Administration of a live, attenuated vaccine within 4 weeks before Cycle 1, Day 1 or anticipation that such a live, attenuated vaccine will be required during the study and up to 5 months after the last dose of atezolizumab. Influenza vaccination should be given during influenza season only (approximately October to March). Patients must not receive live, attenuated influenza vaccine within 4 weeks prior to Cycle 1, Day 1 or at any time during the study.
24) Additional exclusion criteria include: history of leptomeningeal disease, uncontrolled tumor-related pain, patients requiring pain medication must be on a stable regimen at study entry, symptomatic lesions (e.g., bone metastases or metastases causing nerve impingement) amenable to palliative radiotherapy should be treated prior to enrollment (Patients should be recovered from the effects of radiation. There is no required minimum recovery period.), asymptomatic metastatic lesions that would likely cause functional deficits or intractable pain with further growth (e.g., epidural metastasis that is not currently associated with spinal cord compression) should be considered for loco-regional therapy if appropriate prior to enrollment, uncontrolled pleural effusion, pericardial effusion, or ascites requiring recurrent drainage procedures (once monthly or more frequently), patients with indwelling catheters (e.g., PleurX®) are allowed, uncontrolled or symptomatic hypercalcemia (ionized calcium >1.5 mmol/L, calcium >12 mg/dL or corrected serum calcium >ULN), history of malignancy within 3 years prior to screening, with the exception of malignancies with a negligible risk of metastasis or death (e.g., 5-year OS rate >90%), such as adequately treated carcinoma in situ of the cervix, non-melanoma skin carcinoma, localized prostate cancer, ductal carcinoma in situ, or Stage I uterine cancer, prior allogeneic stem cell or solid organ transplantation, and any other disease, metabolic dysfunction, physical examination finding, or clinical laboratory finding that contraindicates the use of an investigational drug, may affect the interpretation of the results, or may render the patient at high risk from treatment complications.

### C. Bevacizumab-Specific Exclusion Criteria

The following exclusion criteria, in addition to those listed above, apply to the atezolizumab+bevacizumab combination arm:
1. Age <18 years.
2. Prior disease progression on an immune checkpoint inhibitor plus tyrosine kinase inhibitor combination therapy.
3. Major surgery within 6 weeks of enrollment. Procedures (e.g. port placement, endoscopy with intervention) within 7 days of enrollment (excluding biopsy procedures described in Biopsy Collection and shipment).
4. Chronic daily treatment with a non-steroidal anti-inflammatory drug (NSAID), clopidogrel, dypiridamole, or aspirin therapy >81 mg/day. Treatment with any such agents within 7 days prior to Cycle 1 Day 1.
5. 2+ protein on urinalysis, followed by 24-hour protein of >1 g.
6. Patients with a history of bleeding varices within 1 year of enrollment.
7. Thromboembolic event within 6 months of enrollment (including cerebrovascular accident (CVA) and myocardial infarction (MI).
8. Evidence of bleeding diathesis or significant coagulopathy (with or without current therapeutic anticoagulation).
9. International normalized ratio (INR) ≥1.5 (or >therapeutic range if patient is receiving warfarin). PTT ≥1.5×ULN.
10. History of hemoptysis (>½ teaspoon of bright red blood per episode) within 1 month of enrollment.
11. Serious, non-healing wound, active ulcer, or untreated bone fracture.
12. Congestive heart failure, baseline LVEF <50%, transmural myocardial infarction, uncontrolled hypertension (defined as systolic blood pressure >140 mmHg and/or diastolic blood pressure >90 mmHg in patients ≥18 years of age), angina pectoris requiring medication, clinically significant valvular disease, high-risk arrhythmia within 12 months of enrollment. Prior history of hypertensive crisis or hypertensive encephalopathy.
13. History of abdominal fistula or gastrointestinal perforation within 6 months prior to enrollment.
14. History of allergic reactions attributed to compounds of similar chemical or biologic composition to bevacizumab.
15. For patients not receiving therapeutic anticoagulation: INR or aPTT ≤1.5×; for patients receiving therapeutic anticoagulation: stable anticoagulant regimen.
16. Significant vascular disease (e.g., aortic aneurysm requiring surgical repair or recent arterial thrombosis) within 6 months prior to randomization.
17. Core biopsy or other minor surgical procedure, excluding placement of a vascular access device, within 7 days prior to initiation of study treatment. Placement of a vascular access device should be at least 2 days prior to initiation of study treatment.
18. Active infection requiring IV antibiotics at the time of initiation of study treatment.
19. Current or recent (<10 days prior to initiation of study treatment) use of aspirin (>325 mg/day), or clopidogrel (>75 mg/day).
20. The use of full-dose oral or parenteral anticoagulants for therapeutic purpose is permitted as long as the INR and/or aPTT is within therapeutic limits (according to institution standards) within 7 days prior to initiation of study treatment and the patient has been on a stable dose of anticoagulants for ≥2 weeks prior to initiation of study treatment. Prophylactic use of anticoagulants is allowed. However, the use of direct oral anticoagulant therapies such as dabigatran (Pradaxa®) and rivaroxaban (Xarelto®) is not recommended due to bleeding risk.

### D. Eligibility Screening Evaluation

Patient eligibility screening tests will be performed as described below. Eligibility history, physical examination, laboratory evaluations, urinalysis, and EKG are to be conducted within 8 days prior to enrollment (or crossover) to either treatment arm. As noted in the “Treatment Plan” section below, if protocol therapy is started within 8 days of these eligibility screening evaluations, values from the screening evaluations may be used as baseline measurements; if >8 days have passed since the screening evaluations, the medical history, physical examination, laboratory evaluations, urinalysis, and EKG must be repeated prior to starting protocol therapy. Eligibility and baseline imaging scans and ECHO must be done within 28 days prior to enrollment and prior to the start of protocol therapy, respectively.
6. Histologic confirmation: A block or stained slides of primary tumor tissue or of known recurrence will be required from each participant to confirm diagnosis. Pathology confirmation will be performed by the Laboratory of Pathology, NIH for patients enrolled at the NCI or by the department of pathology at the site where the patient is enrolled.
7. History and physical examination: Complete history and physical examination (including height, weight, vital signs, and ECOG performance score) will be conducted within 8 days prior to both patient enrollment and the start of protocol therapy (monotherapy or the combination).
8. Imaging Studies: Every participant should have an evaluation of known sites of disease. Scans for tumor measurements must be done within 28 days prior to both patient enrollment and the start of protocol therapy (monotherapy or the combination).
9. Laboratory Evaluation: Laboratory tests to establish eligibility are to be performed within 8 days prior to both patient enrollment and the start of protocol therapy (monotherapy or the combination):
   Hematological Profile: CBC with differential, platelets
   Biochemical Profile: ACTH and AM cortisol*, albumin, alkaline phosphatase, amylase, total bilirubin, BUN, bicarbonate, calcium, chloride, creatinine, glucose, LDH, lipase, phosphorus, potassium, total protein, SGOT (AST), SGPT (ALT), sodium, TSH
      The requirements for ACTH and AM cortisol monitoring are waived while a patient is on exogenous or replacement steroid therapy; ACTH and AM cortisol may be measured at the PI's discretion.
   Urinalysis
   Serum or urine pregnancy test (B-HCG) for female participants of childbearing potential
   Prothrombin time (PT), partial thromboplastin time (PTT), or international normalized ratio (INR) test for participants on the combination arm only.
10. Cardiac Evaluation to establish eligibility. (a) EKG within 21 days prior to both patient enrollment and start of protocol therapy (monotherapy or the combination), (b) Echocardiogram (ECHO) within 28 days prior to both patient enrollment and the start of protocol therapy (monotherapy or the combination)

Patients who crossover or re-enroll to the combination treatment arm must be screened within 28 days of initiating the combination and must meet all of the patient selection criteria outlined in the Patient Selection Section, with the exception of the prohibition against prior anti-PD-L1 therapy. Currently enrolled patients who meet all of the selection criteria will crossover to the combination arm without being re-registered; they will retain their study ID number. Previously enrolled patients who re-enroll and meet all selection criteria will be registered for the combination arm as new patients with new ID numbers.

### V. Treatment Plan

This is an open-label phase 2 trial of atezolizumab in patients with advanced ASPS in adult subjects ≥18 years of age and in pediatric/adolescent subjects ≥2 years of age. This trial is also evaluating the combination of atezolizumab plus bevacizumab in adult subjects with advanced ASPS that progressed (definitive clinical progression or iCPD) on P10005 atezolizumab monotherapy.

Reported adverse events and potential risks for atezolizumab are described in the Adverse events section. Doses and drug administration are defined in this section. There are no dose modifications for this agent. Each cycle is 21 days (±3 days due to scheduling conflicts). The start of a new cycle may be delayed up to 2 weeks to accommodate scheduling conflicts and other unexpected events.

Patient evaluations will be performed throughout the study as described below. Baseline history, physical examination, laboratory evaluations, urinalysis, and EKG are to be conducted within 8 days prior to the start of protocol therapy. If protocol therapy is started within 8 days of the eligibility screening evaluations (see “Eligibility Screening Evaluation”), values from the screening evaluations may be used as baseline measurements; if >8 days have passed since the screening evaluations, the medical history, physical examination, laboratory evaluations, and urinalysis, must be repeated prior to starting protocol therapy (21 days are allowed for EKG). Baseline imaging scans and ECHO must be done within 28 days prior to the start of protocol therapy.

Baseline history and physical examination will be done within 8 days of cycle 1 day 1. History and physical examination will then be done within 8 days of day 1 of all subsequent cycles. Labs (CBC with differential; serum chemistries) will be performed at baseline (within 8 days of cycle 1 day 1) and then up to 3 days before the start of each subsequent cycle (LDH and protein do not need to be checked again after baseline). TSH will be checked at baseline (within 8 days of cycle 1 day 1) and then up to 3 days before the start of each subsequent cycle. Baseline EKG (within 21 days prior to start of protocol therapy) and baseline ECHO (within 28 days prior to start of protocol therapy) will be performed, and EKG and ECHO will be repeated as clinically indicated thereafter. Urinalysis will be done at baseline (within 8 days of cycle 1 day 1); urinalysis will be repeated up to 3 days before the start of each subsequent cycle for the atezolizumab+bevacizumab arm only.

Patients will have a CT scan (or other imaging test; see “Measurement of Effect” section) at baseline (within 28 days prior to start of protocol therapy in either arm). Patients in the monotherapy arm will have a CT scan at the end of cycle 1, which will be used to assess for early response only; no treatment decisions will be made unless clinically warranted.

All patients will undergo a CT scan at the end of cycle 3 and every two cycles thereafter (every 3 cycles for patients on study for >1 year; every 4 cycles for patients on study for >2 years). Given the concern of pseudoprogression (“tumor flare”), patients will only be taken off treatment if two scans at normal intervals show progression along with worsening clinical picture.

Patients have the option to receive protocol therapy between restaging visits at an ETCTN or EDDOP study site other than their enrolling site if the protocol is open at that site and provided the following conditions are met:
They have completed 8 cycles of protocol therapy (or they have the PI's approval to exercise this option sooner).
There is an investigator at the non-enrolling study site who is willing to assume responsibility for periodically treating the patient and maintaining communication with the site where the patient is enrolled.
They have the approval of the Clinical Investigator at their site of enrollment.

Patients will need to visit their enrolling site for restaging visits (e.g., end of cycle 9, end of cycle 11, etc.) and will start the subsequent treatment cycle at their enrolling site (e.g., start of cycle 10, start of cycle 12, etc.). Research blood collections that coincide with a patient's treatment at a non-enrolling site may be waived (CTCs) or postponed to the next restaging visit (PMBCs and PK+ADA collections).

Research biopsies will be mandatory for adult patients at all sites, provided the biopsy is deemed safe and feasible. Patients that cannot be safely biopsied may be considered for the study (see “Pharmacodymanic Assays in Tumor Tissue” section). Blood samples will be collected for PD studies from patients ≥14 years of age (some mandatory and some optional) and blood samples for PK+ADA studies (mandatory) will be collected from patients who are <12 years of age at enrollment, as described in “Blood Collection and Shipment” section.

### A. Agent Administration

Treatment will be generally administered on an outpatient basis. Reported adverse events and potential risks are described the “Adverse Events” section. Administration of atezolizumab will be performed in a setting with emergency management capabilities and staff who are trained to monitor for and respond to medical emergencies.

No investigational or commercial agents or therapies other than those described below may be administered with the intent to treat the patient's malignancy. Patients on study may be eligible for palliative radiotherapy to non-targeted lesions after completing 2 cycles of therapy and achieving stable or responding disease at the physician's discretion.

Patients on treatment for >2 years could be given the option of going on a treatment holiday for up to 2 years. Imaging scans will be required every 3 months during the holiday. If scans show signs of disease progression, the patient will be required to resume treatment within 21 days of the scan or go off-study. For disease evaluation purposes, the new scan will be the new baseline scan for all future RECIST comparisons. The post-progression response rate among patients who progress while on holiday and then resume treatment will be assessed as an exploratory endpoint. This endpoint will not include patients who resume therapy after a drug holiday (≤2 years) without progression.

The physician can decide to end the holiday early (before 2 years) with documentation of the rationale. Patients resuming treatment must be reassessed and shown to meet study eligibility criteria 5 through 10 and none of the exclusion criteria (with the exception of the Bavacizumab-specific exclusion criteria 2, prior treatment with an anti-PD-L1 therapeutic antibody); if they do not meet these selection criteria within 3 months, they must go off study. Patients who continue the treatment holiday for 2 full years without disease progression will be required to go off treatment and will undergo follow up every 6 months. Monotherapy and combination regiment descriptions are included in Tables 4 and 5. Note dose reduction is prescribed for the management of select AEs, as specified in Management of Specific AEs and FIG. 19.

For adult and pediatric patients, the initial dose of atezolizumab will be delivered as defined in the Agent Administration section. On the combination arm, bevacizumab will be administered after atezolizumab. On Cycle 1 Day 1 and Cycle 2 Day 1, bevacizumab will be administered 30 (±10) minutes after atezolizumab. If no atezolizumab-infusion-related reactions occur, the drugs can be administered sequentially in subsequent cycles.

The initial dose of bevacizumab will be delivered over 90 (±15) minutes. If the first infusion is tolerated without infusion-associated AEs, the second infusion may be delivered over 60 (±10) minutes. If the 60-minute infusion is well tolerated, all subsequent infusions may be delivered over 30 (±10) minutes.

For the first infusion of either agent, the patient's vital signs (heart rate, respiratory rate, blood pressure, and temperature) should be determined within 60 minutes before the first infusion. If clinically indicated, the patient's vital signs should be measured every 15 [±5] minutes during the first infusion and 30 (±10) minutes after the infusion. For subsequent infusions, vital signs will be collected within 60 minutes before the infusion. If the patient experienced an infusion-related reaction with the previous infusion or if clinically indicated, vital signs should be measured during the infusion and at 30 (±10) minutes after the infusion. Patients will be informed about the possibility of delayed post-infusion symptoms and instructed to contact their study physician if they develop such symptoms.

### B. Atezolizumab Infusion Related Reactions

For anaphylaxis precautions, see the management guidelines. Atezolizumab infusions will be administered per the instructions outlined in Table 6:

For anaphylaxis precautions, use the following procedure:

### Equipment Needed

Monitoring devices: ECG monitor, blood pressure monitor, oxygen saturation monitor, and thermometer
Oxygen
Epinephrine for subcutaneous, intravenous, and/or endotracheal use in accordance with standard practice
Antihistamines
Corticosteroids
IV infusion solutions, tubing, catheters, and tape

Procedures: In the event of a suspected anaphylactic reaction during atezolizumab infusion, the following procedures should be performed:
Stop the study drug infusion.
Call for additional medical assistance.
Ensure that appropriate monitoring is in place, with continuous ECG and pulse oximetry monitoring, if possible.
Administer antihistamines, epinephrine, or other medications as required by patient status and directed by the physician in charge.
Continue to observe the patient and document observation.
Draw serum/plasma samples for immunogenicity testing.
Ask participant to return for washout immunogenicity sample if appropriate.

### C. Bevacizumab Infusion Related Reactions

If a subject experiences an infusion-associated adverse event, he or she may be pre-medicated for the next study drug infusion; however, the infusion time may not be decreased for the subsequent infusion. If the next infusion is well tolerated with premedication, the subsequent infusion time may then be decreased by 30±10 minutes as long as the subject continues to be pre-medicated. If a subject experience an infusion-associated adverse event with the 60-minute infusion, all subsequent doses should be given over 90±15 minutes. Similarly, if a subject experience an infusion-associated adverse event with the 30-minute infusion, all subsequent doses should be given over 60±10 minutes.

### D. General Concomitant Medication and Supportive Care Guidelines

Because there is a potential for interaction of atezolizumab or bevacizumab with other concomitantly administered drugs, the case report form must capture the concurrent use of all other drugs, over-the-counter medications, or alternative therapies. A physician can be alerted if the patient is taking any agent known to affect or with the potential for drug interactions. The study team should check a frequently-updated medical reference for a list of drugs to avoid or minimize use of.

Palliative radiotherapy to non-targeted lesions may be permitted for patients receiving atezolizumab (alone or in combination with bevacizumab) after completing 2 cycles of therapy and achieving stable or responding disease, at the physician's discretion. Any authorized vaccines for the prevention of SARS-CoV-2 are allowed at any time prior to trial enrollment or during participation on the trial. Any vaccine for seasonal influenza is allowed as well. Bacillus Calmette-Guérin vaccine may be considered, if appropriate.

### Drugs to be Avoided:

Chronic daily treatment with a non-steroidal anti-inflammatory drug (NSAID), clopidogrel, dypiridamole, or aspirin therapy >81 mg/day.
Traditional herbal or homeopathic or natural medicines should be limited and used at the discretion of the investigator
Ingredients for such medicines have not been fully studied, and their use may result in unanticipated drug drug interactions that may cause or confound assessment of toxicity.
Immunostimulatory agents, including but not limited to interferon (IFN)-α, IFN-γ, anti TNF-α, or IL-2 (aldesleukin) (prohibited prior to and during the study and for 10 weeks after the last dose of atezolizumab; see Section Exclusion Criteria). These agents, in combination with atezolizumab, could potentially increase the risk for autoimmune conditions.
Immunosuppressive medications, including but not limited to cyclophosphamide, azathioprine, methotrexate, and thalidomide. These agents could potentially alter the activity and the safety of atezolizumab.
Live vaccines and live, attenuated vaccines (prohibited during the study and for 100 days after the last dose of study drug).
Initiation of granulocyte colony stimulating factors (e.g., filgrastim and biosimilar products, sargramostim, and/or pegfilgrastim) should be discussed with the Medical Monitor.

### E. Duration of Therapy

In the absence of treatment delays due to adverse event(s) or treatment holiday, treatment may continue for until one of the following criteria applies:
Disease progression,
Intercurrent illness that prevents further administration of treatment,
Unacceptable adverse event(s),
Patient decides to withdraw from the study, or
General or specific changes in the patient's condition render the patient unacceptable for further treatment in the judgment of the investigator.
   Patients who show evidence of clinical benefit may be permitted to continue study treatment after disease progression, if they meet the criteria outlined in the Measurement of Effect section.

### F. Duration of Follow Up

Safety Follow Up: Patients will be followed for 30 days after the last dose is administered or until the patient enrolls on another protocol or death, whichever comes first. The follow up will consist of a phone call between Days 27-30 after the last dose to evaluate adverse events that were ongoing and any new events that might be deemed related to the therapy. Toxicities felt to be possibly, probably, or definitely related to the study drugs that have not resolved or stabilized by Day 30 post-treatment will be followed until stabilization or resolution via biweekly phone calls.

Response Follow Up: Patients who reach the end of a 2-year treatment holiday will undergo a CT scan (or other imaging test; see “Methods for Evaluation of Measurable Disease” below). Those who remain progression-free at the end of the 2-year treatment holiday will be formally taken off treatment and will be ineligible to resume that treatment on this study. These patients will be followed for response evaluation every 6 months (±1 month) until disease progression, death, patient withdrawal, or enrollment in another protocol, whichever comes first. Follow up will consist of a scan every 6 months (±1 month).

Criteria for Removal from Treatment and/or Study: Patients will be removed from treatment when any of the criteria listed in above applies.

### VI. Dosing Delays/Dose Modifications

### C. General AE Management and Dose Modification Guidelines

Atezolizumab: There will be no dose reduction for atezolizumab in this study. Patients may temporarily suspend study treatment for up to 84 days (12 weeks) beyond the scheduled date of delayed infusion if study drug-related toxicity requiring dose suspension is experienced. If atezolizumab is held because of AEs for >84 days beyond the scheduled date of infusion, the patient will be discontinued from atezolizumab and will be followed for safety and efficacy as specified in this protocol. If the AE resolves within 84 days and the patient is receiving corticosteroid therapy for the event, atezolizumab may be held for longer than 84 days (up to 4 weeks) to allow tapering of the steroid dose to ≤10 mg oral prednisone or equivalent. Dose interruptions for reasons other than toxicity, such as surgical procedures, may be allowed. The acceptable length of interruption will be at the discretion of the study PI in consultation with CTEP.

The primary approach to grade 1 to 2 irAEs is supportive and symptomatic care with continued treatment with atezolizumab; for higher-grade irAEs, atezolizumab should be withheld and oral and/or parenteral steroids administered. Recurrent grade 2 irAEs may also mandate withholding atezolizumab or the use of steroids. Assessment of the benefit risk balance should be made by the investigator, with consideration of the totality of information as it pertains to the nature of the toxicity and the degree of clinical benefit a given patient may be experiencing prior to further administration of atezolizumab. Atezolizumab should be permanently discontinued in patients with life threatening irAEs.

Systemic Immune Activation: Systemic immune activation is a rare condition characterized by an excessive immune response. Given the mechanism of action of atezolizumab, systemic immune activation is considered a potential risk when given in combination with other immunomodulating agents. Systemic immune activation should be included in the differential diagnosis for patients who, in the absence of an alternative etiology, develop a sepsis-like syndrome after administration of atezolizumab, and the initial evaluation should include the following:
CBC with peripheral smear
PT, PTT, fibrinogen, and D-dimer
Ferritin
Triglycerides
AST, ALT, and total bilirubin
LDH
Complete neurologic and abdominal examination (assess for hepatosplenomegaly)

If systemic immune activation is still suspected after the initial evaluation, a physician can be contacted for additional recommendations.

Bevacizumab: For detailed information regarding management of adverse events associated with bevacizumab, please refer to the Management of Specific AEs section.

Bevacizumab must be discontinued, and may NOT be resumed, if the patient experiences any of the following events, regardless of benefit:
gastrointestinal perforation, tracheoesophageal fistula, or any Grade 4 fistula
fistula formation involving any internal organ
wound healing complications requiring medical intervention
necrotizing fasciitis
Grades 3-4 hemorrhage
arterial thromboembolic events (ATE)
Grade 4 venous thromboembolic events (VTE), including pulmonary embolism
hypertensive crisis or hypertensive encephalopathy
posterior reversible encephalopathy syndrome (PRES)
nephrotic syndrome
severe infusion-related reaction
congestive heart failure (CHF)
   If treatment with bevacizumab is discontinued, patients may continue to receive atezolizumab after discussion between the patient and PI.

### D. Management of Specific AEs

Atezolizumab: Management of certain AEs of concern, including immune-related pneumonitis, hepatitis, colitis, endocrinopathies, pancreatitis, neuropathies, meningoencephalitis, and potential ocular toxicities are presented in the Atezolizumab Investigator's Brochure. See Agent Administration for guidelines for the management of infusion-related reactions (IRRs) and anaphylaxis.

Atezolizumab has been associated with risks such as the following: IRRs and immune-related hepatitis, pneumonitis, colitis, pancreatitis, diabetes mellitus, hypothyroidism, hyperthyroidism, adrenal insufficiency, hypophysitis, Guillain-Barré syndrome, myasthenic syndrome or myasthenia gravis, facial paresis, myelitis, meningoencephalitis, myocarditis, pericardial disorders, nephritis, myositis, and severe cutaneous adverse reactions. In addition, immune-mediated reactions may involve any organ system and may lead to hemophagocytic lymphohistiocytosis (HLH). Neutropenia and lymphopenia associated with chemotherapy may increase the risk for developing an infection in patients receiving atezolizumab in combination with chemotherapy.

Pleural and pericardial effusion: Patients experiencing dyspnea, chest pain, or unexplained tachycardia should be evaluated for the presence of a pericardial effusion. Patients with pre-existing pericardial effusion should be followed closely for pericardial fluid volume measurements and impact on cardiac function. When intervention is required for pericardial or pleural effusions, atezolizumab should be held, and appropriate workup includes cytology, lactate dehydrogenase (LDH), glucose, cholesterol, protein concentrations (with pleural effusions), and cell count.

Pulmonary events: Dyspnea, cough, fatigue, hypoxia, pneumonitis, and pulmonary infiltrates have been associated with the administration of atezolizumab. Patients will be assessed for pulmonary signs and symptoms throughout the study and will have computed tomography (CT) scans of the chest performed at every tumor assessment.

All pulmonary events should be thoroughly evaluated for other commonly reported etiologies such as pneumonia or other infection, lymphangitic carcinomatosis, pulmonary embolism, heart failure, chronic obstructive pulmonary disease, or pulmonary hypertension. Management guidelines for pulmonary events are provided in Table 7.

Hepatic events: Immune-mediated hepatitis has been associated with the administration of atezolizumab. Eligible patients must have adequate liver function, as manifested by measurements of total bilirubin and hepatic transaminases, and liver function will be monitored throughout study treatment. Management guidelines for hepatic events are provided in Table 8.

Patients with right upper-quadrant abdominal pain and/or unexplained nausea or vomiting should have liver function tests (LFTs) performed immediately and reviewed before administration of the next dose of study drug.

For patients with elevated LFTs, concurrent medication, viral hepatitis, and toxic or neoplastic etiologies should be considered and addressed, as appropriate.

Gastrointestinal events: Immune-mediated colitis has been associated with the administration of atezolizumab. Management guidelines for diarrhea or colitis are provided in Table 9.

All events of diarrhea or colitis should be thoroughly evaluated for other more common etiologies. For events of significant duration or magnitude or associated with signs of systemic inflammation or acute-phase reactants (e.g., increased C-reactive protein, platelet count, or bandemia): Perform sigmoidoscopy (or colonoscopy, if appropriate) with colonic biopsy, with three to five specimens for standard paraffin block to check for inflammation and lymphocytic infiltrates to confirm colitis diagnosis.

Endocrine disorders: Thyroid disorders, adrenal insufficiency, diabetes mellitus, and pituitary disorders have been associated with the administration of atezolizumab. Management guidelines for endocrine events are provided in Table 10.

Patients experiencing one or more unexplained AEs possibly indicative of endocrine dysfunction (including headache, fatigue, myalgias, impotence, mental status changes, and constipation) should be investigated for the presence of thyroid, pituitary, or adrenal endocrinopathies. The patient should be referred to an endocrinologist if an endocrinopathy is suspected. Thyroid stimulating hormone (TSH) and free T3 and T4 levels should be measured to determine whether thyroid abnormalities are present. Pituitary hormone levels and function tests [e.g., TSH, growth hormone, luteinizing hormone, follicle-stimulating hormone, testosterone, prolactin, adrenocorticotropic hormone (ACTH) levels, and ACTH stimulation test] and MRI of the brain (with detailed pituitary sections) may help to differentiate primary pituitary insufficiency from primary adrenal insufficiency. Table 10 describes dose management guidelines for hyperthyroidism, hypothyroidism, symptomatic adrenal insufficiency, and hyperglycemia.

Ocular events: An ophthalmologist should evaluate visual complaints (e.g., uveitis, retinal events). Management guidelines for ocular events are provided in Table 11.

Immune-mediated Cardiac Events: Immune-mediated myocarditis and pericarditis have been associated with the administration of atezolizumab. Management guidelines for cardiac events are provided in Table 34.

Immune-mediated Myocarditis: Immune-mediated myocarditis has been associated with the administration of atezolizumab. Immune-mediated myocarditis should be suspected in any patient presenting with signs or symptoms suggestive of myocarditis, including, but not limited to, laboratory (e.g., B-NP [B-Natriuretic Peptide]) or cardiac imaging abnormalities, dyspnea, chest pain, palpitations, fatigue, decreased exercise tolerance, or syncope. Myocarditis may also be a clinical manifestation of myositis or associated with pericarditis (see section on pericardial disorders below) and should be managed accordingly. Immune-mediated myocarditis needs to be distinguished from myocarditis resulting from infection (commonly viral, e.g., in a patient who reports a recent history of gastrointestinal illness), ischemic events, underlying arrhythmias, exacerbation of preexisting cardiac conditions, or progression of malignancy.

All patients with possible myocarditis should be urgently evaluated by performing cardiac enzyme assessment, an electrocardiogram (ECG), a chest X-ray, an echocardiogram, and a cardiac MRI as appropriate per institutional guidelines. A cardiologist should be consulted. An endomyocardial biopsy may be considered to enable a definitive diagnosis and appropriate treatment, if clinically indicated.

Patients with signs and symptoms of myocarditis, in the absence of an identified alternate etiology, should be treated according to the guidelines in Table 34.

Immune-mediated Pericardial Disorders: Immune-mediated pericarditis should be suspected in any patient presenting with chest pain and may be associated with immune-mediated myocarditis (see section on myocarditis above)._Immune-mediated pericardial effusion and cardiac tamponade should be suspected in any patient presenting with chest pain associated with dyspnea or hemodynamic instability. Patients should be evaluated for other causes of pericardial disorders such as infection (commonly viral), cancer related (metastatic disease or chest radiotherapy), cardiac injury related (post myocardial infarction or iatrogenic), and autoimmune disorders, and should be managed accordingly. All patients with suspected pericardial disorders should be urgently evaluated by performing an ECG, chest X-ray, transthoracic echocardiogram, and cardiac MRI as appropriate per institutional guidelines. A cardiologist should be consulted. Pericardiocentesis should be considered for diagnostic or therapeutic purposes, if clinically indicated. Patients with signs and symptoms of pericarditis, pericardial effusion, or cardiac tamponade, in the absence of an identified alternate etiology, should be treated according to the guidelines in the table below. Withhold treatment with atezolizumab for Grade 1 pericarditis and conduct a detailed cardiac evaluation to determine the etiology and manage accordingly.

TABLE 34
Management of Immune-mediated Myocarditis
and Pericardial Disorder Events

| Event | Management |
|---|---|
| Immune- | Permanently discontinue atezolizumab. |
| mediated | Refer patient to cardiologist. |
| myocarditis, | Initiate treatment as per institutional guidelines |
| Grade 2-4 | and consider antiarrhythmic drugs, temporary pacemaker, |
| Immune- | ECMO, VAD, or pericardiocentesis as appropriate. |
| mediated | Initiate treatment with corticosteroids equivalent to |
| pericardial | 1-2 mg/kg/day IV methylprednisolone and convert to 1-2 |
| disorders, | mg/kg/day oral prednisone or equivalent upon |
| Grade 2-4 | improvement. |
| | If event does not improve within 48 hours after |
| | initiating corticosteroids, consider adding an |
| | immunosuppressive agent. |
| | If symptoms resolve to below Grade 2, taper |
| | corticosteroids over ≥1 month. |

ECMO = extracorporeal membrane oxygenation; VAD = ventricular assist device.

Infusion-Related Reactions and Cytokine-Release Syndrome: No premedication is indicated for the administration of Cycle 1 of atezolizumab. However, patients who experience an infusion-related reaction (IRR) or cytokine-release syndrome (CRS) with atezolizumab may receive premedication with antihistamines, antipyretics, and/or analgesics (e.g., acetaminophen) for subsequent infusions. Metamizole (dipyrone) is prohibited in treating atezolizumab-associated IRRs because of its potential for causing agranulocytosis.

IRRs are known to occur with the administration of monoclonal antibodies and have been reported with atezolizumab. These reactions, which are thought to be due to release of cytokines and/or other chemical mediators, occur within 24 hours of atezolizumab administration and are generally mild to moderate in severity.

CRS is defined as a supraphysiologic response following administration of any immune therapy that results in activation or engagement of endogenous or infused T cells and/or other immune effector cells. Symptoms can be progressive, always include fever at the onset, and may include hypotension, capillary leak (hypoxia), and end-organ dysfunction (Lee et al., 2019). CRS has been well documented with chimeric antigen receptor T-cell therapies and bispecific T-cell engager antibody therapies but has also been reported with immunotherapies that target PD-1 or PD-L1 (Rotz et al., 2017; Adashek and Feldman 2019) including atezolizumab.

There may be significant overlap in signs and symptoms of IRRs and CRS, and in recognition of the challenges in clinically distinguishing between the two, consolidated guidelines for medical management of IRRs and CRS are provided in Table 13.

Pancreatic events: Symptoms of abdominal pain associated with elevations of amylase and lipase, suggestive of pancreatitis, have been associated with the administration of atezolizumab. The differential diagnosis of acute abdominal pain should include pancreatitis. Appropriate workup should include an evaluation for ductal obstruction, as well as serum amylase and lipase tests. Management guidelines for pancreatic events, including pancreatitis, are provided in Table 14.

Dermatologic events: Treatment-emergent rash has been associated with atezolizumab. The majority of cases of rash were mild in severity and self-limited, with or without pruritus. A dermatologist should evaluate persistent and/or severe rash or pruritus. Although uncommon, cases of severe cutaneous adverse reactions such as Stevens-Johnson syndrome and toxic epidermal necrolysis have been reported with atezolizumab. A biopsy should be considered unless contraindicated. Management guidelines for dermatologic events are provided in Table 15.

Neurologic disorders: Myasthenia gravis and Guillain-Barré syndrome have been observed with single agent atezolizumab. Patients may present with signs and symptoms of sensory and/or motor neuropathy. Diagnostic work-up is essential for an accurate characterization to differentiate between alternative etiologies. Management guidelines for neurologic disorders, and specific guidelines for myelitis, are provided in Tables 35 and 36.

TABLE 35
Management Guidelines for Neurologic Disorders

| Event | Management |
|---|---|
| Immune-related | Continue atezolizumab. |
| neuropathy, Grade 1 | Investigate etiology. |
| | Any cranial nerve disorder (including facial paresis) should be managed as |
| | per Grade 2 management guidelines below. |
| Immune-related | Withhold atezolizumab for up to 12 weeks after event onset. ^{a} |
| neuropathy, | Investigate etiology. |
| including facial | Initiate treatment as per institutional guidelines. |
| paresis, Grade 2 | For general immune-mediated neuropathy: |
| | If event resolves to Grade 1 or better, resume atezolizumab.^{b} |
| | If event does not resolve to below Grade 1 or better while withholding |
| | atezolizumab, permanently discontinue atezolizumab.^{c} |
| | For facial paresis: |
| | If event resolves fully, resume atezolizumab.^{b} |
| | If event does not resolve fully while withholding atezolizumab, |
| | permanently discontinue atezolizumab.^{c} |
| Immune-related | Permanently discontinue atezolizumab.^{c} |
| neuropathy, | |
| including facial | |
| paresis, Grade 3 or 4 | Initiate treatment as per institutional guidelines. |
| Myasthenia gravis | Permanently discontinue atezolizumab.^{c} |
| and Guillain-Barré | Refer patient to neurologist. |
| syndrome (any | Initiate treatment as per institutional guidelines. |
| grade) | Consider initiation of corticosteroids equivalent to 1-2 mg/kg/day oral or |
| | IV prednisone. |

^{a} Atezolizumab may be withheld for a longer period of time (i.e., >12 weeks after event onset) to allow for corticosteroids (if initiated) to be reduced to the equivalent of ≤10 mg/day oral prednisone. The acceptable length of the extended period of time can be based on an assessment of benefit-risk by the investigator and in alignment with the protocol requirements for the duration of treatment and documented by the investigator.
^{b}If corticosteroids have been initiated, they must be tapered over ≥1 month to the equivalent of ≤10 mg/day oral prednisone before atezolizumab can be resumed.
^{c}Resumption of atezolizumab may be considered in patients who are deriving benefit and have fully recovered from the immune-mediated event. The decision to re-challenge patients with atezolizumab should be based on investigator’s assessment of benefit-risk and documented by the investigator (or an appropriate delegate).

TABLE 36
Management Guidelines for Immune-Mediated Myelitis Events

| Event | Management |
|---|---|
| Immune-mediated | Continue atezolizumab unless symptoms worsen or do |
| myelitis, | not improve. |
| Grade 1 | Investigate etiology and refer patient to a neurologist. |
| Immune-mediated | Permanently discontinue atezolizumab. |
| myelitis, | Investigate etiology and refer patient to a neurologist. |
| Grade 2 | Rule out infection. |
| | Initiate treatment with corticosteroids equivalent to |
| | 1-2 mg/kg/day oral prednisone. |
| Immune-mediated | Permanently discontinue atezolizumab. |
| myelitis, | Refer patient to a neurologist. |
| Grade 3 or 4 | Initiate treatment as per institutional guidelines. |

Immune-Mediated Meningoencephalitis: Immune-mediated meningoencephalitis is an identified risk associated with the administration of atezolizumab. Immune-mediated meningoencephalitis should be suspected in any patient presenting with signs or symptoms suggestive of meningitis or encephalitis, including, but not limited to, headache, neck pain, confusion, seizure, motor or sensory dysfunction, and altered or depressed level of consciousness. Encephalopathy from metabolic or electrolyte imbalances needs to be distinguished from potential meningoencephalitis resulting from infection (bacterial, viral, or fungal) or progression of malignancy, or secondary to a paraneoplastic process.

All patients being considered for meningoencephalitis should be urgently evaluated with a CT scan and/or MRI scan of the brain to evaluate for metastasis, inflammation, or edema. If deemed safe by the treating physician, a lumbar puncture should be performed, and a neurologist should be consulted.

Patients with signs and symptoms of meningoencephalitis, in the absence of an identified alternate etiology, should be treated according to the guidelines in Table 17.

Renal events: Immune-mediated nephritis has been associated with the administration of atezolizumab. Eligible patients must have adequate renal function. Renal function, including serum creatinine, should be monitored throughout study treatment. Patients with abnormal renal function should be evaluated and treated for other more common etiologies (including prerenal and postrenal causes, and concomitant medications such as non-steroidal anti-inflammatory drugs). Refer the patient to a renal specialist if clinically indicated. A renal biopsy may be required to enable a definitive diagnosis and appropriate treatment.

Patients with signs and symptoms of nephritis, in the absence of an identified alternate etiology, should be treated according to the guidelines in Table 18.

Immune-Mediated Myositis: Immune-mediated myositis has been associated with the administration of atezolizumab. Myositis or inflammatory myopathies are a group of disorders sharing the common feature of inflammatory muscle injury; dermatomyositis and polymyositis are among the most common disorders. Initial diagnosis is based on clinical (muscle weakness, muscle pain, skin rash in dermatomyositis), biochemical (serum creatine kinase increase), and imaging (electromyography/MRI) features, and is confirmed with a muscle biopsy.

Patients with signs and symptoms of myositis, in the absence of an identified alternate etiology, should be treated according to the guidelines in Table 37.

TABLE 37
Management Guidelines for Immune-Mediated Myositis

| Event | Management |
|---|---|
| Immune-mediated | Continue atezolizumab. |
| myositis, Grade 1 | Refer patient to rheumatologist or neurologist. |
| | Initiate treatment as per institutional guidelines. |
| Immune-mediated | Withhold atezolizumab for up to 12 weeks after event onset ^{a} and contact |
| myositis, Grade 2 | Medical Monitor. |
| | Refer patient to rheumatologist or neurologist. |
| | Initiate treatment as per institutional guidelines. |
| | Consider treatment with corticosteroids equivalent to 1-2 mg/kg/day IV |
| | methylprednisolone and convert to 1-2 mg/kg/day oral prednisone or |
| | equivalent upon improvement. |
| | If corticosteroids are initiated and event does not improve within 48 hours |
| | after initiating corticosteroids, consider adding an immunosuppressive |
| | agent. |
| | If event resolves to Grade 1 or better, resume atezolizumab. ^{b} |
| | If event does not resolve to Grade 1 or better while withholding |
| | atezolizumab, permanently discontinue atezolizumab and contact Medical |
| | Monitor. |
| Immune-mediated | Withhold atezolizumab for up to 12 weeks after event onset ^{a} and contact |
| myositis, Grade 3 | Medical Monitor. |
| | Refer patient to rheumatologist or neurologist. |
| | Initiate treatment as per institutional guidelines. |
| | Respiratory support may be required in more severe cases. |
| | Initiate treatment with corticosteroids equivalent to 1-2 mg/kg/day IV |
| | methylprednisolone, or higher-dose bolus if patient is severely |
| | compromised (e.g., cardiac or respiratory symptoms, dysphagia, or |
| | weakness that severely limits mobility); convert to 1-2 mg/kg/day oral |
| | prednisone or equivalent upon improvement. |
| | If event does not improve within 48 hours after initiating corticosteroids, |
| | consider adding an immunosuppressive agent. |
| | If event resolves to Grade 1 or better, resume atezolizumab. ^{b} |
| | If event does not resolve to Grade 1 or better while withholding |
| | atezolizumab, permanently discontinue atezolizumab and contact Medical |
| | Monitor. |
| | For recurrent events, treat as a Grade 4 event. |
| Immune-mediated | Permanently discontinue atezolizumab and contact Medical Monitor. |
| myositis, Grade 4 | Refer patient to rheumatologist or neurologist. |
| | Initiate treatment as per institutional guidelines. |
| | Respiratory support may be required in more severe cases. |
| | Initiate treatment with corticosteroids equivalent to 1-2 mg/kg/day IV |
| | methylprednisolone, or higher-dose bolus if patient is severely |
| | compromised (e.g., cardiac or respiratory symptoms, dysphagia, or |
| | weakness that severely limits mobility); convert to 1-2 mg/kg/day oral |
| | prednisone or equivalent upon improvement. |
| | If event does not improve within 48 hours after initiating corticosteroids, |
| | consider adding an immunosuppressive agent. |
| | If event resolves to Grade 1 or better, taper corticosteroids over ≥1 month. |

^{a} Atezolizumab may be withheld for a longer period of time (i.e., >12 weeks after event onset) to allow for corticosteroids (if initiated) to be reduced to the equivalent of ≤10 mg/day oral prednisone. The acceptable length of the extended period of time must be agreed upon by the investigator and the Medical Monitor.
^{b} If corticosteroids have been initiated, they must be tapered over ≥1 month to the equivalent of ≤10 mg/day oral prednisone before atezolizumab can be resumed.

Hemophagocytic Lymphohistiocytosis and Macrophage Activation Syndrome: Immune-mediated reactions may involve any organ system and may lead to hemophagocytic lymphohistiocytosis (HLH) and macrophage activation syndrome (MAS).

Patients with suspected HLH should be diagnosed according to published criteria by McClain and Eckstein (2017). A patient should be classified as having HLH if five of the following eight criteria are met:
Fever ≥38.5° C.
Splenomegaly
Peripheral blood cytopenia consisting of at least two of the following:
Hemoglobin <90 g/L (9 g/dL) (<100 g/L [10 g/dL] for infants <4 weeks old)
Platelet count <100×10⁹/L (100,000/mcL)
ANC <1.0×10⁹/L (1000/mcL)
Fasting triglycerides >2.992 mmol/L (265 mg/dL) and/or fibrinogen <1.5 g/L (150 mg/dL)
Hemophagocytosis in bone marrow, spleen, lymph node, or liver
Low or absent natural killer cell activity
Ferritin >500 mg/L (500 ng/mL)
Soluble interleukin 2 (IL-2) receptor (soluble CD25) elevated >2 standard deviations above age-adjusted laboratory-specific norms
   Patients with suspected MAS should be diagnosed according to published criteria for systemic juvenile idiopathic arthritis by Ravelli et al. (2016). A febrile patient should be classified as having MAS if the following criteria are met:
Ferritin >684 mg/L (684 ng/mL)
At least two of the following:
Platelet count ≤181×10⁹/L (181,000/mcL)
AST ≥48 U/L
Triglycerides >1.761 mmol/L (156 mg/dL)
Fibrinogen ≤3.6 g/L (360 mg/dL)
   Patients with suspected HLH or MAS should be treated according to the guidelines described in Table 20.

### Bevacizumab

Surgery and wound healing complications: Discontinue bevacizumab in patients with wound healing complications requiring medical intervention. Withhold for at least 28 days prior to elective surgery. Do not administer for at least 28 days following surgery and until the wound is fully healed. Necrotizing fasciitis including fatal cases, has been reported in patients receiving bevacizumab, usually secondary to wound healing complications, gastrointestinal perforation or fistula formation. Discontinue bevacizumab in patients who develop necrotizing fasciitis.

Hypertension: Monitor blood pressure every two to three weeks during treatment with bevacizumab. Repeat blood pressure measurements, treat with appropriate anti-hypertensive therapy, and continue, reduce, or discontinue protocol therapy according to the guidelines specified in FIG. 19. Discontinue bevacizumab in patients who develop hypertensive crisis or hypertensive encephalopathy. Continue to monitor blood pressure at regular intervals in patients with bevacizumab-induced or -exacerbated hypertension after discontinuing bevacizumab.

Renal injury and proteinuria: At the start of each cycle, monitor proteinuria by urinalysis for the development or worsening of proteinuria with serial urinalyses during bevacizumab therapy. Patients with 2+ proteinuria on urinalysis should undergo further assessment with a 24-hour urine collection. Withhold for proteinuria greater than or equal to 2 grams per 24 hours and resume when less than 2 grams per 24 hours. Discontinue in patients who develop nephrotic syndrome.

Data from a postmarketing safety study showed poor correlation between UPCR (Urine Protein/Creatinine Ratio) and 24-hour urine protein [Pearson Correlation 0.39 (95% CI: 0.17, 0.57)].

Infusion-related reactions: Decrease the rate of infusion for mild, clinically insignificant infusion-related reactions. For subsequent infusions, see the premedication and infusion time guidance in the Agent Administration Section. Interrupt the infusion in patients with clinically significant infusion-related reactions and consider resuming at a slower rate following resolution. Discontinue in patients who develop a severe infusion-related reaction and administer appropriate medical therapy (e.g., epinephrine, corticosteroids, intravenous antihistamines, bronchodilators and/or oxygen). Management guidelines are provided in Table 21.

### VI. Pharmaceutical Information

### A. Atezolizumab (NSC #783608); Other Names: Tecentriq™, MPDL3280A; Classification: Monoclonal Antibody; M.W.: 150 KD; Mode of Action: Anti-PD-L1

Description: Atezolizumab is a humanized IgG1 monoclonal antibody consisting of two heavy chains (448 amino acids) and two light chains (214 amino acids). Atezolizumab targets human PD-L1 and inhibits its interaction with its receptor PD-1. Atezolizumab also blocks the binding of PD-L1 to B7.1, an interaction that is reported to provide additional inhibitory signals to T cells (Butte et al. 2007).

How Supplied: Atezolizumab is provided by Genentech/F.Hoffmann-La Roche LTD and distributed by the Pharmaceutical Management Branch, CTEP, NCI. The agent is supplied in a single-use, 20-mL glass vial as a colorless-to-slightly-yellow, sterile, preservative-free clear liquid solution intended for IV administration. Each 20 mL vial contains 1200 mg of atezolizumab and is formulated in glacial acetic acid (16.5 mg), L-histidine (62 mg), polysorbate 20 (8 mg), and sucrose (821.6 mg), with a pH of 5.8. The vial is designed to deliver 20 mL (1200 mg) of atezolizumab solution but may contain more than the stated volume to enable delivery of the entire 20 mL volume.

Preparation: The prescribed dose of atezolizumab should be diluted in 0.9% NaCl to a concentration between 3.2 mg/mL and 16.8 mg/mL and infused with or without a low-protein binding 0.2 or 0.22 micrometer in-line filter. The IV bag may be constructed of polyvinyl chloride (PVC), polyolefin (PO), or polyethylene (PE). The prepared solution may be stored at 2° C.-8° C. for up to 24 hours or at ambient <25° C. (77° F.) for 6 hours from the time of preparation. If the dose solution is stored at 2□C-8□C (36 □ F-46° F.), it should be removed from refrigeration and allowed to reach room temperature prior to administration. These times include the storage and administration times for the infusion. Do not shake or freeze infusion bags containing the dose solution.

Storage: 2° C.-8° C. (36° F.-46° F.) Vial contents should not be frozen or shaken and should be protected from direct sunlight.

If a storage temperature excursion is identified, promptly return atezolizumab to 2° C.-8° C. (36° F.-46° F.) and quarantine the supplies. Provide a detailed report of the excursion (including documentation of temperature monitoring and duration of the excursion) to PMBAfterHours@mail.nih.gov for determination of suitability.

Stability: Stability studies are ongoing.

CAUTION: No preservative is used in atezolizumab; therefore, the vial is intended for single use only.

Discard any unused portion of drug remaining in a vial.

Route of Administration: IV infusion

Method of Administration: Atezolizumab is administered as an intravenous infusion over 60 minutes. If the first infusion is tolerated, all subsequent infusions may be delivered over 30 minutes. Do not administer atezolizumab as an intravenous push or bolus. No premedication is indicated for administration of Cycle 1 of atezolizumab. Patients who experience an infusion related reaction with Cycle 1 of atezolizumab may receive premedications with subsequent infusions.

Potential Drug Interactions: Cytochrome P450 enzymes as well as conjugation/glucuronidation reactions are not involved in the metabolism of atezolizumab. No drug interaction studies for atezolizumab have been conducted or are planned. There are no known interactions with other medicinal products or other form of interactions.

Patient Care Implications: Female patients of childbearing potential should utilize contraception and take active measures to avoid pregnancy while undergoing atezolizumab treatment and for at least 150 days after the last dose of atezolizumab.

B. Bevacizumab (NSC #704865); Other Names: rhuMAb VEGF, Avastin®; Classification: Recombinant Humanized Monoclonal Antibody; Molecular Weight:Approximate Molecular Weight is 149,000 Daltons

Mode of Action: Bevacizumab blocks the binding of vascular endothelial growth factor (VEGF) to its receptors resulting in inhibition of angiogenesis.

Description: Bevacizumab is a recombinant humanized anti-VEGF monoclonal antibody consisting of 93% human and 7% murine amino acid sequences. The agent is composed of human IgG framework and murine antigen-binding complementarity-determining regions

How Supplied: Bevacizumab is supplied as a clear to slightly opalescent, sterile liquid for parenteral administration. Each 400 mg (25 mg/ml-16 mL fill) glass vial contains bevacizumab with phosphate, trehalose, polysorbate 20, and Sterile Water for Injection, USP.

Preparation: Vials contain no preservatives and are intended for single use only. The calculated dose should be diluted in 0.9% sodium chloride for injection to a final concentration of bevacizumab between 1.4 and 16.5 mg/ml.

Storage: Upon receipt, refrigerate bevacizumab (2° to 8° C.). Keep vial in the outer carton due to light sensitivity. Do not freeze. Do not shake.

If a storage temperature excursion is identified, promptly return bevacizumab to 2° C.-8° C. (36° F.-46° F.) and quarantine the supplies. Provide a detailed report of the excursion (including documentation of temperature monitoring and duration of the excursion) to PMBAfterHours@mail.nih.gov for determination of suitability.

Stability: Please see the product label for expiration information. The sterile single use vials contain no antibacterial preservatives. Discard vials 8 hours after initial entry.

Once diluted in 0.9% sodium chloride, the product should be used immediately. If not used immediately, in-use storage times are the responsibility of the user and should not be longer than 24 hours at 2° C.-8° C.

Route of Administration: Intravenous

Method of Administration: Administer the initial dose over a minimum of 90 minutes. If no adverse reactions occur, administer the second dose over a minimum of 60 minutes. If no adverse reactions occur after the second dose, administer subsequent doses over a minimum of 30 minutes. If infusion-related adverse reactions occur, all subsequent infusions should be administered over the shortest period that was well tolerated.

Patient Care Implications: Male patients and female patients of childbearing potential should utilize contraception and take active measures to avoid pregnancy prior to study entry, while undergoing bevacizumab treatment, and for at least 6 months (180 days) after the last dose of bevacizumab.

### VII. Correlative Studies

The plan is to evaluate the effect of the study drugs on immune cell components of the tumor and microenvironment. The correlative studies that will be carried out on the paired (pre- and post-treatment) biopsies will help elucidate the mechanism of action of atezolizumab monotherapy and the atezolizumab plus bevacizumab combination on signaling pathways mediating the immune response, focusing on subsets of effector cells. Additionally, the samples will be used to evaluate the tumor microenvironment to see if any changes or lack thereof are associated with either response or resistance to therapy. For patients who cross over directly from the monotherapy arm to the combination therapy arm, the baseline biopsy collected for the combination arm will provide valuable insight into the biochemical signaling and genetic composition of their tumor at the time of disease progression on atezolizumab monotherapy. If additional tissue is available from any patient and its collection poses no new risks to the patient, the tissue may be collected by NCI DCTD SOPs for analysis by any of the correlative study assays described in the correlative studies section, provided appropriate consent is obtained.

An optional tumor biopsy may be collected from adult patients (≥18 years of age), on either treatment arm, at the time of disease progression (or pre-progression, following any restaging at which a 10-19% increase in tumor volume is observed) for analysis by any of the correlative study assays described in the “Correlative Studies” section, provided appropriate consent is obtained. At the PI's discretion, a progression biopsy collected while a patient is on the monotherapy arm may be utilized as the baseline biopsy for the combination arm, provided the patient does not receive any anti-cancer agents or immunotherapy in the interim.

Blood samples will also be collected for the purification and analysis of circulating tumor cells (CTCs). One additional optional blood sample will be collected at baseline (or any other time on study) from consenting patients ≥18 years of age and the nucleic acid from the mononuclear cells will be sequenced to serve as a germline control for calling somatic alterations in WES and RNA-Seq results from the patient's tumor specimen, or to enable the assessment of somatic alterations in the blood that may arise from clonal hematopoiesis of indeterminant potential.

For patients who consent to genetic sequencing of blood samples, plasma from CTC blood samples correlating to clinically informative time points (minimally, before treatment and at restaging scans suggesting changes in tumor burden) will be analyzed for circulating tumor DNA (ctDNA) by the TSO500 panel in order to identify potential mechanisms of response and resistance in cell free DNA (cfDNA) and to identify TFE3 fusions in plasma. If a sufficient proportion of the circulating cfDNA detected is ctDNA, then WES of cfDNA will also be considered to uncover additional relevant changes in the tumor genome. The results of these genomic analyses of blood samples will not be returned to patients.

Peripheral blood mononuclear cells (PBMCs) can be collected from patients ≥14 years of age who consent to the PBMC collection and genetic sequencing component.

Blood (mandatory) will be collected from pediatric patients <12 years of age at multiple time points for measurement of PK and ADA, as described in Table 38.

TABLE 38
Tissue Specimen Prioritization Table

| | Baseline and C3D1 Biopsies |
|---|---|
| Collection Information | All cores flash frozen |
| First Priority Assay(s) | First 2 cores |
| | Immuno-PD, Apoptosis IFA, IHC |
| | (PD-1, PD-L1) |
| Second Priority Assay(s) | 3^{rd} core (and 4^{th} if needed) |
| | WES*, RNA-Seq*, spatial profiling of RNA |
| | and protein expression, targeted sequencing* |
| | (e.g., Archer panel) |
| Third Priority Assay(s) - | 5^{th} (or other remaining) core |
| hold core until | Luminex multiplex immunoassay for apoptosis/ |
| sequencing results | cell death |
| obtained | |
| Reporting to Patients | None |

*Provided informed consent for genetic sequencing is obtained from the patient.

### A. Pharmacodynamic Assays in Tumor Tissue

Tumor biopsies (mandatory at baseline and cycle 3 day 1 for patients in whom biopsies are deemed safe and feasible, and optional at time of disease progression) will be collected for research from adult patients (≥18 years of age). Pharmacodynamic assays will be performed on these tumor specimens to assess the number of activated CD8+ T cells infiltrating the tumor before and after atezolizumab treatment. Efforts will be made to correlate any observed treatment-induced changes with clinical response.

Activated CD8+ T cells will be defined by the expression of phosphorylated Zap70 (pY493) and SHP2 (pY580); CD8+ cells present within the tumor section that are positive for these markers will be quantified. Other targets of PD assays may include PD-L1 and PD-1 levels in cells present in the biopsy, TCR activation (Zeta chain phosphorylation), T-cell Ki67 expression, the presence of T-reg cells (FOXP3+) and MDSCs, and other potential markers of response or resistance.

These biomarkers will be measured through multiplex immunofluorescence assays designed and validated by PADIS. Measurements on the Nikon Ai/Definiens platform will include cell enumeration and proximity of tumor cells (by pathology analysis and ASPL-TFE3 fusion-protein IFA) to T-cells. Monoclonal antibodies to all markers have been validated by the standard PADIS approach, and PADIS has previously validated and reported on the tumor cell marker set.

Mandatory biopsies will be collected from adult patients on both treatment arms at: Baseline (prior to the first dose of study agent(s) on that arm) Prior to cycle 3 day 1 (±3 days) or at any point where there is clinical evidence that the patient is responding to drug

An optional tumor biopsy may be collected from adult patients (≥18 years of age), enrolled to either arm, at the time of disease progression (or pre-progression, following any restaging at which a 10-19% increase in tumor volume is observed).

For adult patients who progress on atezolizumab monotherapy and elect to cross over to the combination arm, the baseline biopsy for the combination arm should be collected after coming off monotherapy treatment due to progressive disease (see Measurement of Effect Section) but before initiating treatment on the combination arm. However, if the patient progressed on atezolizumab monotherapy after <4 cycles and had a biopsy collected on cycle 3 day 1 (±3 days) of atezolizumab monotherapy treatment, that cycle 3 day 1 biopsy will be used as their baseline biopsy for the combination treatment arm; a biopsy will not be collected between disease progression and initiation of the combination treatment. If a patient on the monotherapy arm elects to undergo an optional progression biopsy, that biopsy may, at the PI's discretion, serve as the baseline biopsy for the combination arm, provided the patient does not receive any anti-cancer agents or immunotherapy in the interim.

The post-treatment biopsy on the atezolizumab+bevacizumab arm will be collected prior to cycle 3 day 1 (±3 days) of the combination treatment (or at any point after initiating bevacizumab where there is clinical evidence that the patient is responding to drug).

### B. Genetic Assays in Tumor Tissue

Exploratory genetic sequencing will be performed on biopsy tissue from patients who consent to this optional component, with the goals of 1) classifying tumors by ASPL-TFE3 fusion type (Type 1 or Type 2), 2) characterizing genomic features associated with each fusion type, and 3) identifying potential markers of atezolizumab response and resistance. Tumor specimens that remain after the completion of the pharmacodynamic analysis described in the Correlative Studies section will be analyzed by whole exome and whole RNA transcriptome sequencing. Germline DNA obtained from a whole blood sample will be used as a point of comparison to identify somatic mutations. Targeted sequencing may also be performed (e.g., Archer panel to identify ASPL-TFE3 fusion types if they cannot be called from RNA-Seq data), as well as spatial profiling of RNA and protein expression. Single cell RNA sequencing may also be performed if appropriately preserved tissue is available.

### C. Biopsy Collection and Shipment

Serial tumor biopsies will be obtained by the Interventional Radiology team by a percutaneous approach, a dermatologist for skin lesions, or an ENT for lesions that are easily biopsiable through ENT exam. If a site is deemed appropriate for biopsy with minimal risk to the participant by agreement between the investigators and the biopsy team, an attempt for biopsy will be made. Because approximately 20% of tumor biopsies collected on research trials are not usable due to the presence of stroma or normal and/or necrotic tissue and paired biopsies are necessary for analysis, up to 5 core biopsies ≥18-gauge in diameter and >1 cm in length, or the equivalent, will be obtained during each procedure to try and ensure adequate tumor content and quality. If possible, the lesion from which each biopsy is taken will be documented. Acceptable biopsy procedures include percutaneous biopsy with local anesthetic, excisional cutaneous biopsy with local anesthetic, and other biopsy with local anesthetic and/or sedation that has been shown to have a risk of severe complications <2%.

The use of imaging to facilitate biopsies will be decided by members of the biopsy team and may include ultrasound, CT scan, or MRI. Should a CT scan be needed for biopsy, the number of scans for each procedure will be limited to the minimum number needed to safely obtain a biopsy. Tumor biopsies and local anesthesia will be administered only if they are considered to be of low risk to the participant, as determined by the investigators and the biopsy team. The clinical, radiologic, dermatologic, ENT, and pharmacodynamic members of the research team will meet monthly to review the adequacy of the biopsy specimens for analysis.

Tumor biopsies are mandatory at all participating sites. Baseline biopsies will be performed following patient enrolling on study, prior to initiating study treatment (on the monotherapy arm and the combination arm). If an initial attempt at biopsy is unsuccessful, the patient will be given an option to proceed with a repeated attempt. A separate consent form must be signed for each biopsy procedure, so patients may choose not to undergo subsequent biopsies. If the baseline biopsy is unsuccessful or the patient refuses to undergo subsequent biopsies, no further biopsies will be performed but the patient will remain on study, receive study medication, and other correlative studies will be performed.

Briefly, the biopsy cores or, equivalent tissue, will be transferred into 1.5-mL pre-chilled cryovials and then flash frozen in liquid nitrogen. Biopsies should be collected, placed in pre-chilled cryogenic vials, and frozen within 2 minutes of collection. The frozen biopsy specimens are transferred to PADIS in pairs on dry ice, where they are stored at −80° C. or colder, and subsequently processed within 7-10 days for analysis or as directed by a physician. Biopsy samples will be analyzed as described above; any additional samples will be kept for future analysis in liquid nitrogen freezers. Additional studies, if performed, will be conducted following an amendment to the current protocol.

Baseline and post-treatment biopsy samples should be held and shipped to PADIS together upon completion of the second biopsy. If a patient crosses over from the monotherapy arm to the combination arm, the pre-treatment biopsy for the combination arm (collected after disease progression on monotherapy) should be held and shipped with the post-combination treatment biopsy.

Labeling: Biopsy specimens should be labeled with:
Sample type (e.g., Biopsy)
Time point (e.g., C01D01 pre dose)
Collection date and time
Sample ID, containing CTEP protocol number, Unique patient ID, 500-series sample collection number (e.g., 500, 501, 502, etc.), and Pass/Core identifier (e.g., A, B, C, etc.)

The sample shipping manifest and batch record, both of which are included in NCI DCTD SOP340507, should be completed and included with the shipment. NCI DCTD SOP340507 for biopsy collection, processing, and shipment.

### D. Blood Collection and Shipment

In some aspects, no more than 5 mL/kg is drawn for research purposes in a single day, and no more than 9.5 mL/kg may be drawn over any eight-week period.

Circulating Tumor Cells (CTCs): Collection of blood samples for PD studies in CTCs will be mandatory. Blood specimens will be obtained from patients ≥14 years of age. CTCs will be isolated from specimens associated with clinically significant time points and analyzed for various biomarkers, such as ASPL-TFE3 fusion protein, beta-Catenin, cytokeratin, and vimentin, as well as PD-L1 and apoptosis markers γH2AX and activated caspase 3 and 8. Plasma obtained during the isolation of CTCs at the PADIS laboratory will be retained for genetic analysis of cell-free DNA (see “Blood Collection and Shipment” section). This does not involve any changes to CTC collection or processing procedures at clinical sites. CTC specimens may also be used for identification of ASPL-TFE3 fusions via RT-PCR.

Blood for CTC analysis can be shipped at room temperature to the PADIS laboratory on the day it is collected.

Collection: Blood samples (7.5 mL) can be collected at the following times on both treatment arms:
at baseline (on cycle 1 day 1 prior to drug administration)
on cycle 2 day 1
on the first day of every subsequent cycle prior to drug administration
at disease progression
at the time of each response assessment during a treatment holiday
at the time of each response assessment during the response follow-up period after completion of a treatment holiday
   In some aspects, while CTC collection is required at these timepoints, the requirement is waived if a collection time point falls on a Friday or too late on a Thursday to ensure same-day FedEx pickup, due to shipment and processing requirements.

CTC collections can be repeated if a patient crosses over or re-enrolls to the combination arm.

In some aspects, blood is collected aseptically by venipuncture or from a venous port into one 10 mL Streck Cell-Free DNA BCT blood collection tube (Streck, Cat #218962). The collected blood samples are stable for up to 48 hours at room temperature (15° C. to 30° C.) prior to processing.

In some aspects, each blood specimen can be collected into one 10 mL Streck tube (catalog number 218962). Tubes can be inverted 8 times to ensure adequate mixing of the additive.

### Labeling:

Blood Specimens should be Labeled with:
Sample type (e.g., Whole blood)
Time point (e.g., C1D1 pre dose)
Collection date and time
Sample ID, containing CTEP protocol number, Unique patient ID, 400-series sample collection number (e.g., 400, 401, 402, etc.)
   All shipments should include a description of contents on the outside label/shipping slip, and a detailed packing slip should be included with the samples.

### Blood for Germline Sequencing

Collection: Beginning with the 01/07/21 protocol amendment, one whole blood sample (at least 7.5 mL in a 10-mL Streck tube) will be collected at baseline (or at any point while the patient is on study) from patients who consent to genomic analyses, and mononuclear cells will be isolated from whole blood by the MoCha Laboratory for nucleic acid extraction and exploratory WES to allow for germline and somatic variants to be accurately identified, and to enable the accurate assessment of mutational signatures in tumor specimens. The results of these studies will not be returned to patients.

### Labeling:

Blood Specimens should be Labeled with:
Sample type (e.g., Whole blood)
Time point (e.g., C1D1 pre dose)
Collection date and time
Sample ID, containing CTEP protocol number, Unique patient ID (Do NOT include patient name, medical record number, or initials), and 800-series sample collection number (i.e., 899)

### Cryopreserved Peripheral Blood Mononuclear Cells (PBMCs)

In some aspects, PBMCs will be isolated and cryopreserved for genomic analysis of T cell receptor repertoire.

PBMCs will be collected from patients ≥14 years of age who consent to this component. Whole blood samples (at least 4.5 mL in a 6-mL EDTA tube) will be collected at the following times on both treatment arms:

at baseline
on cycle 3 day 1 (or the next restaging visit if C3D1 has already passed)
12 weeks (±1 week) after the C3D1 or restaging collection*
another 12 weeks (±1 week) later (e.g., C11D1 if collected on C3D1)*
at disease progression or response.
In the event that the collection coincides with a patient's treatment at a non-NCI site, the collection will be postponed to the next restaging visit at the NCI.

PBMC collections will be repeated if a patient crosses over or re-enrolls to the combination arm.

Each whole blood specimen will be collected into one standard 6 mL EDTA tube. Within 60 minutes of collection, PBMCs will be isolated using SepMate tubes (kits provided) according to “Cryopreservation of PBMCs” below. Briefly, add density gradient medium through SepMate insert, dilute whole blood sample with an equal volume PBS+2% FBS and pipette slowly down the side of the SepMate tube, centrifuge for 10 minutes with the brake on, pipette off some supernatant above the PBMC layer, pour the remaining supernatant and isolated PBMCs into a 15 mL or 50 mL tube (inverting for ≤2 seconds), wash the PBMCs twice with 10 mL PBS+2% FBS, and resuspend in 2.5 mL of cryopreservation Component #1. Within 60 minutes of isolation, the PBMCs must be aliquoted into five cryovials (0.5 mL per cryovial) containing cryopreservation Component #2 and frozen slowly in a CoolCell container to −80° C. Please see “Cryopreservation of PBMCs” below for detailed guidance.

### Pharmacokinetics and Anti-Drug Antibodies (Ages 2-11 Only)

Serum samples (mandatory) will be collected at multiple time points from pediatric patients who are 2-11 years old at enrollment, in order to measure pharmacokinetics (PK) and the presence of anti-drug (i.e., atezolizumab) antibodies (ADA) in this sparse patient population. If a patient participating in this PK+ADA study turns 12 years old while still receiving study treatment, serum will continue to be collected past age 11. All PK and ADA analyses for this study will be performed by ICON.

### Collection:

Please see “PROCEDURE FOR PEDIATRIC PK+ADA BLOOD COLLECTION” herein for sample collection and labeling instructions. The blood will be collected at the time points described in Table 23.

Kits for sample collection will be shipped to the clinical site and will include specimen labels and a sample collection worksheet/manifest. The sample worksheet/manifest should be completed and included with the return shipment of each serum sample. Serum should be shipped back to Labcorp on the day of collection (or the following day when necessary); samples should be stored at or below −20° C. until shipment to Labcorp.

### Pharmacokinetics and Anti-Drug Antibodies (Monotherapy Arm, Ages >12 Only)

Serum samples (optional) will be collected from patients who were ≥12 years old at enrollment and are enrolled to the monotherapy arm. These samples will be collected on Day 1 of one to a maximum of three cycles from patients, in order to measure steady-state PK in this ASPS patient population. All PK analyses for this study will be performed by ICON.

### Collection:

Please see “Procedure for PK Blood Collection from Patients Ages 12 years and older” for sample collection and labeling instructions. The blood will be collected at the time points as described in Table 24.

Kits for sample collection will be shipped to the clinical site and will include specimen labels and a sample collection worksheet/manifest. The sample worksheet/manifest should be completed and included with the return shipment of each serum sample. Serum should be shipped back to Labcorp on the day of collection (or the following day when necessary); samples should be stored at or below −20° C. until shipment to Labcorp.

Cell-Free DNA Sequencing: For patients who consent to genetic sequencing of blood samples, plasma from CTC blood samples correlating to clinically informative time points (minimally, before treatment and at restaging scans suggesting changes in tumor burden) will be analyzed for circulating tumor DNA (ctDNA) by the TSO500 panel. If a sufficient proportion of the circulating cfDNA detected is ctDNA, then WES of cfDNA will also be considered to uncover additional relevant changes in the tumor genome. The results of these studies will not be returned to patients.

### E. Sample Collection and Processing

Biospecimens will be collected and processed using validated SOPs that will ensure both specimen quality and patient confidentiality pursuant to informed consent provisions. Information about each specimen (e.g., blood, tumor biopsy, per specific protocol) will be recorded on a PK/PD collection worksheet.

Using a computerized inventory system and a backup hardcopy process, all specimen collection and processing steps will be documented and the specific location of each specimen will be tracked. Each new specimen collected will be assigned a unique barcode identifier that can be linked to the original specimen collected and other relevant information within the inventory system. To ensure patient confidentiality, only containers used for the initial specimen collections will be labeled with patient identifiers.

Only the barcode identifier will be applied to all subsequent specimen containers. When specimens are processed and aliquoted, no patient information will be included on the new containers. Original specimen containers will be discarded. Only barcode-labeled specimens without patient identifiers will be shipped for analysis and/or storage. Specimen labels will indicate: CTEP protocol number, unique patient accession number, 3-digit sample number (see list below), collection time, and total volume collected, as appropriate. Samples from sets of at least three patients will be grouped for scientific analysis.

Standardized 3-digit sample collection numbers:
300 series: PBMCs for T cell receptor sequencing
400 series: blood for circulating tumor cells
500 series: tumor biopsies
800 series: blood for genetic/genomic analyses

The inventory process contains other security provisions sufficient to safeguard patient privacy and confidentiality. Access to the inventory system and associated documents will be restricted to appropriate individuals. Requests to use specimens stored in the repository must be approved. The only patient information available in the inventory system will be the patient sex, diagnosis, and level of informed consent given. SOPs ensure that any changes in informed consent made by a patient and relayed to the PI will be reflected in the inventory system to ensure that specimens are destroyed as appropriate. All laboratory personnel will be trained to adhere to SOPs and will be monitored for high-quality performance.

Any new use of these samples will require prospective IRB review and approval. Access to these samples will only be granted following IRB approval of an additional protocol, granting the rights to use the material.

If at any time, a patient withdraws from the study and does not wish for their existing samples to be utilized, the individual must provide a written request. Following receipt of this request, the samples will be destroyed (or returned to the patient, if so requested), and reported as such to the IRB. Any samples lost (in transit or by a researcher) or destroyed due to unknown sample integrity (i.e., broken freezer allows for extensive sample thawing, etc.) will be reported as such to the IRB.

### F. Management of Genomic Results

This trial will collect identifiable genetic data from patients for exploratory studies to classify tumors by ASPL-TFE3 fusion type and associated genomic differences, and to identify potential markers of atezolizumab response and resistance. Designing the study poses challenging questions about informed consent, the privacy of the patient and the patient's family, the researchers' obligation to disclose genetic information to the patient, and the use and storage of research data [Dressler et al., Clin Pharmacol Ther, 2009. 86(5): p. 472-5; Kass et al., Am J Med Genet A, 2004. 128a(3): p. 261-70; Tabor et al., Am J Med Genet A, 2011. 155a(12): p. 2916-24]. In the vast majority of cases, we do not know the medical significance of genetic variants [Berg et al., Genet Med, 2011. 13(6): p. 499-504; Caulfield et al., PLoS Biol, 2008. 6(3): p. e73].

### VIII. Study Calendar

Eligibility screening evaluations are to be conducted within 8 days prior to patient enrollment to either treatment arm, with the exception of informed consent, echocardiogram (ECHO), and imaging, which must be done within 28 days prior to patient enrollment. Baseline history, physical examination, laboratory evaluations, and urinalysis are to be conducted within 8 days prior to the start of protocol therapy (21 days allowed for EKG). If protocol therapy is started within 8 days of the eligibility screening evaluations, values from the screening evaluations may be used as baseline measurements; if >8 days have passed since the screening evaluations, the medical history, physical examination, laboratory evaluations, urinalysis, and EKG must be repeated prior to starting protocol therapy. Baseline imaging scans and ECHO must be done within 28 days prior to the start of protocol therapy.

Each cycle is 21 days (±3 days due to scheduling conflicts). The start of a new cycle may be delayed up to 2 weeks to accommodate scheduling conflicts and other unexpected events. In the event that the patient's condition is deteriorating, laboratory evaluations can be repeated within 48 hours prior to initiation of the next cycle of therapy.

### IX. Measurement of Effect

Antitumor Effect-Solid Tumors: All patients will have a CT scan (or other imaging test; see Methods for Evaluation of Measurable Disease below) at baseline, at the end of cycle 3, and every two cycles (6 weeks) thereafter (every 3 cycles for patients on study for >1 year; every 4 cycles for patients on study for >2 years). Confirmatory scans should also be obtained at ≥4 weeks following initial documentation of objective response. Patients on the monotherapy arm will have a CT scan at the end of cycle 1 which will be used to assess for early response only; no treatment decisions will be made unless clinically warranted.

Given the concern of pseudoprogression (“tumor flare”), patients who show evidence of clinical benefit will be permitted to continue their monotherapy or combination treatment following RECIST progression, as long as the increase in disease burden does not meet the definition of confirmed PD by immune response criteria and if they meet all of the following additional criteria:
Evidence of clinical benefit as assessed by the investigator and agreed upon by the Medical Monitor
Absence of symptoms or signs (including worsening laboratory values) indicating unequivocal progression of disease
No significant decline in performance status that can be attributed to disease progression
Absence of tumor progression at critical anatomical sites (e.g., leptomeningeal disease) that cannot be managed by protocol-allowed medical interventions
The investigator must discuss with the patient the risks and benefits of treatment continuation after possible worsening disease and must document the patient's agreement to continue receiving treatment (atezolizumab monotherapy or atezolizumab+bevacizumab, as enrolled) in the medical record.

Adult patients who do not meet the criteria for continuing atezolizumab monotherapy may cross over to the atezolizumab+bevacizumab combination arm, provided they meet the patient selection criteria.

Response and progression will be evaluated in this study using the new international criteria proposed by the revised Response Evaluation Criteria in Solid Tumors (RECIST) guideline (version 1.1) [Eisenhaurer et al., Eur J Cancer, 2009. 45(2): p. 228-47]. Immune RECIST (iRECIST) [Seymour et al., Lancet Oncol, 2017. 18(3): p. e143-e152] will be used only as an exploratory endpoint, for comparison to RECIST 1.1 (see Immune Recist Criteria). Changes in the largest diameter (unidimensional measurement) of the tumor lesions and the shortest diameter in the case of malignant lymph nodes are used in the RECIST criteria.

### Definitions

Evaluable for toxicity. All patients will be evaluable for toxicity from the time of their first treatment with atezolizumab

Evaluable for objective response. All patients who met the eligibility criteria (with the possible exception of those who received no study medication) will be considered evaluable for response. These patients will have their response classified according to the definitions stated below.

Evaluable Non-Target Disease Response. Patients who have lesions present at baseline that are evaluable but do not meet the definitions of measurable disease, have received at least one cycle of therapy, and have had their disease re-evaluated will be considered evaluable for non-target disease. The response assessment is based on the presence, absence, or unequivocal progression of the lesions.

### Disease Parameters

Measurable disease. Measurable lesions are defined as those that can be accurately measured in at least one dimension (longest diameter to be recorded) as ≥20 mm (≥2 cm) by chest x-ray or as ≥10 mm (≥1 cm) with CT scan, MRI, or calipers by clinical exam. All tumor measurements must be recorded in millimeters (or decimal fractions of centimeters).

Malignant lymph nodes. To be considered pathologically enlarged and measurable, a lymph node must be ≥15 mm (≥1.5 cm) in short axis when assessed by CT scan (CT scan slice thickness recommended to be no greater than 5 mm [0.5 cm]). At baseline and in follow-up, only the short axis will be measured and followed.

Non-measurable disease. All other lesions (or sites of disease), including small lesions (longest diameter <10 mm [<1 cm] or pathological lymph nodes with ≥10 to <15 mm [≥1 to <1.5 cm] short axis), are considered non-measurable disease. Bone lesions, leptomeningeal disease, ascites, pleural/pericardial effusions, lymphangitis cutis/pulmonitis, inflammatory breast disease, and abdominal masses (not followed by CT or MRI), are considered as non-measurable.

Note: Cystic lesions that meet the criteria for radiographically defined simple cysts should not be considered as malignant lesions (neither measurable nor non-measurable) since they are, by definition, simple cysts.

‘Cystic lesions’ thought to represent cystic metastases can be considered as measurable lesions, if they meet the definition of measurability described above. However, if non-cystic lesions are present in the same patient, these are preferred for selection as target lesions.

Target lesions. All measurable lesions up to a maximum of 2 lesions per organ and 5 lesions in total, representative of all involved organs, should be identified as target lesions and recorded and measured at baseline. Target lesions should be selected on the basis of their size (lesions with the longest diameter), be representative of all involved organs, but in addition should be those that lend themselves to reproducible repeated measurements. It may be the case that, on occasion, the largest lesion does not lend itself to reproducible measurement in which circumstance the next largest lesion which can be measured reproducibly should be selected. A sum of the diameters (longest for non-nodal lesions, short axis for nodal lesions) for all target lesions will be calculated and reported as the baseline sum diameters. If lymph nodes are to be included in the sum, then only the short axis is added into the sum. The baseline sum diameters will be used as reference to further characterize any objective tumor regression in the measurable dimension of the disease.

Non-target lesions. All other lesions (or sites of disease) including any measurable lesions over and above the 5 target lesions should be identified as non-target lesions and should also be recorded at baseline. Measurements of these lesions are not required, but the presence, absence, or in rare cases unequivocal progression of each should be noted throughout follow-up.

Methods for Evaluation of Measurable Disease: All measurements should be taken and recorded in metric notation using a ruler or calipers. All baseline evaluations should be performed as closely as possible to the beginning of treatment and never more than 4 weeks before the beginning of the treatment.

The same method of assessment and the same technique should be used to characterize each identified and reported lesion at baseline and during follow-up. Imaging-based evaluation is preferred to evaluation by clinical examination unless the lesion(s) being followed cannot be imaged but are assessable by clinical exam.

Clinical lesions: Clinical lesions will only be considered measurable when they are superficial (e.g., skin nodules and palpable lymph nodes) and >10 mm (≥1 cm) diameter as assessed using calipers (e.g., skin nodules). In the case of skin lesions, documentation by color photography, including a ruler to estimate the size of the lesion, is recommended.

Chest x-ray: Lesions on chest x-ray are acceptable as measurable lesions when they are clearly defined and surrounded by aerated lung. However, CT is preferable.

Conventional CT and MRI: This guideline has defined measurability of lesions on CT scan based on the assumption that CT slice thickness is 5 mm (0.5 cm) or less. If CT scans have slice thickness greater than 5 mm (0.5 cm), the minimum size for a measurable lesion should be twice the slice thickness. MRI is also acceptable in certain situations (e.g. for body scans).

Use of MRI remains a complex issue. MRI has excellent contrast, spatial, and temporal resolution; however, there are many image acquisition variables involved in MRI, which greatly impact image quality, lesion conspicuity, and measurement. Furthermore, the availability of MRI is variable globally. As with CT, if an MRI is performed, the technical specifications of the scanning sequences used should be optimized for the evaluation of the type and site of disease. Furthermore, as with CT, the modality used at follow-up should be the same as was used at baseline and the lesions should be measured/assessed on the same pulse sequence. It is beyond the scope of the RECIST guidelines to prescribe specific MRI pulse sequence parameters for all scanners, body parts, and diseases. Ideally, the same type of scanner should be used and the image acquisition protocol should be followed as closely as possible to prior scans. Body scans should be performed with breath-hold scanning techniques, if possible.

PET-CT: At present, the low dose or attenuation correction CT portion of a combined PET-CT is not always of optimal diagnostic CT quality for use with RECIST measurements. However, if the site can document that the CT performed as part of a PET-CT is of identical diagnostic quality to a diagnostic CT (with IV and oral contrast), then the CT portion of the PET-CT can be used for RECIST measurements and can be used interchangeably with conventional CT in accurately measuring cancer lesions over time. Note, however, that the PET portion of the CT introduces additional data which may bias an investigator if it is not routinely or serially performed.

Ultrasound: Ultrasound is not useful in assessment of lesion size and should not be used as a method of measurement. Ultrasound examinations cannot be reproduced in their entirety for independent review at a later date and, because they are operator dependent, it cannot be guaranteed that the same technique and measurements will be taken from one assessment to the next. If new lesions are identified by ultrasound in the course of the study, confirmation by CT or MRI is advised. If there is concern about radiation exposure at CT, MRI may be used instead of CT in selected instances.

Endoscopy, Laparoscopy: The utilization of these techniques for objective tumor evaluation is not advised. However, such techniques may be useful to confirm complete pathological response when biopsies are obtained or to determine relapse in trials where recurrence following complete response (CR) or surgical resection is an endpoint.

Tumor markers: Tumor markers alone cannot be used to assess response. If markers are initially above the upper normal limit, they must normalize for a patient to be considered in complete clinical response. Specific guidelines for both CA-125 response (in recurrent ovarian cancer) and PSA response (in recurrent prostate cancer) have been published [JNCI 96:487-488, 2004; J Clin Oncol 17, 3461-3467, 1999; J Clin Oncol 26:1148-1159, 2008]. In addition, the Gynecologic Cancer Intergroup has developed CA-125 progression criteria which are to be integrated with objective tumor assessment for use in first-line trials in ovarian cancer [JNCI 92:1534-1535, 2000].

Cytology, Histology: These techniques can be used to differentiate between partial responses (PR) and complete responses (CR) in rare cases (e.g., residual lesions in tumor types, such as germ cell tumors, where known residual benign tumors can remain).

The cytological confirmation of the neoplastic origin of any effusion that appears or worsens during treatment when the measurable tumor has met criteria for response or stable disease is mandatory to differentiate between response or stable disease (an effusion may be a side effect of the treatment) and progressive disease.

FDG-PET: While FDG-PET response assessments need additional study, it is sometimes reasonable to incorporate the use of FDG-PET scanning to complement CT scanning in assessment of progression (particularly possible ‘new’ disease). New lesions on the basis of FDG-PET imaging can be identified according to the following algorithm:
5. Negative FDG-PET at baseline, with a positive FDG-PET at follow-up is a sign of PD based on a new lesion.
6. No FDG-PET at baseline and a positive FDG-PET at follow-up: If the positive FDG-
7. PET at follow-up corresponds to a new site of disease confirmed by CT, this is PD. If the positive FDG-PET at follow-up is not confirmed as a new site of disease on CT, additional follow-up CT scans are needed to determine if there is truly progression occurring at that site (if so, the date of PD will be the date of the initial abnormal FDG-PET scan). If the positive FDG-PET at follow-up corresponds to a pre-existing site of disease on CT that is not progressing on the basis of the anatomic images, this is not PD.
8. FDG-PET may be used to upgrade a response to a CR in a manner similar to a biopsy in cases where a residual radiographic abnormality is thought to represent fibrosis or scarring. The use of FDG-PET in this circumstance should be prospectively described in the protocol and supported by disease-specific medical literature for the indication. However, it must be acknowledged that both approaches may lead to false positive CR due to limitations of FDG-PET and biopsy resolution/sensitivity.
   Note: A ‘positive’ FDG-PET scan lesion means one which is FDG avid with an uptake greater than twice that of the surrounding tissue on the attenuation corrected image.

### Response Criteria

### Evaluation of Target Lesions

Complete Response (CR): Disappearance of all target lesions. Any pathological lymph nodes (whether target or non-target) must have reduction in short axis to <10 mm (<1 cm).

Partial Response (PR): At least a 30% decrease in the sum of the diameters of target lesions, taking as reference the baseline sum diameters.

Progressive Disease (PD): At least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm (0.5 cm). (Note: the appearance of one or more new lesions is also considered progressions).

Stable Disease (SD): Neither sufficient shrinkage to qualify for PR nor sufficient increase to qualify for PD, taking as reference the smallest sum diameters while on study.

### Evaluation of Non-Target Lesions

Complete Response (CR): Disappearance of all non-target lesions and normalization of tumor marker level. All lymph nodes must be non-pathological in size (<10 mm [<1 cm] short axis).

Note: If tumor markers are initially above the upper normal limit, they must normalize for a patient to be considered in complete clinical response.

Non-CR/Non-PD: Persistence of one or more non-target lesion(s) and/or maintenance of tumor marker level above the normal limits.

Progressive Disease (PD): Appearance of one or more new lesions and/or unequivocal progression of existing non-target lesions. Unequivocal progression should not normally trump target lesion status. It must be representative of overall disease status change, not a single lesion increase.

Although a clear progression of “non-target” lesions only is exceptional, the opinion of the treating physician should prevail in such circumstances, and the progression status can be confirmed at a later time.

### Evaluation of Non-Measurable Lesions

Complete Response (CR): Complete disappearance of disease.

Non-CR/Non-PD: Persistence of evidence of disease.

Progressive Disease (PD): Unequivocal disease progression.

Evaluation of Best Overall Response: The best overall response is the best response recorded from the start of the treatment until disease progression/recurrence (taking as reference for progressive disease the smallest measurements recorded since the treatment started). The patient's best response assignment will depend on the achievement of both measurement and confirmation criteria. Best overall response confirmation timeline for patients with measurable and non-measurable disease are described in Tables 25 and 26.

For those patients that go on treatment holiday, tumor response and progression will continue to be assessed among those patients who have not yet progressed. In addition, the post-progression response rate among patients who progress while on holiday and then resume treatment will be assessed as an exploratory endpoint. This endpoint will not include patients who resume therapy after a drug holiday (≤2 years) without progression.

### Duration of Response

Duration of overall response: The duration of overall response is measured from the time measurement criteria are met for CR or PR (whichever is first recorded) until the first date that recurrent or progressive disease is objectively documented (taking as reference for progressive disease the smallest measurements recorded since the treatment started).

The duration of overall CR is measured from the time measurement criteria are first met for CR until the first date that progressive disease is objectively documented.

Duration of stable disease: Stable disease is measured from the start of the treatment until the criteria for progression are met, taking as reference the smallest measurements recorded since the treatment started, including the baseline measurements.

### X. Study Oversight

This protocol is monitored at several levels.

During the Phase 2 portion of the study, the Protocol Principal Investigator will have, at a minimum, quarterly conference calls with the Study Investigators and the CTEP Medical Officer(s) to review accrual, progress, and pharmacovigilance. Decisions to proceed to the second stage of a Phase 2 trial will require sign-off by the Protocol Principal Investigator and the Protocol Statistician.

All Study Investigators at participating sites who register/enroll patients on a given protocol are responsible for timely submission of data via Medidata Rave and timely reporting of adverse events for that particular study. This includes timely review of data collected on the electronic CRFs submitted via Medidata Rave.

All studies are also reviewed in accordance with the enrolling institution's data safety monitoring plan.

### XI. Statistical Considerations

### A. Study Design/Endpoints

For the atezolizumab monotherapy arm, a Phase 2 Simon two stage design will be employed. Accrual to the monotherapy arm will be halted at 9 patients to evaluate for response before further enrollment. If no responses are observed among the initial 9 patients, the study will be terminated early and declared negative; if at least one response is observed, the study will continue. If at least 3 responses (at least 12.5%) are observed among the initial 24 evaluable patients, this regimen will be considered worthy of further testing in this disease. If the study does not meet its primary objective with the initial 24 evaluable patients, accrual will stop and no further patients will be added to the study. This design yields at least 90% power to detect a true response rate of at least 25%. It yields at least 0.91 probability of a negative result if the true response rate is no more than 5%, with at least 0.63 probability of early negative stopping.

A partial response was observed in the initial 9 patients treated in the monotherapy arm. The study continued to enroll to meet accrual goals.

Eight partial responses had been observed among the initial 19 evaluable patients in the monotherapy arm, indicating that the regimen is worthy of further testing. The accrual ceiling was raised to allow for the collection of research biopsies for pharmacodynamic analysis.

Eighteen partial responses and 1 complete response had been observed among 52 patients, research biopsies were subjected to pharmacodynamic analysis, results were published [Chen et al., N Engl J Med, 2023. 389(10): p. 911-921], and atezolizumab was approved for patients ≥2 years of age with advanced ASPS.

For the atezolizumab+bevacizumab arm, nine patients will be enrolled (via crossover or re-enrollment). If zero responses are observed among these nine patients, the combination arm will not be pursued further on this trial. If at least one response is observed among the first nine patients, an amendment will be submitted to CTEP to request that an additional 15 patients be enrolled to this arm, with 3 responses (out of 24 total patients) being required for a positive trial in this arm. Note that this is the same design employed for the atezolizumab monotherapy cohort. It should also be noted that, regardless of the number of responses observed in the first nine patients, no additional patients will be enrolled to this cohort unless our request (via an amendment) for additional patients is approved by CTEP. Interim and final analyses for this arm will be modified ITT, i.e. all eligible patients who receive at least one dose of treatment will be included.

### B. Sample Size/Accrual Rate

To allow for the collection of research biopsies for pharmacodynamic studies, additional patients may be enrolled to the monotherapy arm beyond the initial 24 evaluable patients (initial accrual ceiling of 26 patients).

Mandatory biopsies will be collected for pharmacodynamic analysis from adult patients only. With biopsies collected from 15 adult patients, there will be 95% likelihood of getting at least 10 evaluable biopsy pairs. For a given PD endpoint, this will yield 82% power to detect a difference in change from baseline to post-treatment, between the clinical responders and the non-responders, corresponding to at least 2 SD's (with respect to the baseline measures), at the 1-sided 0.01 significance level (to accommodate multiple comparisons).

The monotherapy arm was to remain open to pediatric patients until 5 pediatric patients had contributed Cycle 3 Day 1 blood samples for the optional PK+ADA study, 13 pediatric patients had enrolled (with or without blood sample collections), or the atezolizumab plus bevacizumab arm was closed to accrual, whichever happened first. Pediatric patients will not provide research biopsies, but will have the opportunity to be treated on the monotherapy arm due to the possibility of therapeutic benefit; patients <12 years of age may choose to participate in the optional PK+ADA study.

To successfully obtain biopsies from 15 adult participants, and to allow patient slots for the enrollment of pediatric participants, the accrual ceiling for the monotherapy arm was increased from 26 patients to 53 patients. It was subsequently raised to 63 to allow enrollment of an additional 10 pediatric patients, thus offering pediatric patients the possibility of therapeutic benefit and facilitating the collection of blood samples for the pediatric PK+ADA study. By Apr. 19, 2024, the monotherapy arm was closed to all pediatric and adult patient accrual because enrollment was sufficient to meet study endpoints and atezolizumab is now approved for those patient populations.

In addition to the monotherapy arm, a maximum of 9 adult patients will be enrolled to the combination arm (all of whom were previously enrolled to the monotherapy arm). Thus, the anticipated maximum combined sample size is 72 enrollments, comprised of a maximum of 63 unique patients.

With an anticipated accrual of 12-24 monotherapy patients per year, it was expected that 2.5-4.5 years would be required to enroll 53 adult and pediatric patients to the monotherapy arm. With an anticipated accrual of 3 pediatric patients per year, it was expected that an additional 2-4 years would be required to enroll the 5-10 additional pediatric patients to the monotherapy arm. Assuming an accrual rate of 2-3 combination arm patients per year, accrual to the combination arm is expected to take approximately 4 years.

### C. Analysis of Secondary and Exploratory Endpoints

As secondary analyses, response analyses will be stratified by prior tyrosine kinase inhibitor/anti-angiogenic agent-naïve patients versus those with prior tyrosine kinase inhibitor or anti-angiogenic agent exposure.

The analysis of pediatric PK+ADA data from this trial will be descriptive in nature; the anticipated sample size is 5 patients. The PK+ADA data may also be included with data from the G029664 trial (from 25 and 24 patients, respectively) for pooled population PK modeling efforts, in which ADA is a covariate. The data from this trial in ASPS, where responses are likely, is expected to augment the limited exposure-response data from G029664.

The analysis of adolescent and adult (≥12 years old) PK data from this trial will be descriptive in nature; the anticipated sample size is 8 patients. Serum concentrations of atezolizumab may be reported as individual values and summarized (mean, standard deviation, coefficient of variation, median, range, geometric mean, and geometric mean coefficient of variation) by treatment arm and cycle, when appropriate and as data allow. Individual and mean serum atezolizumab concentrations may be plotted. Atezolizumab concentration data may be compared with data from other studies as warranted by the data.

Exploratory evaluations will be performed, with results reported with appropriate caveats about the exploratory nature of the analysis, and without formal adjustment for multiple comparisons.

### D. Reporting and Exclusions

Evaluation of Toxicity: All patients will be evaluable for toxicity from the time of their first treatment with atezolizumab.

Evaluation of Response: All patients included in the study must be assessed for response to treatment, even if there are major protocol treatment deviations or if they are ineligible. Each patient will be assigned one of the following categories: 1) complete response, 2) partial response, 3) stable disease, 4) progressive disease, 5) early death from malignant disease, 6) early death from toxicity, 7) early death because of other cause, or 9) unknown (not assessable, insufficient data). [Note: By arbitrary convention, category 9 usually designates the “unknown” status of any type of data in a clinical database.]

All of the patients who met the eligibility criteria (with the possible exception of those who received no study medication) should be included in the main analysis of the response rate. Patients in response categories 4-9 should be considered to have a treatment failure (disease progression). Thus, an incorrect treatment schedule or drug administration does not result in exclusion from the analysis of the response rate. Precise definitions for categories 4-9 will be protocol specific.

All conclusions should be based on all eligible patients. Subanalyses may then be performed on the basis of a subset of patients, excluding those for whom major protocol deviations have been identified (e.g., early death due to other reasons, early discontinuation of treatment, major protocol violations, etc.). However, these subanalyses may not serve as the basis for drawing conclusions concerning treatment efficacy, and the reasons for excluding patients from the analysis should be clearly reported. The 95% confidence intervals should also be provided.

### XII. Immune RECIST Criteria

Increasing clinical experience indicates that traditional response criteria (e.g., Response Evaluation Criteria in Solid Tumors, Version 1.1 [RECIST v1.1] and World Health Organization [WHO]) may not be sufficient to fully characterize activity in the new era of target therapies and/or biologics. In studies with cytokines, cancer vaccines, and monoclonal antibodies, complete response, partial response, or stable disease has been shown to occur after an increase in tumor burden as characterized by progressive disease by traditional response criteria. Therefore, conventional response criteria may not adequately assess the activity of immunotherapeutic agents because progressive disease (by initial radiographic evaluation) does not necessarily reflect therapeutic failure. Long-term effect on the target disease must also be captured. The immune RECIST (iRECIST) criteria, by incorporating specific response patterns that have been observed with immunotherapeutic agents, have been developed to address these issues and provide standardized response criteria for use with immunotherapy [Seymour et al., Lancet Oncol, 2017. 18(3): p. e143-e152].

iRECIST Assessment of Response

All patients will have their BEST RESPONSE from the start of study treatment until the end of treatment classified as outlined below:

Complete Response (CR): disappearance of target and non-target lesions and normalization of tumor markers. Pathological lymph nodes must have short axis measures <10 mm (Note: continue to record the measurement even if <10 mm and considered CR). Residual lesions (other than nodes <10 mm) thought to be non-malignant should be further investigated (by cytology specialized imaging or other techniques as appropriate for individual cases [Eisnhauer et al., Eur J Cancer, 2009. 45(2): p. 228-47] before CR can be accepted.

Partial Response (PR): at least a 30% decrease in the sum of measures (longest diameter for tumor lesions and short axis measure for nodes) of target lesions, taking as reference the baseline sum of diameters. Non target lesions must be non-PD.

Stable Disease (SD): Neither sufficient shrinkage to qualify for PR nor sufficient increase to qualify for PD taking as reference the smallest sum of diameters on study.

Progressive Disease (PD): at least a 20% increase in the sum of diameters of measured lesions taking as references the smallest sum of diameters recorded on study (including baseline) AND an absolute increase of ≥5 mm. Appearance of new lesions will also constitute progressive disease (including lesions in previously unassessed areas). In exceptional circumstances, unequivocal progression of non-target disease may be accepted as evidence of disease progression, where the overall tumor burden has increased sufficiently to merit discontinuation of treatment or where the tumor burden appears to have increased by at least 73% in volume. Modest increases in the size of one or more non-target lesions are NOT considered unequivocal progression. If the evidence of PD is equivocal (target or non-target), treatment may continue until the next assessment, but if confirmed, the earlier date must be used.

Patients that are clinically well may continue on therapy following progression with new lesions or increase in target lesions if the increase in disease burden does not meet the definition of PD by immune response criteria [Seymour et al., Lancet Oncol, 2017. 18(3): p. e143-e152]. In this situation, patients do not have unequivocal progression until immune response criteria are met (see Table 27).

iRECIST Assessment of Progression

Immunotherapeutics may result in infiltration of immune cells leading to transient increase in the size in malignant lesions, or undetectable lesions becoming detectable. Investigators should continue treatment, as appropriate, in the absence of unacceptable toxicity, until unequivocal disease progression. This is particularly important for patients in whom pseudoprogression may have occurred. Follow up response assessments must be continued until unequivocal disease progression has occurred.

The iRECIST criteria are identical to those of RECIST 1.1 in many respects but have been adapted to account for instances where an increase in tumor burden, or the appearance of new lesions, does not reflect true tumor progression.

Key differences are described below. All responses defined using iRECIST criteria are designated with a prefix. iRECIST time-point and best overall responses will be recorded separately. Unlike RECIST 1.1, iRECIST requires the confirmation of progression and uses the terms iUPD (unconfirmed progression) and iCPD (confirmed progression). Confirmatory scans should be performed at least 4 weeks, but no longer than 8 weeks after iUPD.

### Confirming Progression

iCPD is confirmed if further increase in tumor burden, compared to the last assessment, is seen as evidenced by one or more of the following:

Continued increase in tumor burden (from iUPD) where RECIST 1.1 definitions of progression had been met (from nadir) in target, non-target disease or new lesions Progression in target disease worsens with an increase of at least 5 mm in the absolute value of the sum

Continued unequivocal progression in non-target disease with an increase in tumor burden

Increase in size of previously identified new lesion (s) (an increase of at least 5 mm in the absolute value of the sum of those considered to be target new lesions) or additional new lesions. RECIST 1.1 criteria are met in lesions types (target or non-target or new lesions) where progression was not previously identified, including the appearance of additional new lesions.

If iUPD is not confirmed at the next assessment, then the appropriate response will be assigned (iUPD if the criteria are still met, but no worsening, or iSD, iPR or iCR if those criteria are met compared to baseline). As can be seen Table 28, below, prior documentation of iUPD does not preclude assigning iCR, iPR, or iSD in subsequent time-point assessments or as best overall response (BOR) providing that iCPD is not documented at the next assessment after iUPD.

New lesions: New lesions should be assessed and measured as they appear using RECIST 1.1 criteria (maximum of 5 lesions, no more than 2 per site, at least 10 mm in long axis (or 15 mm in short axis for nodal lesions), and recorded as New Lesions-Target (NLT) and New Lesion-Non-Target (NLNT) to allow clear differentiation from baseline target and non-target lesions.

New lesions may either meet the criteria of NLT or NLNT to drive iUPD (or iCPD). However, the measurements of target lesions should NOT be included in the sum of measures of original target lesions identified at baseline. Rather, these measurements will be collected on a separate table in the case record form.

PD is confirmed in the New Lesion category if the next imaging assessment, conducted at least 4 weeks (but not more than 8 weeks) after iUPD confirms further progression from iUPD with either an increase of at least 5 mm in the absolute value of the sum of NLT OR an increase (but not necessarily unequivocal increase) in the size of NLNT lesions OR the appearance of additional new lesions.

All patients will have their iBOR from the start of study treatment until the end of treatment classified as outlined in Table 29.

Response and Stable Disease Duration (RECIST 1.1 and iRECIST)

Response duration will be measured from the time measurement criteria for CR/PR or iCR/iPR (whichever is first recorded) are first met until the first date that recurrent or progressive disease is objectively documented, taking as reference the smallest measurements recorded on study (including baseline).

Stable disease duration will be measured from the time of start of treatment until the criteria for progression are met, taking as reference the smallest sum on study (including baseline).

XIII. Procedure for Isolation and Cryopreservation of PBMCs from Whole Blood

### C. Isolation of PBMCs

Equipment: 10 mL EDTA tube, SepMate 50 mL tube, PipetAid, centrifuge

Reagents/Materials: LymphoPrep (500 mL), Dulbecco's PBS with 2% FBS (500 mL), Gibco™ Advanced DMEM/F12 media, 25 mL disposable serological pipets, 10 mL disposable serological pipets, 50 mL conical tubes, sterile polystyrene aspirating pipets

Notes: Lab coats, safety glasses, and gloves must be worn at all times when handling hazardous or sensitive equipment, samples, reagents, and materials. These safety measures must also be followed when in close proximity to those who are working with these items.

Ensure that the whole blood, PBS+2% FBS, Lymphoprep density gradient medium, and centrifuge are all at room temperature (15-25° C.).

Before isolating PBMCs, prepare cryopreservation Component #1, as described in the Cryopreservation section below, for resuspending PBMCs after isolation.

Procedure: Blood will be collected aseptically by venipuncture or from a venous port into one 6 mL EDTA tube; the collected blood samples are stable for up to 60 minutes at room temperature (15° C. to 30° C.) prior to PMBC isolation. Obtain correct size SepMate tubes for volume of blood needing to be isolated as described in Table 30.

Keeping the SepMate™ tube completely upright, add Lymphoprep density gradient media by carefully pipetting it through the central hole of the SepMate™ insert. Refer to Table 1 for required volumes of Lymphoprep density gradient medium per sample; for 6 mL whole blood, use a 50 mL SepMate tube and 15 mL of density gradient. The top of the Lymphoprep density gradient medium will be above the insert. NOTE: Small bubbles may be present in the density gradient medium after pipetting. These bubbles will not affect performance.

Dilute all whole blood samples with an equal volume of PBS+2% FBS in new 50 mL conical tube and mix gently by pipetting up and down. Keeping the SepMate™ tube vertical, add the diluted sample by pipetting it slowly down the side of the tube. Repeat for each sample to be processed. The sample will mix with the density gradient medium above the insert.

Centrifuge at 1200×g for 10 minutes at room temperature, with the brake on. Note: For samples older than 24 hours, a centrifugation time of 20 minutes is recommended. To reduce platelet contamination in the enriched PBMCs, pipette off some of the supernatant above the PBMC layer, leaving 1 inch above the insert or RBC pack. Pour off the rest of the supernatant which contains the enriched PBMCs, into a new 50 mL tube. Do not hold the SepMate™ tube in the inverted position for longer than 2 seconds. If there is substantial carryover of red blood cells (RBCs) in the PBMC supernatant repeat steps 8.1 to 8.6 using another SepMate tube with Lymphoprep density gradient media.

Add 10 mL PBS+2% FBS to the 50 mL tube containing PBMCs. Centrifuge at 120×g for 10 minutes at room temperature, with the brake off. Aspirate supernatant without aspirating the PBMC pellet. This will remove platelets that are present Wash enriched PBMCs by adding another 10 mL PBS+2% FBS to the 50 mL tube. Centrifuge at 300×g for 8 minutes at room temperature, with the brake on. Aspirate supernatant without aspirating the PBMC pellet. Resuspend PBMCs in 2.5 mL of cryopreservation Component #1.

### D. Cryopreservation of PBMCs

Equipment: 1000 μL single channel pipet, CoolCell® LX, cryo-resistant label supplies, PipetAid, Nalgene™ Rapid-Flow™ Sterile Disposable Filter Units with CN Membrane, 2 microns

Reagents/Materials: FBS, DMSO, Gibco™ Advanced DMEM/F12 media, Thermo Scientific Nunc 368632 CryoTube, free standing round bottom; 1.8 mL, 50/bag, 25 mL disposable serological pipets, 10 mL disposable serological pipets, 3′ Core Polyester Chemical Resistant labels, 1000 μL filtered pipet tips (RTL-1000F)

Notes: Lab coats, safety glasses, and gloves must be worn at all times when handling hazardous or sensitive equipment, samples, reagents, and materials. These safety measures must also be followed when in close proximity to those who are working with these items. Ensure that all reagents and cryopods are all at room temperature (15-25° C.).

Procedure: Prepare tumor derived cell cryopreservation media, Component #1 and Component #2, as described in Table 31. After Components #1 and #2 are combined, Advanced DMEM/F12 will have a final concentration of 70%, FBS 20% and DMSO 10%.

Combine cryopreservation media with PBMCs. Label cryotubes using labels compatible with cryogenic storage for each sample. Add 500 μL of Component #2 to each cryotube. Slowly add 500 μL of PBMCs resuspended in Component #1 to a cryotube containing 500 uL. Component #2. Do not mix. Place all cryotubes in a cryopod. Place cryopod in a −80° C. freezer for 1 to 3 days. Note: Longer incubation at −80 C results in progressive loss of viability. For longer storage, place cryopods on dry ice and quickly transfer cryotubes from the cryopod to a box in liquid nitrogen storage.

For shipment to MoCha, transfer cryotubes to a box or plastic bag prechilled on dry ice in a styrofoam container, cover with a sufficient amount of dry ice for an overnight shipment and close and tape container tightly.

### XIV. Procedure for Pediatric PK+ADA Blood Collection

Overview: Blood will be collected from pediatric patients <12 years of age for assessment of pediatric PK+ADA response (Table 32). Blood samples will be collected at the time points found in Table 32. Kits for sample collection will be provided to study sites by Labcorp; kits will include red top serum separator tubes for blood collection and cryovial specimen transport tubes for the PK and ADA serum samples. Serum samples will be shipped back to Labcorp on the day of collection or, if necessary, on the following day. Labcorp will then ship batches of samples to ICON, who will complete the PK and ADA analyses.

Collection and Processing: Obtain one 2.5 mL red top serum separator tube per time point. Draw blood into a 2.5 mL red top serum separator tube using standard venipuncture techniques. Document the blood volume and actual time of collection for each sample. Mix the blood with the clotting activation agent by gently inverting the tube 10 times. Place the serum separator tube upright to clot at room temperature for 30 minutes until centrifugation. Centrifuge for 15 minutes at 1500-2000×g (or minimum of 3000 rpm). Separate serum from blood sample within 20 minutes of the end of centrifugation. Use pipette provided to transfer approximately half of the serum (at least 150 μL) into an appropriately labeled PK transport tube. For all predose time points, use the pipette provided to transfer at least 550 μL of the remaining serum into an appropriately labeled ADA transport tube. Ensure that the patient ID is recorded on the label of the transport tube(s). Immediately freeze and store the sample(s) upright at or below −20° C. until shipment to Labcorp. Ship FROZEN to Labcorp on dry ice the day of collection (DOC) or the following day if DOC is not possible.

Labeling: The time point (e.g., C1D1 predose), actual collection date and time, protocol number, and patient ID should be documented for each sample collected. The sample collection worksheet/manifest provided by Labcorp also be returned with the samples.

Shipping Method: Frozen serum samples will be shipped on dry ice to Labcorp on the day of collection, or the following day, using the return shipping label provided with the collection kit. Frozen specimens should NOT be shipped on Fridays, weekends, or the day before an observed holiday. If a specimen collection is scheduled for a Friday or too late on a Thursday to ensure same-day FedEx pickup, the collection is waived.

XV. Procedure for PK Blood Collection from Patients Ages 12 Years and Older

Overview: Blood will be collected from patients ≥12 years of age who are enrolled to the monotherapy arm and who consent to this optional PK study. Kits for sample collection will be provided to study sites by Labcorp; kits will include red top serum separator tubes for blood collection and cryovial specimen transport tubes for the PK serum samples. Serum samples will be shipped back to Labcorp on the day of collection or, if necessary, on the following day. Labcorp will then ship batches of samples to ICON, who will complete the PK analyses. These optional blood collections will begin on Day 1 of the next cycle that a patient begins once collection kits are available and the patient's consent for this optional collection is obtained. The collections may continue for up to three cycles, total, as detailed in Table 33.

Collection and Processing: Obtain one 2.5 mL red top serum separator tube per time point. Draw blood into a 2.5 mL red top serum separator tube using standard venipuncture techniques. Document the blood volume and actual time of collection for each sample. Mix the blood with the clotting activation agent by gently inverting the tube 10 times. Place the serum separator tube upright to clot at room temperature for 30 minutes until centrifugation. Centrifuge for 15 minutes at 1500-2000×g (or minimum of 3000 rpm). Separate serum from blood sample within 20 minutes of the end of centrifugation. Use pipette provided to transfer the serum (at least 150 μL) into an appropriately labeled PK transport tube. Ensure that the patient ID is recorded on the label of the transport tube(s). Immediately freeze and store the sample(s) upright at or below −20° C. until shipment to Labcorp. Ship FROZEN to Labcorp on dry ice the day of collection (DOC) or the following day if DOC is not possible.

Labeling: The time point (e.g., C1D1 predose), actual collection date and time, protocol number, and patient ID should be documented for each sample collected. The sample collection worksheet/manifest provided by Labcorp should also be returned with the samples.

Shipping Method: Frozen serum samples will be shipped on dry ice to Labcorp on the day of collection, or the following day, using the return shipping label provided with the collection kit. Frozen specimens should NOT be shipped on Fridays, weekends, or the day before an observed holiday. If a specimen collection is scheduled for a Friday or too late on a Thursday to ensure same-day FedEx pickup, the collection is waived.

It will be apparent that the precise details of the methods or compositions described may be varied or modified without departing from the spirit of the described aspects of the disclosure. We claim all such modifications and variations that fall within the scope and spirit of the claims below.

1. A method of treating unresectable alveolar soft part sarcoma (ASPS) in a human pediatric subject less than 18 years of age, comprising intravenously administering to the subject a therapeutically effective amount of atezolizumab at a dose of 15 mg per kilogram of body weight every 21 days, thereby treating the unresectable ASPS.

2. A method of treating unresectable alveolar soft part sarcoma (ASPS) in a human adult subject at least 18 years of age, comprising intravenously administering to the subject a therapeutically effective amount of atezolizumab at a dose of 1200 mg every 3 weeks, at a dose of 840 mg every 2 weeks, or at a dose of 1680 mg every 4 weeks, thereby treating the unresectable ASPS.

3. (canceled)

4. The method of claim 1, wherein no more than 1200 mg of atezolizumab is administered per dose.

5. The method of claim 1, wherein the human pediatric subject is at least 2 years of age.

6. The method of claim 2, wherein each dose is administered as a single intravenous injection.

7. The method of claim 2, wherein the atezolizumab is administered over 30 minutes (±10 minutes) or 60 minutes (±15 minutes).

8. The method of claim 2, wherein the atezolizumab is not administered as a push or bolus.

9. The method of claim 1, wherein prior to the administering, the atezolizumab is diluted to 3.2 mg/ml to 16.8 mg/ml into an infusion bag containing 0.9% sodium chloride injection, USP.

10. The method of claim 2, wherein the method does not include co-administering other drugs through an intravenous line used for the intravenously administering.

11. The method of claim 1 wherein the mammalian subject does not have a known primary central nervous system (CNS) malignancy or symptomatic CNS metastases, known clinically significant liver disease, or history of idiopathic pulmonary fibrosis, pneumonitis, organizing pneumonia, or evidence of active pneumonitis on screening chest computed tomography (CT) scan.

12. The method of claim 1, wherein the mammalian subject was previously treated with
surgery for ASPS,
radiotherapy, chemotherapy, or both, or
a tyrosine kinase inhibitor.

13.-17. (canceled)

18. The method of claim 1, further comprising obtaining circulating tumor cells (CTCs) from the subject.

19.-23. (canceled)

24. The method of claim 1, wherein the ASPS comprises Type 1 ASPL-TFE3 fusion transcripts.

25. The method of claim 1, wherein the ASPS comprises Type 2 ASPL-TFE3 fusion transcripts.

26.-35. (canceled)

36. The method of claim 1, wherein the method further comprises administering a therapeutically effective amount of bevacizumab to the subject.

37.-38. (canceled)

39. The method of claim 36, wherein the method comprises administering bevacizumab intravenously after administering atezolizumab.

40. The method of claim 36, wherein the bevacizumab is administered at dose of 10 mg/kg or 15 mg/kg.

41. The method of claim 36, wherein the bevacizumab is administered 30 minutes (±10 min) after administering atezolizumab.

42. The method of claim 36, wherein the bevacizumab is administered over 90 minutes (±15 minutes), over 60 minutes (±10 minutes) or over 30 minutes (±10 minutes).

43.-44. (canceled)

45. The method of claim 1, wherein the atezolizumab is administered as a single dose for at least 2 cycles, at least 3 cycles, at least 4 cycles, at least 5 cycles, at least 10 cycles, at least 20 cycles, at least 100 cycles, or more.

46. The method of claim 1, wherein the atezolizumab is administered over a period of at least 12 months, at least 24 months, at least 36 months, at least 48 months, or at least 60 months.

47.-51. (canceled)
